(19) United States
(12) Patent Application Publication
(10) Pub. No.: US 2025/0135043 A1
(43) Pub. Date: May 1, 2025

(54) DUAL-TARGETING COMPOUND FOR FIBROBLAST ACTIVATION PROTEIN (FAP) AND INTEGRIN avB3, PREPARATION METHOD THEREFOR AND USE THEREOF

(71) Applicant: YANTAI LANNACHENG BIOTECHNOLOGY CO., LTD., Yantai, Shandong (CN)

(72) Inventors: Xiaoyuan CHEN, Yantai, Shandong (CN); Pengfei XU, Yantai, Shandong (CN); Xiaoming WU, Yantai, Shandong (CN); Zhide GUO, Yantai, Shandong (CN); Qingbao YANG, Yantai, Shandong (CN); Xuejun WEN, Yantai, Shandong (CN)

(21) Appl. No.: 18/709,298

(22) PCT Filed: Sep. 26, 2023
(86) PCT No.: PCT/CN2023/121284

§ 371 Date: May 10, 2024

(30) Foreign Application Priority Data
Sep. 29, 2022 (CN) 202211203390.3

(51) Int. Cl. A61K 51/04; C07B 59/00

(52) U.S. Cl. CPC A61K 51/0455; A61K 51/0482; C07B 59/002; A61K 2121/00; A61K 2123/00; C07B 2200/05

(57) ABSTRACT

The present invention relates to a dual-targeting compound capable of targeting fibroblast activation protein (FAP) and integrin αvβ. The targeting compound of the present invention and a radionuclide marker thereof can synergistically target an FAP target and an integrin αᵥβ₃ target in tumors, such that the number and utilization efficiency of effective receptors in tumors can be improved. The present invention further provides a radionuclide marker based on the targeting compound, a preparation method therefor and use thereof in diagnosis or treatment of diseases characterized by overexpression of fibroblast activation protein (FAP) and/or integrin αᵥβ₃.

### TECHNICAL FIELD

The present invention relates to the fields of nuclear medicine and molecular imaging, and specifically relates to a compound, a pharmaceutical composition containing or composed of the compound, a kit containing or composed of the compound or the pharmaceutical composition and use of the compound or the pharmaceutical composition in diagnosis or treatment of diseases characterized by overexpression of fibroblast activation protein (FAP) and/or integrin αᵥβ₃.

### BACKGROUND

Fibroblast activation protein (FAP) is a transmembrane serine peptidase that is expressed on surfaces of tumor stroma activated fibroblasts and plays an important role in oncogenesis and progression processes of tumors. Previous studies show that the FAP is generally not expressed in normal tissues of humans, but is selectively highly expressed on surfaces of stromal fibroblasts of more than 90% of epithelial malignant tumors, including breast cancer, ovarian cancer, lung cancer, colorectal cancer, gastric cancer, pancreatic cancer and the like. In view of wide expression and important role in tumors, the FAP has become an important target for imaging and treatment of tumors. A radionuclide-labeled fibroblast activation protein inhibitor (FAPI), represented by a quinolinic acid derivative, has made important progress in the field of accurate imaging of tumors. For example, PET/CT imaging agents, such as FAPI-02 and FAPI-04, have realized specific imaging of more than 30 different types of tumors.

Integrin (αᵥβ₃) is a heterodimeric receptor located on cell surfaces, which is rarely expressed in normal vascular endothelial and epithelial cells, but is expressed at a high level on cell surfaces of a variety of solid tumors, including lung cancer, osteosarcoma, neuroblastoma, breast cancer, prostate cancer, bladder cancer, glioblastoma, invasive melanoma and the like, and is highly expressed in neovascular endothelial cell membranes of all tumor tissues, indicating that the integrin αᵥβ₃ plays an important role in the processes of growth, invasion and metastasis of tumors. Polypeptides containing an arginine-glycine-aspartic acid (RGD) sequence can specifically bind to the integrin αᵥβ₃. A variety of radionuclide-labeled RGD peptides have been successfully used in research of imaging of a variety of tumor-bearing animal models. In clinical application, ¹⁸F-Galacto-RGD has become a first non-invasive integrin αᵥβ₃ targeted tumor imaging agent in clinical trials, has been successfully used in PET diagnosis of patients with tumors, and has good biological distribution and specific target recognition in clinical trials of glioblastoma.

Considering the heterogeneity of tumors, in order to further improve the diagnosis and treatment efficiency of tumors, development of a drug capable of achieving a targeting effect on the two targets, namely the FAP and the integrin αᵥβ₃, is necessary.

### SUMMARY

In view of the above background, a primary purpose of the present invention is to develop a novel compound with a structure. The novel compound can synergistically target an FAP target and an integrin αᵥβ₃ target in tumors, such that the number and utilization efficiency of effective receptors in tumors can be improved, thereby improving the tumor uptake efficiency and the positive tumor detection efficiency and/or treatment efficiency.

Another purpose of the present invention is to provide a method for preparing the novel compound, so as to synthesize the compound capable of synergistically targeting the FAP target and the integrin αᵥβ₃ target in tumors by a convenient and readily available synthesis route.

Another purpose of the present invention is to provide use of the compound in diagnosis or treatment of diseases characterized by overexpression of fibroblast activation protein (FAP) and/or integrin αᵥβ₃.

The above purposes of the present invention are achieved by adopting the following technical solutions.

In a first aspect, the present invention provides a dual-targeting compound having a structure containing a specific binding ligand structure of both FAP and integrin αᵥβ₃ (the structure is referred to as an “FAPI-RGD” structure in the present invention), the dual-targeting compound can simultaneously target FAP and integrin αᵥβ₃, and the structure of the dual-targeting compound is shown in Formula (I) or Formula (II) below:

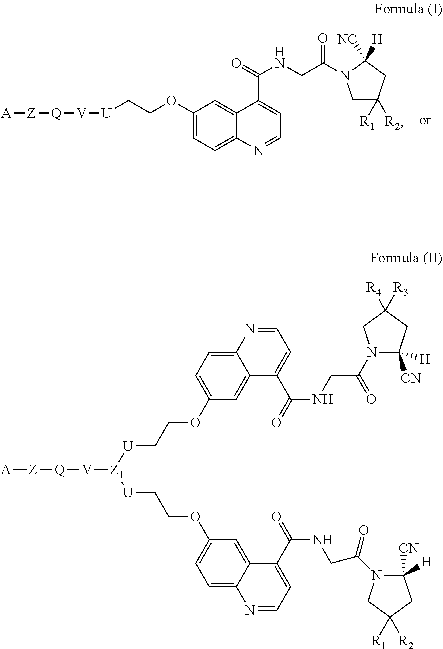

where
R₁, R₂, R₃ and R₄ may be independently selected from H or F, and the R₁, the R₂, the R₃ and the R₄ may be the same or different.
Z, Q, V and U are a same connection structure or different connection structures, and are independently selected from —NH—,

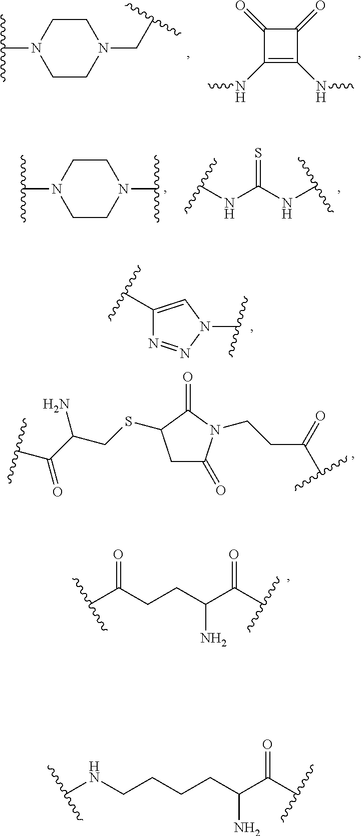

or substitution structures based on —(CH₂)ₙ—, respectively.

Further, when the Z, the Q, the V and/or the U are substitution structures based on —(CH₂)ₙ—, each —CH₂— is separately substituted with or without —O—, —NH—, —(CO)—, —NH—(CO)—, —CH(NH₂)— or —(CO)—NH—, and substitution occurs when no two adjacent —CH₂— groups are substituted.
n is an integer selected from 0 to 30 (such as 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30).
Z₁ is

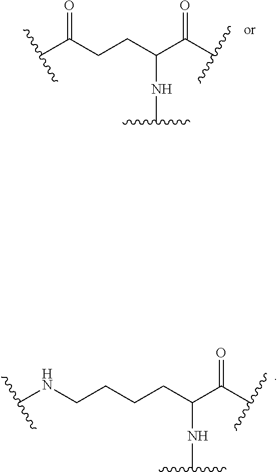 [SMILES: *NC(CCC(*)=O)C(*)=O ; *NCCCCC(N*)C(*)=O]

A is a ligand structure specifically bound to integrin αᵥβ₃, and the structure is shown in Formula (III) or Formula (IV):

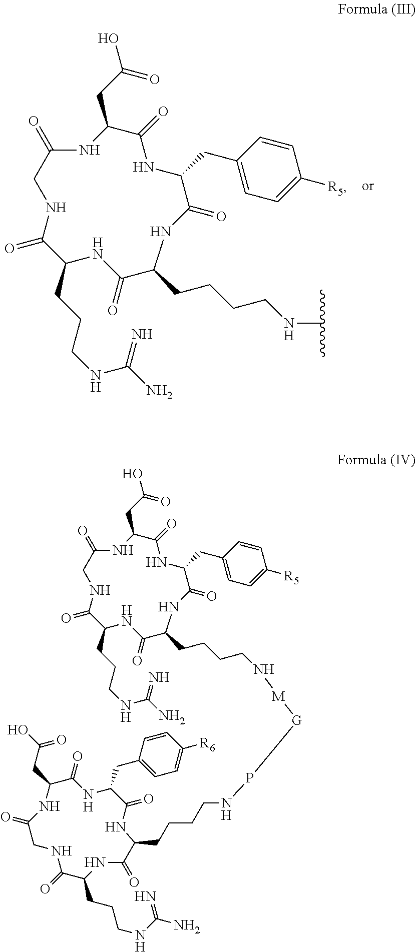

where in Formula (III), R₅ is selected from H or OH.

In Formula (IV), R₅ and R₆ are the same or different, and are independently selected from H or OH.

In Formula (IV), M and P are substitution structures based on —(CH₂)ₙ—, each —CH₂— is separately substituted with or without —O—, —NH—, —(CO)—, —NH—(CO)—, —CH(NH₂)— or —(CO)—NH—, and substitution occurs when no two adjacent —CH₂— groups are substituted; and n is an integer selected from 0 to 30 (such as 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30).

In Formula (IV), G is selected from

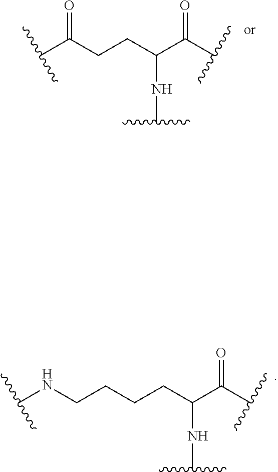 [SMILES: *NC(CCC(*)=O)C(*)=O ; *NCCCCC(N*)C(*)=O]

In some preferred embodiments, where the Z, the Q, the V, the U, the M and/or the P according to any one of the above descriptions are substitution structures based on —(CH₂)ₙ—, the Z, the Q, the V, the U, the M and/or the P may be independently selected from the following structures: —NH—CH₂—(CH₂—O—CH₂)₂—CH₂—(CO)—, —NH—CH₂—(CH₂—O—CH₂)₃—CH₂—(CO)—, —NH—CH₂—(CH₂—O—CH₂)₄—CH₂—(CO)—, —(CO)—NH— or —(CH₂)₀— (namely, an “empty” structure).

In some preferred embodiments, the R₁ and the R₂ in the structure of Formula (I) are simultaneously H (namely, the R₁ is H, and the R₂ is H); in other preferred embodiments, the R₁ and the R₂ in the structure of Formula (I) are simultaneously F (namely, the R₁ is F, and the R₂ is F); and in other preferred embodiments, one of the R₁ and the R₂ in the structure of Formula (I) is H, and the other one is F (namely, the R₁ is H, and the R₂ is F; or the R₁ is F, and the R₂ is H).

In some preferred embodiments, the R₁, the R₂, the R₃ and the R₄ in the structure of Formula (II) are simultaneously H; in other preferred embodiments, the R₁, the R₂, the R₃ and the R₄ in the structure of Formula (II) are simultaneously F; in other preferred embodiments, the R₁ and the R₂ in the structure of Formula (II) are simultaneously H, and the R₃ and the R₄ are simultaneously F; in other preferred embodiments, the R₁ and the R₂ in the structure of Formula (II) are simultaneously H, one of the R₃ and the R₄ is H, and the other one is F (namely, the R₃ is H, and the R₄ is F; or the R₃ is F, and the R₄ is H); and in other preferred embodiments, the R₁ and the R₂ in the structure of Formula (II) are simultaneously F, one of the R₃ and the R₄ is H, and the other one is F (namely, the R₃ is H, and the R₄ is F; or the R₃ is F, and the R₄ is H).

In some preferred embodiments, the R₅ in Formula (III) is H; and in other preferred embodiments, the R₅ in Formula (III) is OH.

In some preferred embodiments, the R₅ and the R₆ in Formula (IV) are simultaneously H (namely, the R₅ is H, and the R₆ is H); in other preferred embodiments, the R₅ and the R₆ in Formula (IV) are simultaneously F (namely, the R₅ is F, and the R₆ is F); in other preferred embodiments, the R₅ in Formula (IV) is H, and the R₆ is F; and in other preferred embodiments, the R₅ in Formula (IV) is F, and the R₆ is H.

In some preferred embodiments, the Z in Formula (I) or Formula (II) is selected from —NH—CH₂—(CH₂—O—CH₂)₂—CH₂—(CO)—, —NH—CH₂—(CH₂—O—CH₂)₃—CH₂—(CO)—, —NH—CH₂—(CH₂—O—CH₂)₄—CH₂—(CO)—,

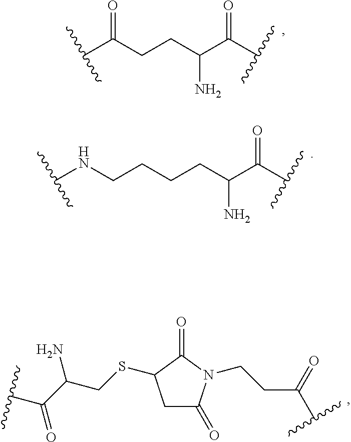 [SMILES: *C(=O)CCC(N)C(*)=O ; *NCCCCC(N)C(*)=O ; *C(=O)CCN1C(=O)CC(SCC(N)C(*)=O)C1=O]

—(CO)—NH— or —(CH₂)₀—; and more preferably, the Z in Formula (I) or Formula (II) is selected from —NH—CH₂—(CH₂—O—CH₂)₂—CH₂—(CO)—, —NH—CH₂—(CH₂—O—CH₂)₃—CH₂—(CO)—, —NH—CH₂—(CH₂—O—CH₂)₄—CH₂—(CO)—, —(CO)—NH— or —(CH₂)₀—.

In some preferred embodiments, the Q in Formula (I) or Formula (II) is selected from

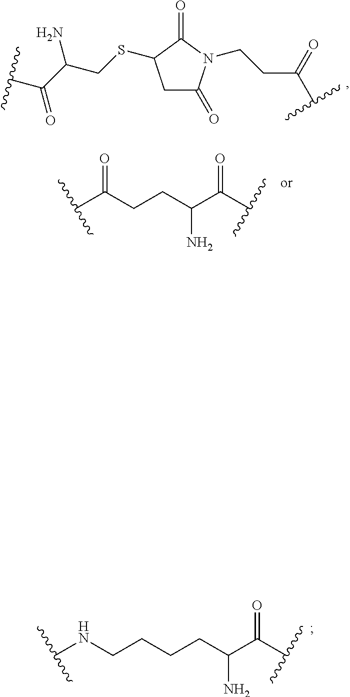

and more preferably, the Q in Formula (I) or Formula (II) is selected from

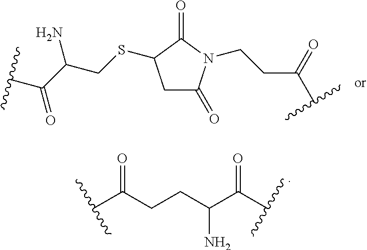

In some preferred embodiments, the V in Formula (I) or Formula (II) is selected from —NH—CH₂—(CH₂—O—CH₂)₂—CH₂—(CO)—, —NH—CH₂—(CH₂—O—CH₂)₃—CH₂—(CO)—, —NH—CH₂—(CH₂—O—CH₂)₄—CH₂—(CO)—, —(CH₂)₀— or —NH—(CO)—.

In some preferred embodiments, the U in Formula (I) or Formula (II) is selected from —NH—,

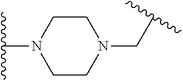 [SMILES: *CN1CCN(*)CC1]

### or —NH—CH₂—.

In some preferred embodiments, the Z₁ in Formula (II) is

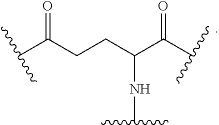 [SMILES: *NC(CCC(*)=O)C(*)=O]

In some preferred embodiments, the M in Formula (IV) is selected from —NH—CH₂—(CH₂—O—CH₂)₂—CH₂—(CO)—, —NH—CH₂—(CH₂—O—CH₂)₃—CH₂—(CO)—, —NH—CH₂—(CH₂—O—CH₂)₄—CH₂—(CO)— or —(CH₂)₀—; and more preferably, the M in Formula (IV) is selected from —NH—CH₂—(CH₂—O—CH₂)₂—CH₂—(CO)— or —NH—CH₂—(CH₂—O—CH₂)₄—CH₂—(CO)—.

In some preferred embodiments, the P in Formula (IV) is selected from —NH—CH₂—(CH₂—O—CH₂)₂—CH₂—(CO)—, —NH—CH₂—(CH₂—O—CH₂)₃—CH₂—(CO)—, —NH—CH₂—(CH₂—O—CH₂)₄—CH₂—(CO)— or —(CH₂)₀—; and more preferably, the P in Formula (IV) is selected from —NH—CH₂—(CH₂—O—CH₂)₂—CH₂—(CO)— or —NH—CH₂—(CH₂—O—CH₂)₄—CH₂—(CO)—.

In furthermore preferred embodiments, the dual-targeting compound is selected from the following structures:

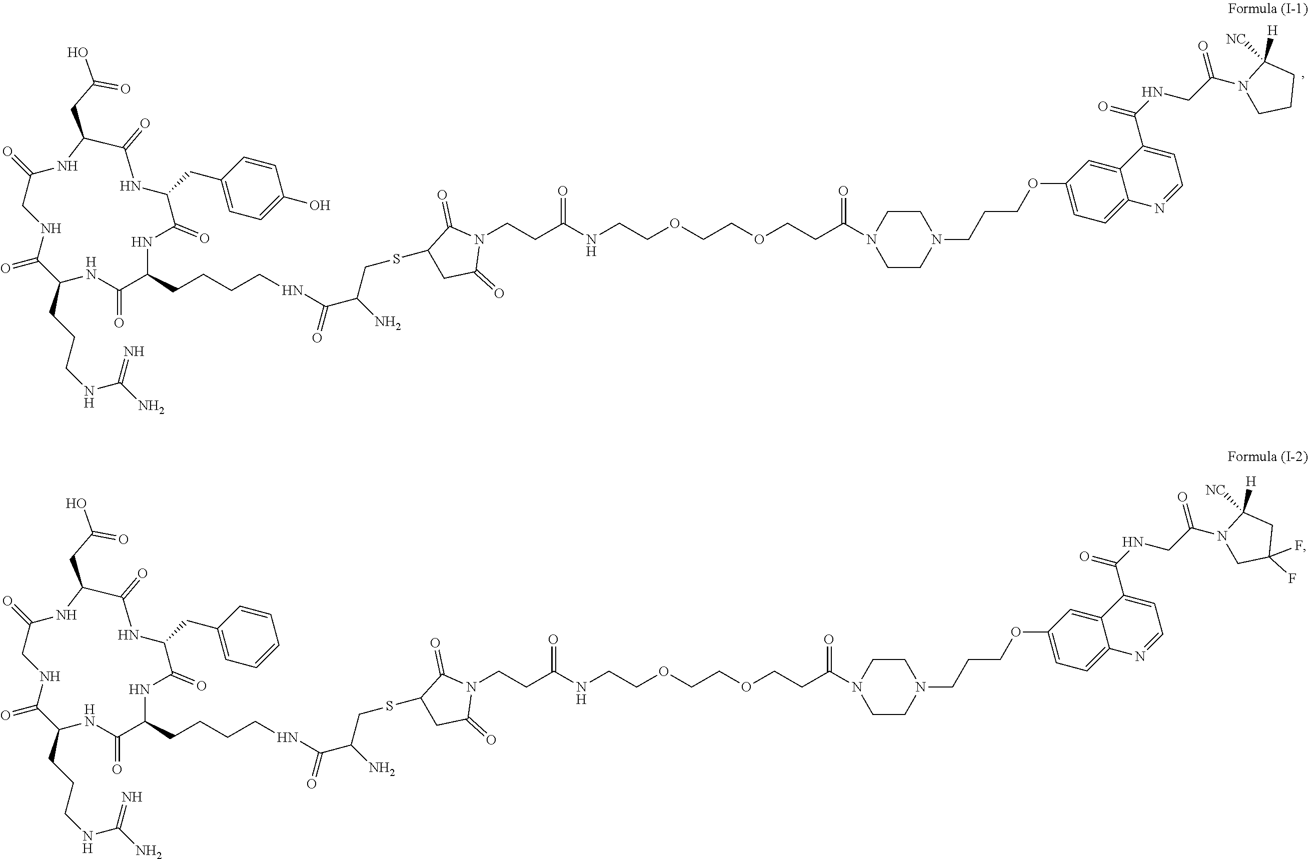 [SMILES: N#C[C@@H]1CCCN1C(=O)CNC(=O)c1ccnc2ccc(OCCCN3CCN(C(=O)CCOCCOCCNC(=O)CCN4C(=O)CC(SCC(N)C(=O)NCCCC[C@@H]5NC(=O)[C@@H](Cc6ccc(O)cc6)NC(=O)[C@H](CC(=O)O)NC(=O)CNC(=O)[C@H](CCCNC(=N)N)NC5=O)C4=O)CC3)cc12 ; N#C[C@@H]1CC(F)(F)CN1C(=O)CNC(=O)c1ccnc2ccc(OCCCN3CCN(C(=O)CCOCCOCCNC(=O)CCN4C(=O)CC(SCC(N)C(=O)NCCCC[C@@H]5NC(=O)[C@@H](Cc6ccccc6)NC(=O)[C@H](CC(=O)O)NC(=O)CNC(=O)[C@H](CCCNC(=N)N)NC5=O)C4=O)CC3)cc12]

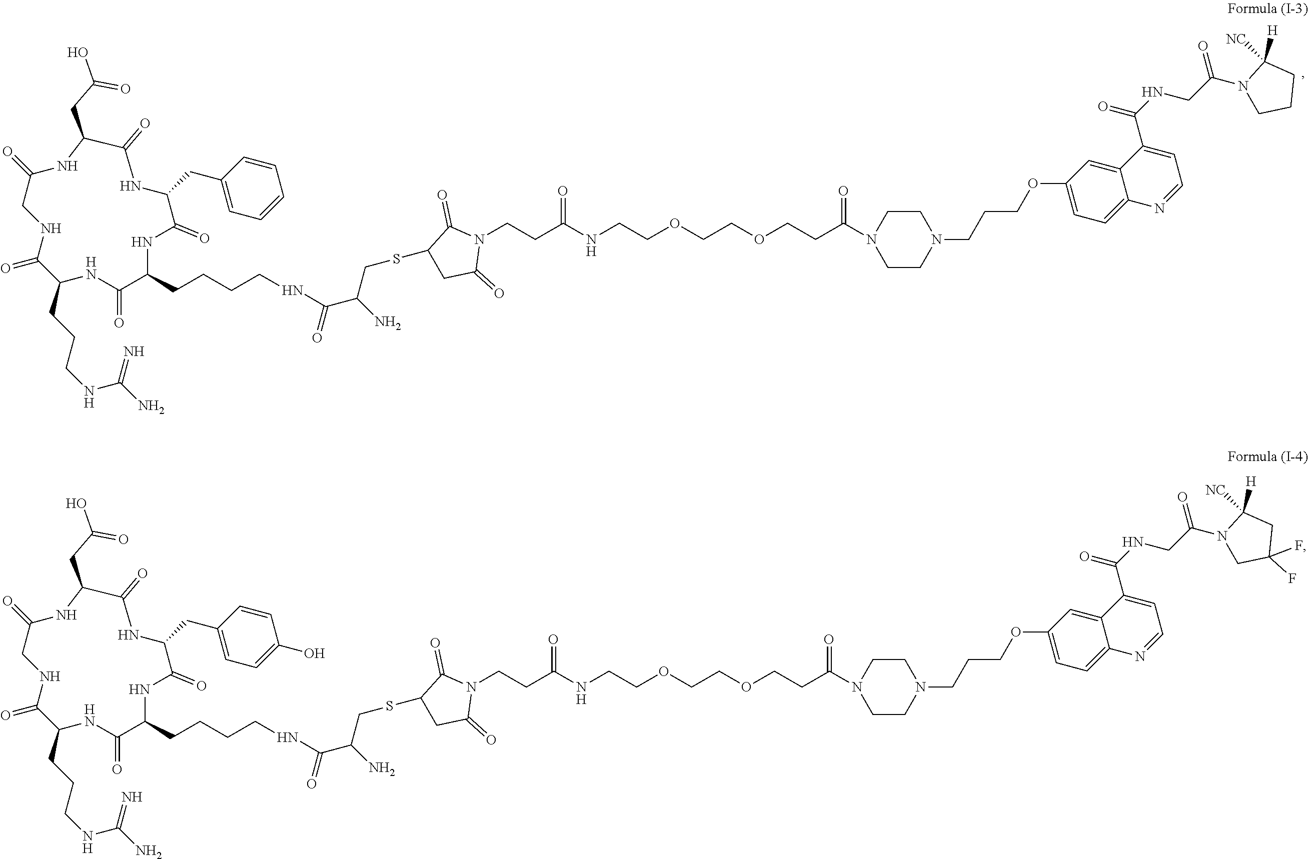 [SMILES: N#C[C@@H]1CCCN1C(=O)CNC(=O)c1ccnc2ccc(OCCCN3CCN(C(=O)CCOCCOCCNC(=O)CCN4C(=O)CC(SCC(N)C(=O)NCCCC[C@@H]5NC(=O)[C@@H](Cc6ccccc6)NC(=O)[C@H](CC(=O)O)NC(=O)CNC(=O)[C@H](CCCNC(=N)N)NC5=O)C4=O)CC3)cc12 ; N#C[C@@H]1CC(F)(F)CN1C(=O)CNC(=O)c1ccnc2ccc(OCCCN3CCN(C(=O)CCOCCOCCNC(=O)CCN4C(=O)CC(SCC(N)C(=O)NCCCC[C@@H]5NC(=O)[C@@H](Cc6ccc(O)cc6)NC(=O)[C@H](CC(=O)O)NC(=O)CNC(=O)[C@H](CCCNC(=N)N)NC5=O)C4=O)CC3)cc12]

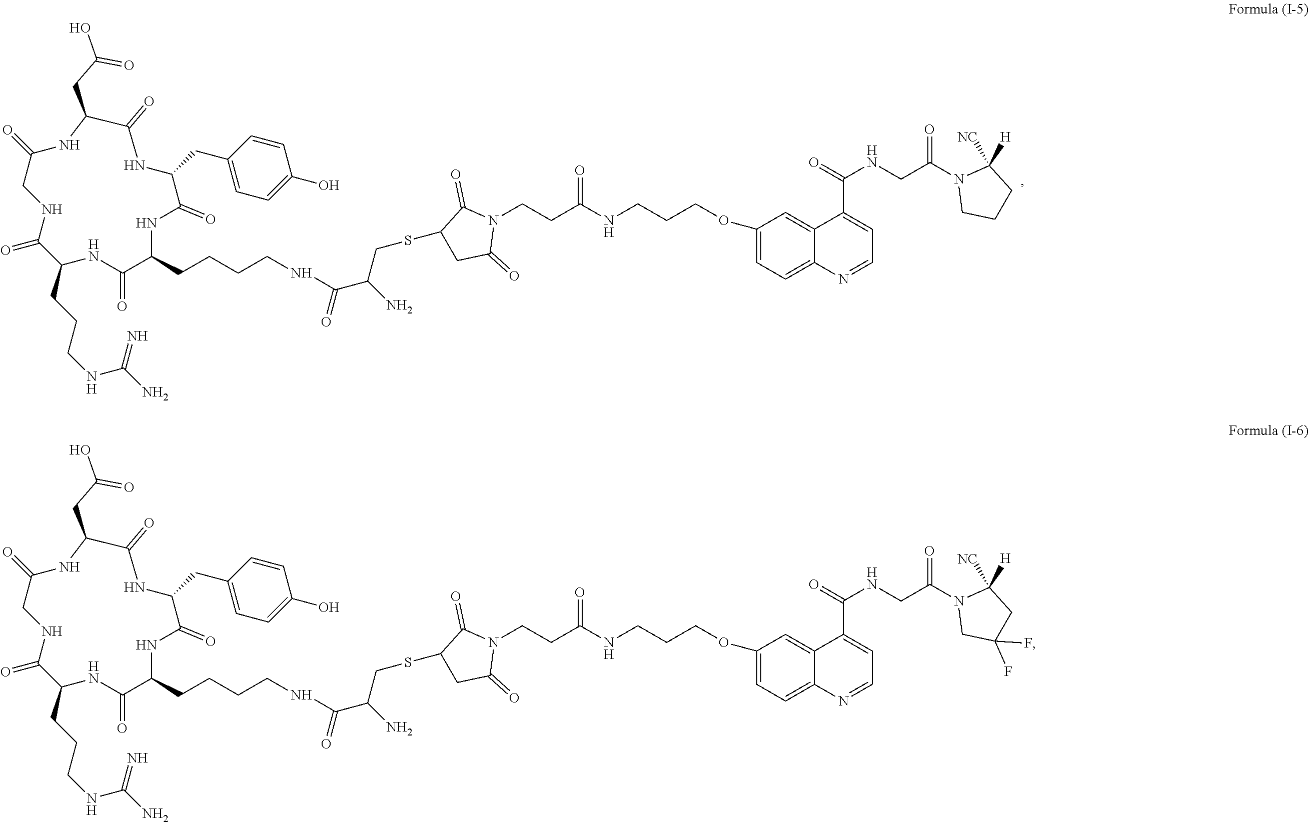 [SMILES: N#C[C@@H]1CCCN1C(=O)CNC(=O)c1ccnc2ccc(OCCCNC(=O)CCN3C(=O)CC(SCC(N)C(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)C3=O)cc12 ; N#C[C@@H]1CC(F)(F)CN1C(=O)CNC(=O)c1ccnc2ccc(OCCCNC(=O)CCN3C(=O)CC(SCC(N)C(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)C3=O)cc12]

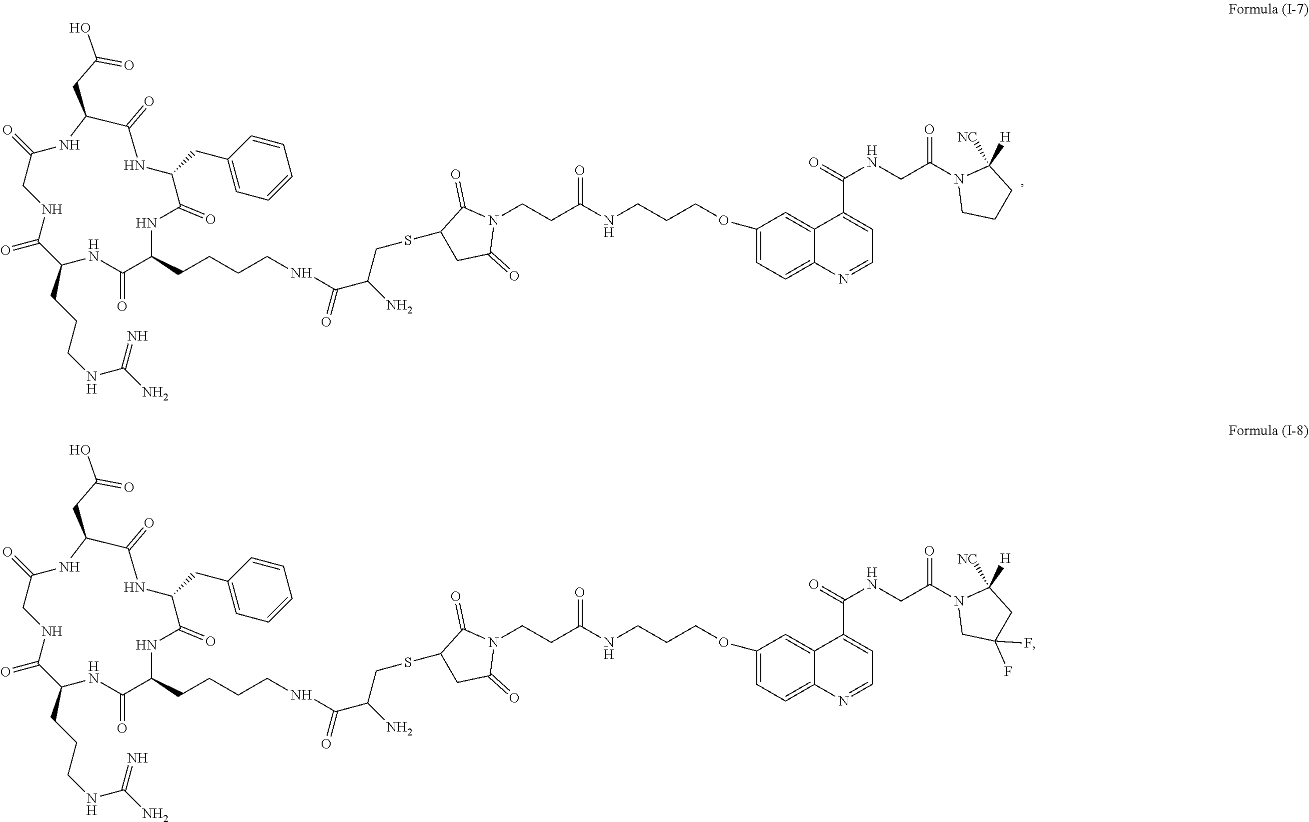 [SMILES: N#C[C@@H]1CCCN1C(=O)CNC(=O)c1ccnc2ccc(OCCCNC(=O)CCN3C(=O)CC(SCC(N)C(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)C3=O)cc12 ; N#C[C@@H]1CC(F)(F)CN1C(=O)CNC(=O)c1ccnc2ccc(OCCCNC(=O)CCN3C(=O)CC(SCC(N)C(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)C3=O)cc12]

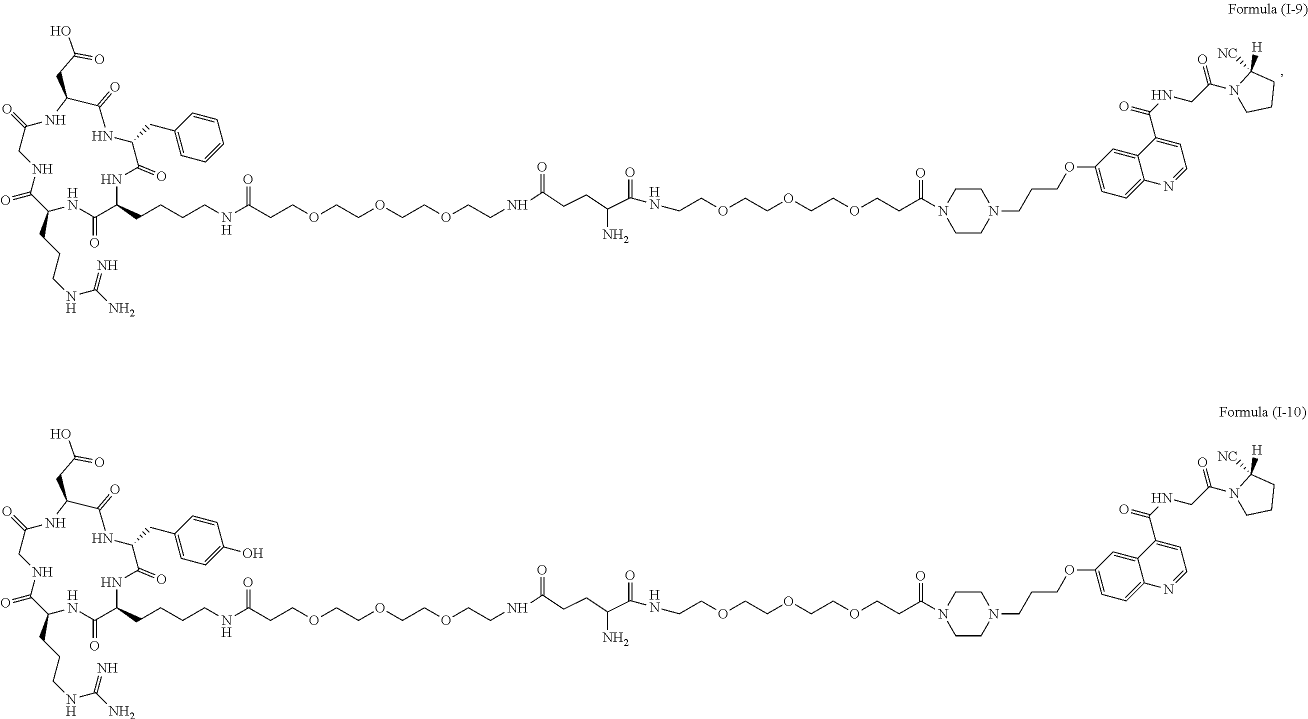 [SMILES: N#C[C@@H]1CCCN1C(=O)CNC(=O)c1ccnc2ccc(OCCCN3CCN(C(=O)CCOCCOCCOCCNC(=O)C(N)CCC(=O)NCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)CC3)cc12 ; N#C[C@@H]1CCCN1C(=O)CNC(=O)c1ccnc2ccc(OCCCN3CCN(C(=O)CCOCCOCCOCCNC(=O)C(N)CCC(=O)NCCOCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)CC3)cc12]

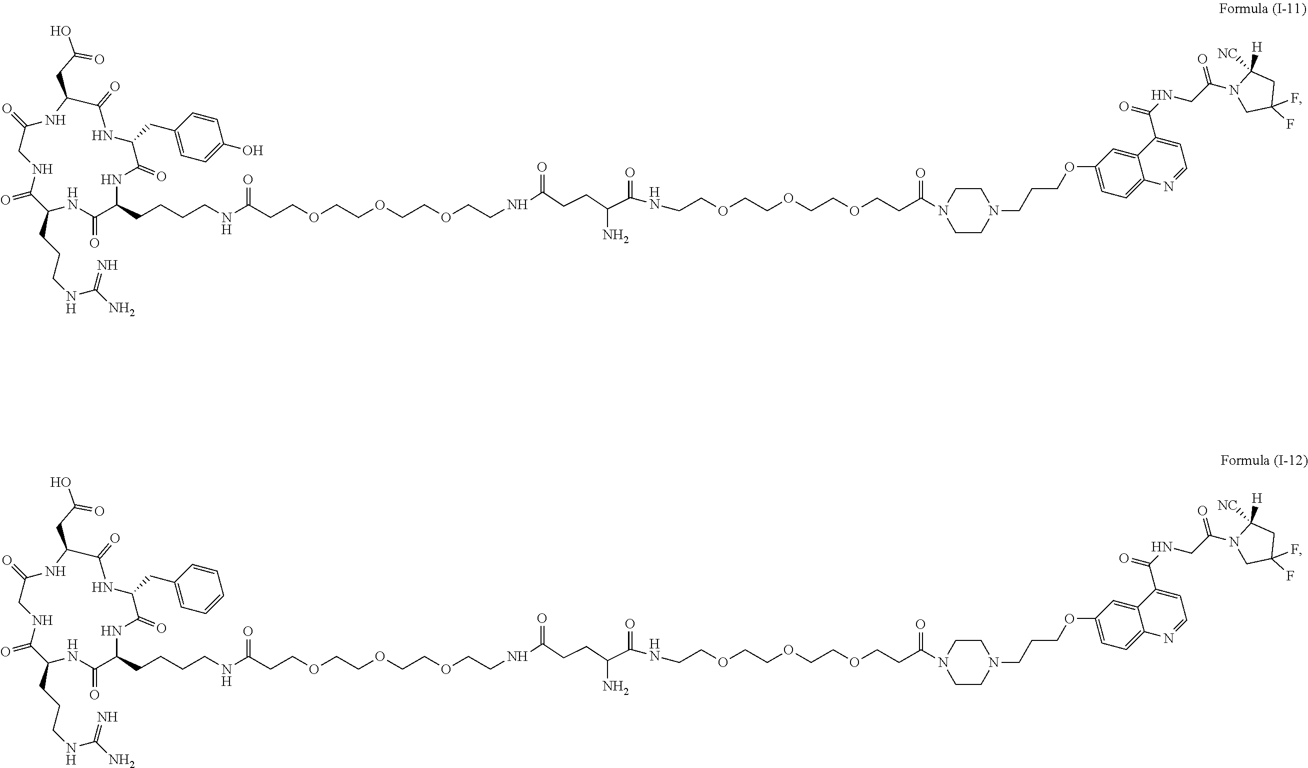 [SMILES: N#C[C@@H]1CC(F)(F)CN1C(=O)CNC(=O)c1ccnc2ccc(OCCCN3CCN(C(=O)CCOCCOCCOCCNC(=O)C(N)CCC(=O)NCCOCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)CC3)cc12 ; N#C[C@@H]1CC(F)(F)CN1C(=O)CNC(=O)c1ccnc2ccc(OCCCN3CCN(C(=O)CCOCCOCCOCCNC(=O)C(N)CCC(=O)NCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)CC3)cc12]

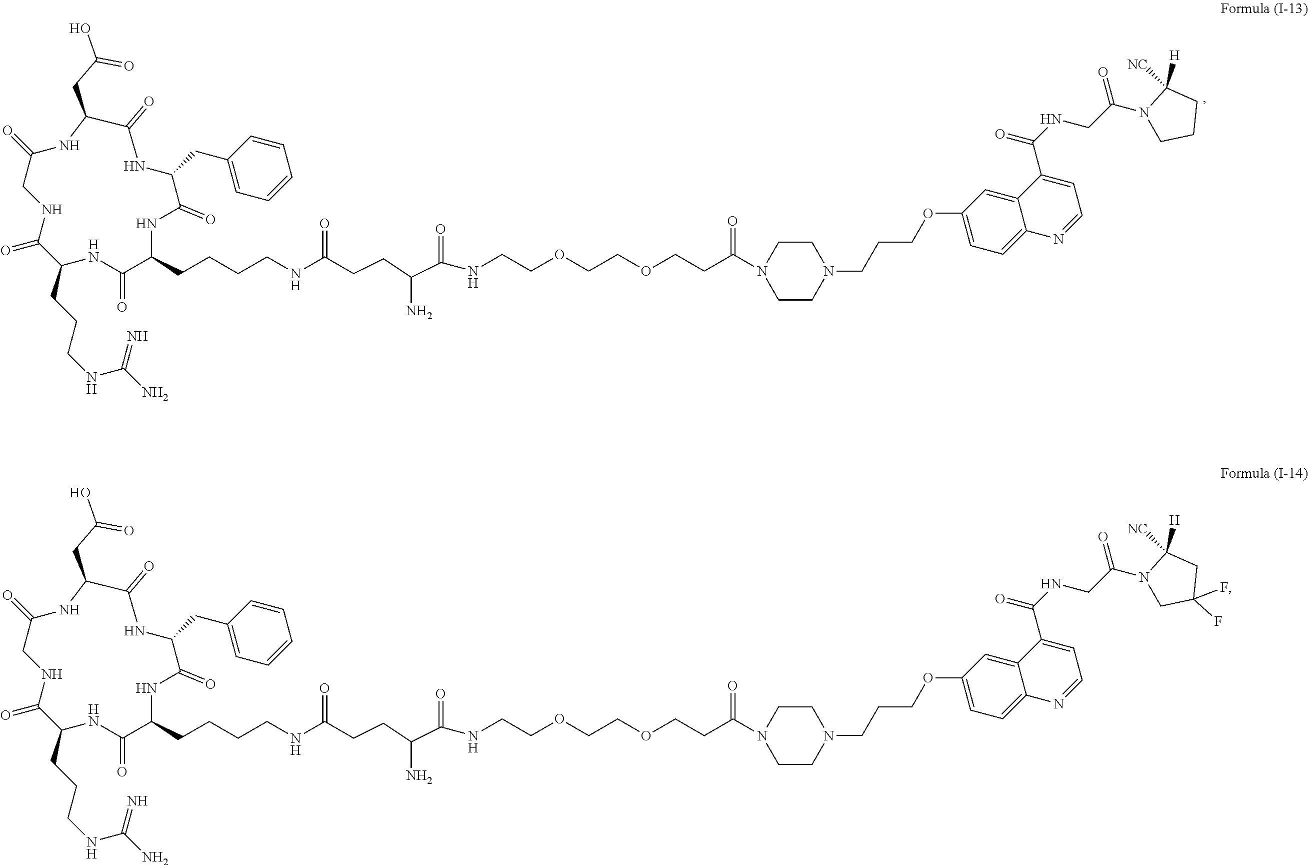 [SMILES: N#C[C@@H]1CCCN1C(=O)CNC(=O)c1ccnc2ccc(OCCCN3CCN(C(=O)CCOCCOCCNC(=O)C(N)CCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)CC3)cc12 ; N#C[C@@H]1CC(F)(F)CN1C(=O)CNC(=O)c1ccnc2ccc(OCCCN3CCN(C(=O)CCOCCOCCNC(=O)C(N)CCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)CC3)cc12]

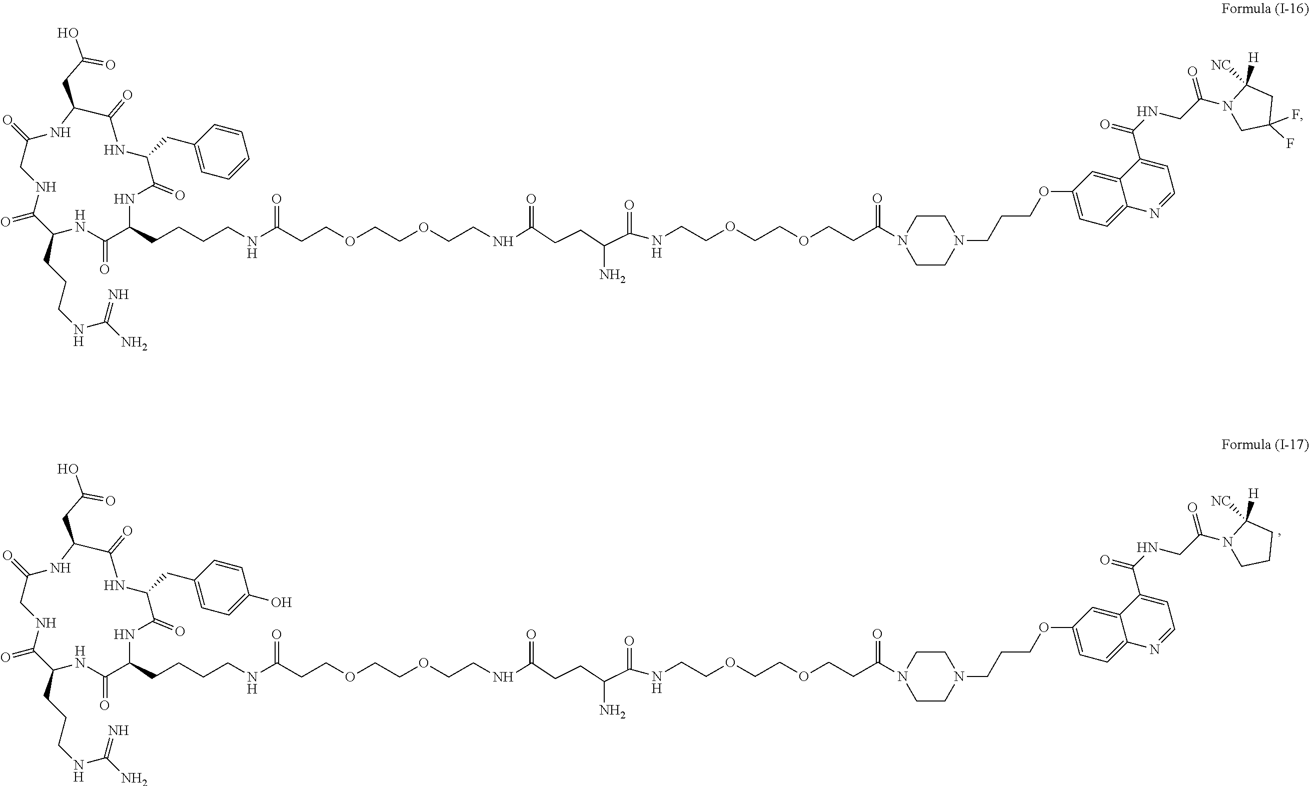 [SMILES: N#C[C@@H]1CC(F)(F)CN1C(=O)CNC(=O)c1ccnc2ccc(OCCCN3CCN(C(=O)CCOCCOCCNC(=O)C(N)CCC(=O)NCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)CC3)cc12 ; N#C[C@@H]1CCCN1C(=O)CNC(=O)c1ccnc2ccc(OCCCN3CCN(C(=O)CCOCCOCCNC(=O)C(N)CCC(=O)NCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)CC3)cc12]

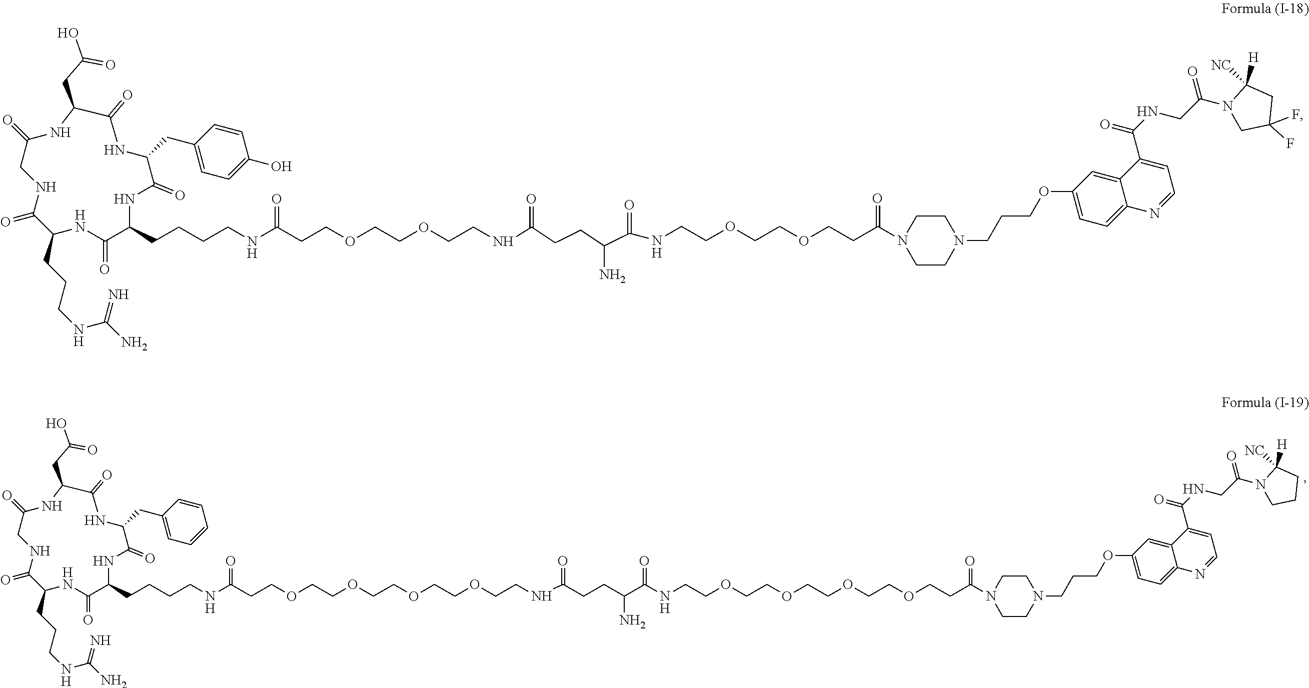 [SMILES: N#C[C@@H]1CC(F)(F)CN1C(=O)CNC(=O)c1ccnc2ccc(OCCCN3CCN(C(=O)CCOCCOCCNC(=O)C(N)CCC(=O)NCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)CC3)cc12 ; N#C[C@@H]1CCCN1C(=O)CNC(=O)c1ccnc2ccc(OCCCN3CCN(C(=O)CCOCCOCCOCCOCCNC(=O)C(N)CCC(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)CC3)cc12]

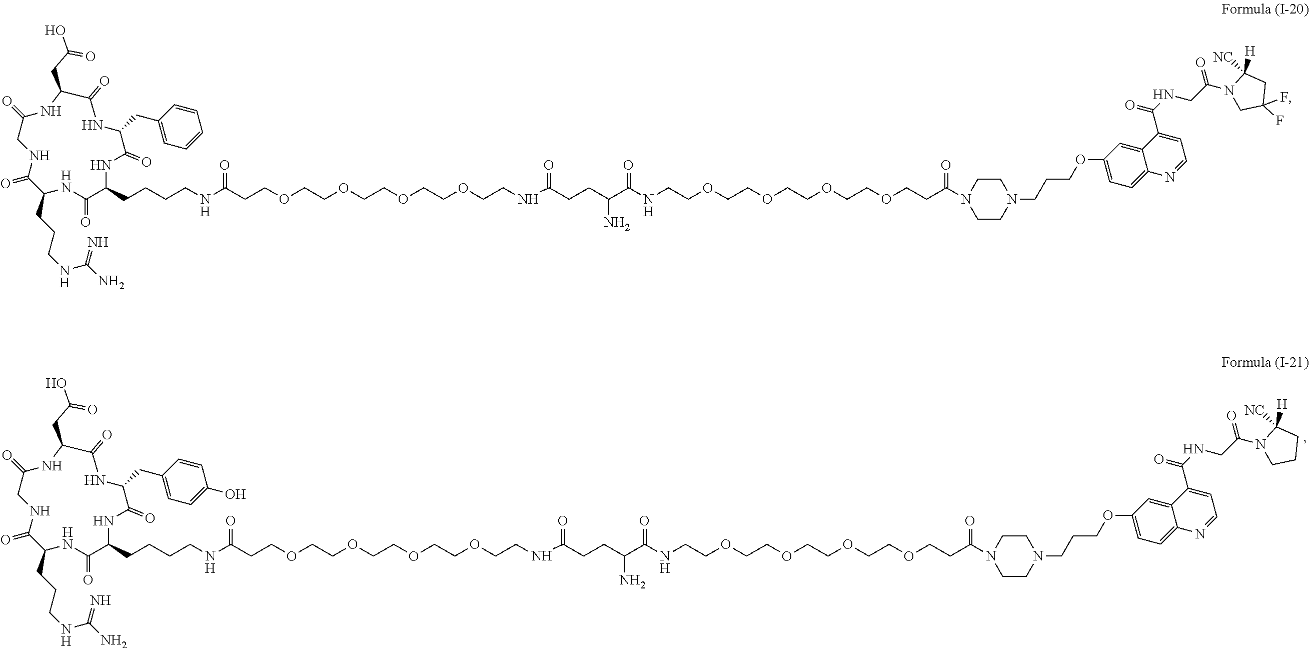 [SMILES: N#C[C@@H]1CC(F)(F)CN1C(=O)CNC(=O)c1ccnc2ccc(OCCCN3CCN(C(=O)CCOCCOCCOCCOCCNC(=O)C(N)CCC(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)CC3)cc12 ; N#C[C@@H]1CCCN1C(=O)CNC(=O)c1ccnc2ccc(OCCCN3CCN(C(=O)CCOCCOCCOCCOCCNC(=O)C(N)CCC(=O)NCCOCCOCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)CC3)cc12]

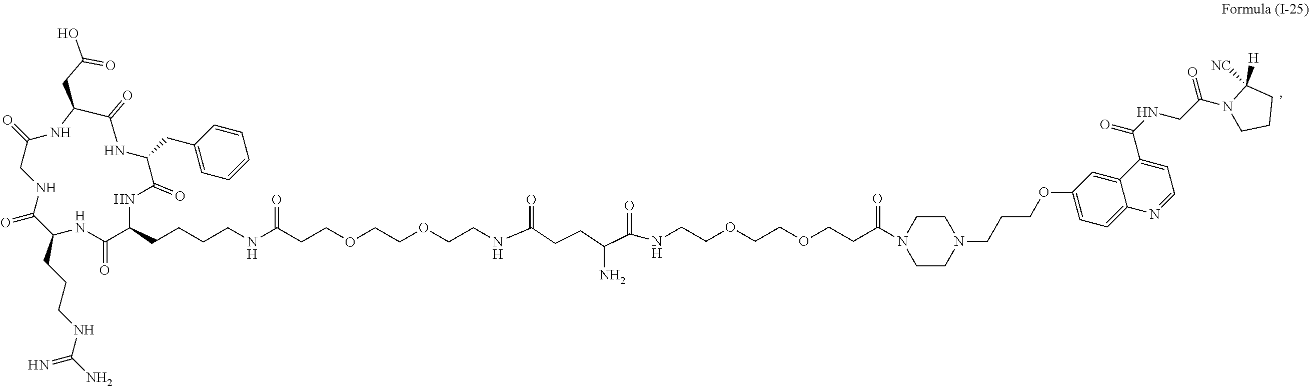 [SMILES: N#C[C@@H]1CCCN1C(=O)CNC(=O)c1ccnc2ccc(OCCCN3CCN(C(=O)CCOCCOCCNC(=O)C(N)CCC(=O)NCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)CC3)cc12]

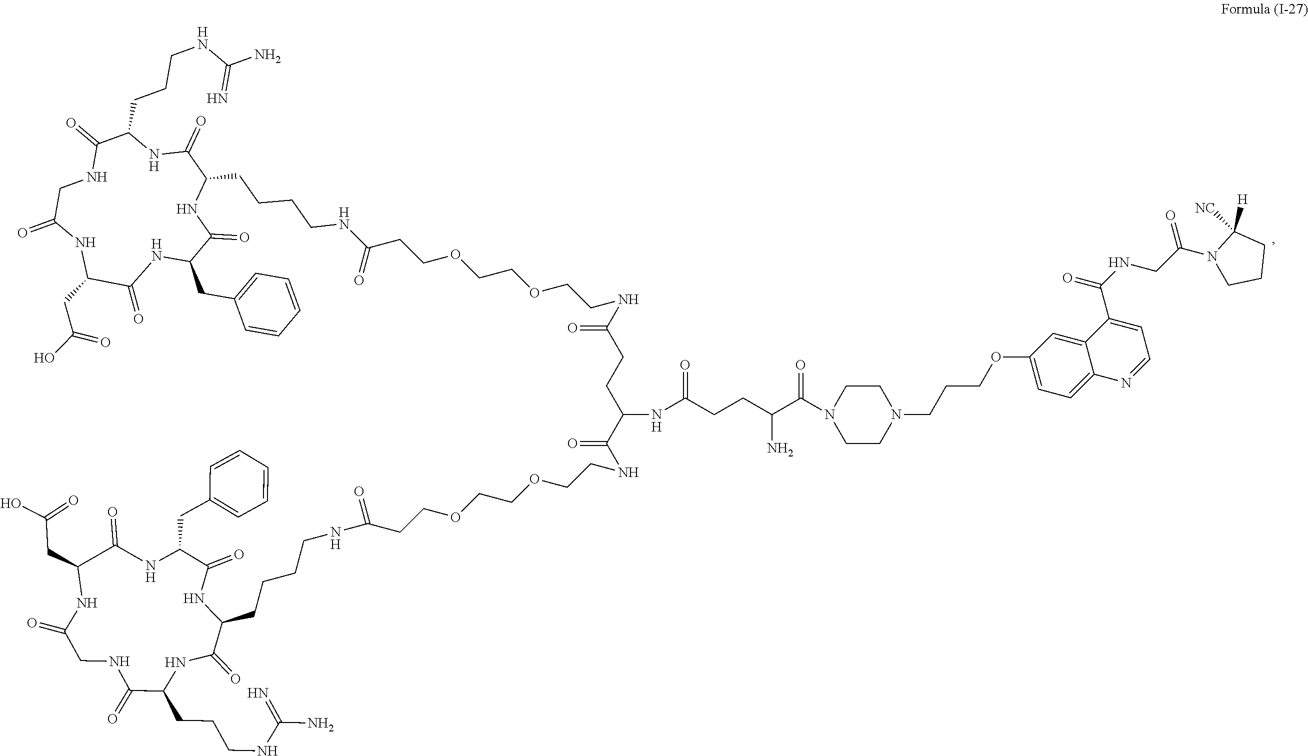 [SMILES: N#C[C@@H]1CCCN1C(=O)CNC(=O)c1ccnc2ccc(OCCCN3CCN(C(=O)C(N)CCC(=O)NC(CCC(=O)NCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)C(=O)NCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)CC3)cc12]

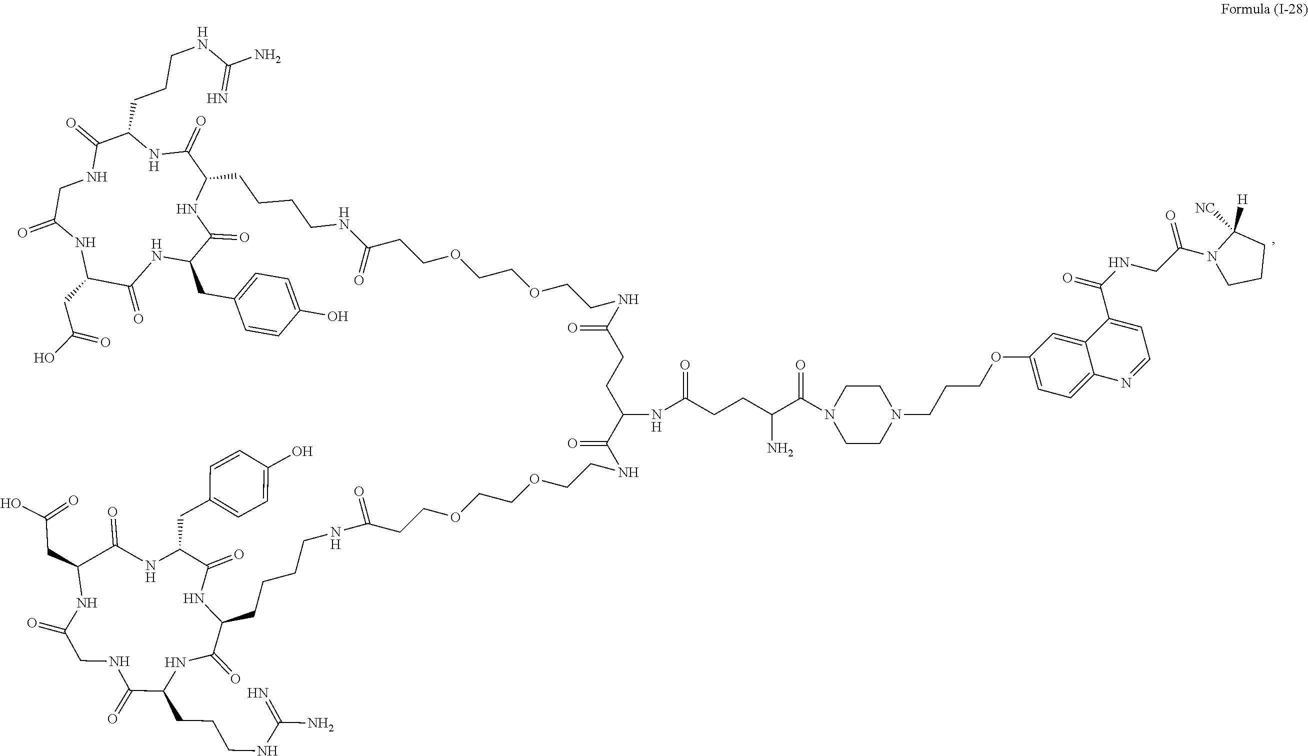 [SMILES: N#C[C@@H]1CCCN1C(=O)CNC(=O)c1ccnc2ccc(OCCCN3CCN(C(=O)C(N)CCC(=O)NC(CCC(=O)NCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)C(=O)NCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)CC3)cc12]

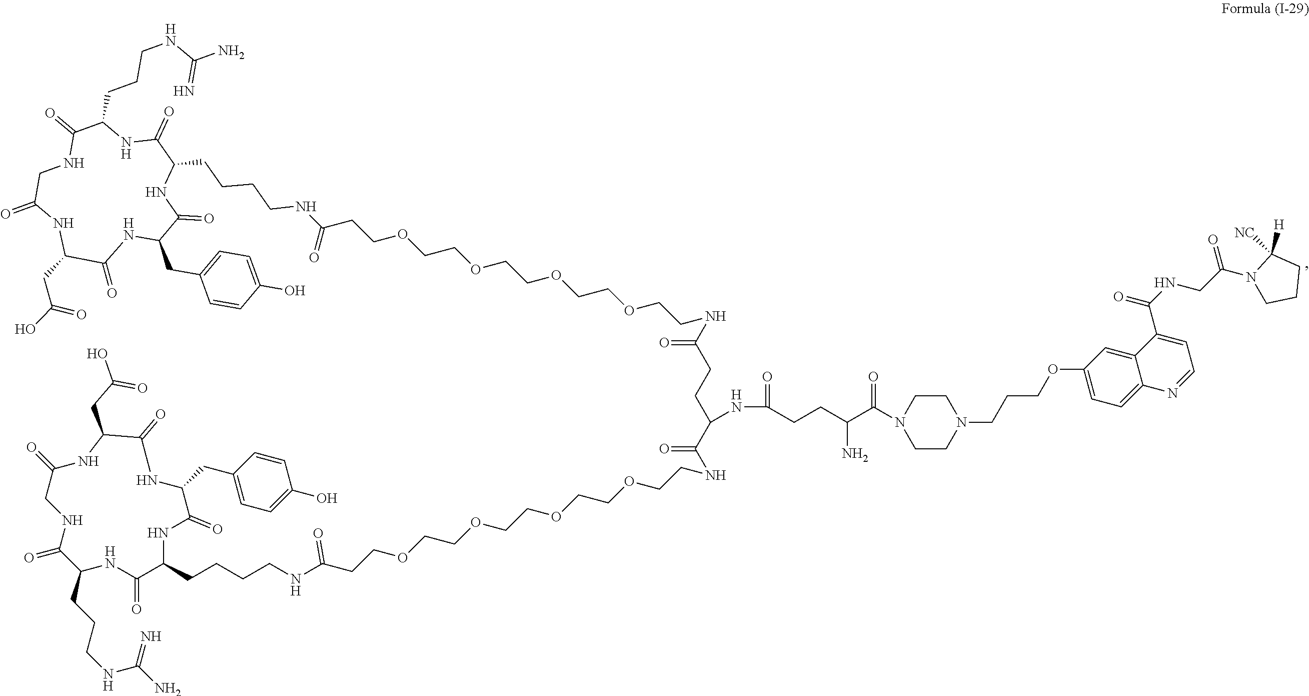 [SMILES: N#C[C@@H]1CCCN1C(=O)CNC(=O)c1ccnc2ccc(OCCCN3CCN(C(=O)C(N)CCC(=O)NC(CCC(=O)NCCOCCOCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)C(=O)NCCOCCOCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)CC3)cc12]

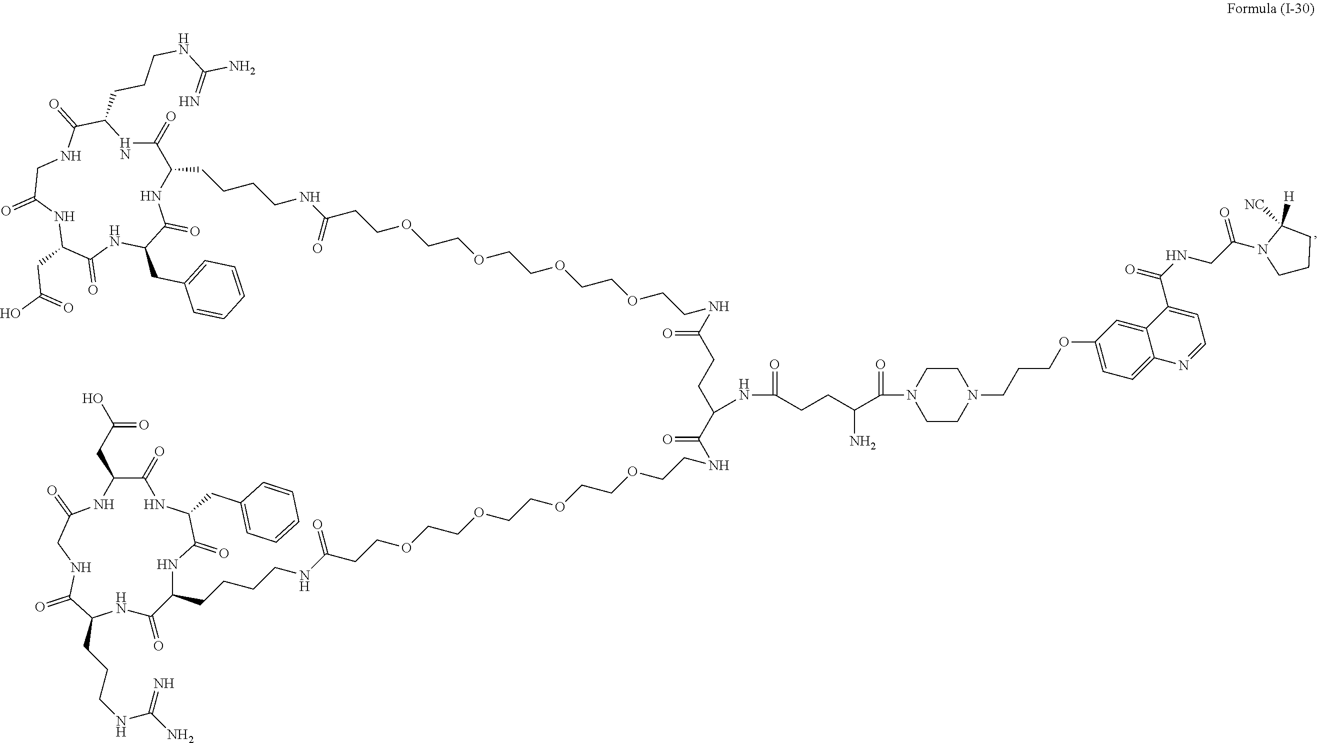 [SMILES: N#C[C@@H]1CCCN1C(=O)CNC(=O)c1ccnc2ccc(OCCCN3CCN(C(=O)C(N)CCC(=O)NC(CCC(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)C(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)CC3)cc12]

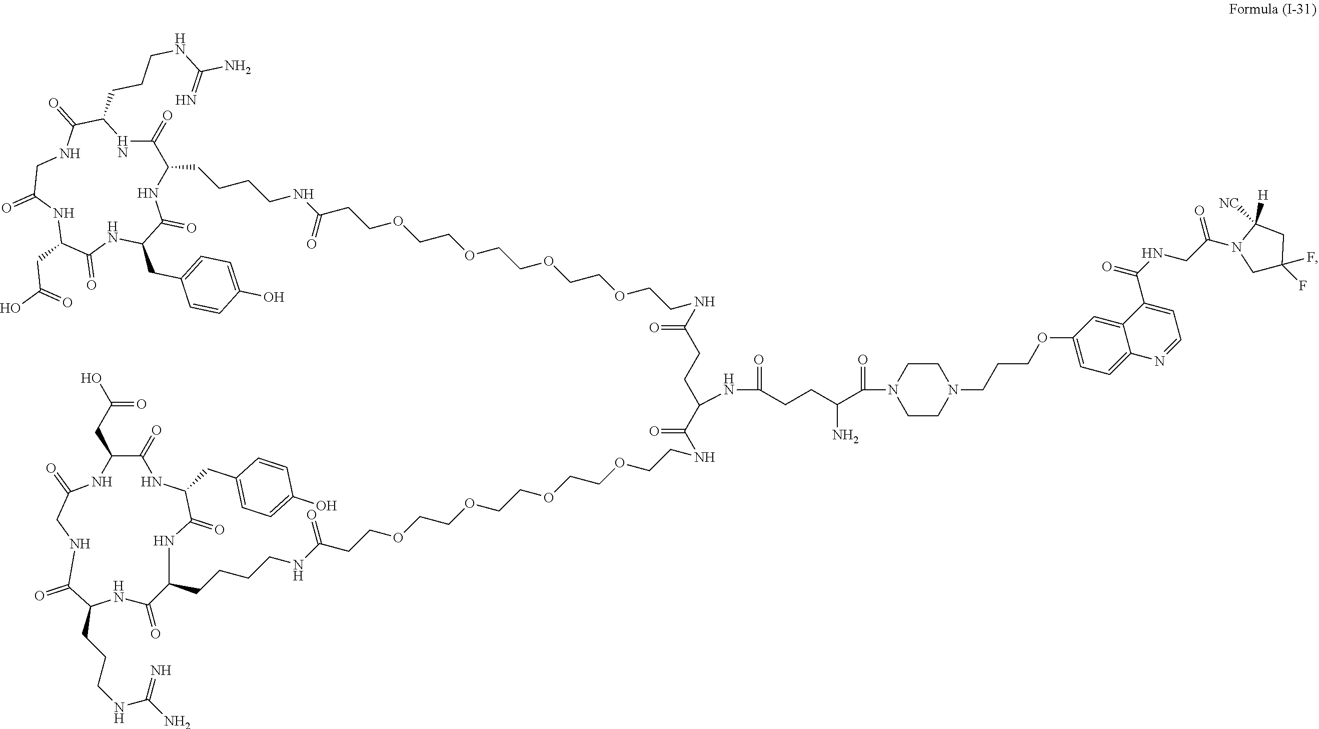 [SMILES: N#C[C@@H]1CC(F)(F)CN1C(=O)CNC(=O)c1ccnc2ccc(OCCCN3CCN(C(=O)C(N)CCC(=O)NC(CCC(=O)NCCOCCOCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)C(=O)NCCOCCOCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)CC3)cc12]

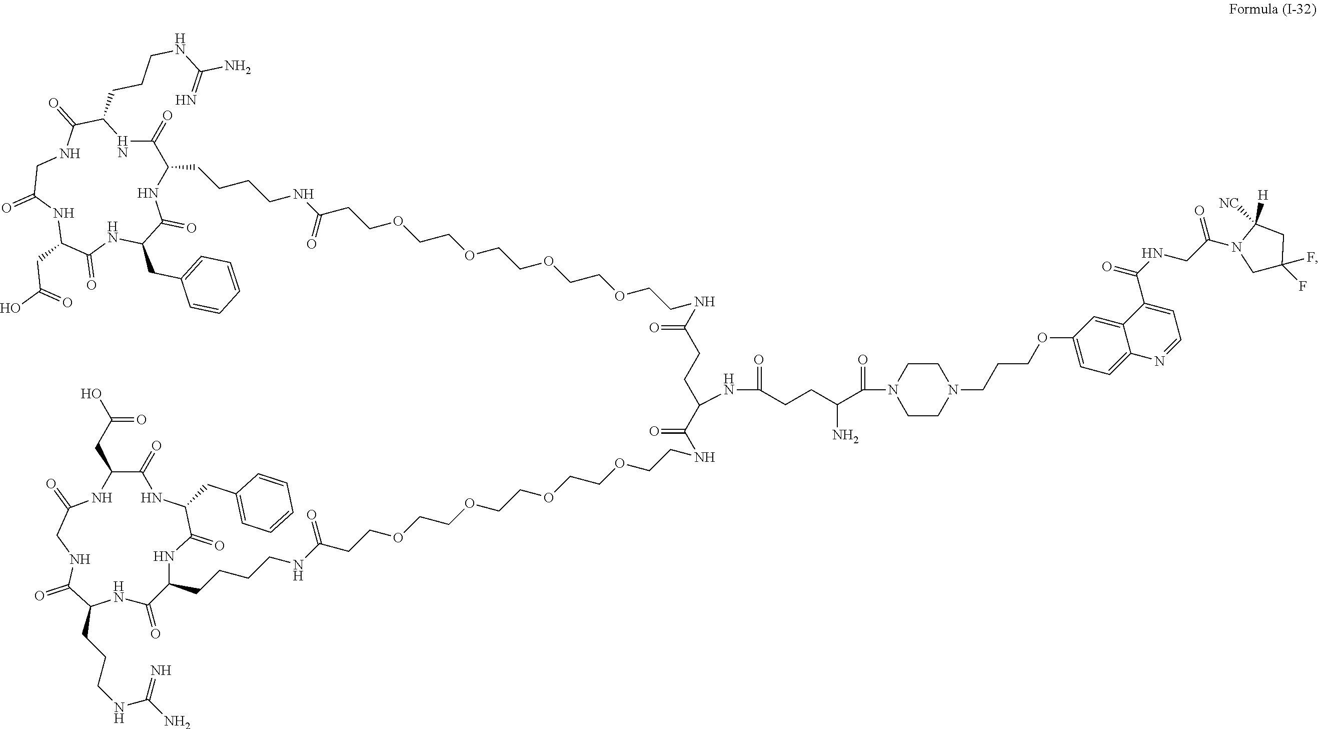 [SMILES: N#C[C@@H]1CC(F)(F)CN1C(=O)CNC(=O)c1ccnc2ccc(OCCCN3CCN(C(=O)C(N)CCC(=O)NC(CCC(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)C(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)CC3)cc12]

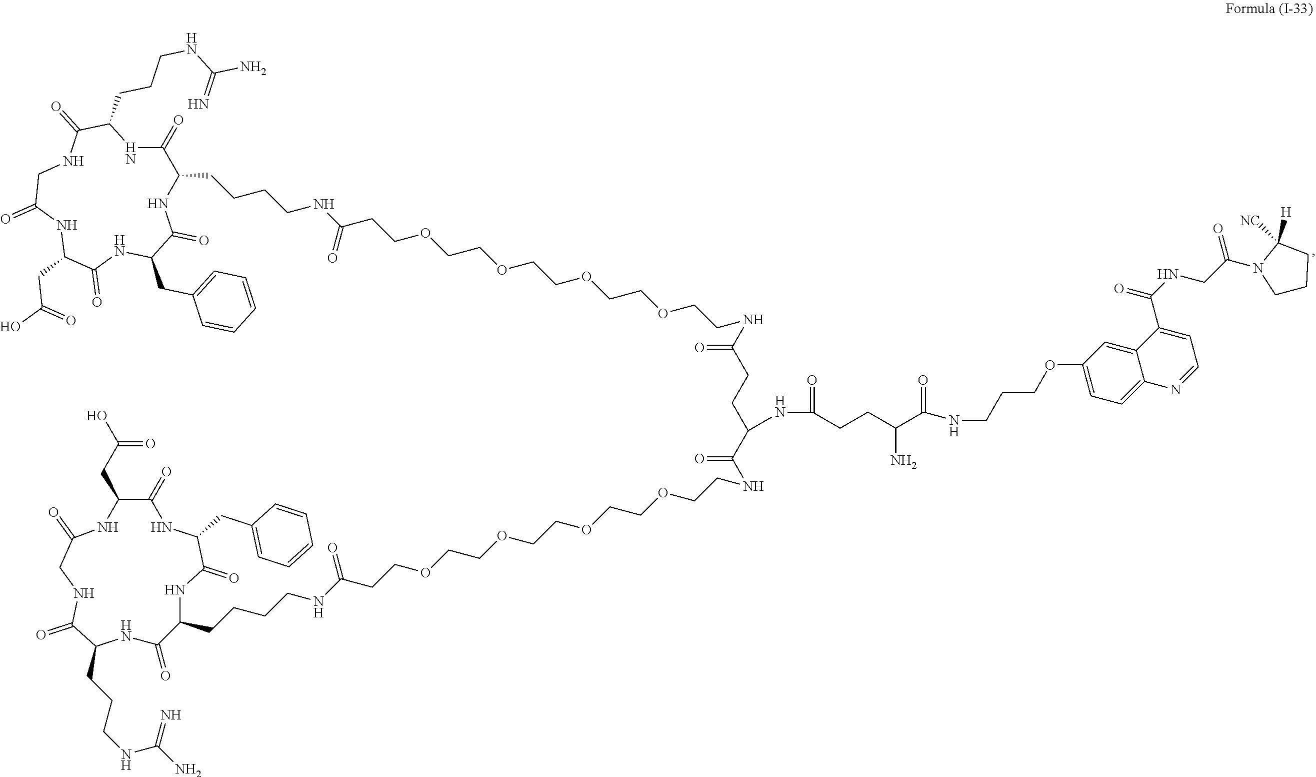 [SMILES: N#C[C@@H]1CCCN1C(=O)CNC(=O)c1ccnc2ccc(OCCCNC(=O)C(N)CCC(=O)NC(CCC(=O)NCCOCCOCCOCCOCCC(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)C(=O)NCCOCCOCCOCCOCCC(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)cc12]

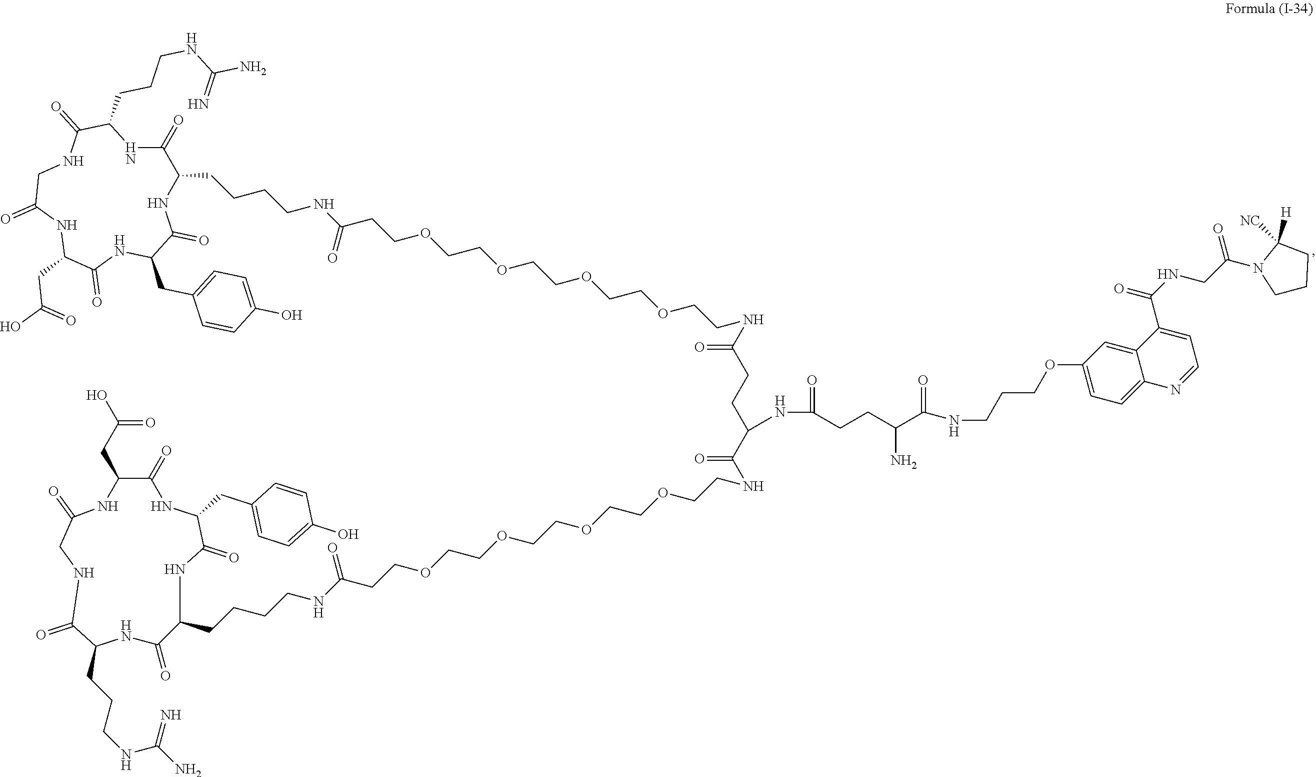 [SMILES: N#C[C@@H]1CCCN1C(=O)CNC(=O)c1ccnc2ccc(OCCCNC(=O)C(N)CCC(=O)NC(CCC(=O)NCCOCCOCCOCCOCCC(=O)NCCCC[C@@H]3NC(=O)[C@@H](Cc4ccc(O)cc4)NC(=O)[C@H](CC(=O)O)NC(=O)CNC(=O)[C@H](CCCNC(=N)N)NC3=O)C(=O)NCCOCCOCCOCCOCCC(=O)NCCCC[C@@H]3NC(=O)[C@@H](Cc4ccc(O)cc4)NC(=O)[C@H](CC(=O)O)NC(=O)CNC(=O)[C@H](CCCNC(=N)N)NC3=O)cc12]

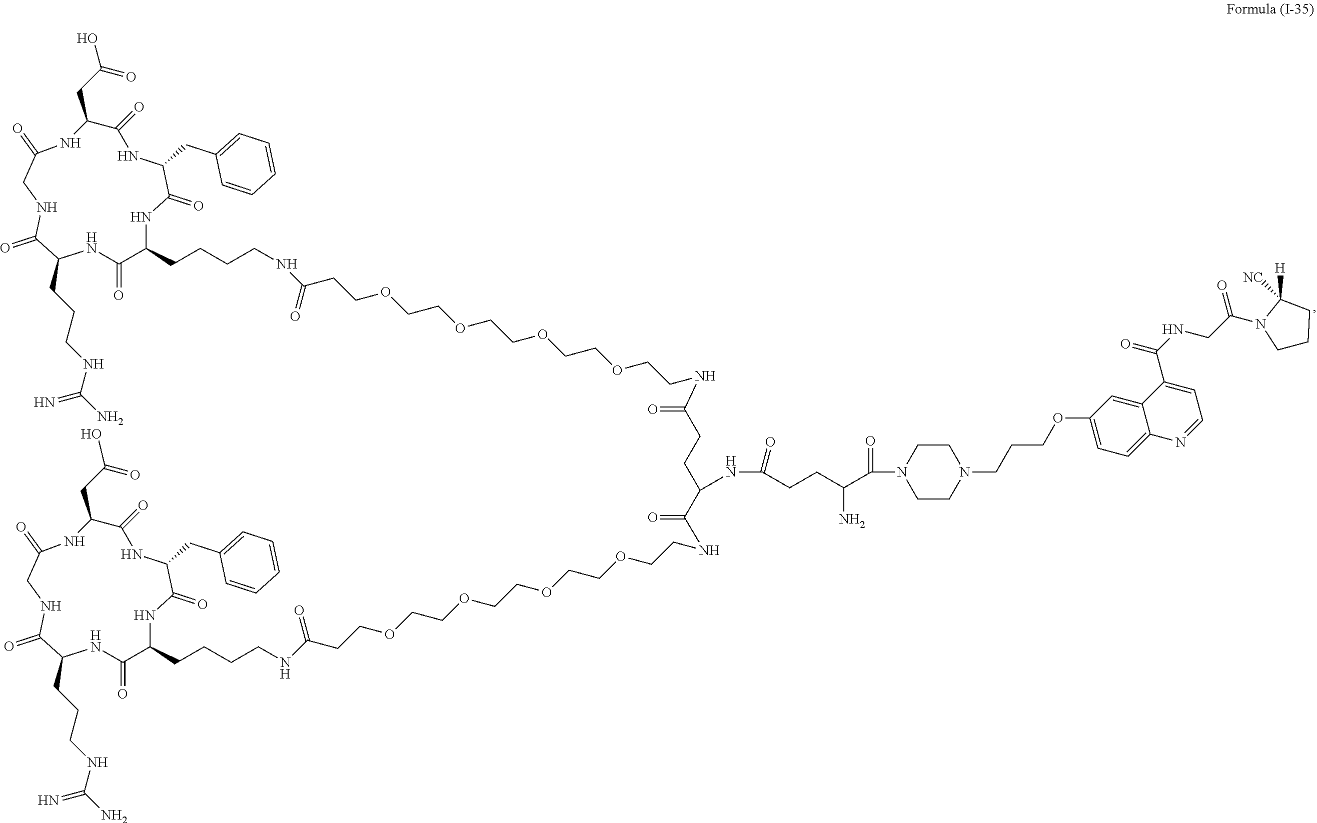 [SMILES: N#C[C@@H]1CCCN1C(=O)CNC(=O)c1ccnc2ccc(OCCCN3CCN(C(=O)C(N)CCC(=O)NC(CCC(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)C(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)CC3)cc12]

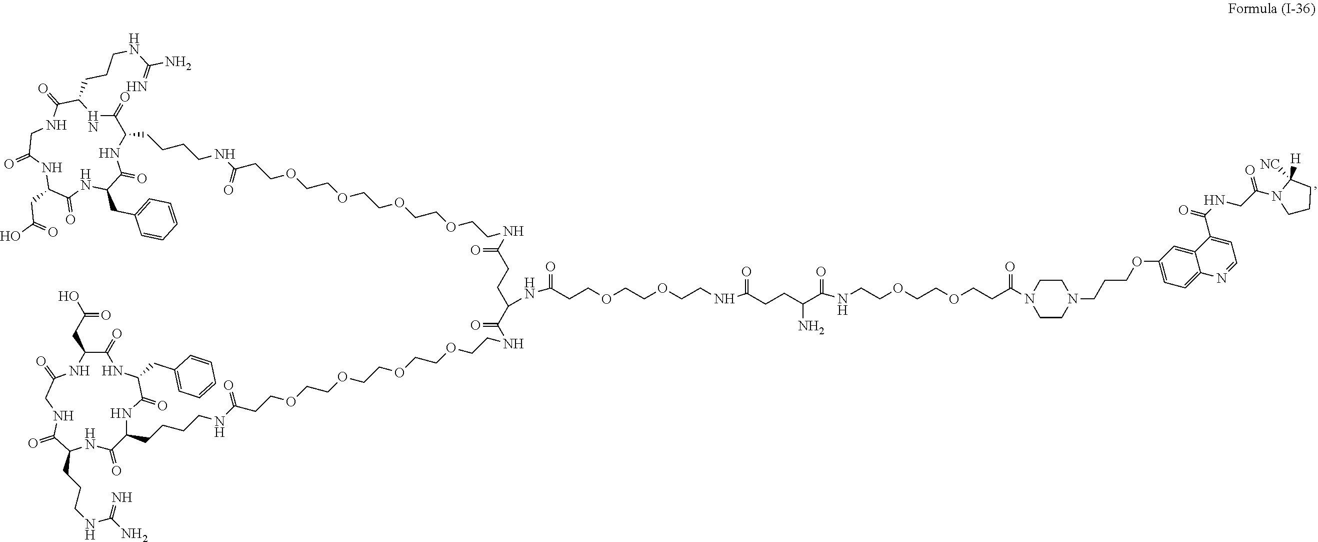 [SMILES: N#C[C@@H]1CCCN1C(=O)CNC(=O)c1ccnc2ccc(OCCCN3CCN(C(=O)CCOCCOCCNC(=O)C(N)CCC(=O)NCCOCCOCCC(=O)NC(CCC(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)C(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)CC3)cc12]

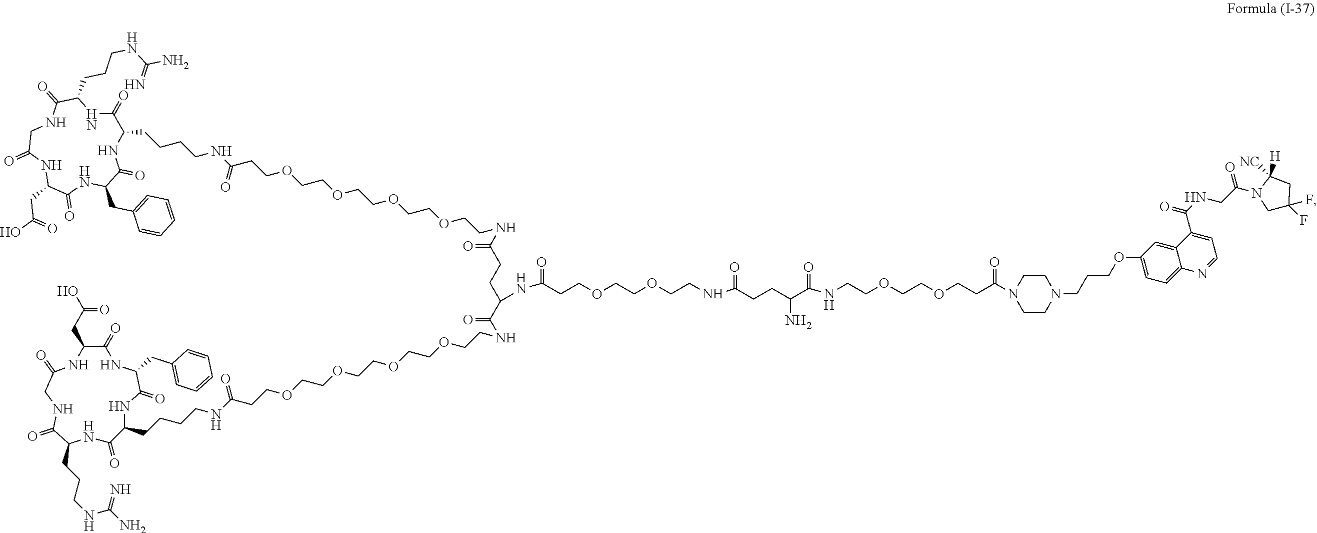 [SMILES: N#C[C@@H]1CC(F)(F)CN1C(=O)CNC(=O)c1ccnc2ccc(OCCCN3CCN(C(=O)CCOCCOCCNC(=O)C(N)CCC(=O)NCCOCCOCCC(=O)NC(CCC(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)C(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)CC3)cc12]

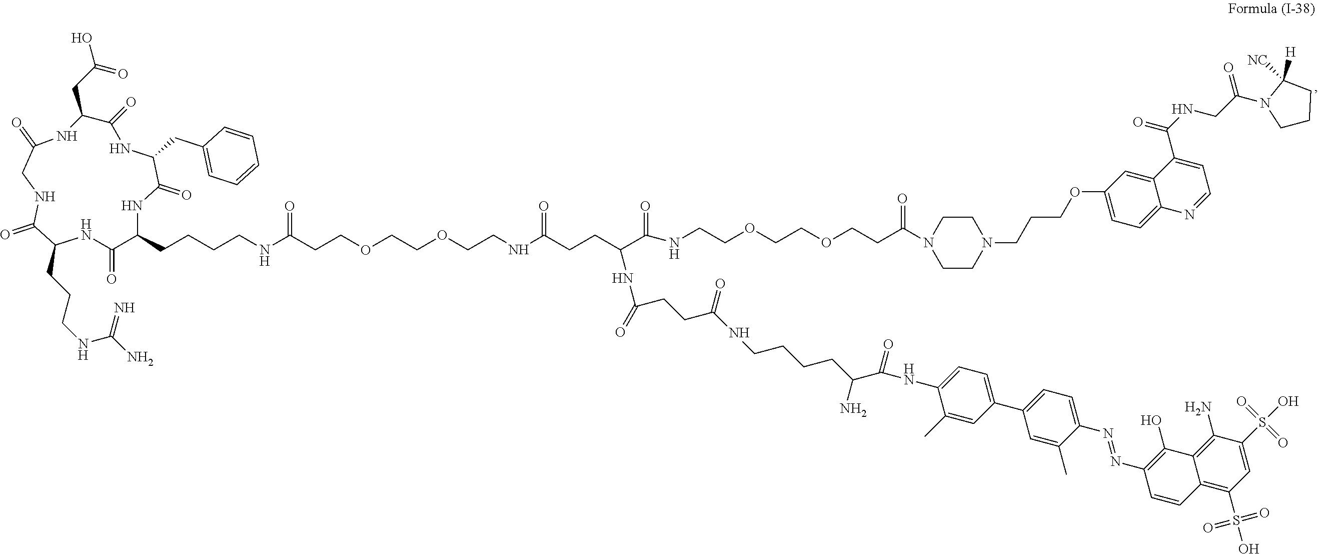 [SMILES: Cc1cc(-c2ccc(NC(=O)C(N)CCCCNC(=O)CCC(=O)NC(CCC(=O)NCCOCCOCCC(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)C(=O)NCCOCCOCCC(=O)N3CCN(CCCOc4ccc5nccc(C(=O)NCC(=O)N6CCC[C@H]6C#N)c5c4)CC3)c(C)c2)ccc1/N=N/c1ccc2c(S(=O)(=O)O)cc(S(=O)(=O)O)c(N)c2c1O]

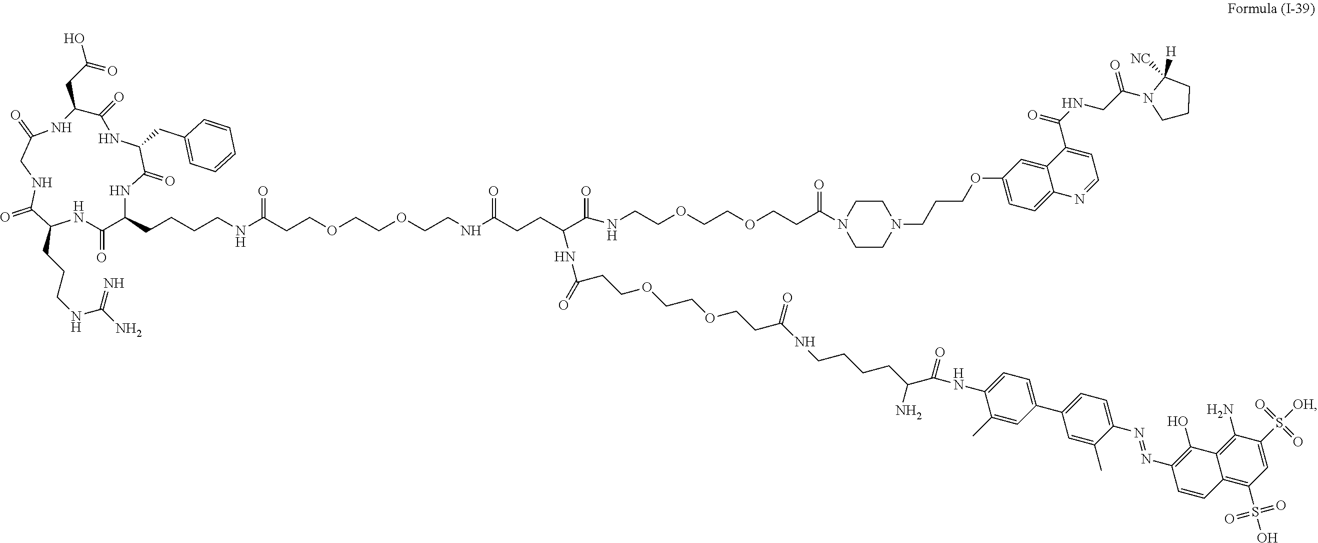 [SMILES: Cc1cc(-c2ccc(NC(=O)C(N)CCCCNC(=O)CCOCCOCCC(=O)NC(CCC(=O)NCCOCCOCCC(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)C(=O)NCCOCCOCCC(=O)N3CCN(CCCOc4ccc5nccc(C(=O)NCC(=O)N6CCC[C@H]6C#N)c5c4)CC3)c(C)c2)ccc1/N=N/c1ccc2c(S(=O)(=O)O)cc(S(=O)(=O)O)c(N)c2c1O]

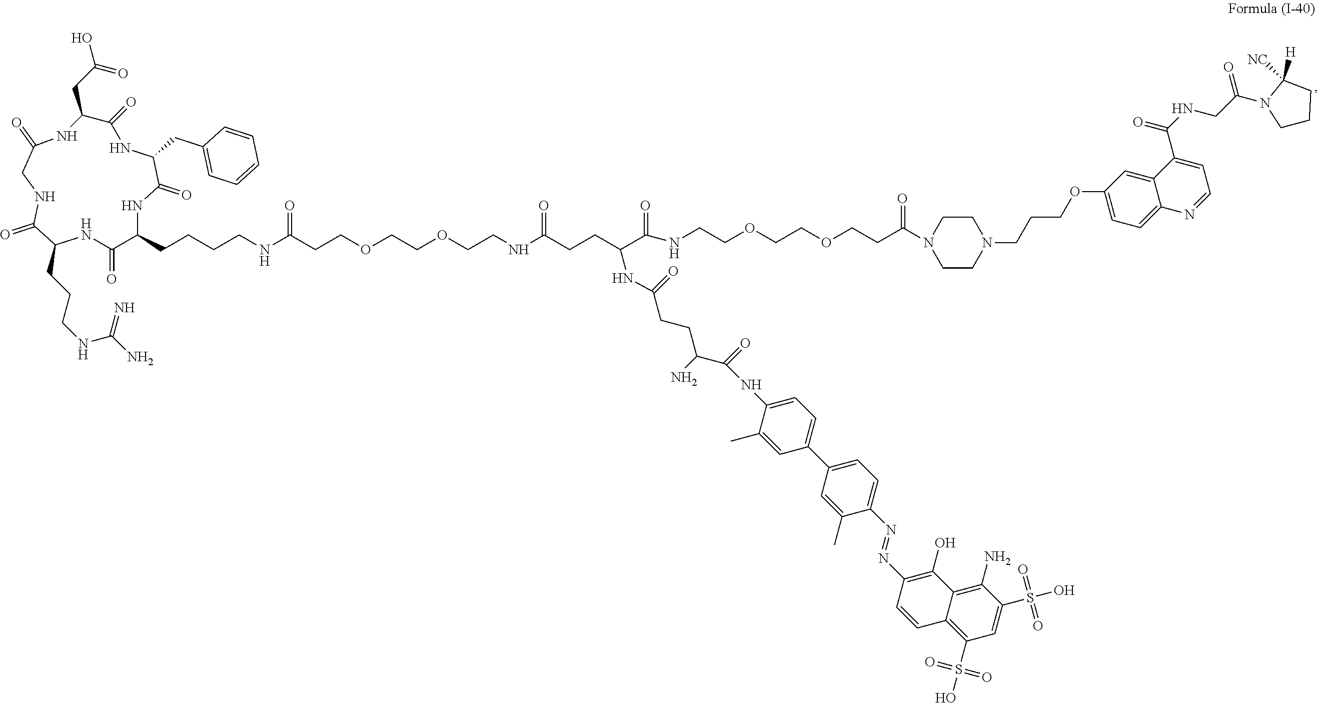 [SMILES: Cc1cc(-c2ccc(NC(=O)C(N)CCC(=O)NC(CCC(=O)NCCOCCOCCC(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)C(=O)NCCOCCOCCC(=O)N3CCN(CCCOc4ccc5nccc(C(=O)NCC(=O)N6CCC[C@H]6C#N)c5c4)CC3)c(C)c2)ccc1/N=N/c1ccc2c(S(=O)(=O)O)cc(S(=O)(=O)O)c(N)c2c1O]

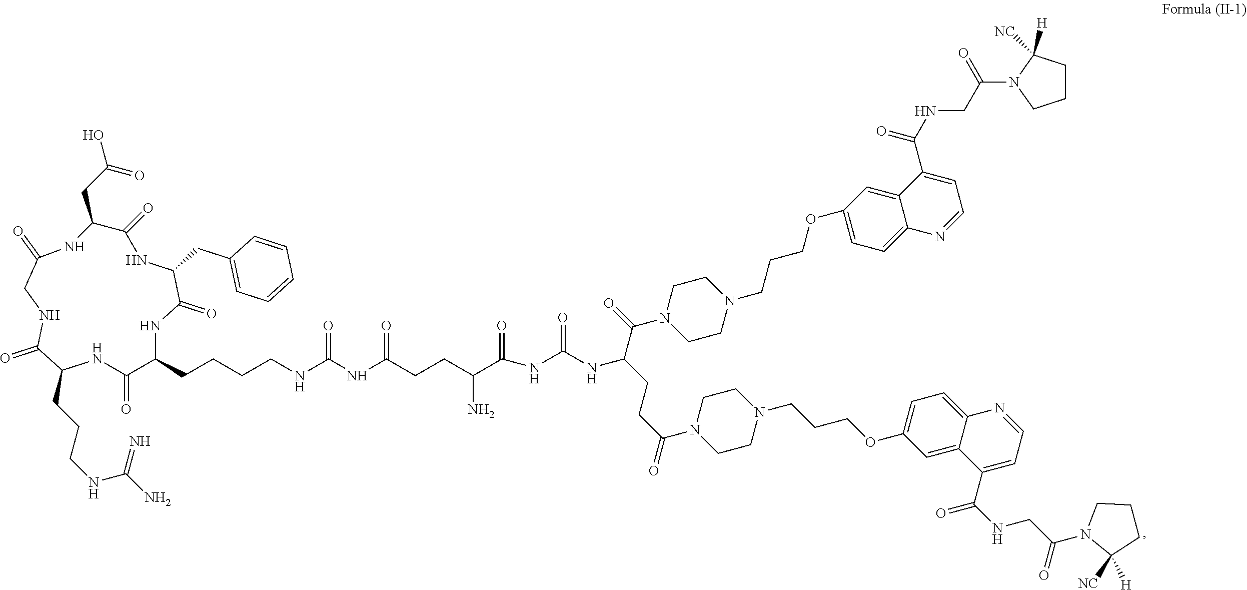 [SMILES: N#C[C@@H]1CCCN1C(=O)CNC(=O)c1ccnc2ccc(OCCCN3CCN(C(=O)CCC(NC(=O)NC(=O)C(N)CCC(=O)NC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)C(=O)N4CCN(CCCOc5ccc6nccc(C(=O)NCC(=O)N7CCC[C@H]7C#N)c6c5)CC4)CC3)cc12]

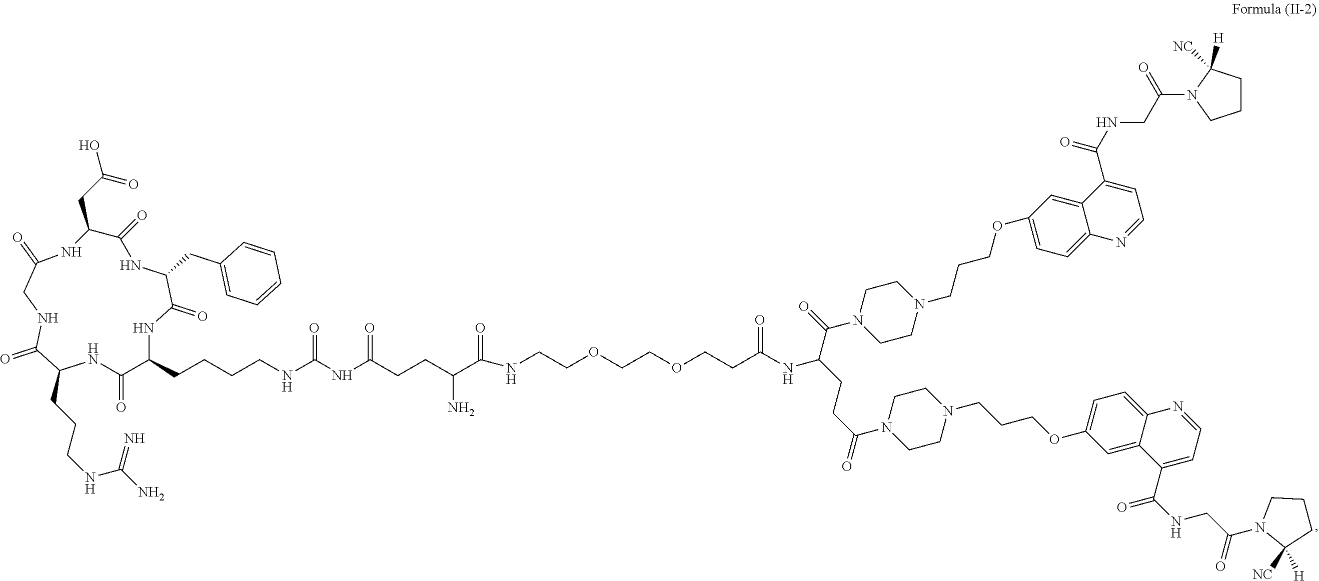 [SMILES: N#C[C@@H]1CCCN1C(=O)CNC(=O)c1ccnc2ccc(OCCCN3CCN(C(=O)CCC(NC(=O)CCOCCOCCNC(=O)C(N)CCC(=O)NC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)C(=O)N4CCN(CCCOc5ccc6nccc(C(=O)NCC(=O)N7CCC[C@H]7C#N)c6c5)CC4)CC3)cc12]

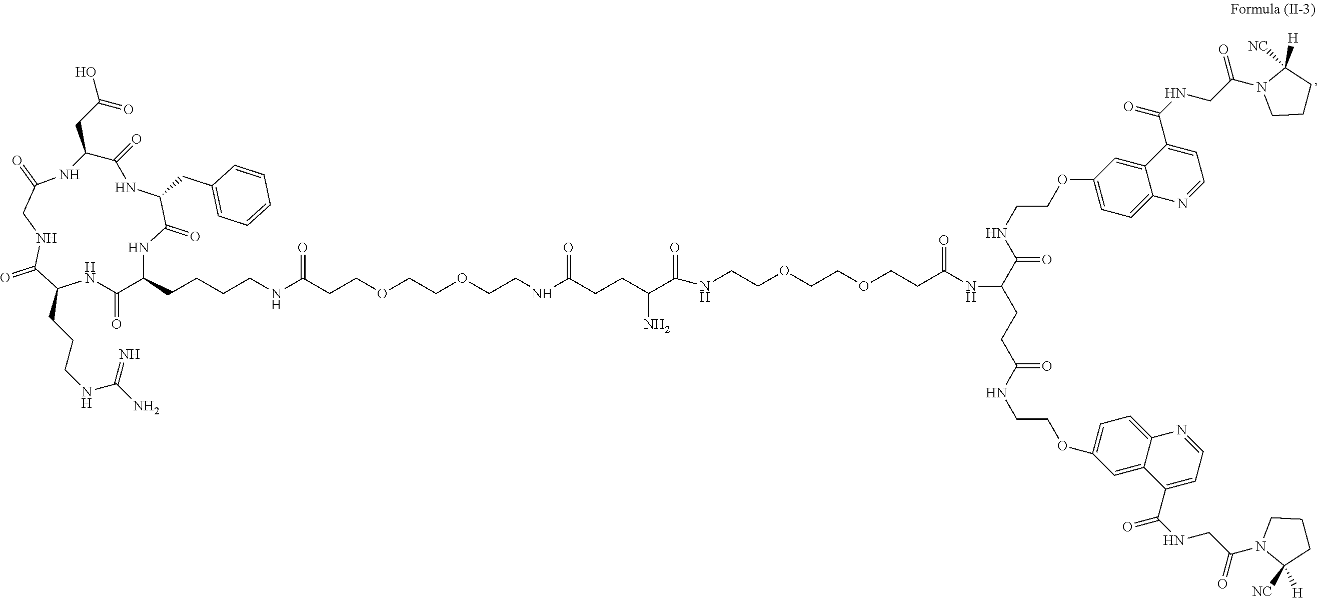 [SMILES: N#C[C@@H]1CCCN1C(=O)CNC(=O)c1ccnc2ccc(OCCNC(=O)CCC(NC(=O)CCOCCOCCNC(=O)C(N)CCC(=O)NCCOCCOCCC(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)C(=O)NCCOc3ccc4nccc(C(=O)NCC(=O)N5CCC[C@H]5C#N)c4c3)cc12]

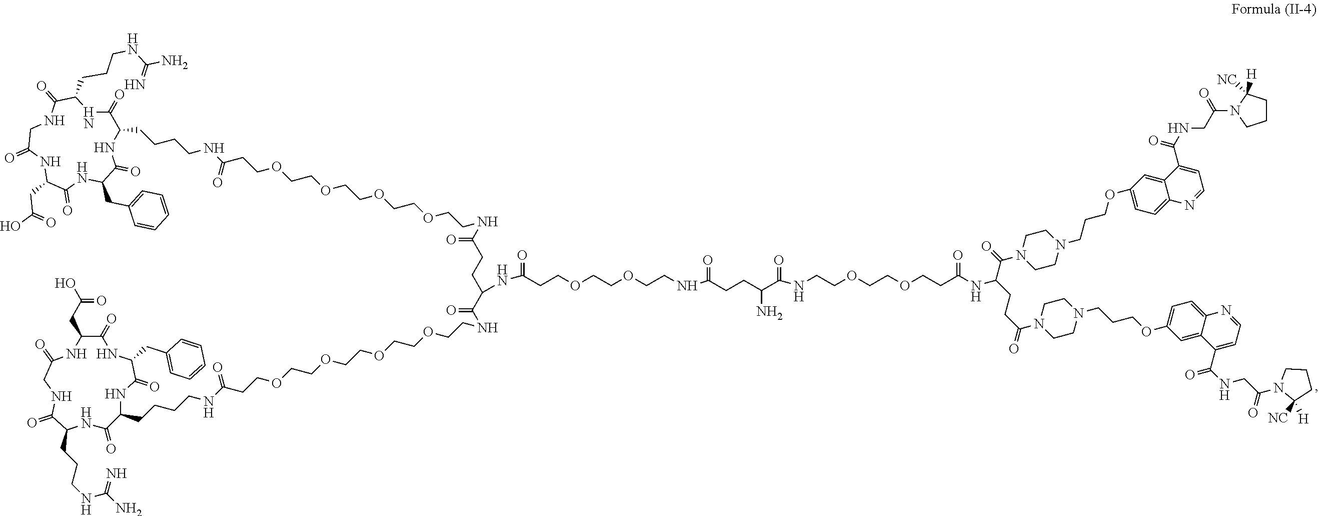 [SMILES: N#C[C@@H]1CCCN1C(=O)CNC(=O)c1ccnc2ccc(OCCCN3CCN(C(=O)CCC(NC(=O)CCOCCOCCNC(=O)C(N)CCC(=O)NCCOCCOCCC(=O)NC(CCC(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)C(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)C(=O)N4CCN(CCCOc5ccc6nccc(C(=O)NCC(=O)N7CCC[C@H]7C#N)c6c5)CC4)CC3)cc12]

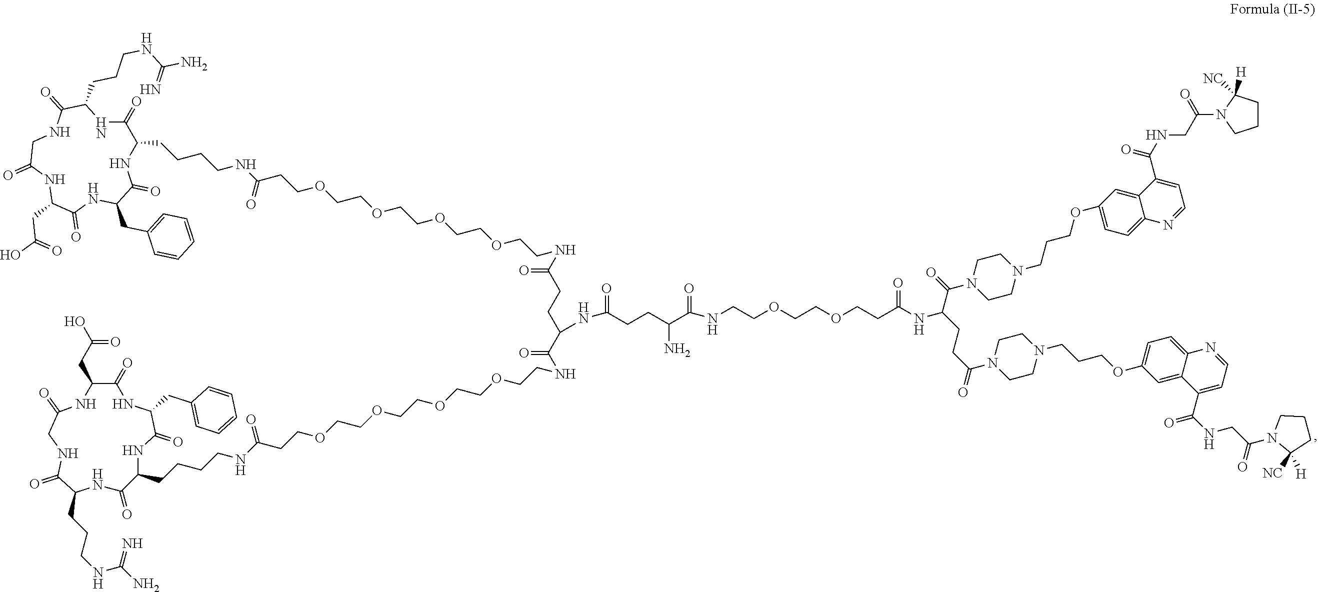 [SMILES: N#C[C@@H]1CCCN1C(=O)CNC(=O)c1ccnc2ccc(OCCCN3CCN(C(=O)CCC(NC(=O)CCOCCOCCNC(=O)C(N)CCC(=O)NC(CCC(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)C(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)C(=O)N4CCN(CCCOc5ccc6nccc(C(=O)NCC(=O)N7CCC[C@H]7C#N)c6c5)CC4)CC3)cc12]

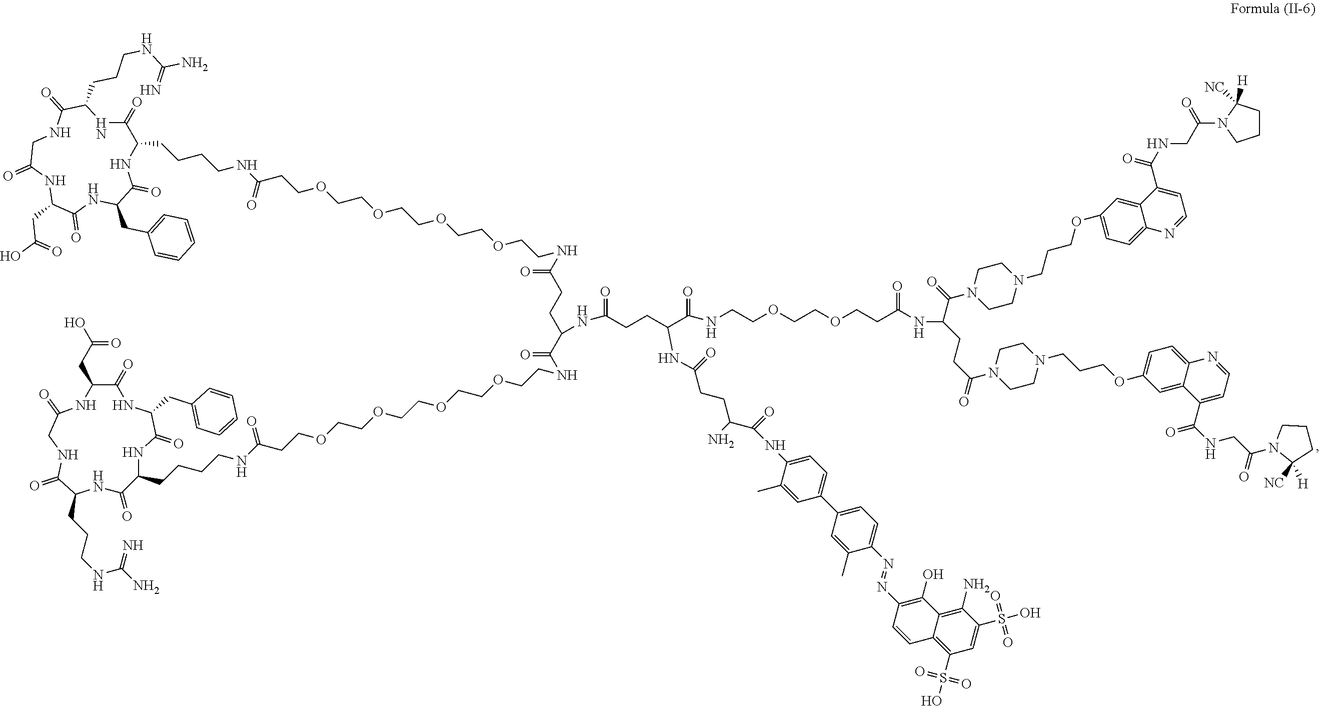 [SMILES: Cc1cc(-c2ccc(NC(=O)C(N)CCC(=O)NC(CCC(=O)NC(CCC(=O)NCCOCCOCCOCCOCCC(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)C(=O)NCCOCCOCCOCCOCCC(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)C(=O)NCCOCCOCCC(=O)NC(CCC(=O)N3CCN(CCCOc4ccc5nccc(C(=O)NCC(=O)N6CCC[C@H]6C#N)c5c4)CC3)C(=O)N3CCN(CCCOc4ccc5nccc(C(=O)NCC(=O)N6CCC[C@H]6C#N)c5c4)CC3)c(C)c2)ccc1/N=N/c1ccc2c(S(=O)(=O)O)cc(S(=O)(=O)O)c(N)c2c1O]

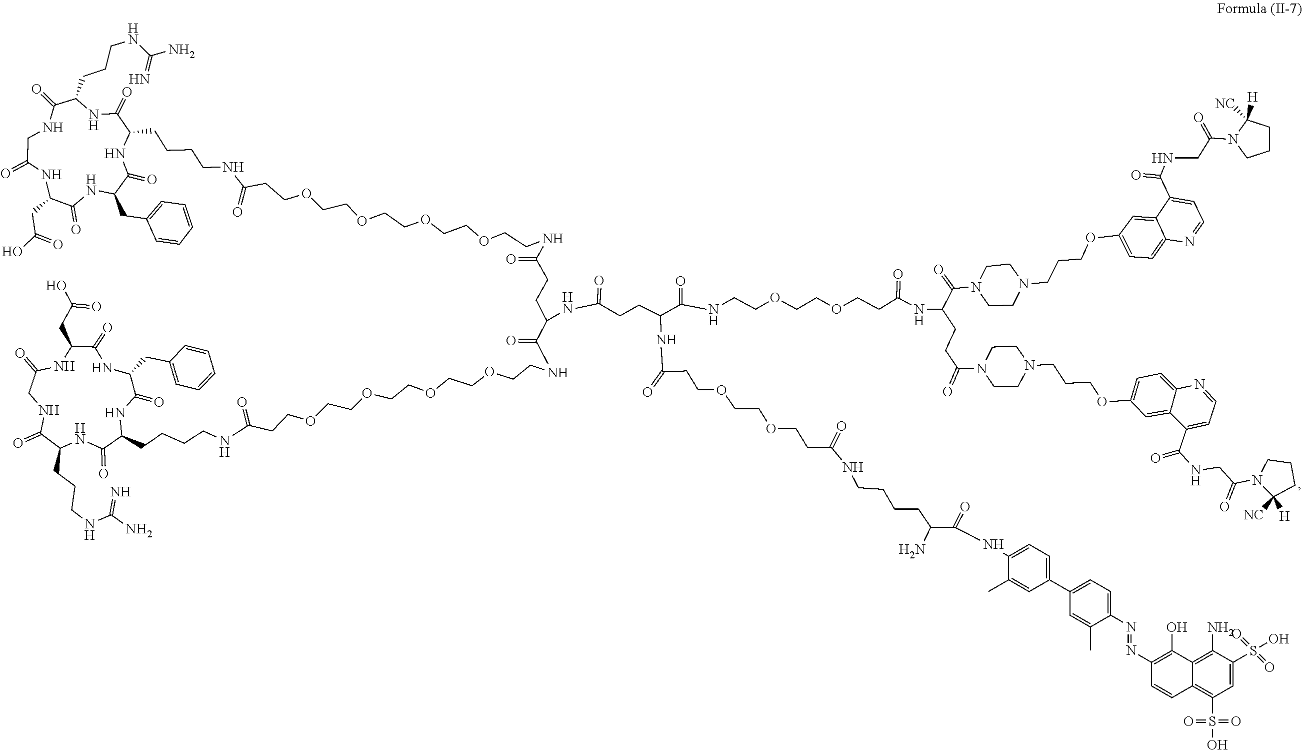 [SMILES: Cc1cc(-c2ccc(NC(=O)C(N)CCCCNC(=O)CCOCCOCCC(=O)NC(CCC(=O)NC(CCC(=O)NCCOCCOCCOCCOCCC(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)C(=O)NCCOCCOCCOCCOCCC(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)C(=O)NCCOCCOCCC(=O)NC(CCC(=O)N3CCN(CCCOc4ccc5nccc(C(=O)NCC(=O)N6CCC[C@@H]6C#N)c5c4)CC3)C(=O)N3CCN(CCCOc4ccc5nccc(C(=O)NCC(=O)N6CCC[C@H]6C#N)c5c4)CC3)c(C)c2)ccc1/N=N/c1ccc2c(S(=O)(=O)O)cc(S(=O)(=O)O)c(N)c2c1O]

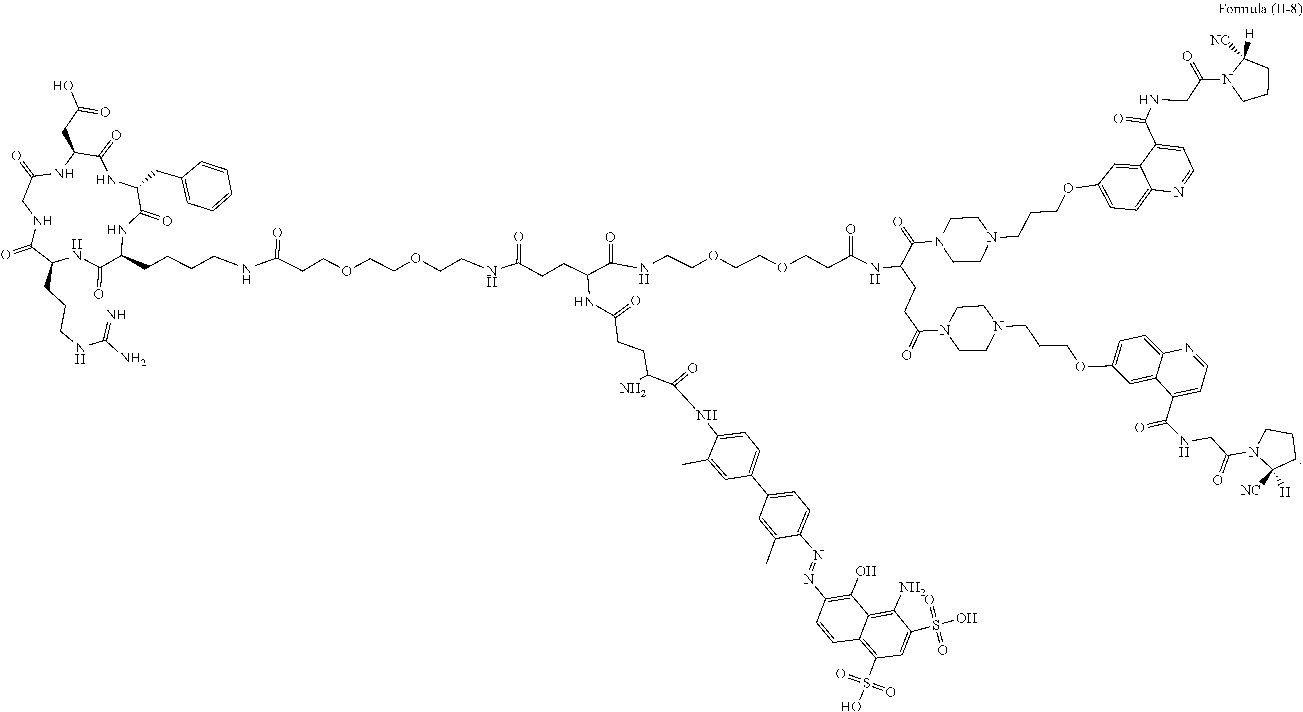 [SMILES: Cc1cc(-c2ccc(NC(=O)C(N)CCC(=O)NC(CCC(=O)NCCOCCOCCC(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)C(=O)NCCOCCOCCC(=O)NC(CCC(=O)N3CCN(CCCOc4ccc5nccc(C(=O)NCC(=O)N6CCC[C@H]6C#N)c5c4)CC3)C(=O)N3CCN(CCCOc4ccc5nccc(C(=O)NCC(=O)N6CCC[C@H]6C#N)c5c4)CC3)c(C)c2)ccc1/N=N/c1ccc2c(S(=O)(=O)O)cc(S(=O)(=O)O)c(N)c2c1O]

In a second aspect, the present invention further provides a compound capable of being labeled with a radionuclide based on the dual-targeting compound according to any one of the above descriptions, where the compound capable of being labeled with a radionuclide is formed by connecting amino in any one of the Z, Q or V structures of Formula (I) or Formula (II) according to any one of the above descriptions with a nuclide chelating group, and the compound capable of being labeled with a radionuclide has a general formula shown in Formula (V) or Formula (VI) below:

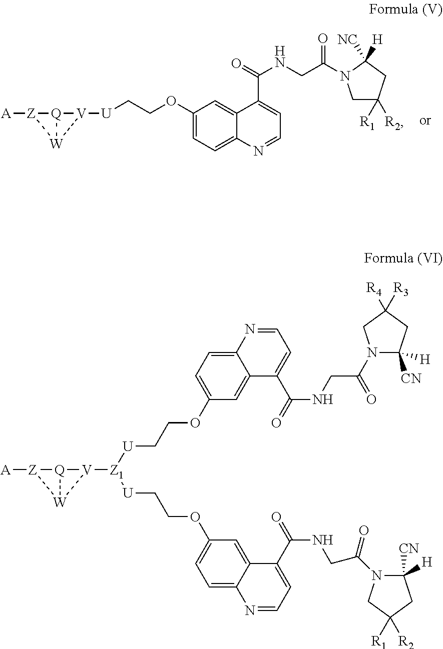

where A, Z, Q, V, U, R₁ and R₂ in Formula (V) are defined the same as the A, the Z, the Q, the V, the U, the R₁ and the R₂ in Formula (I); and A, Z, Q, V, U, Z₁, R₁, R₂, R₃ and R₄ in Formula (VI) are defined the same as the A, the Z, the Q, the V, the U, the Z₁, the R₁, the R₂, the R₃ and the R₄ in Formula (II).
W is a fragment with a nuclide chelating group, and has a structure derived from any one of 1,4,7,10-tetraazacyclododecane-N,N′,N,N′-tetraacetic acid (DOTA), ethylenediamine tetraacetic acid (EDTA), 1,4,7-triazacyclononane-1,4,7-triacetic acid (NOTA), triethylenetetraamine (TETA), iminodiacetic acid, diethylenetriamine-N,N,N′,N′,N″-pentaacetic acid (DTPA), bis(carboxymethyl)imidazole glycinate or 6-hydrazinopyridine-3-carboxylic acid (HYNIC), or has any one of the following structures:

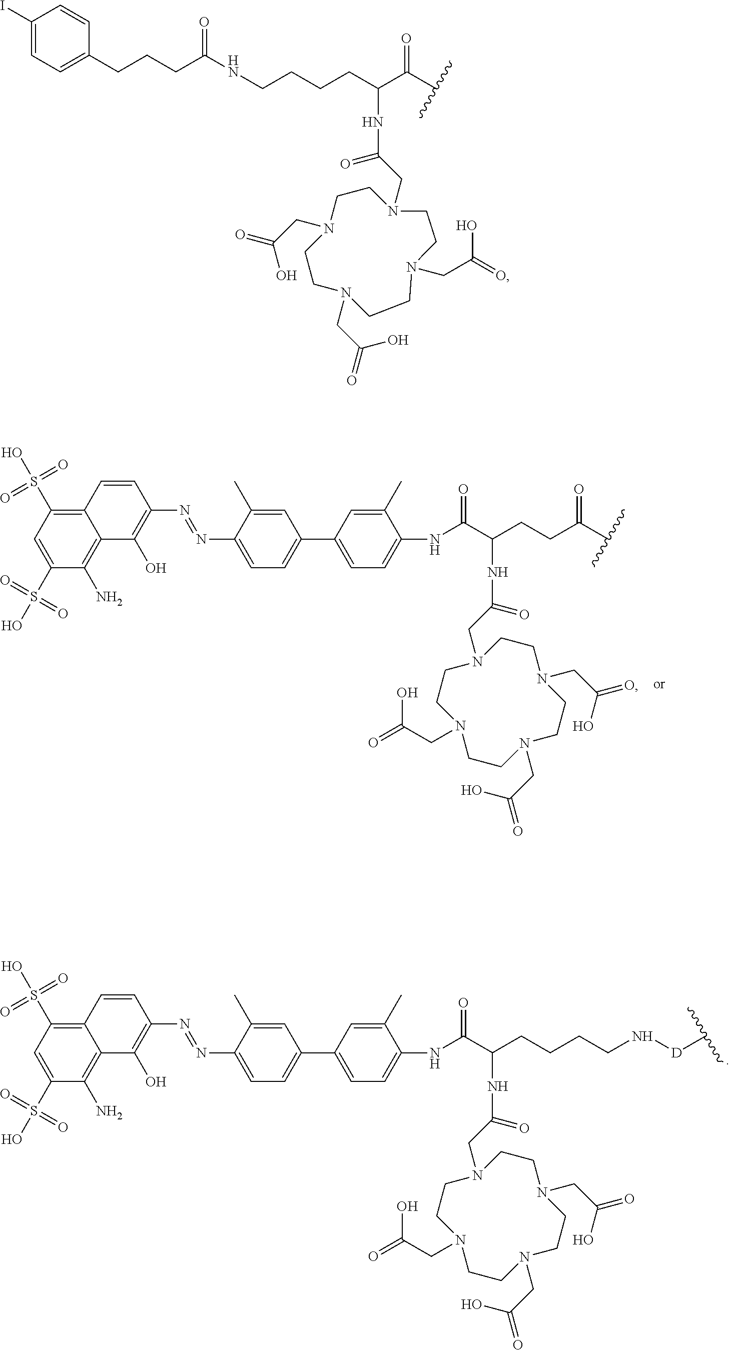

Further, D according to any one of the above descriptions is a substitution structure based on —(CH₂)ₚ—, each —CH₂— is separately substituted with or without —O—, —NH—, —(CO)—, —NH—(CO)—, —CH(NH₂)— or —(CO)—NH—, substitution occurs when no two adjacent —CH₂— groups are substituted, and p is an integer selected from 0 to 30 (such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29 and 30).

In some preferred embodiments, the D according to any one of the above descriptions is selected from —(CO)—CH₂—CH₂—(CO)—, —(CO)—CH₂—(CH₂—O—CH₂)₂—CH₂—(CO)— or —(CH₂)₀— (with reference to structures of Formula (V-38) to Formula (V-40)).

In some preferred embodiments, the W is selected from the following structures:

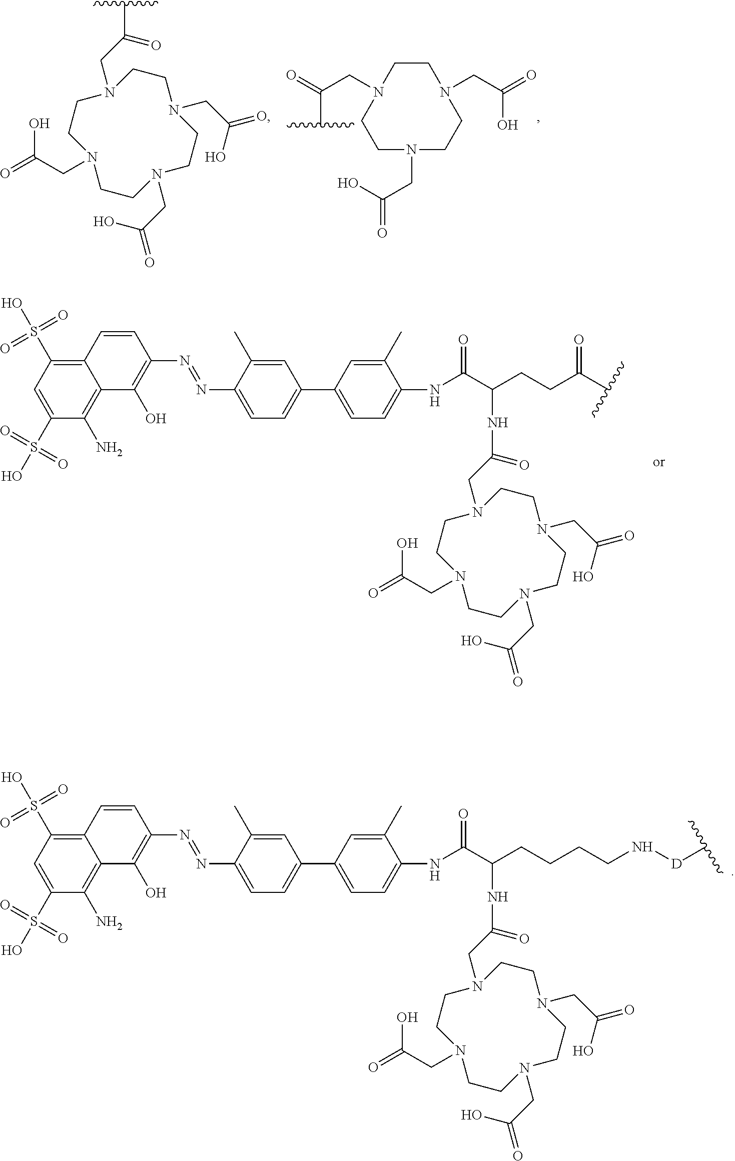

More preferably, the W is selected from the following structures:

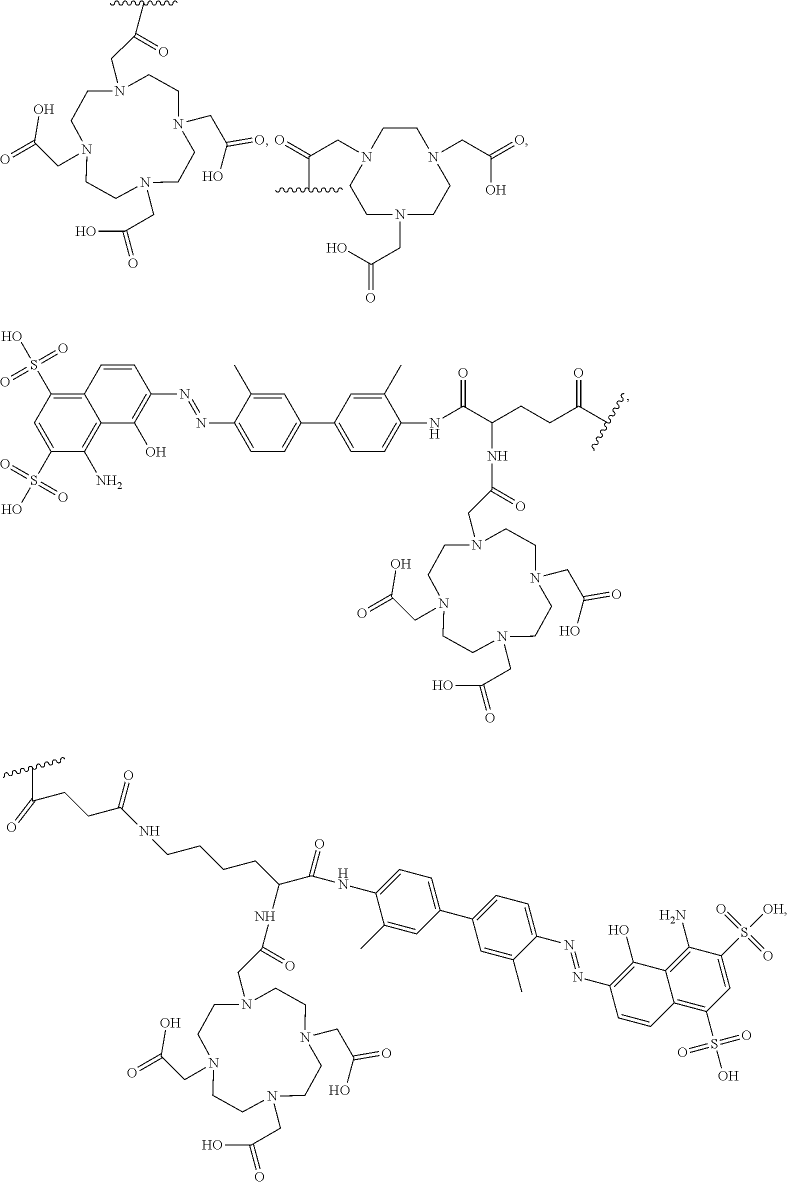

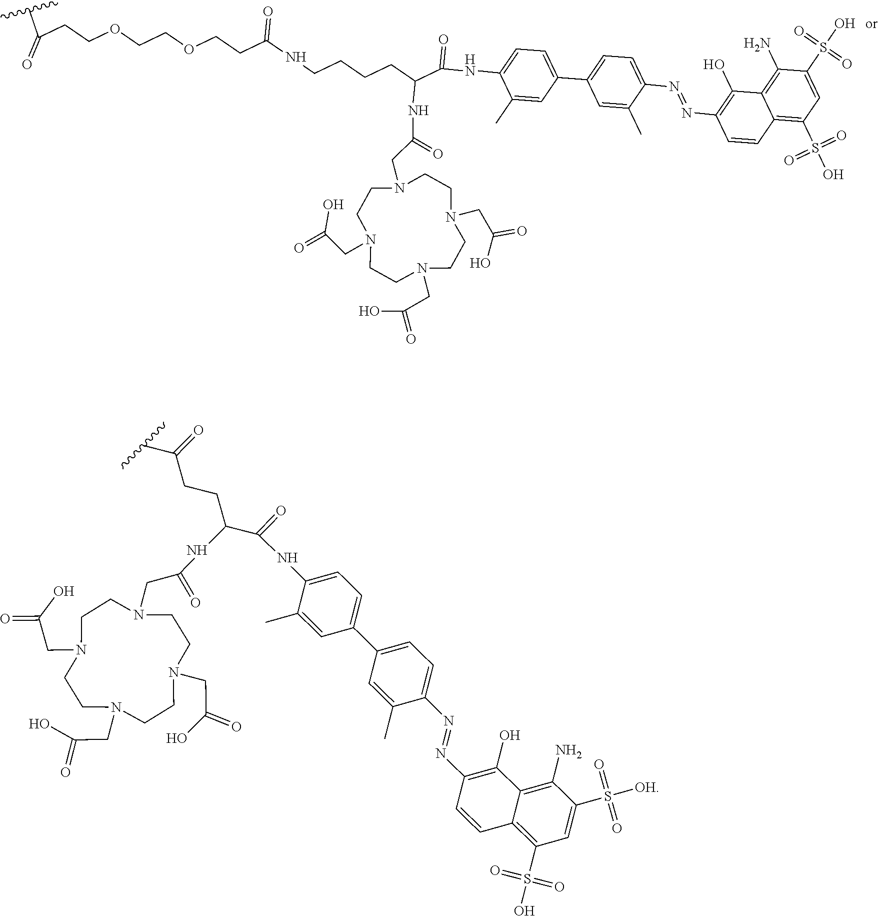 [SMILES: *C(=O)CCOCCOCCC(=O)NCCCCC(NC(=O)CN1CCN(CC(=O)O)CCN(CC(=O)O)CCN(CC(=O)O)CC1)C(=O)Nc1ccc(-c2ccc(/N=N/c3ccc4c(S(=O)(=O)O)cc(S(=O)(=O)O)c(N)c4c3O)c(C)c2)cc1C ; *C(=O)CCC(NC(=O)CN1CCN(CC(=O)O)CCN(CC(=O)O)CCN(CC(=O)O)CC1)C(=O)Nc1ccc(-c2ccc(/N=N/c3ccc4c(S(=O)(=O)O)cc(S(=O)(=O)O)c(N)c4c3O)c(C)c2)cc1C]

In furthermore preferred embodiments, the compound capable of being labeled with a radionuclide is selected from the following structures:

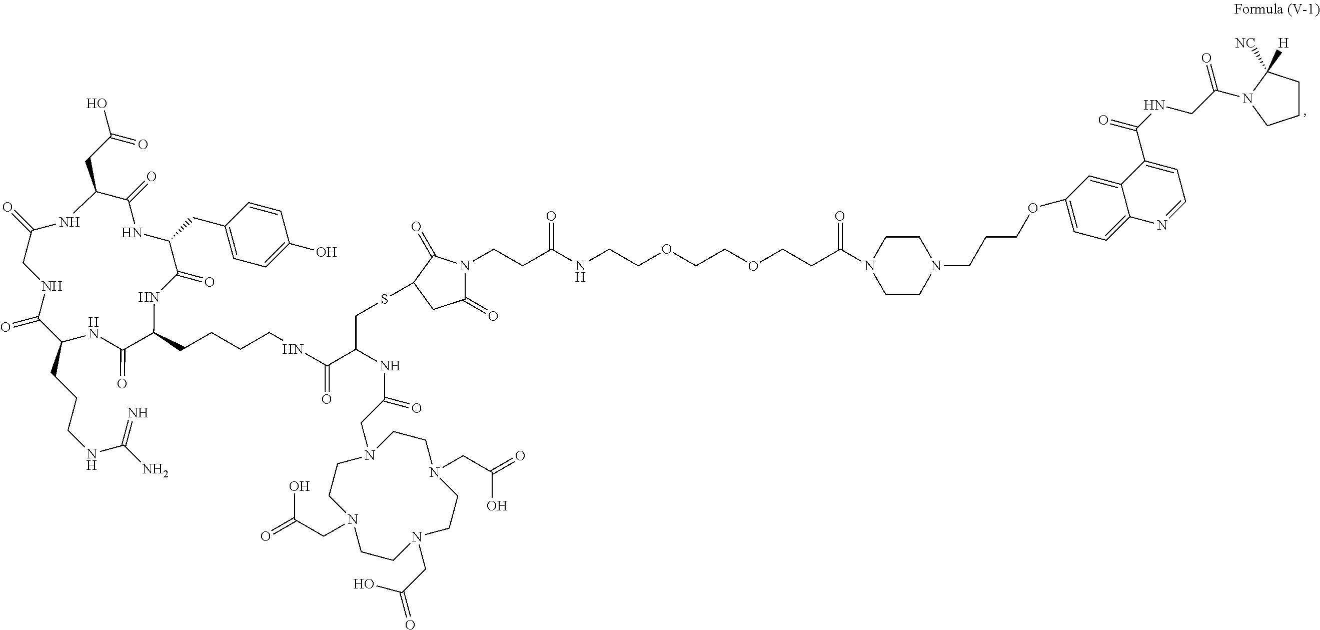 [SMILES: N#C[C@@H]1CCCN1C(=O)CNC(=O)c1ccnc2ccc(OCCCN3CCN(C(=O)CCOCCOCCNC(=O)CCN4C(=O)CC(SCC(NC(=O)CN5CCN(CC(=O)O)CCN(CC(=O)O)CCN(CC(=O)O)CC5)C(=O)NCCCC[C@@H]5NC(=O)[C@@H](Cc6ccc(O)cc6)NC(=O)[C@H](CC(=O)O)NC(=O)CNC(=O)[C@H](CCCNC(=N)N)NC5=O)C4=O)CC3)cc12]

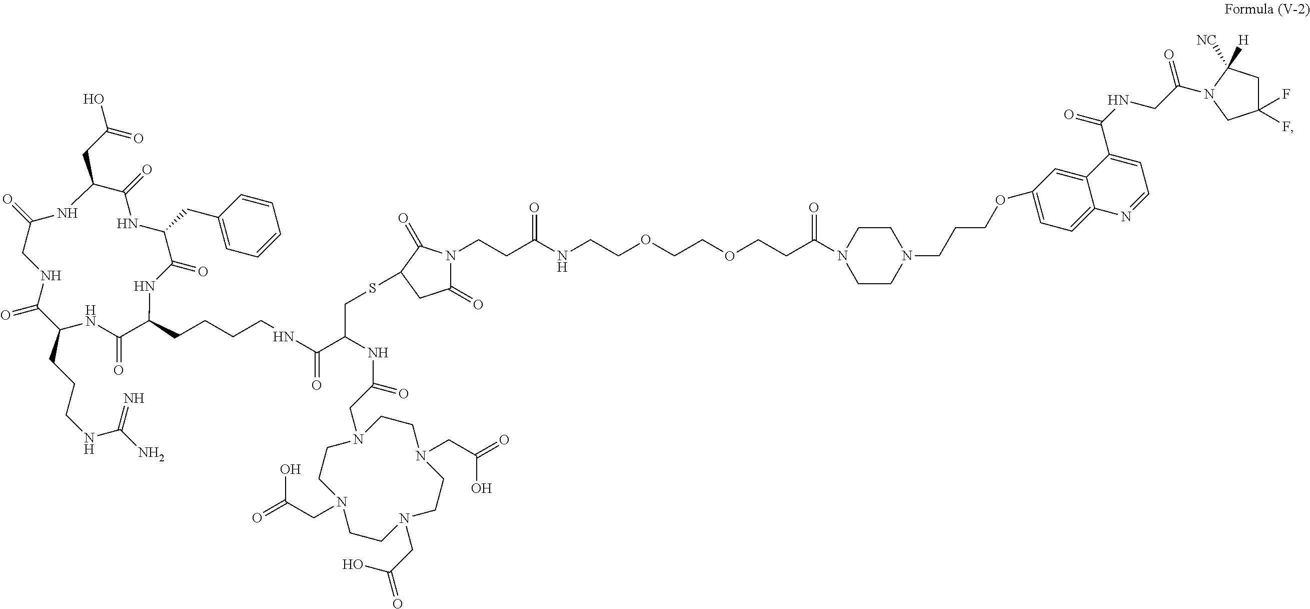 [SMILES: N#C[C@@H]1CC(F)(F)CN1C(=O)CNC(=O)c1ccnc2ccc(OCCCN3CCN(C(=O)CCOCCOCCNC(=O)CCN4C(=O)CC(SCC(NC(=O)CN5CCN(CC(=O)O)CCN(CC(=O)O)CCN(CC(=O)O)CC5)C(=O)NCCCC[C@@H]5NC(=O)[C@@H](Cc6ccccc6)NC(=O)[C@H](CC(=O)O)NC(=O)CNC(=O)[C@H](CCCNC(=N)N)NC5=O)C4=O)CC3)cc12]

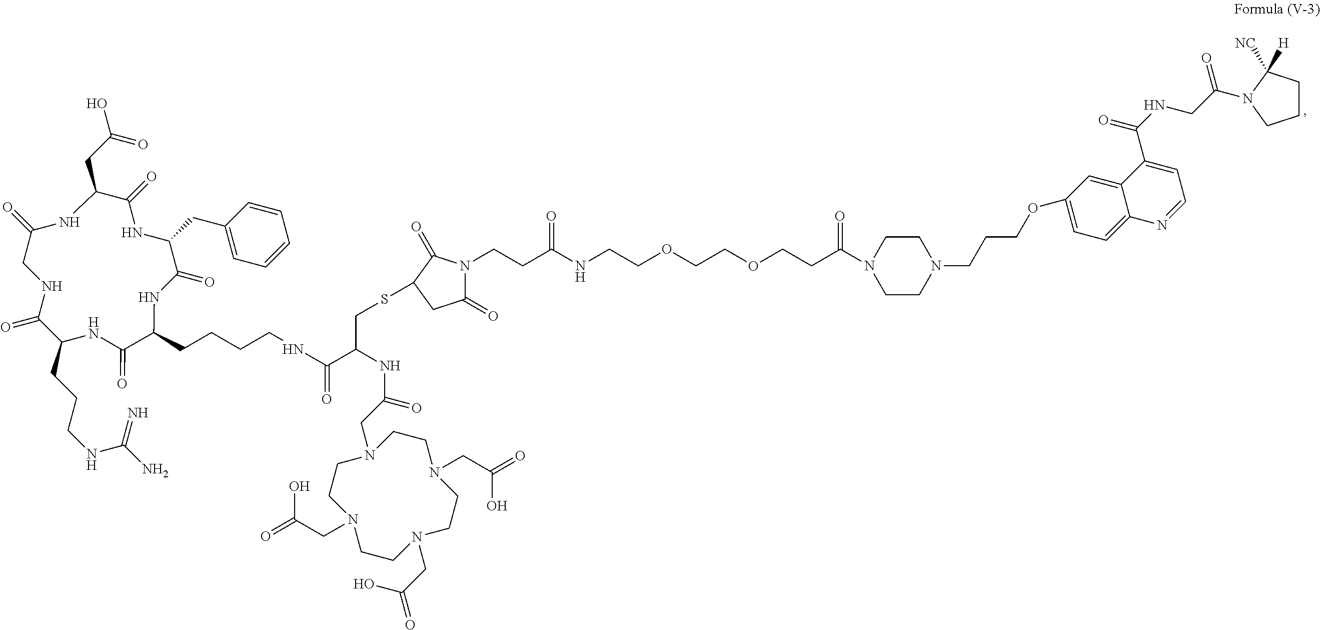 [SMILES: N#C[C@@H]1CCCN1C(=O)CNC(=O)c1ccnc2ccc(OCCCN3CCN(C(=O)CCOCCOCCNC(=O)CCN4C(=O)CC(SCC(NC(=O)CN5CCN(CC(=O)O)CCN(CC(=O)O)CCN(CC(=O)O)CC5)C(=O)NCCCC[C@@H]5NC(=O)[C@@H](Cc6ccccc6)NC(=O)[C@H](CC(=O)O)NC(=O)CNC(=O)[C@H](CCCNC(=N)N)NC5=O)C4=O)CC3)cc12]

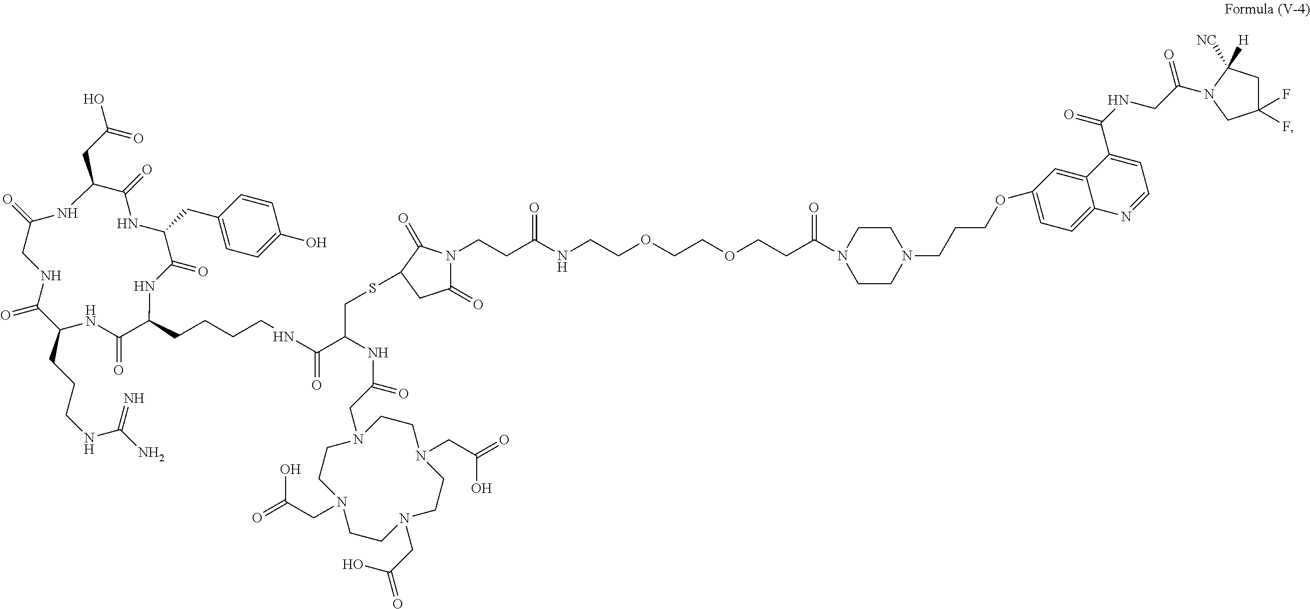 [SMILES: N#C[C@@H]1CC(F)(F)CN1C(=O)CNC(=O)c1ccnc2ccc(OCCCN3CCN(C(=O)CCOCCOCCNC(=O)CCN4C(=O)CC(SCC(NC(=O)CN5CCN(CC(=O)O)CCN(CC(=O)O)CCN(CC(=O)O)CC5)C(=O)NCCCC[C@@H]5NC(=O)[C@@H](Cc6ccc(O)cc6)NC(=O)[C@H](CC(=O)O)NC(=O)CNC(=O)[C@H](CCCNC(=N)N)NC5=O)C4=O)CC3)cc12]

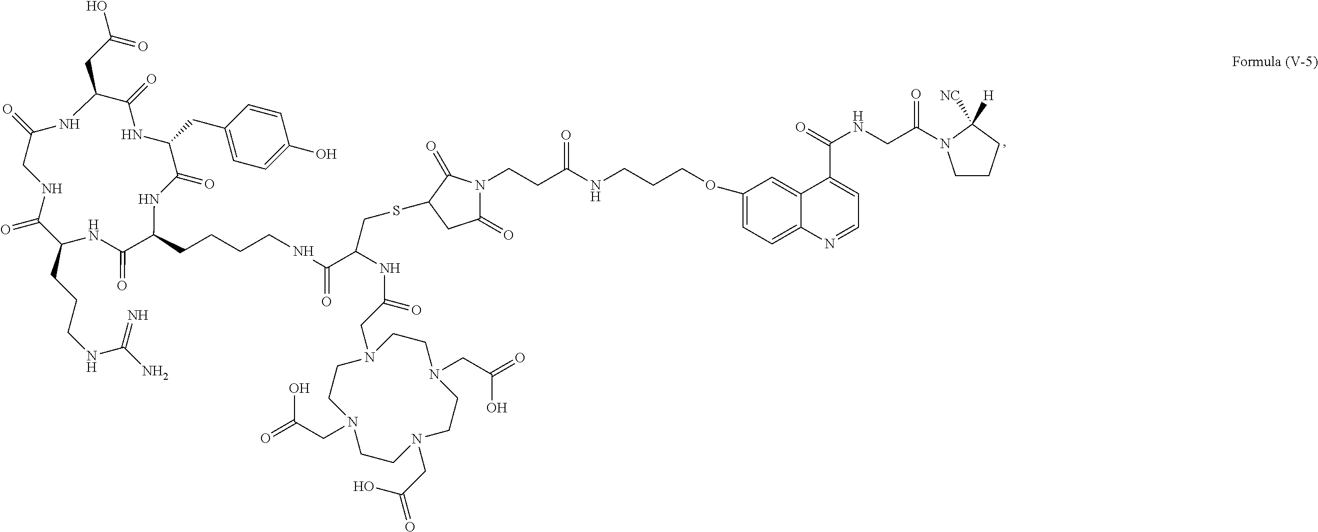 [SMILES: N#C[C@@H]1CCCN1C(=O)CNC(=O)c1ccnc2ccc(OCCCNC(=O)CCN3C(=O)CC(SCC(NC(=O)CN4CCN(CC(=O)O)CCN(CC(=O)O)CCN(CC(=O)O)CC4)C(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)C3=O)cc12]

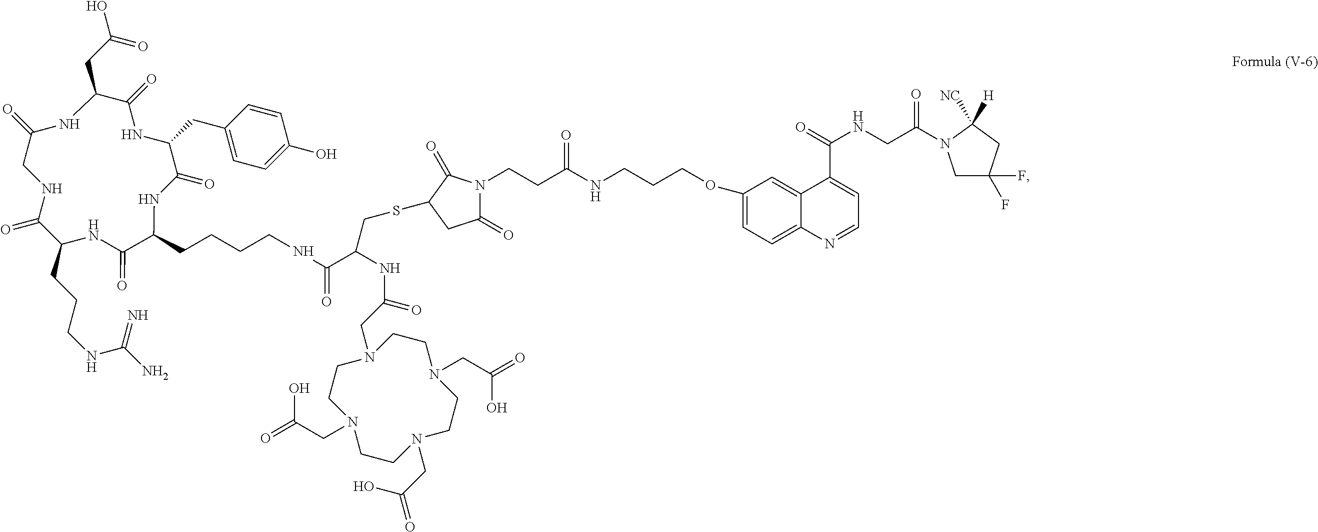 [SMILES: N#C[C@@H]1CC(F)(F)CN1C(=O)CNC(=O)c1ccnc2ccc(OCCCNC(=O)CCN3C(=O)CC(SCC(NC(=O)CN4CCN(CC(=O)O)CCN(CC(=O)O)CCN(CC(=O)O)CC4)C(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)C3=O)cc12]

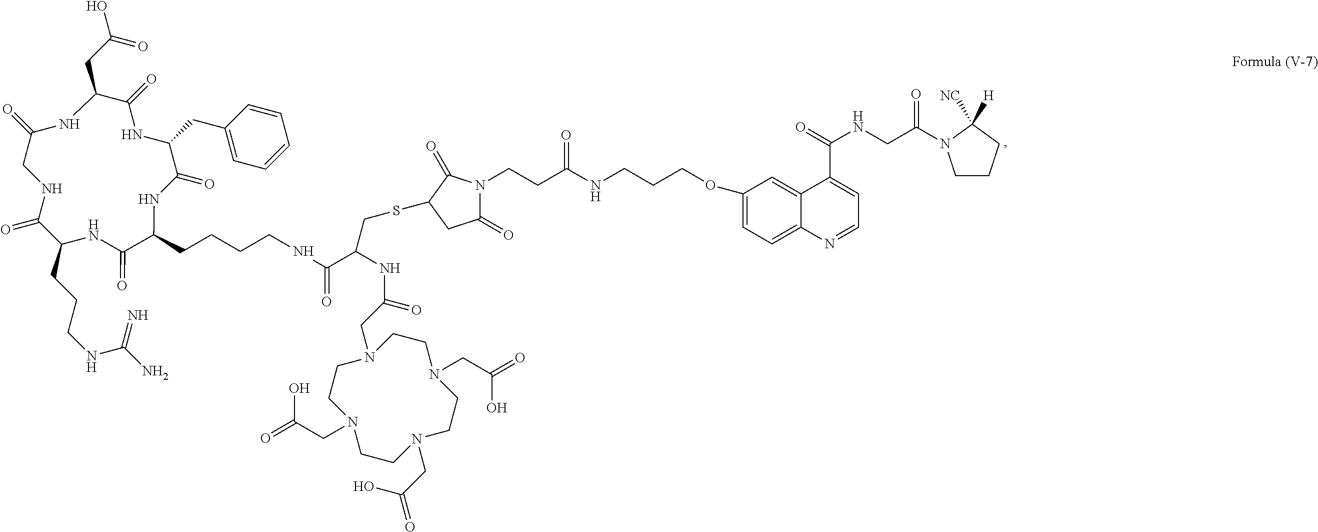 [SMILES: N#C[C@@H]1CCCN1C(=O)CNC(=O)c1ccnc2ccc(OCCCNC(=O)CCN3C(=O)CC(SCC(NC(=O)CN4CCN(CC(=O)O)CCN(CC(=O)O)CCN(CC(=O)O)CC4)C(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)C3=O)cc12]

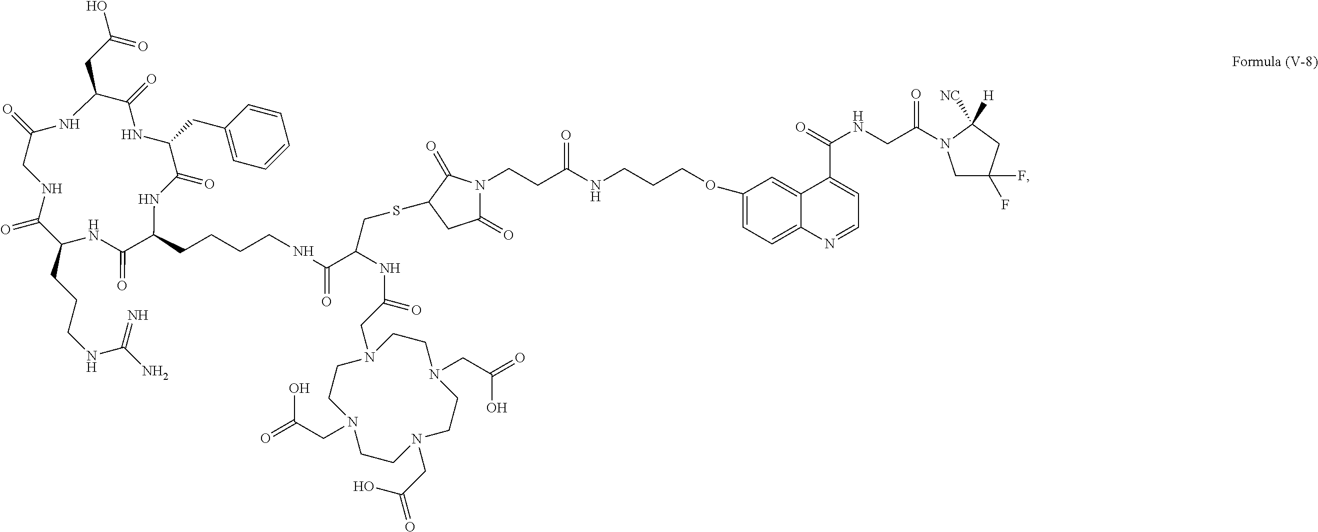 [SMILES: N#C[C@@H]1CC(F)(F)CN1C(=O)CNC(=O)c1ccnc2ccc(OCCCNC(=O)CCN3C(=O)CC(SCC(NC(=O)CN4CCN(CC(=O)O)CCN(CC(=O)O)CCN(CC(=O)O)CC4)C(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)C3=O)cc12]

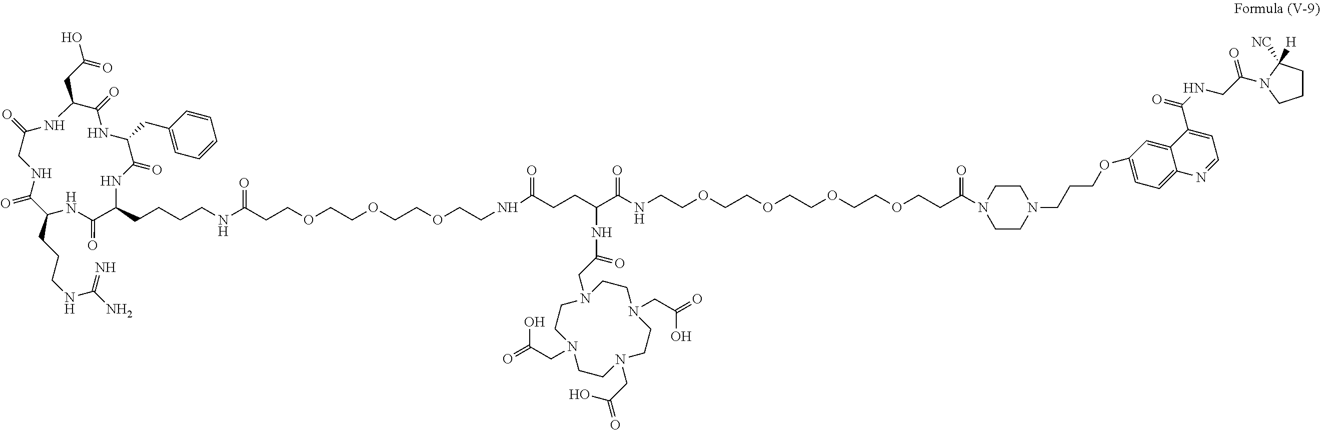 [SMILES: N#C[C@@H]1CCCN1C(=O)CNC(=O)c1ccnc2ccc(OCCCN3CCN(C(=O)CCOCCOCCOCCOCCNC(=O)C(CCC(=O)NCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

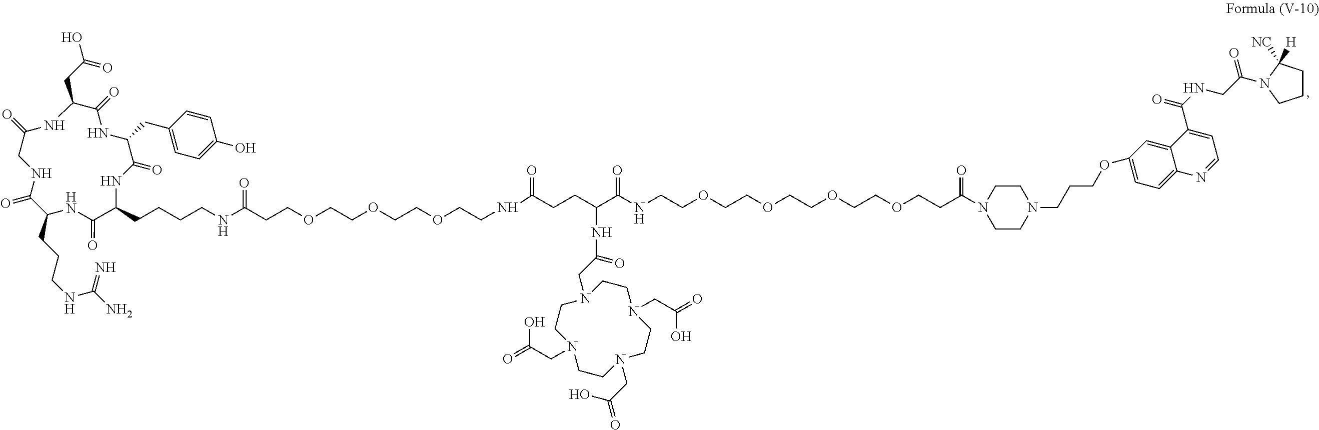 [SMILES: N#C[C@@H]1CCCN1C(=O)CNC(=O)c1ccnc2ccc(OCCCN3CCN(C(=O)CCOCCOCCOCCOCCNC(=O)C(CCC(=O)NCCOCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

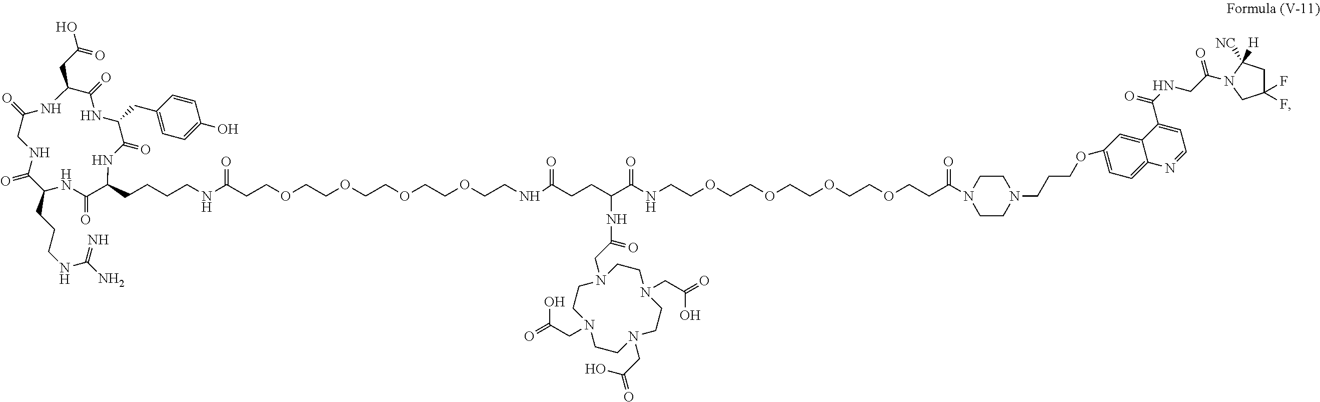 [SMILES: N#C[C@@H]1CC(F)(F)CN1C(=O)CNC(=O)c1ccnc2ccc(OCCCN3CCN(C(=O)CCOCCOCCOCCOCCNC(=O)C(CCC(=O)NCCOCCOCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

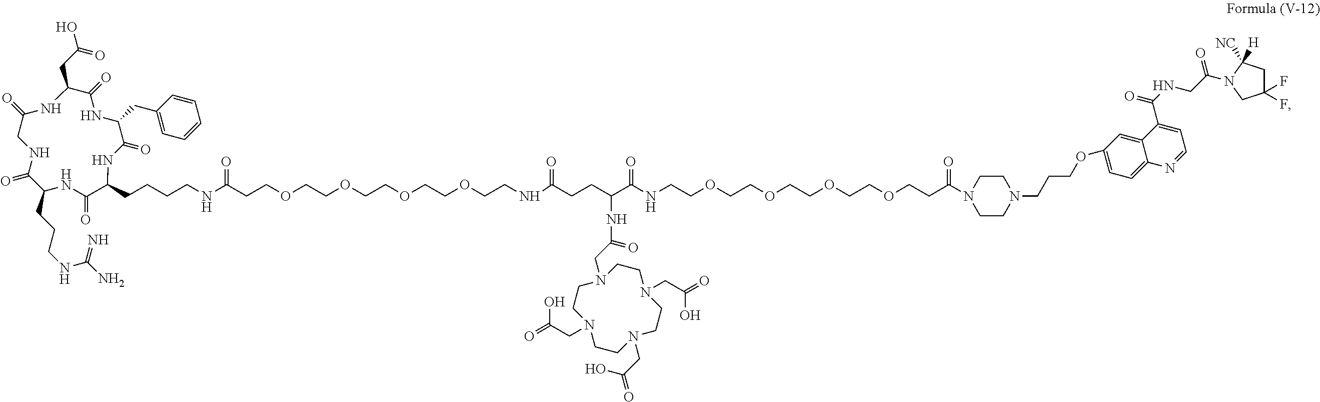 [SMILES: N#C[C@@H]1CC(F)(F)CN1C(=O)CNC(=O)c1ccnc2ccc(OCCCN3CCN(C(=O)CCOCCOCCOCCOCCNC(=O)C(CCC(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

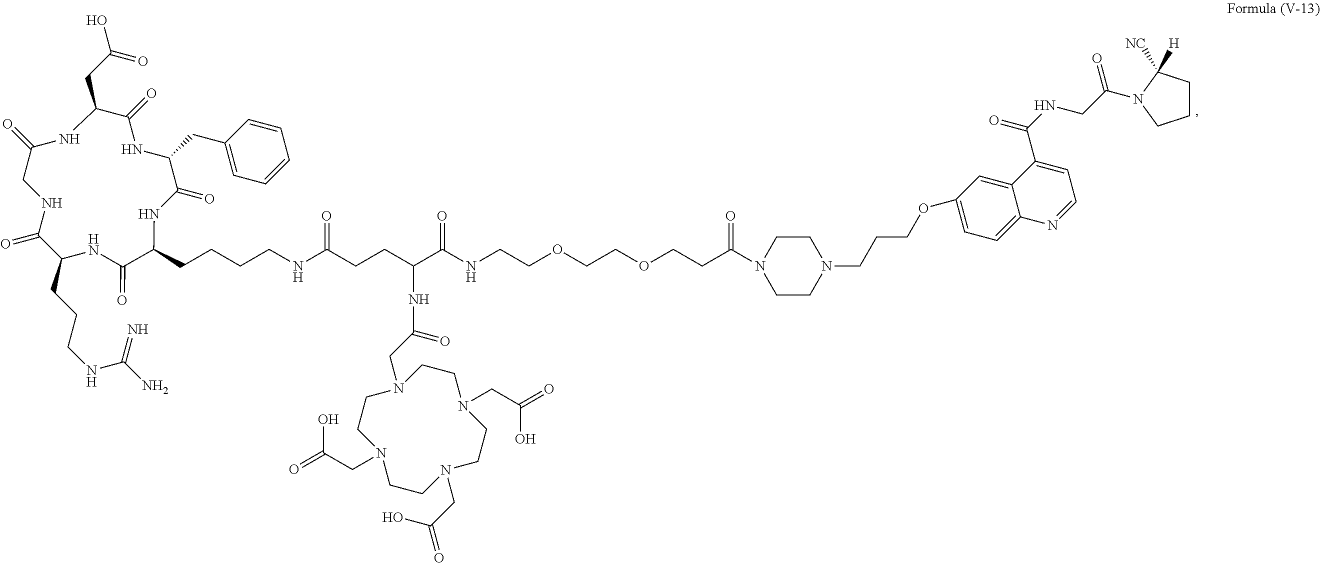 [SMILES: N#C[C@@H]1CCCN1C(=O)CNC(=O)c1ccnc2ccc(OCCCN3CCN(C(=O)CCOCCOCCNC(=O)C(CCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

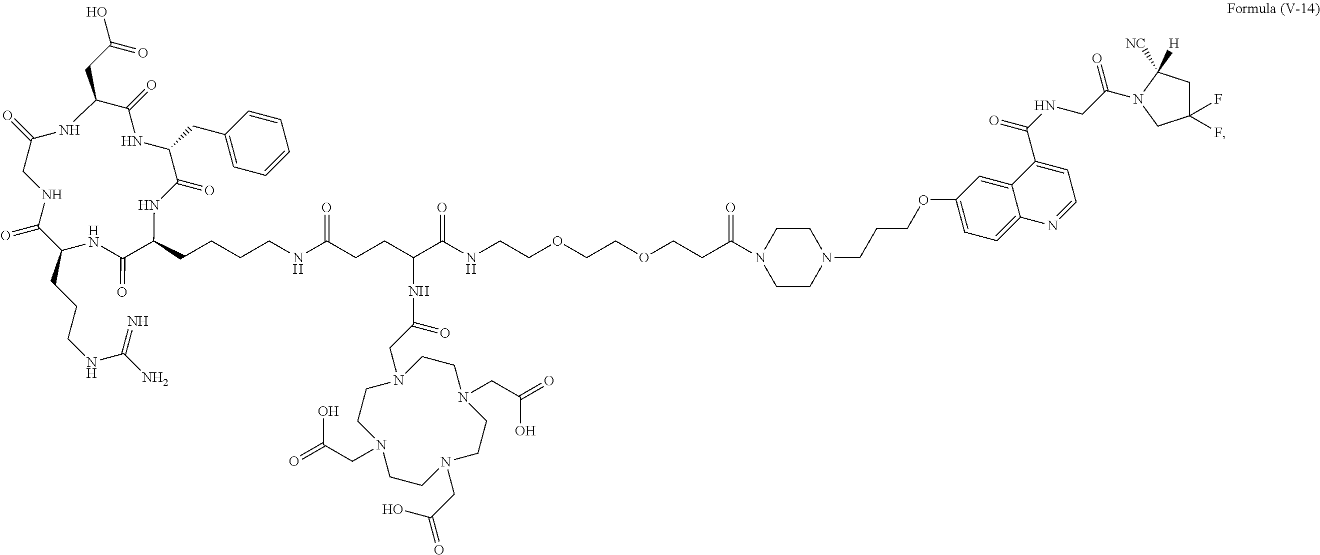 [SMILES: N#C[C@@H]1CC(F)(F)CN1C(=O)CNC(=O)c1ccnc2ccc(OCCCN3CCN(C(=O)CCOCCOCCNC(=O)C(CCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

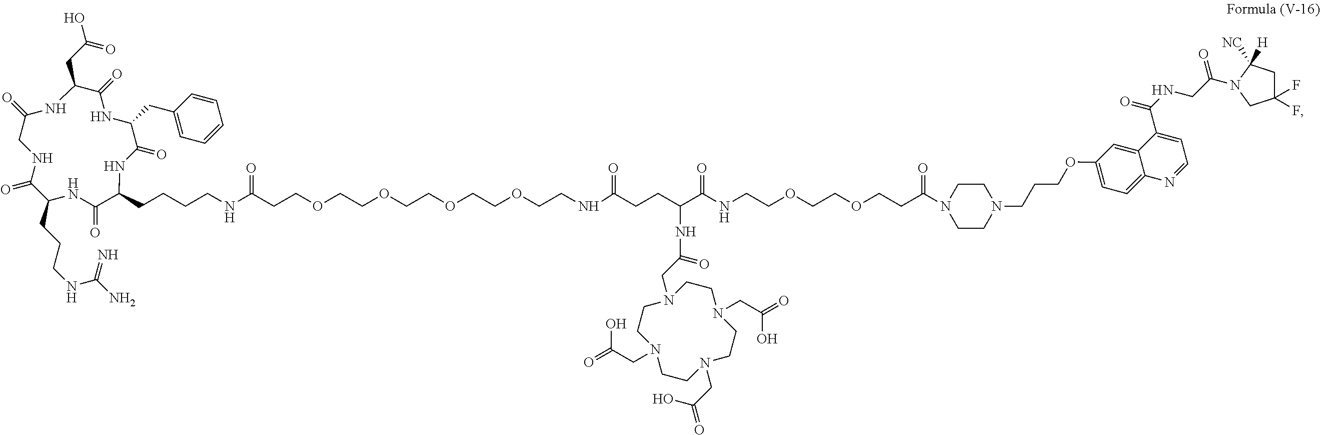 [SMILES: N#C[C@@H]1CC(F)(F)CN1C(=O)CNC(=O)c1ccnc2ccc(OCCCN3CCN(C(=O)CCOCCOCCNC(=O)C(CCC(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

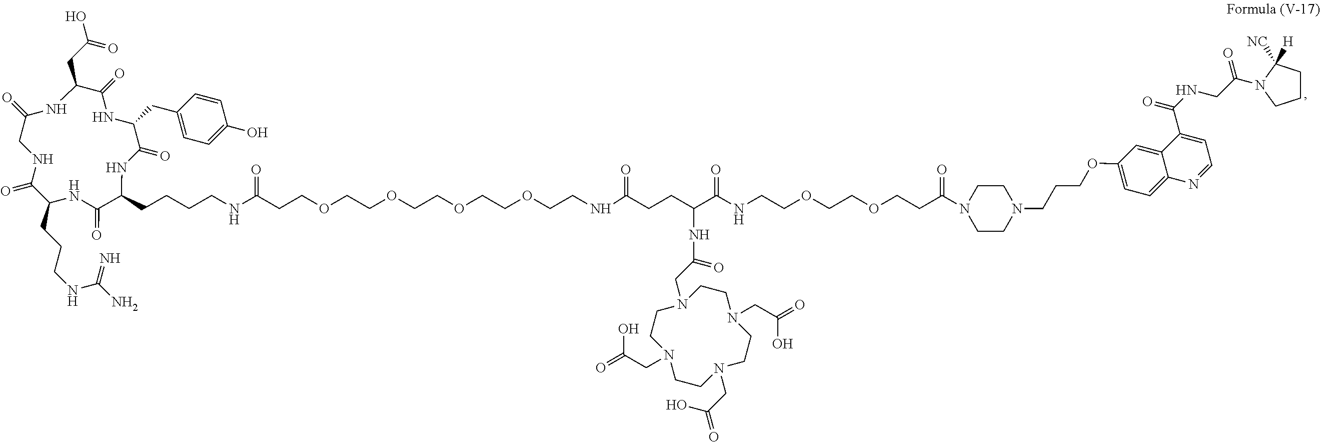 [SMILES: N#C[C@@H]1CCCN1C(=O)CNC(=O)c1ccnc2ccc(OCCCN3CCN(C(=O)CCOCCOCCNC(=O)C(CCC(=O)NCCOCCOCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

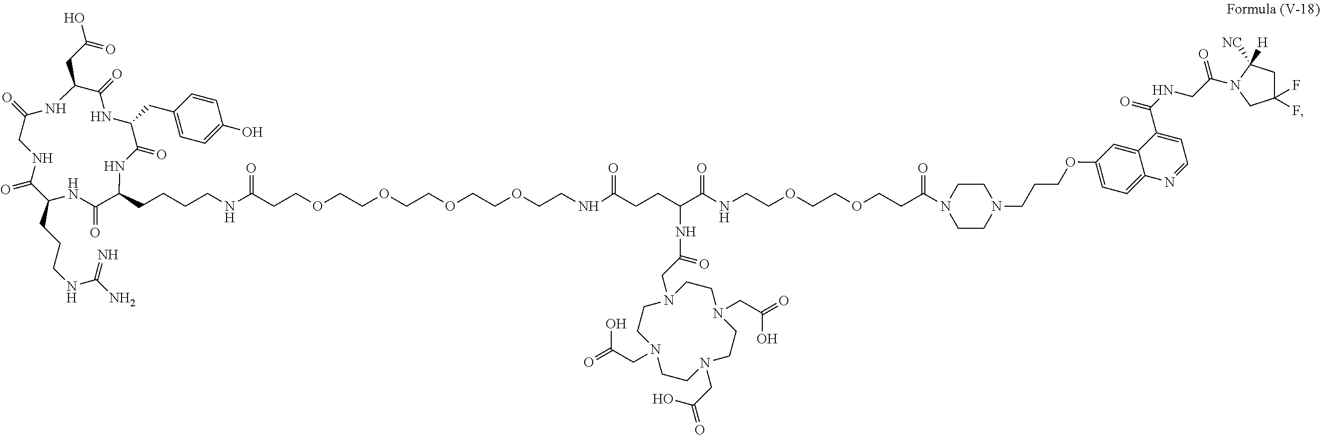 [SMILES: N#C[C@@H]1CC(F)(F)CN1C(=O)CNC(=O)c1ccnc2ccc(OCCCN3CCN(C(=O)CCOCCOCCNC(=O)C(CCC(=O)NCCOCCOCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

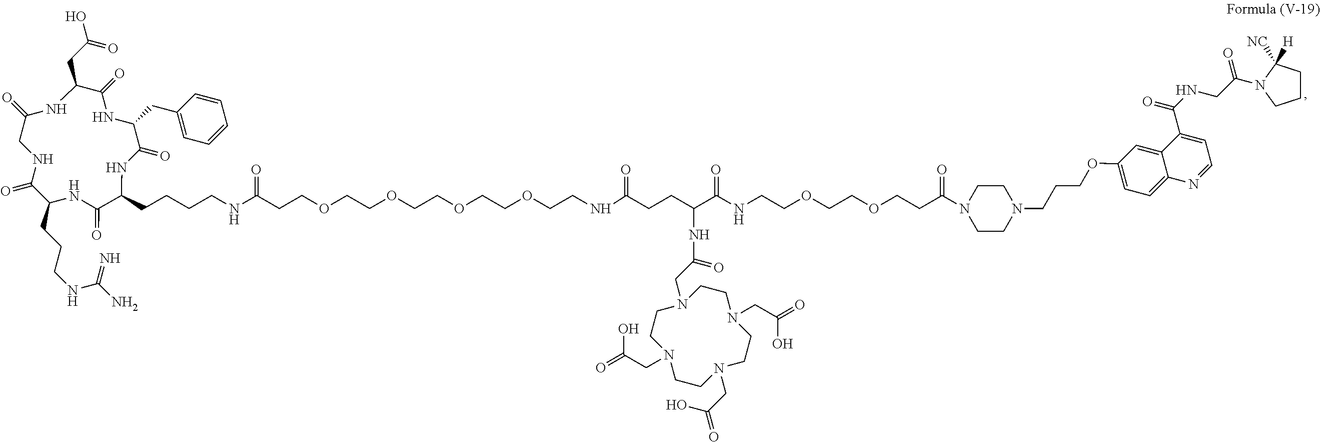 [SMILES: N#C[C@@H]1CCCN1C(=O)CNC(=O)c1ccnc2ccc(OCCCN3CCN(C(=O)CCOCCOCCNC(=O)C(CCC(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

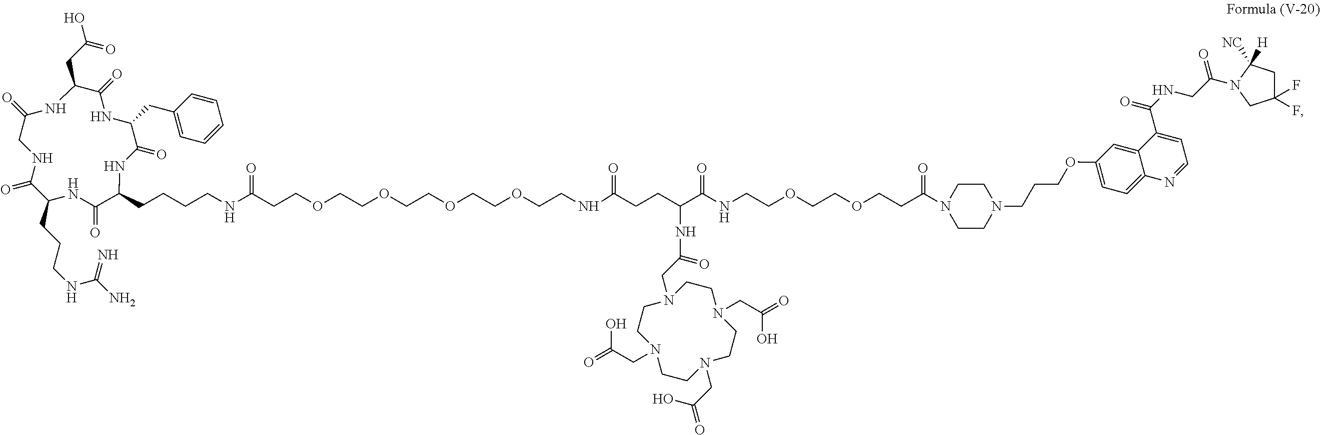 [SMILES: N#C[C@@H]1CC(F)(F)CN1C(=O)CNC(=O)c1ccnc2ccc(OCCCN3CCN(C(=O)CCOCCOCCNC(=O)C(CCC(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

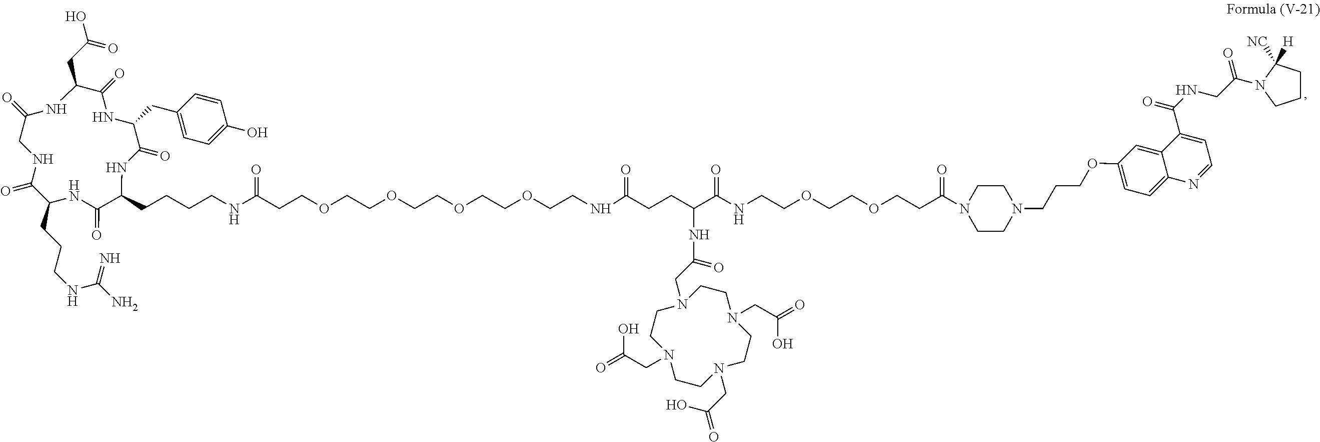 [SMILES: N#C[C@@H]1CCCN1C(=O)CNC(=O)c1ccnc2ccc(OCCCN3CCN(C(=O)CCOCCOCCNC(=O)C(CCC(=O)NCCOCCOCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

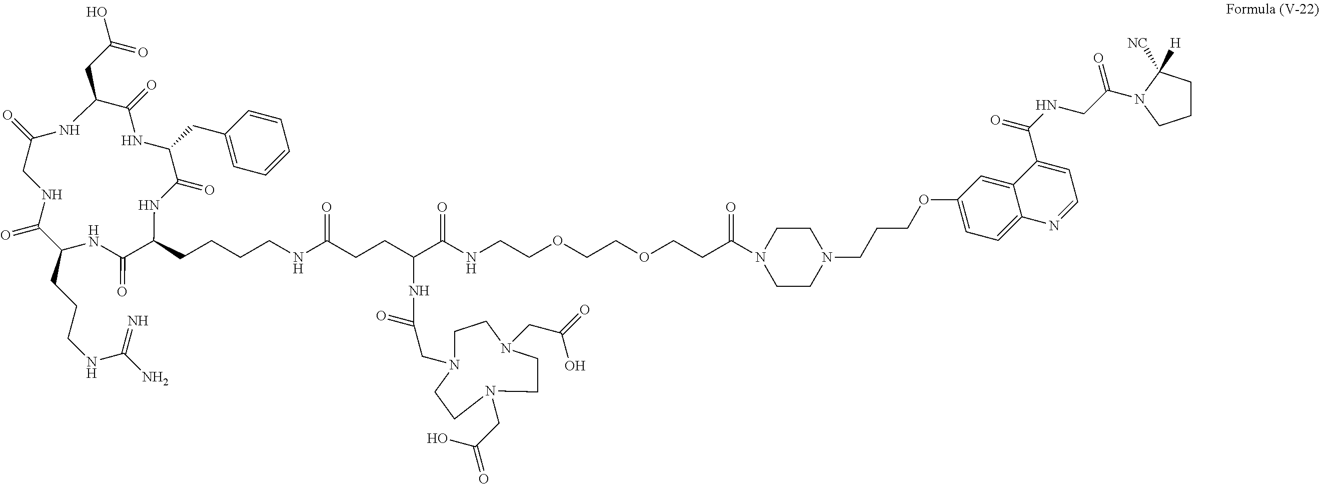 [SMILES: N#C[C@@H]1CCCN1C(=O)CNC(=O)c1ccnc2ccc(OCCCN3CCN(C(=O)CCOCCOCCNC(=O)C(CCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CC4)CC3)cc12]

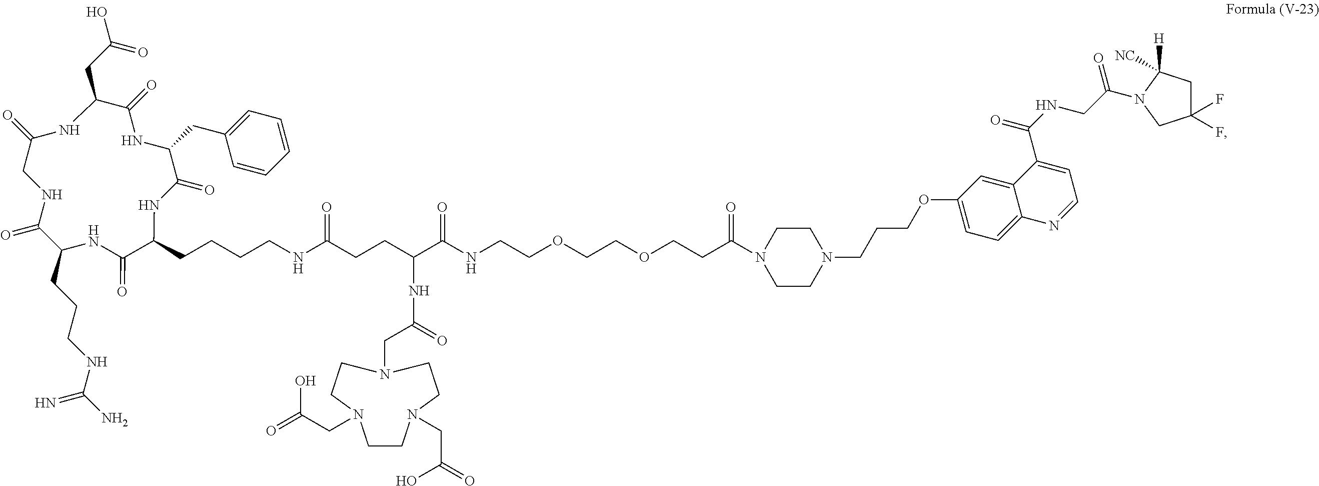 [SMILES: N#C[C@@H]1CC(F)(F)CN1C(=O)CNC(=O)c1ccnc2ccc(OCCCN3CCN(C(=O)CCOCCOCCNC(=O)C(CCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CC4)CC3)cc12]

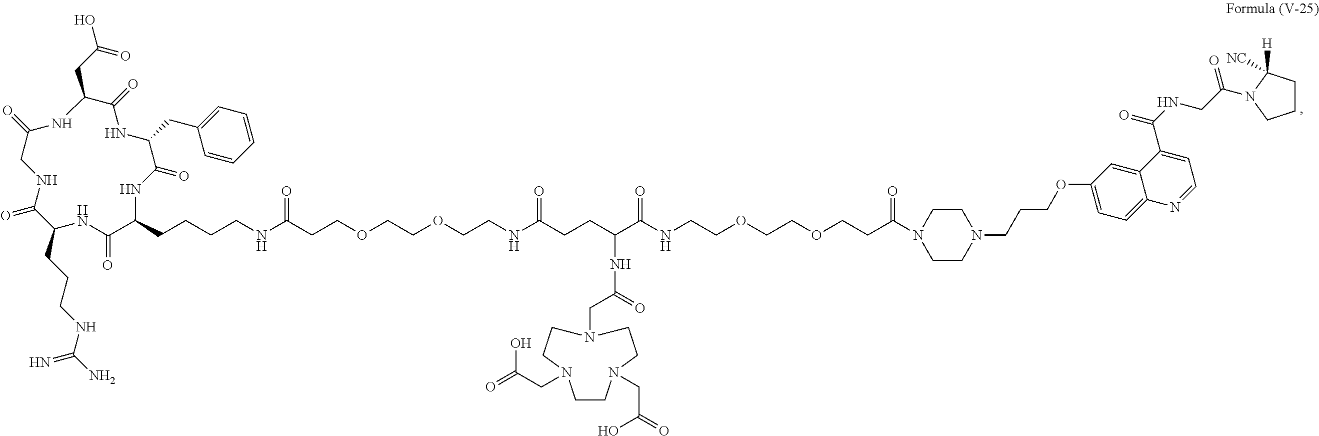 [SMILES: N#C[C@@H]1CCCN1C(=O)CNC(=O)c1ccnc2ccc(OCCCN3CCN(C(=O)CCOCCOCCNC(=O)C(CCC(=O)NCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CC4)CC3)cc12]

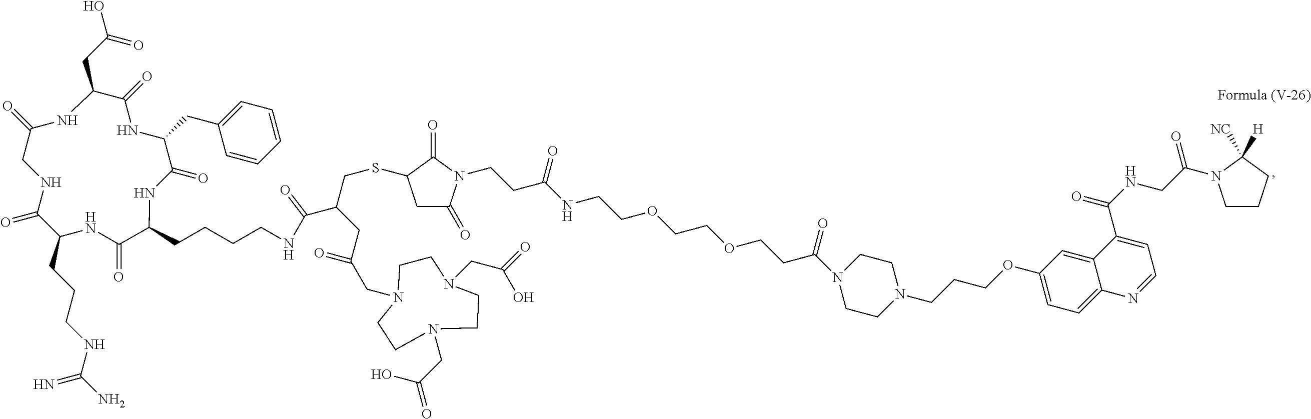 [SMILES: N#C[C@@H]1CCCN1C(=O)CNC(=O)c1ccnc2ccc(OCCCN3CCN(C(=O)CCOCCOCCNC(=O)CCN4C(=O)CC(SCC(CC(=O)CN5CCN(CC(=O)O)CCN(CC(=O)O)CC5)C(=O)NCCCC[C@@H]5NC(=O)[C@@H](Cc6ccccc6)NC(=O)[C@H](CC(=O)O)NC(=O)CNC(=O)[C@H](CCCNC(=N)N)NC5=O)C4=O)CC3)cc12]

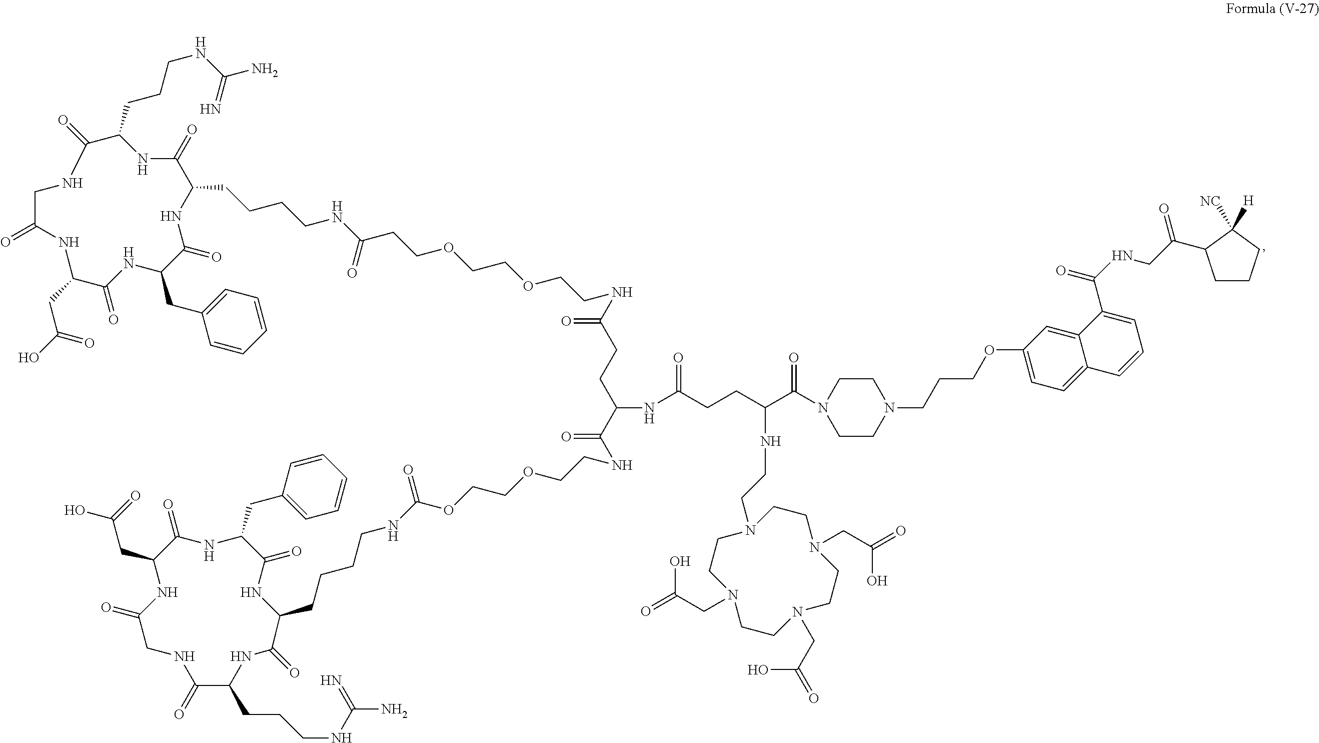 [SMILES: N#C[C@@H]1CCCC1C(=O)CNC(=O)c1cccc2ccc(OCCCN3CCN(C(=O)C(CCC(=O)NC(CCC(=O)NCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)C(=O)NCCOCCOC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NCCN4CCN(CC(=O)O)CCN(CC(=O)O)CCN(CC(=O)O)CC4)CC3)cc12]

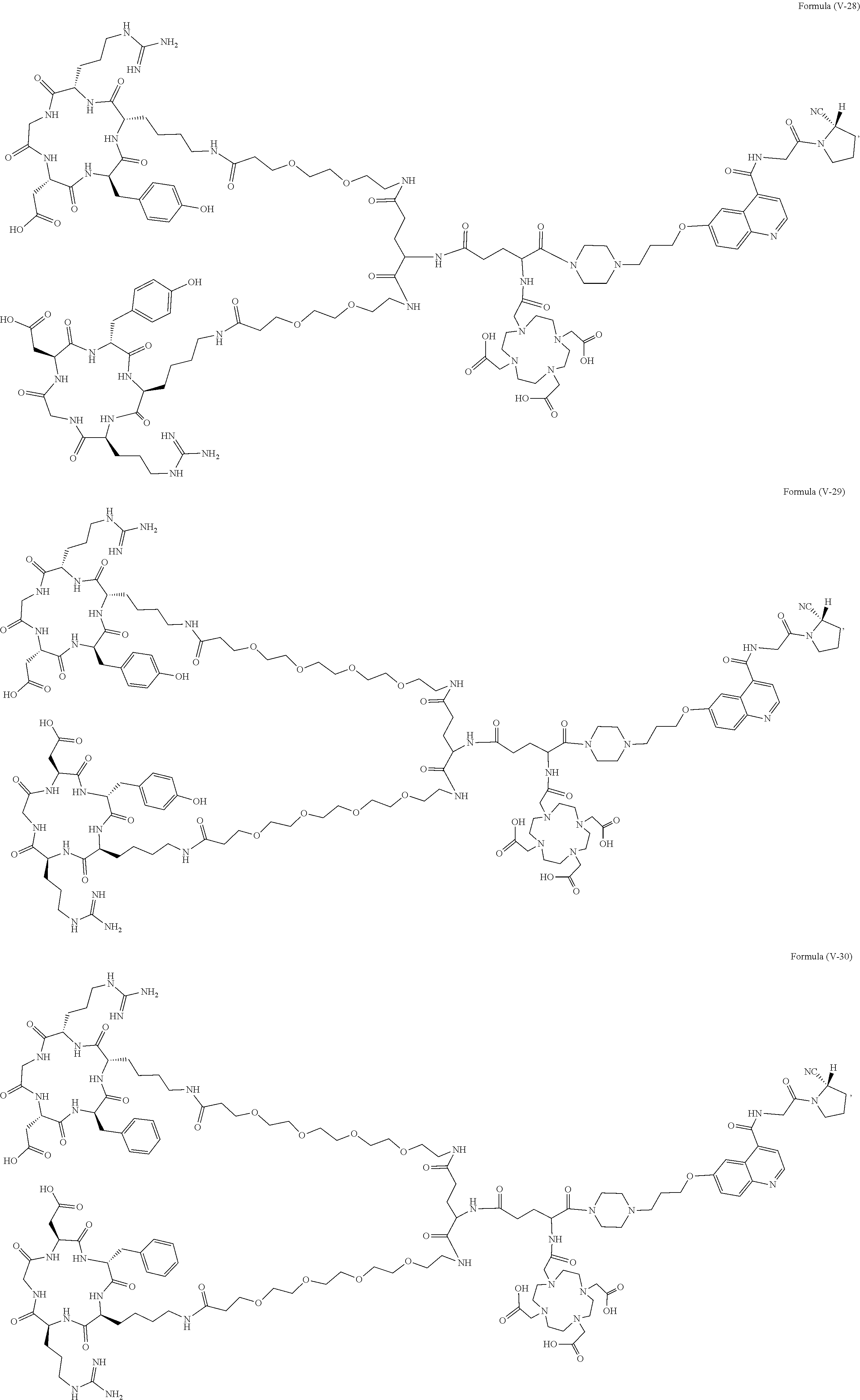

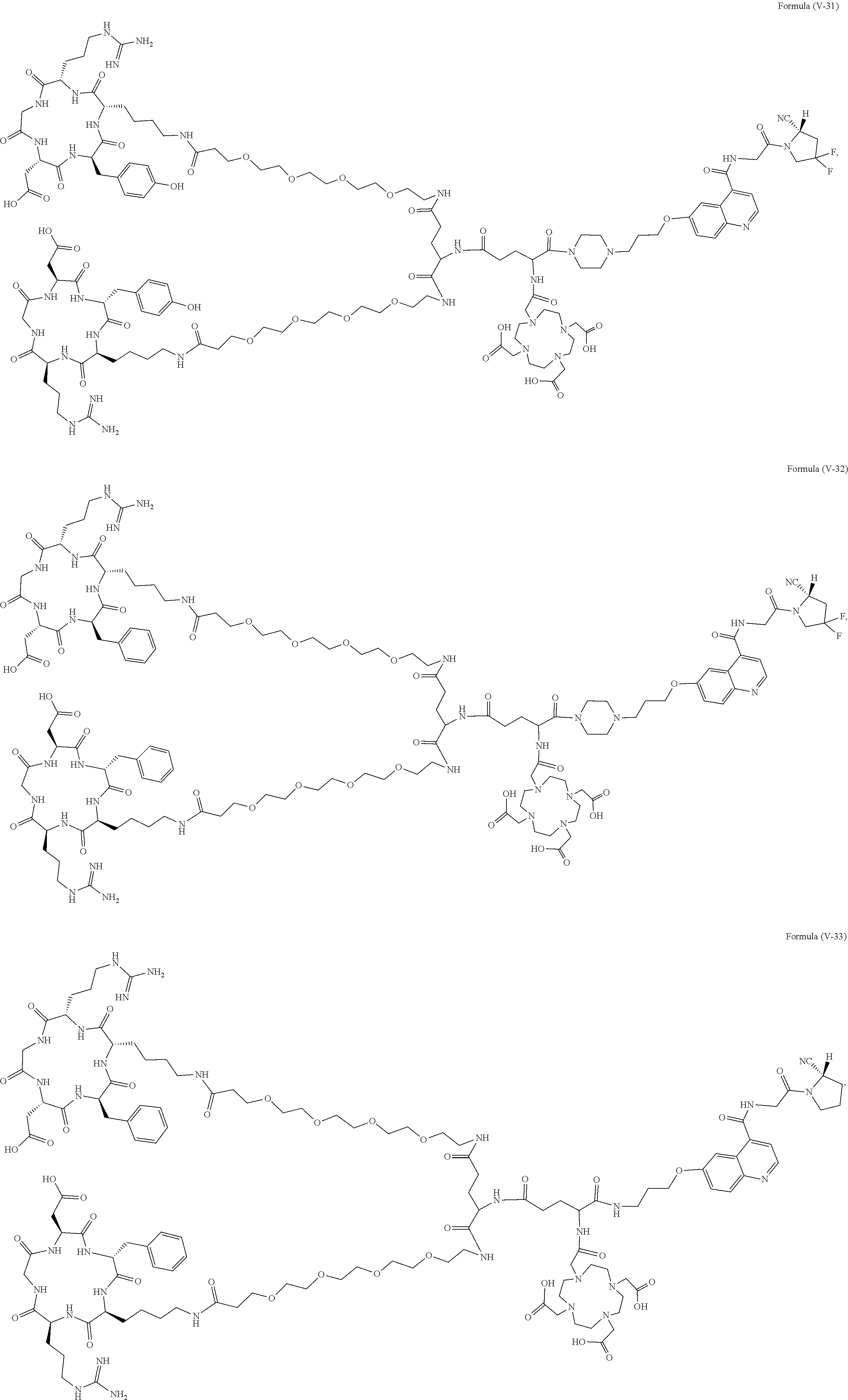

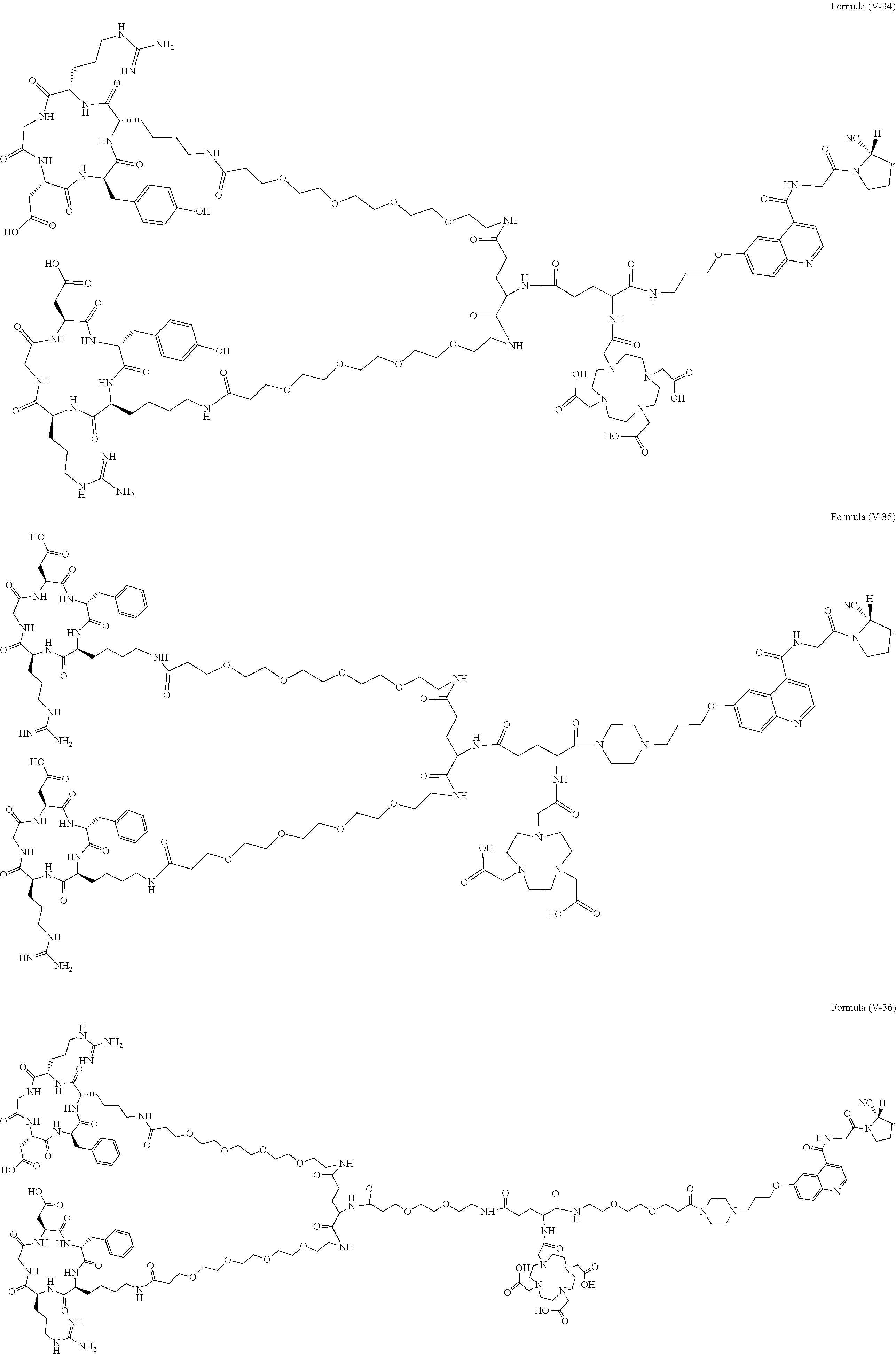

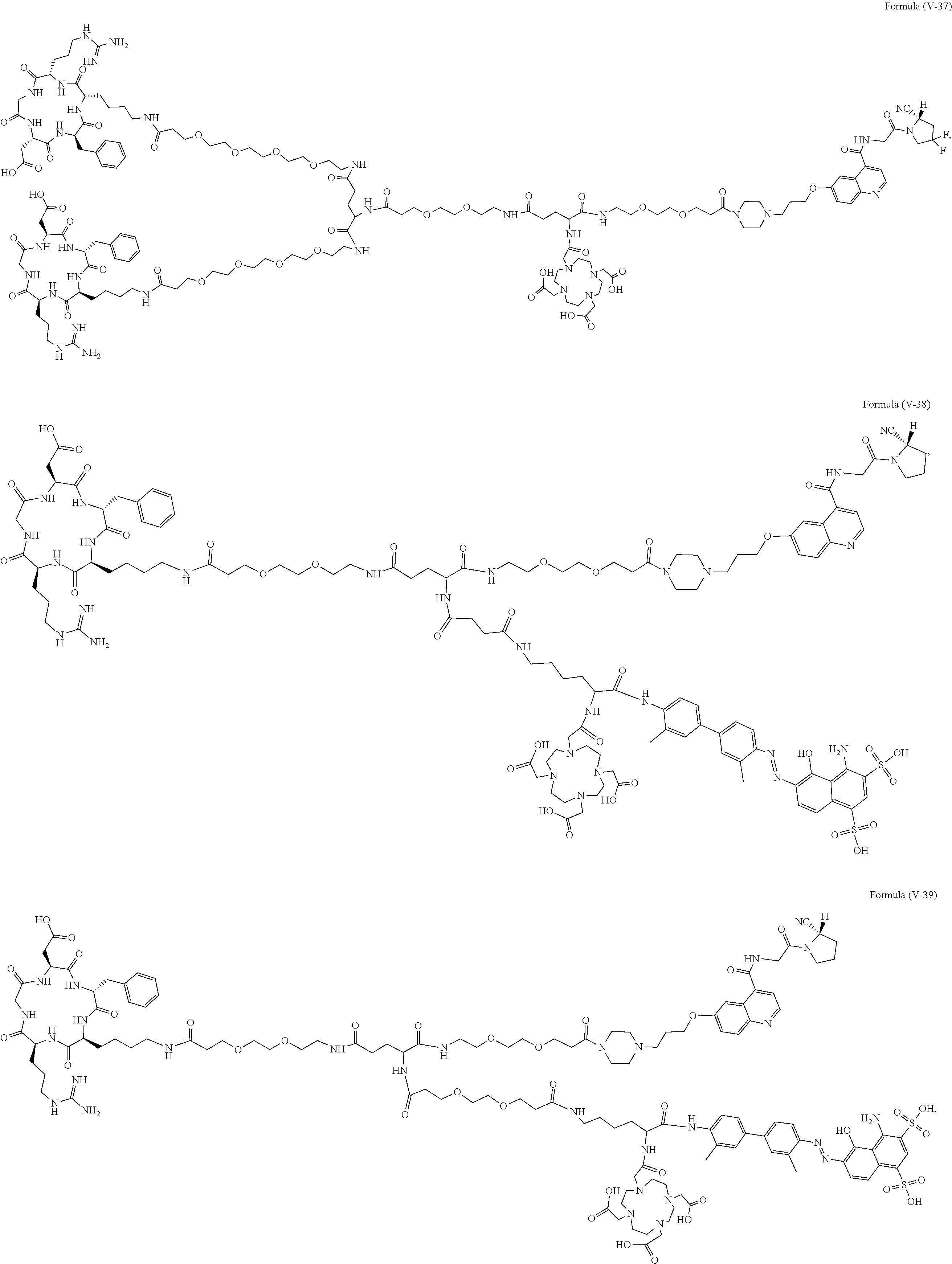

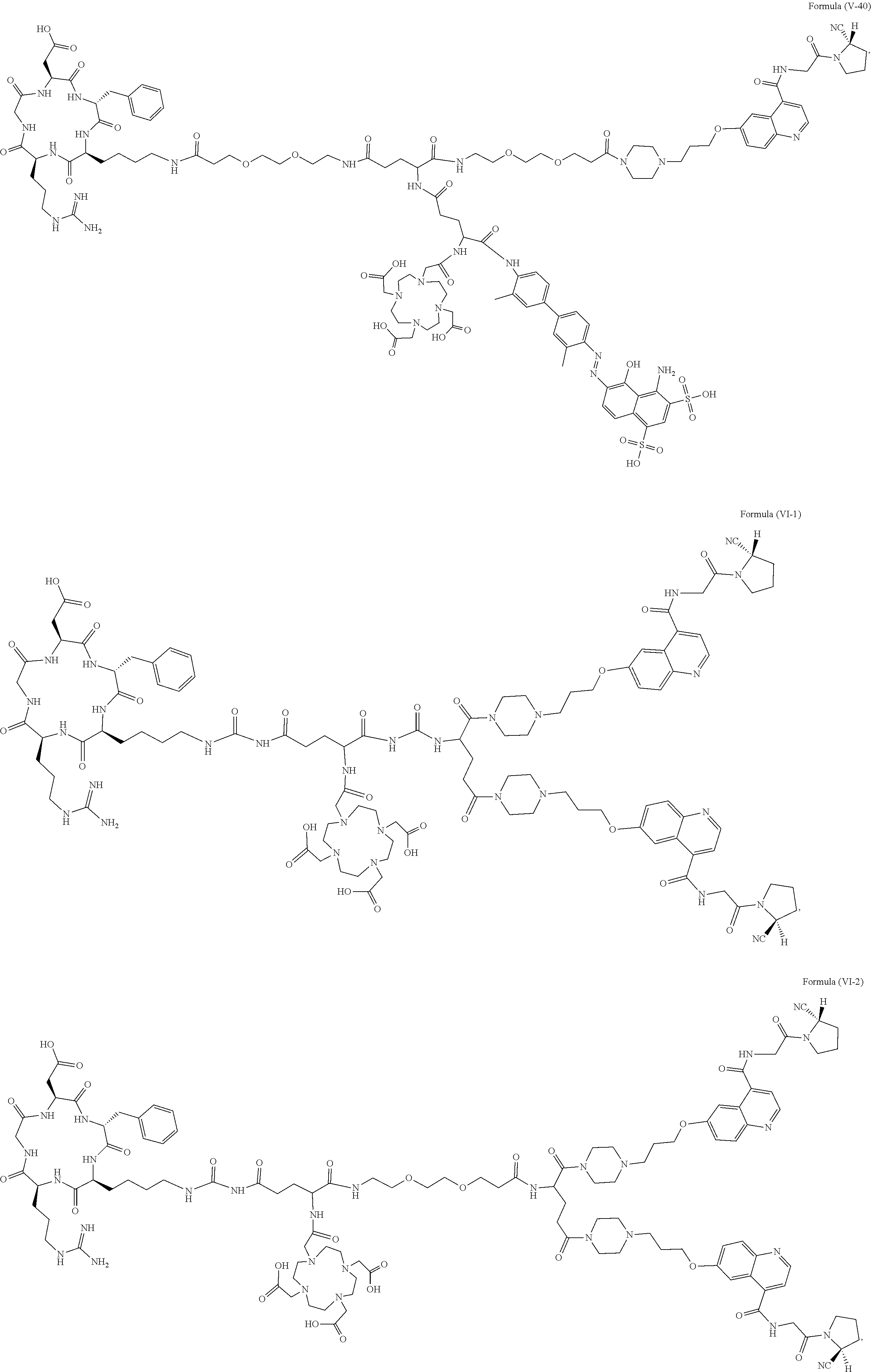

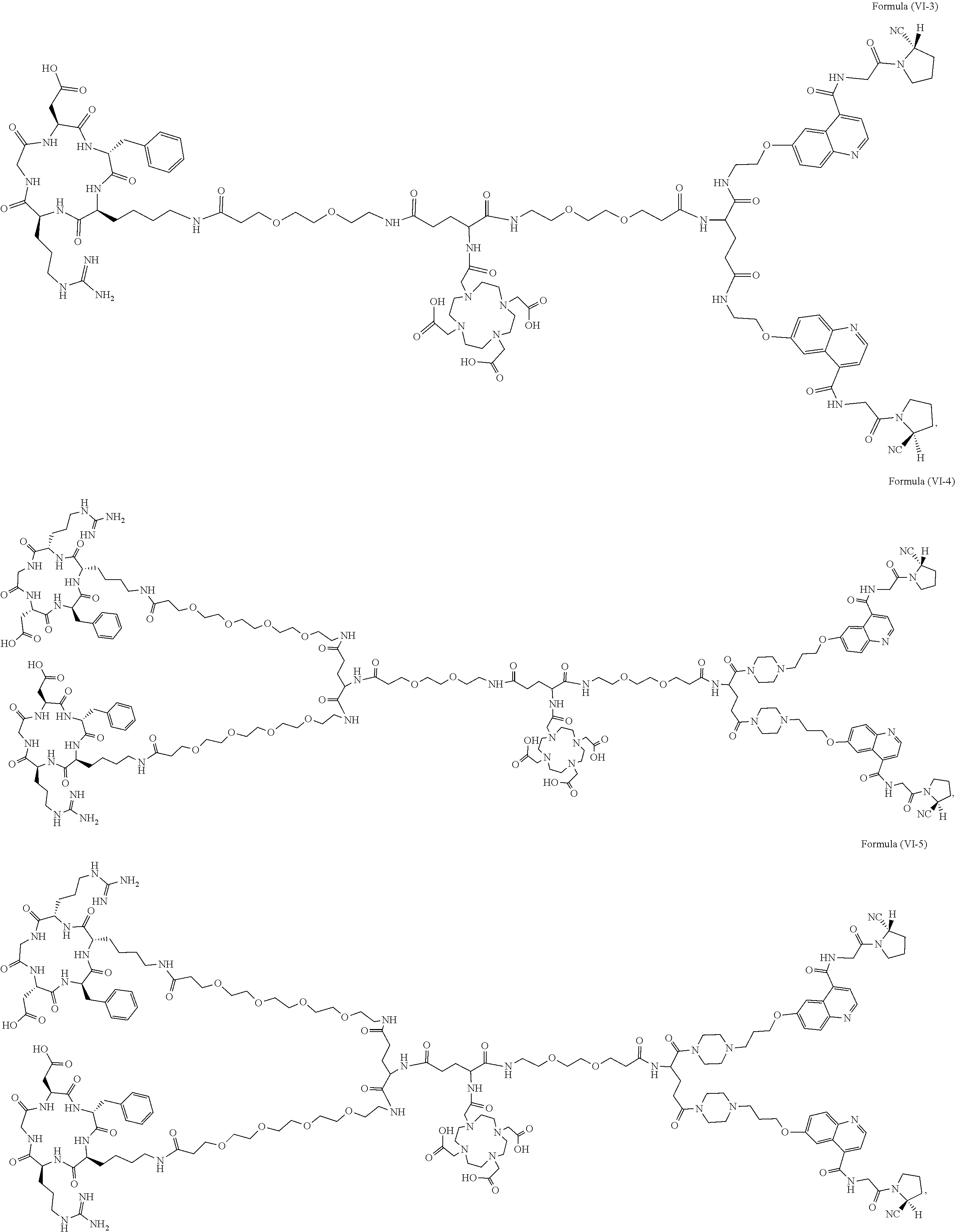

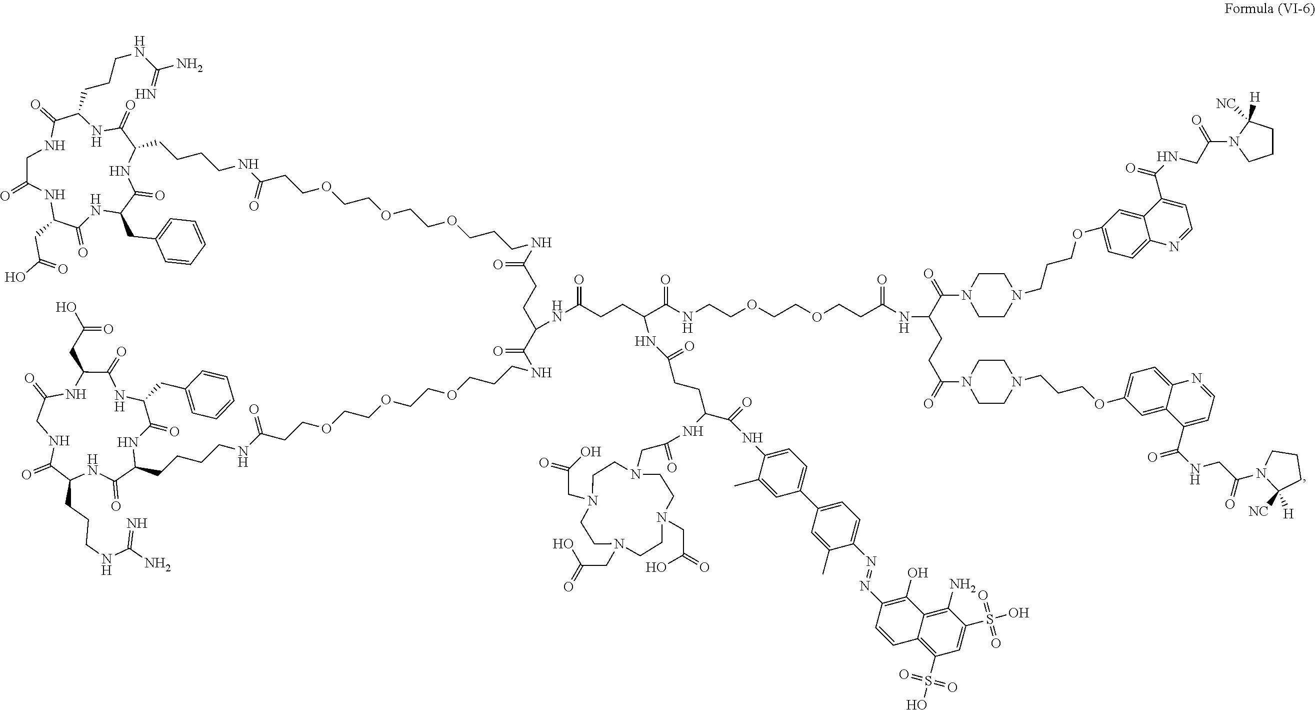

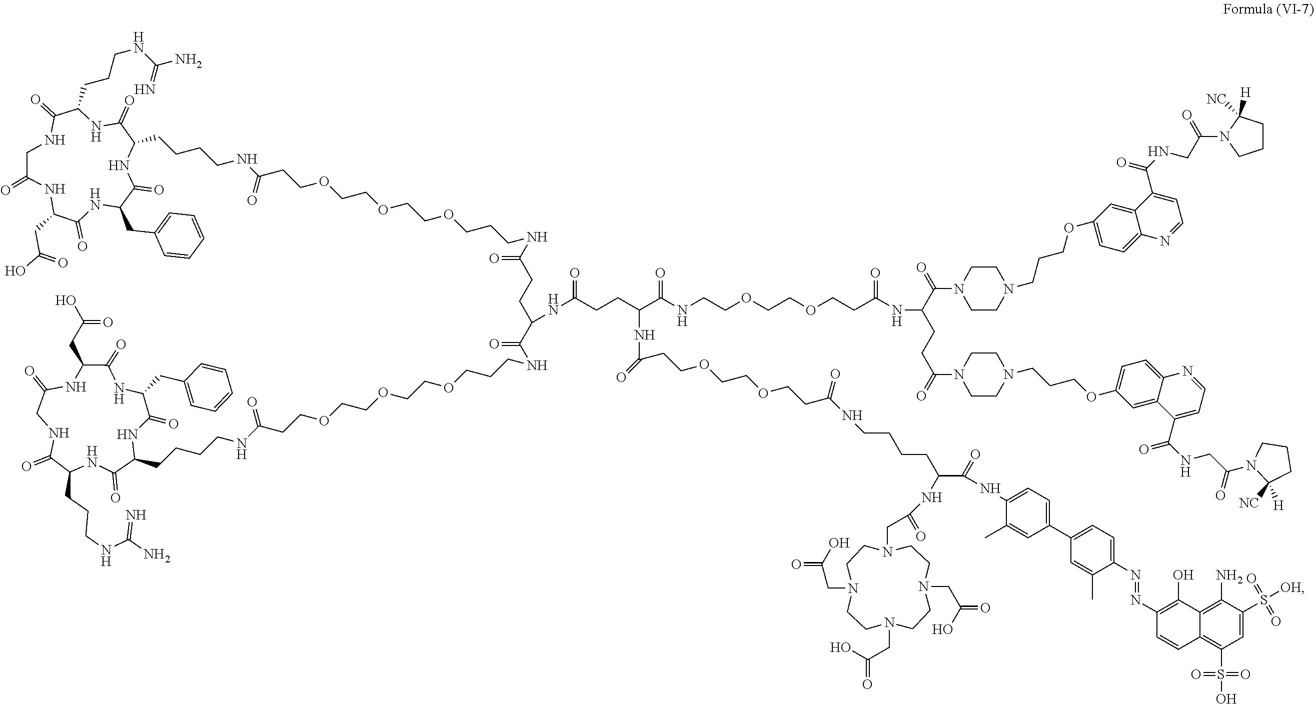

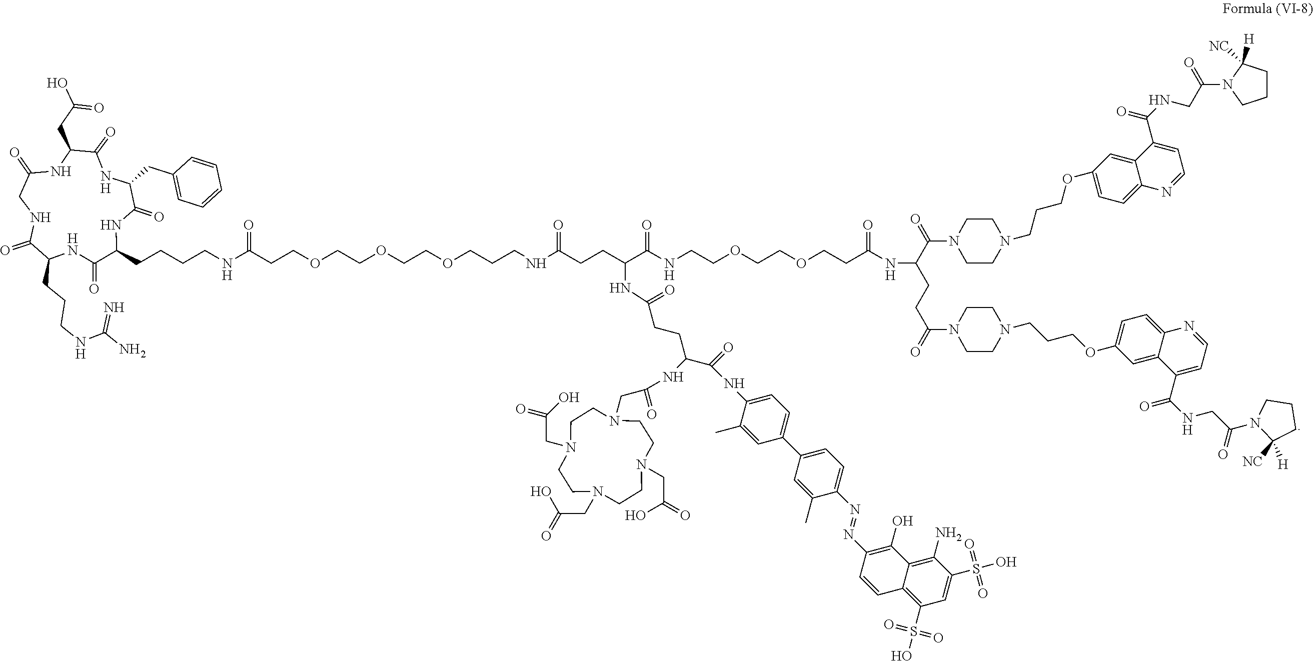 [SMILES: Cc1cc(-c2ccc(NC(=O)C(CCC(=O)NC(CCC(=O)NCCCOCCOCCOCCC(=O)NCCCC[C@@H]3NC(=O)[C@@H](Cc4ccccc4)NC(=O)[C@H](CC(=O)O)NC(=O)CNC(=O)[C@H](CCCNC(=N)N)NC3=O)C(=O)NCCOCCOCCC(=O)NC(CCC(=O)N3CCN(CCCOc4ccc5nccc(C(=O)NCC(=O)N6CCC[C@H]6C#N)c5c4)CC3)C(=O)N3CCN(CCCOc4ccc5nccc(C(=O)NCC(=O)N6CCC[C@H]6C#N)c5c4)CC3)NC(=O)CN3CCN(CC(=O)O)CCN(CC(=O)O)CCN(CC(=O)O)CC3)c(C)c2)ccc1/N=N/c1ccc2c(S(=O)(=O)O)cc(S(=O)(=O)O)c(N)c2c1O]

In a third aspect, the present invention further provides a radionuclide-labeled dual-targeting compound based on the compound capable of being labeled with a radionuclide according to any one of the above descriptions, where the radionuclide-labeled dual-targeting compound is formed by chelating the W group in the compound of Formula (V) or Formula (VI) according to any one of the above descriptions with a radionuclide.

Preferably, the radionuclide may be selected from isotopes emitting α rays, isotopes emitting β rays, isotopes emitting γ rays, isotopes emitting Auger electrons or isotopes emitting X rays and the like.

More preferably, the radionuclide is selected from any one of ¹⁸F, ⁵¹Cr, ⁶⁷Ga, ⁶⁸Ga, ¹¹¹In, ^{99m}Tc, ¹⁸⁶Re, ¹⁸⁸Re, ¹³⁹La, ¹⁴⁰La, ¹⁷⁵Yb, ¹⁵³Sm, ¹⁶⁶Ho, ⁸⁶Y, ⁹⁰Y, ¹⁴⁹Pm, ¹⁶⁵Dy, ¹⁶⁹Er, ¹⁷⁷Lu, ⁴⁷Sc, ¹⁴²Pr, ¹⁵⁹Gd, ²¹²Bi, ²¹³Bi, ⁷²As, ⁷²Se, ⁹⁷Ru, ¹⁰⁹Pd, ¹⁰⁵Rh, ^{101m}Rh, ¹¹⁹Sb, ¹²⁸Ba, ¹²³I, ¹²⁴I, ¹³¹I, ¹⁹⁷Hg, ²¹¹At, ¹⁵¹Eu, ¹⁵³Eu, ¹⁶⁹Eu, ²⁰¹Tl, ²⁰³Pb, ²¹²Pb, ⁶⁴Cu, ⁶⁷Cu, ¹⁹⁸Au, ²²⁵Ac, ²²⁷Th, ⁸⁹Zr or ¹⁹⁹Ag.

More preferably, the radionuclide is any one of ¹⁸F, ⁶⁴Cu, ⁶⁸Ga, ⁸⁹Zr, ⁹⁰Y ¹¹¹In, ^{99m}Tc, ¹⁷⁷Lu, ¹⁸⁸Re or ²²⁵Ac. In some specific embodiments, the radionuclide is ¹⁸F; in other specific embodiments, the radionuclide is ⁶⁴Cu; in other specific embodiments, the radionuclide is ⁶⁸Ga; in other specific embodiments, the radionuclide is ⁸⁹Zr; in other specific embodiments, the radionuclide is ⁹⁰Y; in other specific embodiments, the radionuclide is ¹¹¹In; in other specific embodiments, the radionuclide is ^{99m}Tc; in other specific embodiments, the radionuclide is ¹⁷⁷Lu; in other specific embodiments, the radionuclide is ¹⁸⁸Re; and in other specific embodiments, the radionuclide is ²²⁵Ac.

In a fourth aspect, the present invention further provides pharmaceutically acceptable tautomers, racemates, hydrates, solvates or salts of the dual-targeting compound, the compound capable of being labeled with a radionuclide and the radionuclide-labeled dual-targeting compound according to any one of the above descriptions.

In a fifth aspect, the present invention further provides a method for preparing the targeting compound of Formula (V) according to any one of the above descriptions and a radionuclide-labeled compound thereof. The method includes:
(1) first carrying out an amide condensation reaction between carboxyl of 6-hydroxyquinoline-4-carboxylic acid and amino of glycine tert-butyl ester; then connecting BOC-protected piperazinyl by an alkyl chain at a hydroxyl position of an amide condensation product; removing Boc and tert-butyl protective groups under acidic conditions, and then introducing a Boc protective group to a piperazine ring; then carrying out an amide condensation reaction with (S)-pyrrolidine-2-carbonitrile hydrochloride or (S)-4,4-difluoropyrrolidine-2-carbonitrile hydrochloride; after removing the Boc protective group, carrying out a condensation reaction with N-Boc-3-[2-(2-aminoethoxy)ethoxy]propionic acid; then removing the Boc protective group, and sequentially carrying out a reaction with maleimide propionate and cysteine with a protective group, or then carrying gout a reaction with glutamic acid or lysine with a protective group; and finally, introducing RGD (c(RGDyK), c(RGDfK) or c(RGDyK)/c(RGDfK) with a PEG short chain) by an activated ester reaction to obtain a dual-targeting compound;
(2) carrying out a reaction between the dual-targeting compound obtained in step (1) and a nuclide chelating agent to obtain the compound capable of being labeled with a radionuclide as shown in Formula (V) according to some descriptions, where the nuclide chelating agent is selected from any one of hydroxysuccinimide-tetraazacyclododecane-N,N′,N,N′-tetraacetic acid (DOTA-NHS), NOTA-succinimide ester (NOTA-NHS), succinimide active ester of iminodiacetic acid, succinimide active ester of diethylenetriamine-N,N,N′,N′,N″-pentaacetic acid (DTPA-NHS) and succinimide active ester of bis(carboxymethyl)imidazole glycinate or 6-hydrazinopyridine-3-carboxylic acid (HYNIC-NHS); and
(3) carrying out a reaction between the compound capable of being labeled with a radionuclide obtained in step (2) and a compound containing a radionuclide by an existing wet labeling method or a freeze-drying labeling method to prepare and obtain the radionuclide-labeled targeting compound of the present invention.

In a sixth aspect, the present invention further provides a pharmaceutical composition. The pharmaceutical composition includes the dual-targeting compound, the compound capable of being labeled with a radionuclide, or the radionuclide-labeled dual-targeting compound according to any one of the above descriptions, or any pharmaceutically acceptable tautomers, racemates, hydrates, solvates or salts thereof; or the pharmaceutical composition is composed of the dual-targeting compound, the compound capable of being labeled with a radionuclide, or the radionuclide-labeled dual-targeting compound according to any one of the above descriptions, or any pharmaceutically acceptable tautomers, racemates, hydrates, solvates or salts thereof and any pharmaceutically acceptable carrier and/or excipient.

In a seventh aspect, the present invention further provides use of the dual-targeting compound, the compound capable of being labeled with a radionuclide, or the radionuclide-labeled dual-targeting compound according to any one of the above descriptions in preparation of drugs for diagnosis or treatment of diseases characterized by overexpression of fibroblast activation protein (FAP) and/or integrin αᵥβ₃ in animals or human individuals.

The present invention further provides a method for diagnosing or treating diseases characterized by overexpression of fibroblast activation protein (FAP) and/or integrin αᵥβ₃ in animals or human individuals by using the dual-targeting compound, the compound capable of being labeled with a radionuclide, or the radionuclide-labeled dual-targeting compound according to any one of the above descriptions.

Preferably, the diseases characterized by overexpression of fibroblast activation protein (FAP) and/or integrin αᵥβ₃ include, but are not limited to: cancer, chronic inflammation, atherosclerosis, fibrosis, tissue remodeling and scar diseases; and preferably, the cancer is further selected from breast cancer, pancreatic cancer, small intestine cancer, colon cancer, rectal cancer, lung cancer, head and neck cancer, ovarian cancer, hepatocellular carcinoma, esophageal cancer, hypopharyngeal carcinoma, nasopharyngeal carcinoma, laryngeal carcinoma, myeloma cells, bladder cancer, cholangiocellular carcinoma, clear cell renal cell carcinoma, neuroendocrine neoplasm, carcinogenic osteomalacia, sarcoma, cancer of unknown primary (CUP), thymic carcinoma, glioma, neuroglioma, astrocytoma, cervical cancer or prostate cancer.

In an eighth aspect, the present invention further provides a kit. The kit includes or is composed of the targeting compound shown in Formula (I) or Formula (II), the compound shown in Formula (V) or Formula (VI), the radionuclide-labeled targeting compound of the present invention, or the pharmaceutical composition of the present invention and an instruction for diagnosing diseases.

The compound with the FAPI-RGD structure provided by the present invention can synergistically target an FAP target and an integrin αᵥβ₃ target in tumors, such that the number and utilization efficiency of effective receptors in tumors can be improved. The radionuclide-labeled compound further provided based on the structure is expected to be used for diagnosis or treatment of diseases characterized by overexpression of fibroblast activation protein (FAP) and/or integrin αᵥβ₃.

### BRIEF DESCRIPTION OF DRAWINGS

FIG. 1 is a mass spectrogram of a compound 2 in Example 1 of the present invention.

FIG. 2 shows a nuclear magnetic hydrogen spectrum of the compound 2 in Example 1 of the present invention.

FIG. 3 shows a nuclear magnetic carbon spectrum of the compound 2 in Example 1 of the present invention.

FIG. 4 is a mass spectrogram of a compound 3 in Example 1 of the present invention.

FIG. 5 shows a nuclear magnetic hydrogen spectrum of the compound 3 in Example 1 of the present invention.

FIG. 6 is a mass spectrogram of a compound 4 in Example 1 of the present invention.

FIG. 7 shows a nuclear magnetic hydrogen spectrum of the compound 4 in Example 1 of the present invention.

FIG. 8 shows a nuclear magnetic carbon spectrum of the compound 4 in Example 1 of the present invention.

FIG. 9 is a mass spectrogram of a compound 7 in Example 1 of the present invention.

FIG. 10 shows a nuclear magnetic hydrogen spectrum of the compound 7 in Example 1 of the present invention.

FIG. 11 shows a nuclear magnetic carbon spectrum of the compound 7 in Example 1 of the present invention.

FIG. 12 is a mass spectrogram of a compound 9 in Example 1 of the present invention.

FIG. 13 is a mass spectrogram of a compound 10 in Example 1 of the present invention.

FIG. 14 is a mass spectrogram of a compound 11 in Example 1 of the present invention.

FIG. 15 is a mass spectrogram of a compound of Formula (V-1) in Example 1 of the present invention.

FIG. 16 is a mass spectrogram of an intermediate M in Example 2 of the present invention.

FIG. 17 is a mass spectrogram of an intermediate O in Example 2 of the present invention.

FIG. 18 is a mass spectrogram of an intermediate B in Example 2 of the present invention.

FIG. 19 is a mass spectrogram of an intermediate C in Example 2 of the present invention.

FIG. 20 is a mass spectrogram of an intermediate D in Example 2 of the present invention.

FIG. 21 is a mass spectrogram of an intermediate E in Example 2 of the present invention.

FIG. 22 is a mass spectrogram of an intermediate F in Example 2 of the present invention.

FIG. 23 is a mass spectrogram of an intermediate G in Example 2 of the present invention.

FIG. 24 is a mass spectrogram of an intermediate H in Example 2 of the present invention.

FIG. 25 is a mass spectrogram of an intermediate I in Example 2 of the present invention.

FIG. 26 is a mass spectrogram of an intermediate J in Example 2 of the present invention.

FIG. 27 is a mass spectrogram of an intermediate Q in Example 2 of the present invention.

FIG. 28 is a mass spectrogram of a compound of Formula (V-14) in Example 2 of the present invention.

FIG. 29 is a mass spectrogram of an intermediate K in Example 3 of the present invention.

FIG. 30 is a mass spectrogram of a compound of Formula (V-23) in Example 3 of the present invention.

FIG. 31 is a mass spectrogram of an intermediate B1 in Example 4 of the present invention.

FIG. 32 is a mass spectrogram of an intermediate D1 in Example 4 of the present invention.

FIG. 33 is a mass spectrogram of an intermediate G1 in Example 4 of the present invention.

FIG. 34 is a mass spectrogram of an intermediate H1 in Example 4 of the present invention.

FIG. 35 is a mass spectrogram of an intermediate I1 in Example 4 of the present invention.

FIG. 36 is a mass spectrogram of an intermediate J1 in Example 4 of the present invention.

FIG. 37 is a mass spectrogram of a compound of Formula (V-25) in Example 4 of the present invention.

FIG. 38 is a mass spectrogram of an intermediate H3 in Example 6 of the present invention.

FIG. 39 is a mass spectrogram of an intermediate 12 in Example 6 of the present invention.

FIG. 40 is a mass spectrogram of an intermediate O1 in Example 6 of the present invention.

FIG. 41 is a mass spectrogram of an intermediate P1 in Example 6 of the present invention.

FIG. 42 is a mass spectrogram of a compound of Formula (V-30) in Example 6 of the present invention.

FIG. 43 is a mass spectrogram of an intermediate N2 in Example 7 of the present invention.

FIG. 44 is a mass spectrogram of an intermediate F3 in Example 7 of the present invention.

FIG. 45 is a mass spectrogram of a compound of Formula (V-35) in Example 7 of the present invention.

FIG. 46 shows high performance liquid chromatography (HPLC) quality control results of a compound ⁶⁸Ga-FAPI-RGD (V-1) in the present invention.

FIG. 47 shows MicroPET imaging results of the compound ⁶⁸Ga-FAPI-RGD (V-1) in HepG2-FAP tumor-bearing mice in the present invention.

FIG. 48 shows MicroPET imaging results of the compound ⁶⁸Ga-FAPI-RGD (V-1) in HepG2-FAP tumor-bearing mice after co-injection with FAPI-02 in the present invention.

FIG. 49 shows MicroPET imaging results of the compound ⁶⁸Ga-FAPI-RGD (V-1) in HepG2-FAP tumor-bearing mice after co-injection with RGD in the present invention.

FIG. 50 is a statistical diagram of uptake results of the compound ⁶⁸Ga-FAPI-RGD (V-1) in tumors and vital organs after co-injection with C(RGDfK) or FAPI-02 for 30 min in the present invention (in the figure, the abscissa shows different organs, and histograms for each organ represent, from left to right, uptake of the ⁶⁸Ga-labeled FAPI-RGD complex, uptake of the ⁶⁸Ga-FAPI-RGD after co-injection with the FAPI-02 for blocking FAP and uptake of the ⁶⁸Ga-FAPI-RGD after co-injection with the RGD for blocking integrin, respectively).

FIG. 51 shows stability experiment results of a compound ⁶⁸Ga-FAPI-RGD (V-25) in normal saline in the present invention.

FIG. 52 shows cell uptake and cell binding experiment results of the compound ⁶⁸Ga-FAPI-RGD (V-25) in the present invention.

FIG. 53 shows MicroPET imaging results of the compound ⁶⁸Ga-FAPI-RGD (V-25) and monomers ⁶⁸Ga-FAPI-02 and ⁶⁸Ga-C(RGDfK) in HT1080-FAP tumor-bearing mice in the present invention.

FIG. 54 shows MicroPET imaging results of the compound ⁶⁸Ga-FAPI-RGD (V-25) after co-injection with C(RGDfK) and/or FAPI-02 for 30 min and a statistical diagram of uptake results in tumors and vital organs in the present invention.

FIG. 55 shows PET/CT imaging results of the compound ⁶⁸Ga-FAPI-RGD (V-25), ¹⁸F-FDG and ⁶⁸Ga-FAPI46 in patients with pancreatic cancer, non-small cell lung cancer, small cell lung cancer and nasopharyngeal carcinoma after intravenous injection for 3 h in the present invention.

FIG. 56 is a mass spectrogram of an intermediate G2 in the present invention.

FIG. 57 is a mass spectrogram of an intermediate N1 in the present invention.

FIG. 58 is a mass spectrogram of an intermediate P in the present invention.

FIG. 59 is a mass spectrogram of a compound of Formula (V-26) in the present invention.

FIG. 60 shows SPECT imaging of a ¹⁷⁷Lu-radiolabeled compound V-40 (namely, a compound ¹⁷⁷Lu-FAPI-RGD (V-40)) provided in the present invention.

### DETAILED DESCRIPTION OF EMBODIMENTS

The technical solutions of the present invention are further described in non-restrictive detail in combination with specific embodiments below. It should be noted that examples below are only to illustrate the technical concept and characteristics of the present invention for the purpose of making the contents of the present invention understood and implemented by persons skilled in the art, and are not intended to limit the scope of protection of the present invention. Any equivalent variations or modifications made in accordance with the spirit and essence of the present invention shall fall within the scope of protection of the present invention.

### Example 1: Preparation of a Compound of Formula (I-1) and a Compound of Formula (V-1)

A synthesis route is as follows.

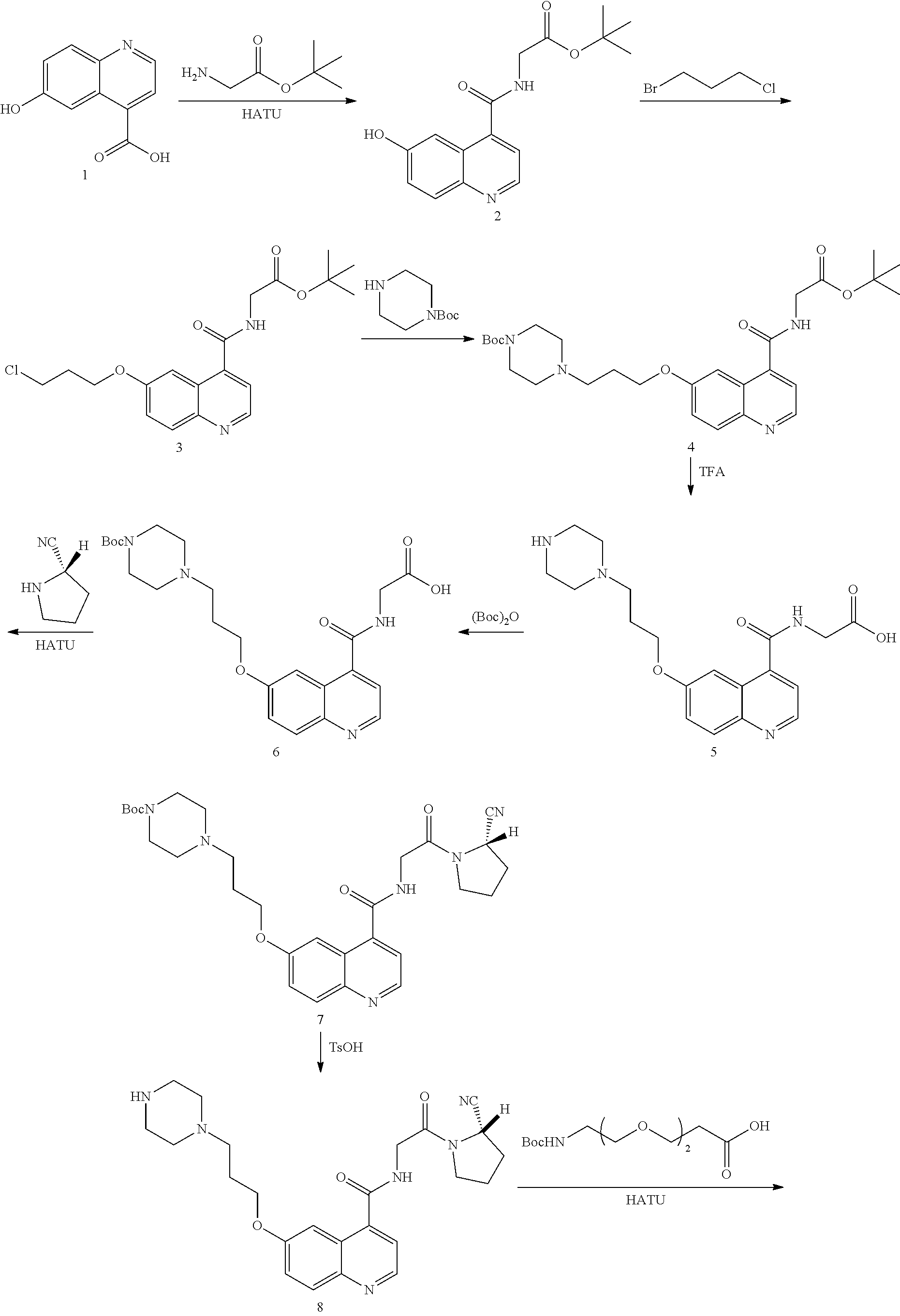

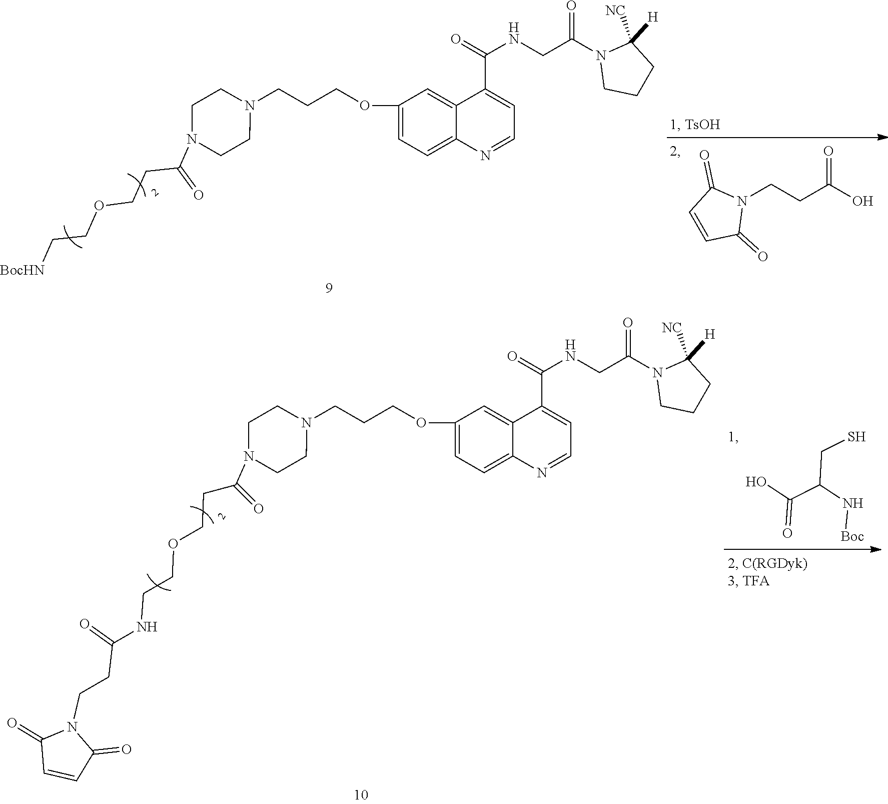

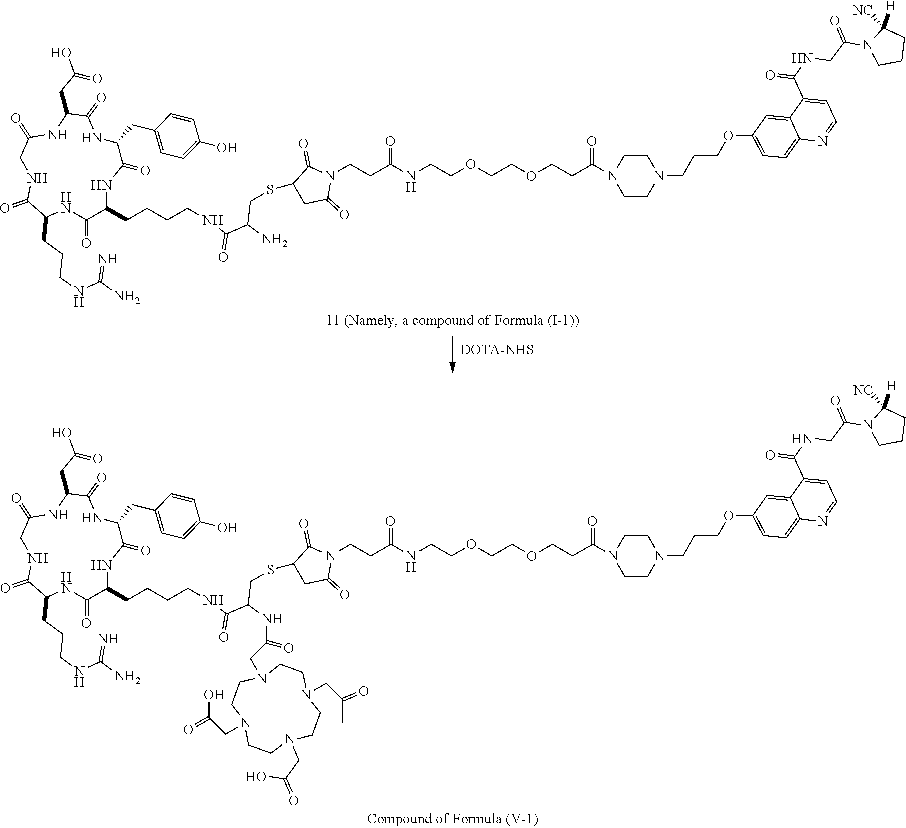

### (1) Synthesis of a Compound 2

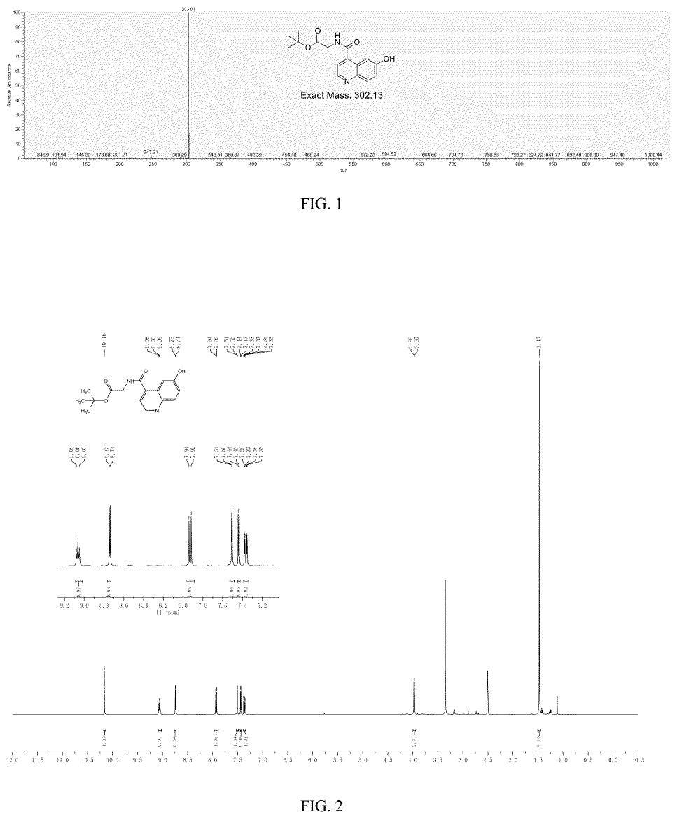

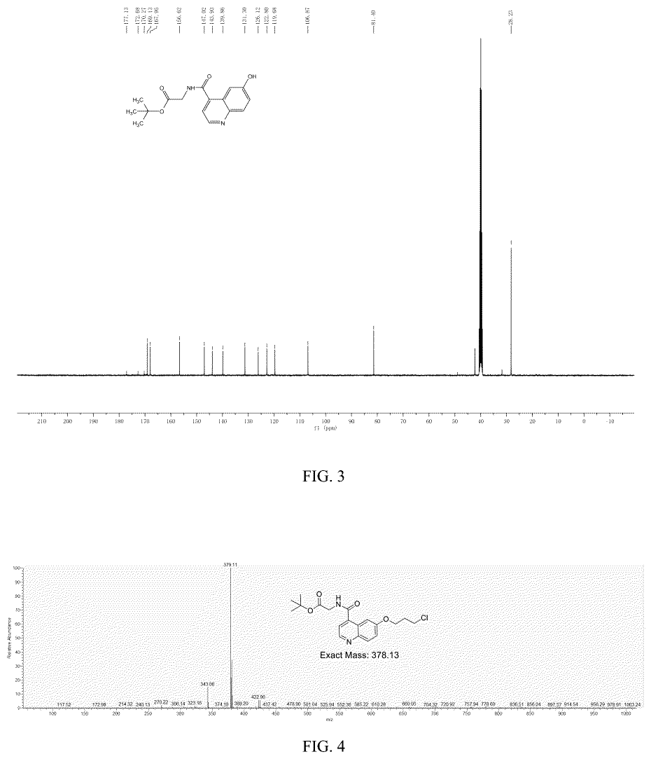

A compound 1 (6-hydroxyquinoline-4-carboxylic acid, 1.89 g, 10.0 mmol), glycine tert-butyl ester (1.89 g, 10.0 mmol), HATU (3.8 g, 10.0 mmol) and N,N-diisopropylethylamine (2.6 g, 20.0 mmol) were sequentially put into 30 mL of N,N-dimethylformamide in a 100 mL flask, respectively. A reaction mixture was stirred overnight and subjected to reduced pressure distillation to remove a solvent so as to obtain a crude product. Then purification was performed by a silica gel column (with dichloromethane and methanol at a ratio of 30:1) to obtain a white solid compound 2 with a yield of 87%. FIG. 1 is a mass spectrogram of the compound 2. FIG. 2 shows a nuclear magnetic hydrogen spectrum of the compound 2. FIG. 3 shows a nuclear magnetic carbon spectrum of the compound 2.

### (2) Synthesis of a Compound 3

The compound 2 (1.51 g, 5.0 mmol), 1-bromo-3-chloropropane (1.55 g, 10.0 mmol) and potassium carbonate (1.38 g, 10.0 mmol) were sequentially put into 50 mL of N,N-dimethylformamide in a 100 mL flask, respectively. The system was heated to 60° C., stirred overnight at 60° C. and subjected to reduced pressure distillation to remove a solvent so as to obtain a crude product. Then purification was performed by a silica gel column (with dichloromethane and methanol at a ratio of 50:1) to obtain a white solid compound 3 with a yield of 63%. FIG. 4 is a mass spectrogram of the compound 3. FIG. 5 shows a nuclear magnetic hydrogen spectrum of the compound 3.

### (3) Synthesis of a Compound 4

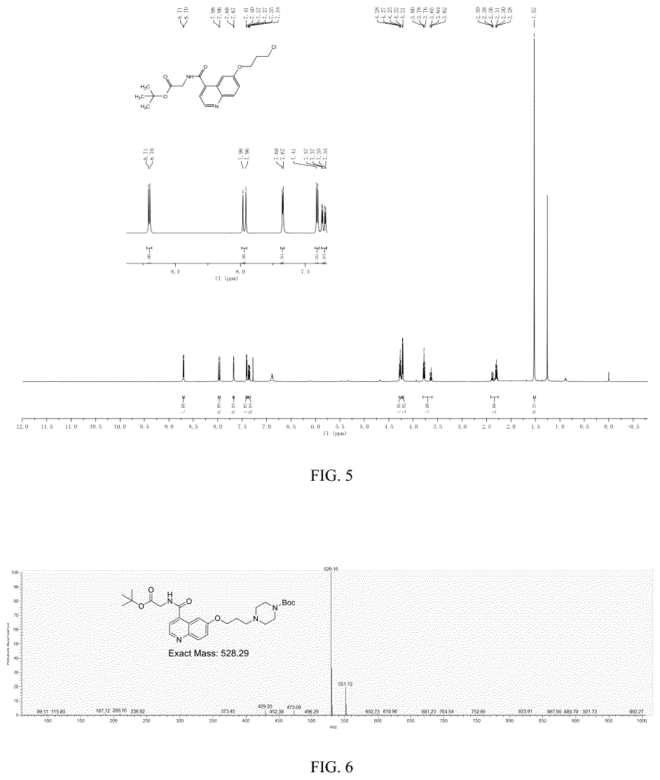

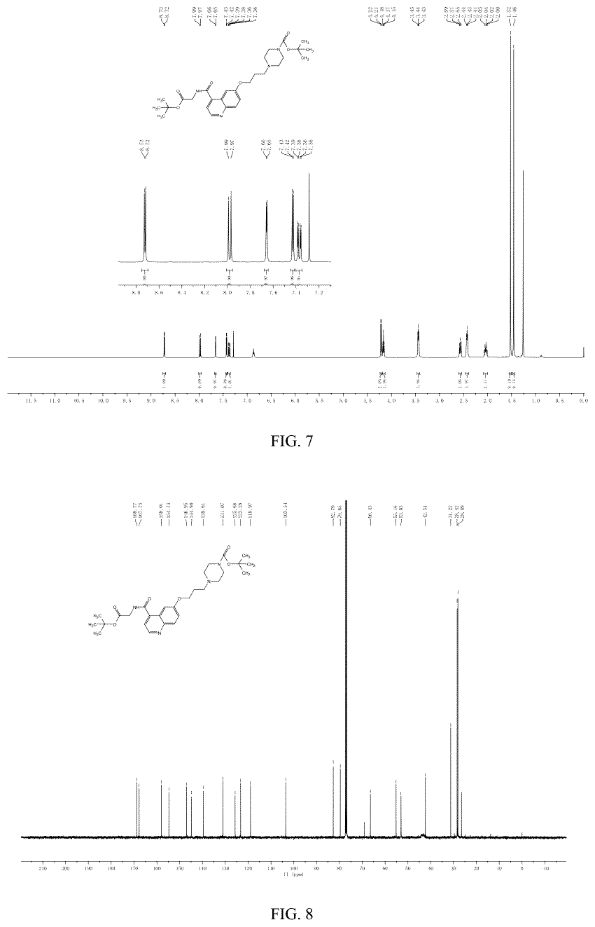

The compound 3 (0.76 g, 2.0 mmol), tert-butyl 1-piperazinecarboxylate (0.55 g, 3.0 mmol) and potassium iodide (0.49 g, 3.0 mmol) were sequentially put into 30 mL of acetonitrile in a 100 mL flask, respectively. The system was heated to 60° C., stirred overnight at 60° C. and subjected to reduced pressure distillation to remove a solvent so as to obtain a crude product. Then purification was performed by a silica gel column (with dichloromethane and methanol at a ratio of 30:1) to obtain a white solid compound 4 with a yield of 58%. MS (ESI) m/z calculated for [C₂₈H₄₀N₄O₆] was: 528.29; found: 529.10 [M+H]⁺. FIG. 6 is a mass spectrogram of the compound 4. FIG. 7 shows a nuclear magnetic hydrogen spectrum of the compound 4. FIG. 8 shows a nuclear magnetic carbon spectrum of the compound 4.

### (4) Synthesis of a Compound 5

The compound 4 (0.52 g, 1.0 mmol) was dissolved in 10 mL of a mixed solution of dichloromethane and trifluoroacetic acid (at a volume ratio of 9:1) under conditions of an ice bath. The system was heated to room temperature to carry out a reaction for 2 h. After the reaction was completed, reduced pressure distillation was performed to remove a solvent, and the system was dissolved in 10 mL of N,N-dimethylformamide to obtain a compound 5 for later use.

### (5) Synthesis of a Compound 6

Di-tert-butyl dicarbonate (0.22 g, 1.0 mmol) and N,N-diisopropylethylamine (0.39 g, 3.0 mmol) were separately added to an N,N-dimethylformamide solution of the compound 5. The system was stirred overnight at room temperature and then subjected to reduced pressure distillation to remove a solvent so as to obtain a crude product. Then purification was performed by a silica gel column (with dichloromethane and methanol at a ratio of 10:1) to obtain a white solid compound 6 with a yield of 72%.

### (6) Synthesis of a Compound 7

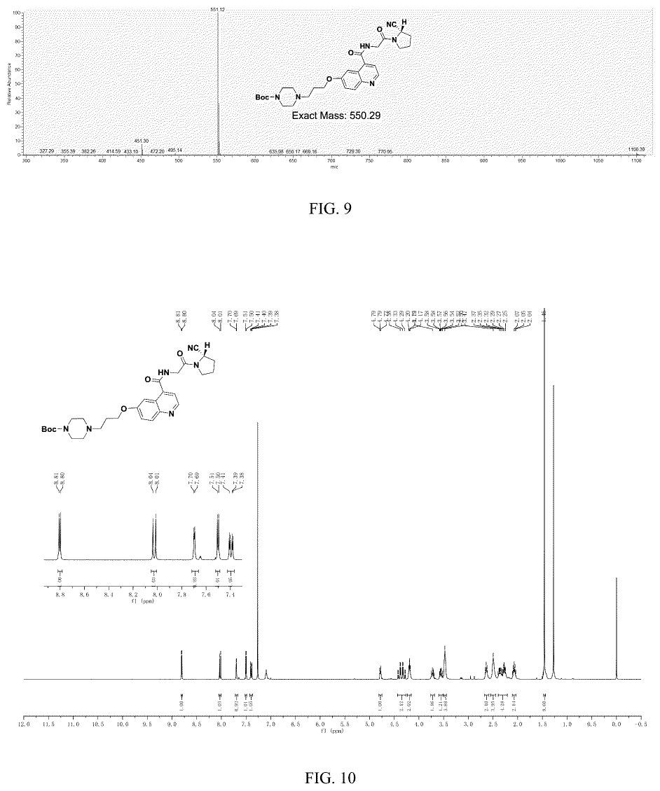

The compound 6 (0.47 g, 1.0 mmol), (S)-pyrrolidine-2-carbonitrile hydrochloride (0.13 g, 1.0 mmol), HATU (0.38 g, 1.0 mmol) and N,N-diisopropylethylamine (0.26 g, 2.0 mmol) were sequentially put into 10 mL of N,N-dimethylformamide in a 100 mL flask, respectively. A reaction mixture was stirred at room temperature until a reaction was completed, and then subjected to reduced pressure distillation to remove a solvent so as to obtain a crude product. Then purification was performed by a silica gel column (with dichloromethane and methanol at a ratio of 50:1) to obtain a white solid compound 7 with a yield of 85%. FIG. 9 is a mass spectrogram of the compound 7. FIG. 10 shows a nuclear magnetic hydrogen spectrum of the compound 7. FIG. 11 shows a nuclear magnetic carbon spectrum of the compound 7.

### (7) Synthesis of a Compound 8

The compound 7 (0.55 g, 1.0 mmol) and p-toluenesulfonic acid monohydrate (0.27 g, 1.5 mmol) were sequentially put into 10 mL of acetonitrile in a 100 mL flask, respectively. The reaction system was heated to 60° C., stirred until a reaction was completed, and subjected to reduced pressure distillation to remove a solvent so as to obtain a crude product of a compound 8.

### (8) Synthesis of a Compound 9

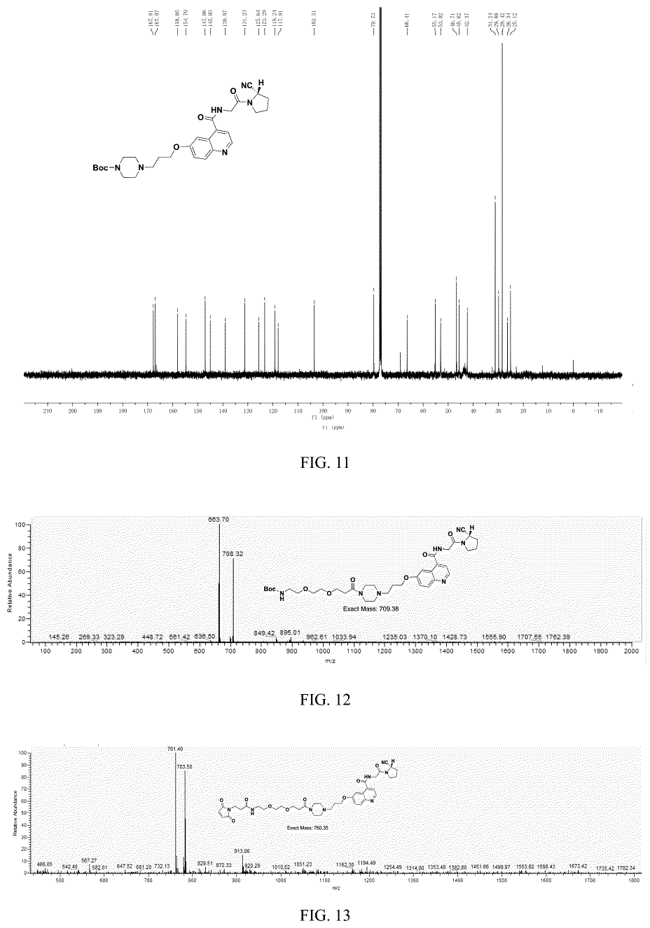

N-Boc-3-[2-(2-aminoethoxy)ethoxy]propionic acid (0.27 g, 1.0 mmol), HATU (0.38 g, 1.0 mmol), N,N-diisopropylethylamine (0.26 g, 2.0 mmol) and 10 mL of N, N-dimethylformamide were put into a reaction flask containing the compound 8, respectively. A reaction mixture was stirred overnight and subjected to reduced pressure distillation to remove a solvent so as to obtain a crude product. Then purification was performed by a silica gel column (with dichloromethane and methanol at a ratio of 50:1) to obtain a white solid compound 9 with a yield of 64%. FIG. 12 is a mass spectrogram of the compound 9.

### (9) Synthesis of a Compound 10

The compound 9 (0.66 g, 1.0 mmol) and p-toluenesulfonic acid monohydrate (0.27 g, 1.5 mmol) were sequentially put into 10 mL of acetonitrile in a 100 mL flask, respectively. The reaction system was heated to 60° C., stirred until a reaction was completed, and subjected to reduced pressure distillation to remove a solvent so as to obtain a crude product. The crude product was dissolved in 10 mL of N,N-dimethylformamide, then HATU (0.38 g, 1.0 mmol), N,N-diisopropylethylamine (0.26 g, 2.0 mmol) and maleimide propionate (0.17 g, 1.0 mmol) were added to carry out a reaction for 3 h, and reduced pressure distillation was performed to remove a solvent so as to obtain a crude product. Then purification was performed by a silica gel column (with dichloromethane and methanol at a ratio of 50:1) to obtain a white solid compound 10 with a yield of 59%. MS(ESI) m/z calculated for [C₃₅H₅₁N₇O₈] was: 697.38; found: 698.43 [M+H]⁺. FIG. 13 is a mass spectrogram of the compound 10.

### (10) Synthesis of a Compound 11 (Namely, a Compound of Formula (I-1))

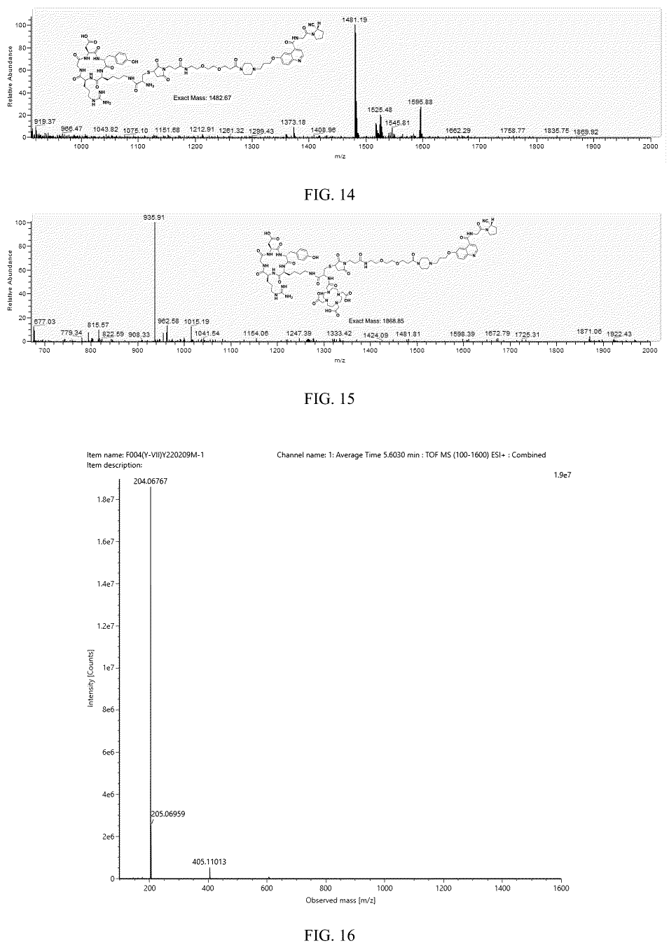

The compound 10 (0.072 g, 0.1 mmol), Boc-protected cysteine (0.022 g, 0.1 mmol) and 10 mL of N, N-dimethylformamide were put into a 25 mL flask, respectively, and stirred at room temperature to carry out a reaction for 3 h. After the reaction was completed based on monitoring, 0.12 mmol of DCC and NHS were added to the system, the system was stirred to carry out a reaction for 12 h, then c(RGDyK) (0.06 g, 0.1 mmol) and N,N-diisopropylethylamine (0.065 g, 0.05 mmol) were added to carry out a reaction for 12 h, and reduced pressure distillation was performed to remove a solvent so as to obtain a crude product. Then the crude product was purified by a reversed phase column, and freeze-dried to obtain a pure compound 11 (namely, a compound of Formula (I-1)) with a yield of 43% in two steps. FIG. 14 is a mass spectrogram of the compound 11.

### (11) Synthesis of a Compound of Formula (V-1)

The compound 11 (0.146 g, 0.1 mmol), and thioanisole, 1,2-ethanedithiol, anisole and TFA (at a ratio of 5:3:2:90) were put into a 25 mL flask, respectively to remove tert-butyl and Boc protective groups at room temperature. After a reaction was completed, the TFA was removed by an argon flow, the system was dissolved in 10 mL of N,N-dimethylformamide, and DOTA-NHS (0.05 g, 0.1 mmol) and N,N-diisopropylethylamine (0.04 g, 0.3 mmol) were sequentially added. The reaction system was stirred to carry out a reaction at room temperature. After the reaction was completed based on monitoring by HPLC, reduced pressure distillation was performed to remove a solvent so as to obtain a crude product. Then the crude product was purified by a reversed phase column, and freeze-dried to obtain a pure compound of Formula (V-1) with a yield of 53%. FIG. 15 is a mass spectrogram of the compound of Formula (V-1).

### Example 2: Preparation of a Compound of Formula (I-14) and a Compound of Formula (V-14)

A synthesis route is as follows.

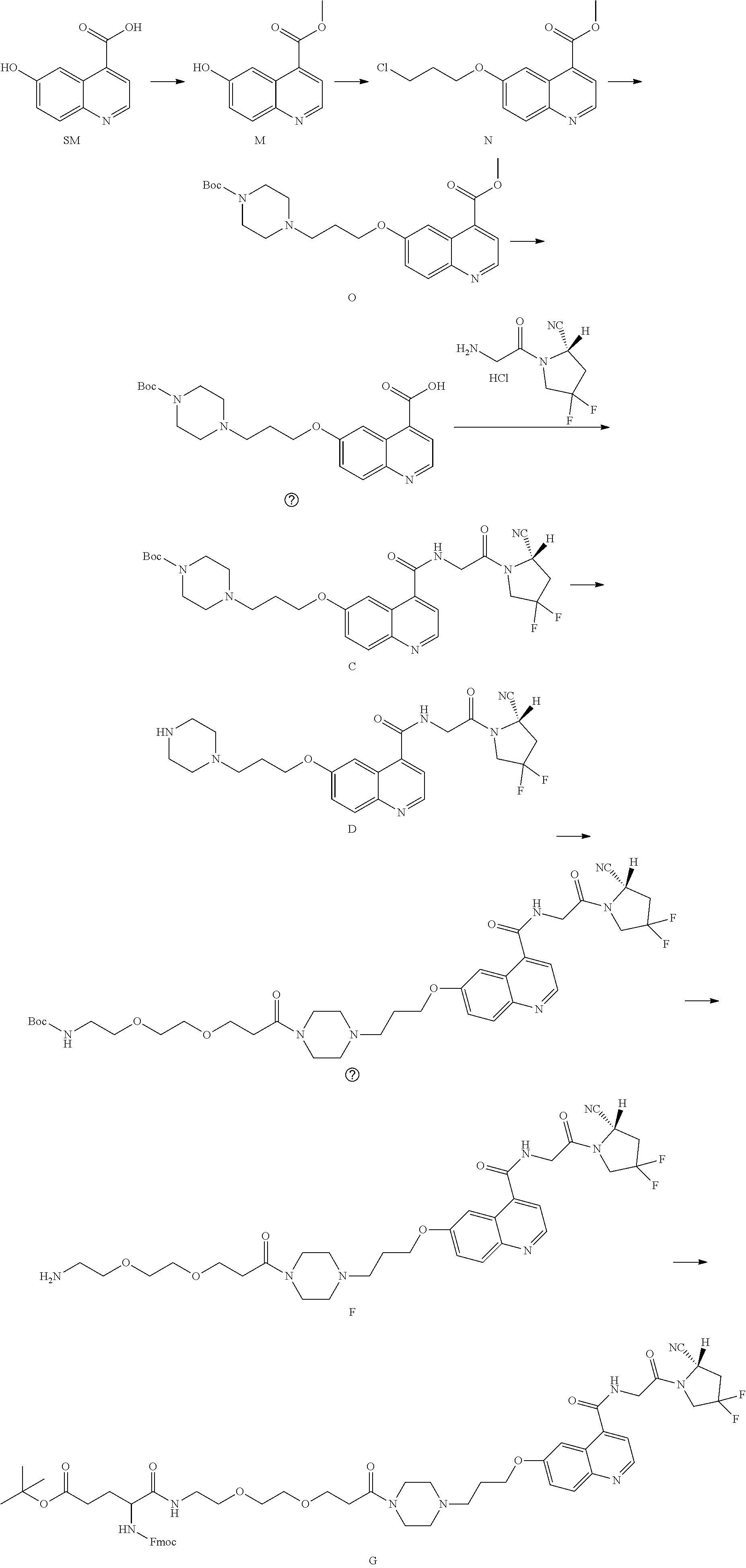

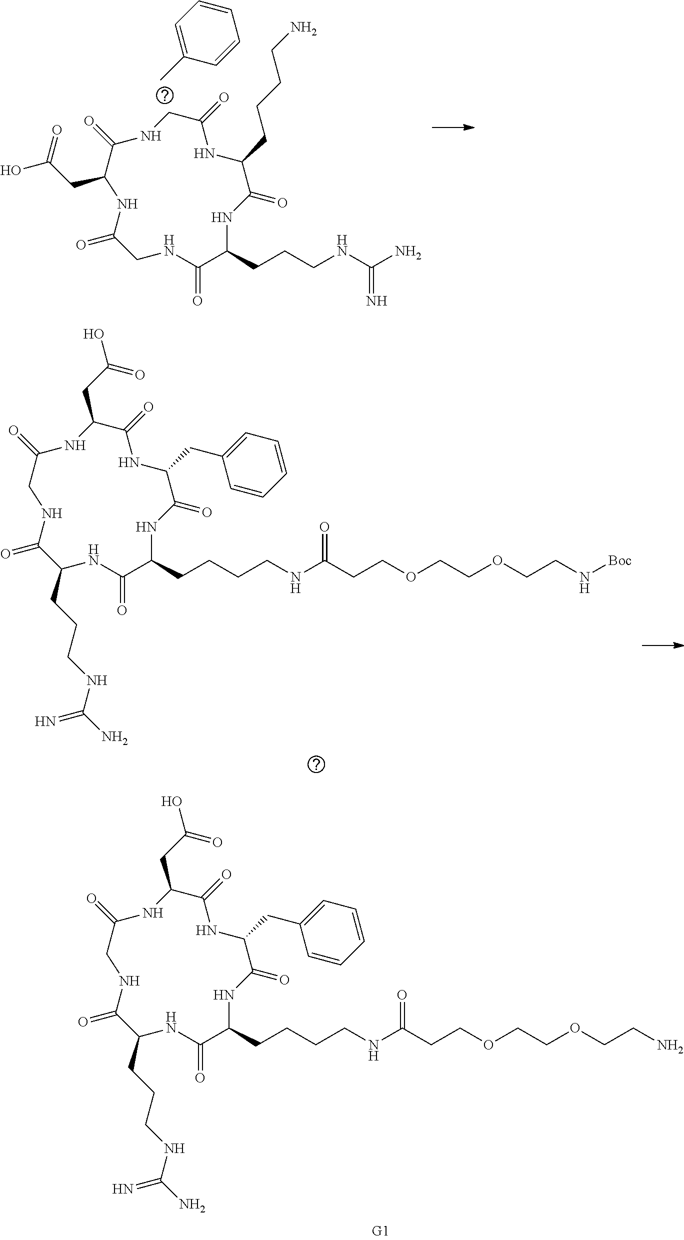

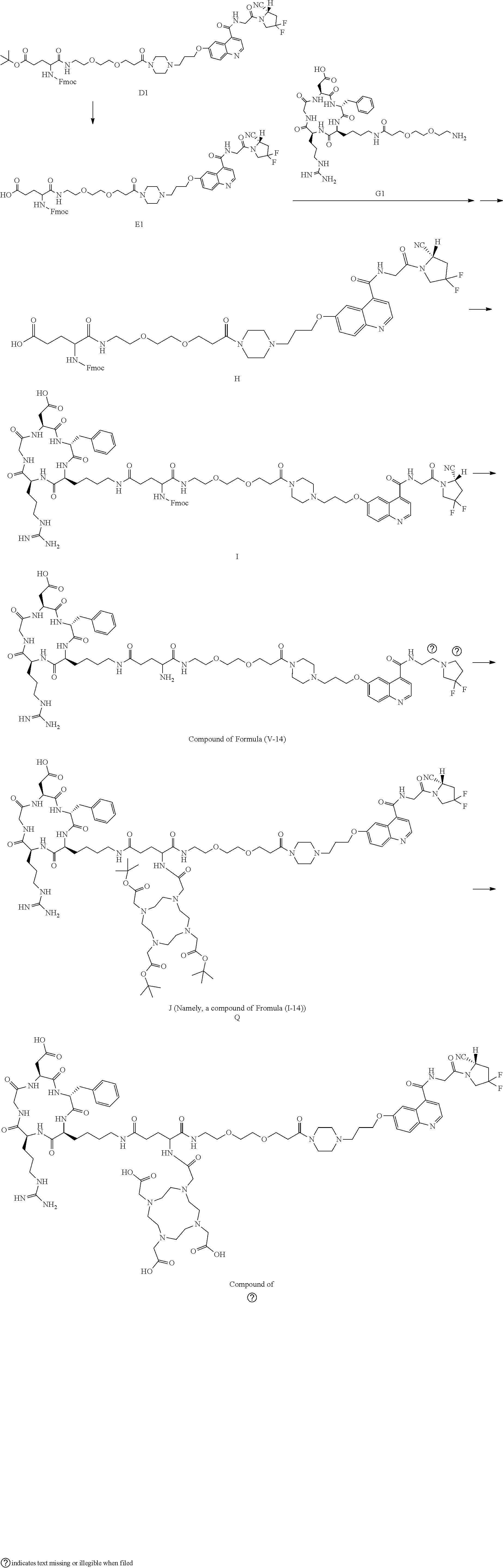

### (1) Preparation of an Intermediate M

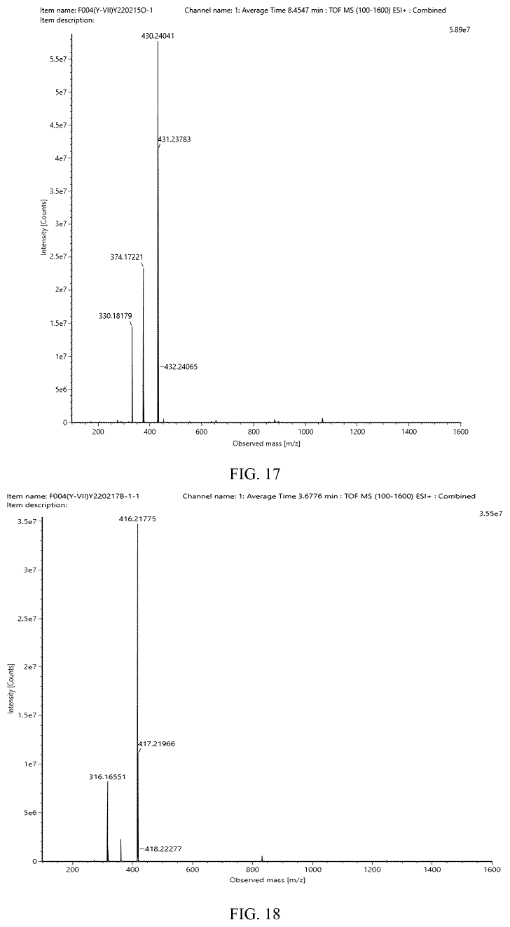

SM (6-hydroxyquinoline-4-carboxylic acid) was dissolved in 100 mL of methanol, 1 mL of concentrated sulfuric acid was added dropwise, a resulting mixture was placed in an external bath at 90° C. to carry out a reaction overnight, and the reaction was completed based on monitoring by thin layer chromatography (TLC). The methanol was concentrated, and the concentrated system was added dropwise into 40 mL of saturated NaHCO₃. After the dropping was completed, the system was stirred and crystallized for 1 h, and filtered and dried to obtain an intermediate M with a yield of 59%. A theoretical molecular weight is 203.0582, a measured molecular weight is 203.06767, and mass spectrometry results are consistent with that of the target product. FIG. 16 is a mass spectrogram of the intermediate M.

### (2) Preparation of an Intermediate N

The intermediate M was dissolved in 30 mL of DMF, and potassium carbonate (2.00 g, 14.5 mmol) and 1-bromo-3-chloropropane (2.19 g, 13.9 mmol) were sequentially added to carry out a reaction overnight in an external bath at 25° C. After the reaction was completed based on monitoring by TLC, 70 mL of purified water was added, extraction was performed with 70 mL of DCM for two times, and organic phases were combined, dried and concentrated to obtain a crude product of an intermediate N with a crude yield of 83.8%.

### (3) Preparation of an Intermediate O

The intermediate N, 1-Boc-piperazine (1.67 g, 6.8 mmol), and KI (1.11 g, 6.7 mmol) were sequentially dissolved in 10 mL of DMF to carry out a reaction in an external bath at 100° C. After the reaction was completed based on monitoring by TLC, 60 mL of purified water was added, extraction was performed with 30 mL of DCM for two times, and organic phases were combined and washed with 30 mL of purified water for two times. Then, the organic phases were dried with anhydrous sodium sulfate and subjected to column purification to obtain a crude product with a yield of 86%. A theoretical molecular weight is 429.2264, a measured molecular weight is 429.24041, and mass spectrometry results are consistent with that of the target product. FIG. 17 is a mass spectrogram of the intermediate O.

### (4) Preparation of an Intermediate B

The intermediate O was dissolved in 10 mL of methanol, and a LiOH/1V aqueous solution was added to carry out a reaction in an external bath at 25° C. After the reaction was completed based on monitoring by TLC, the methanol in the system was concentrated, 2 mL of water was added to the system, and 1 N of HCl was slowly added dropwise to adjust the pH value to 6-7. Then the system was crystallized for 1 h, filtered and dried to obtain an intermediate B with a yield of 85%. A theoretical molecular weight is 415.2107, a measured molecular weight is 415.21775, and mass spectrometry results are consistent with that of the target product. FIG. 18 is a mass spectrogram of the intermediate B.

### (5) Preparation of an Intermediate C

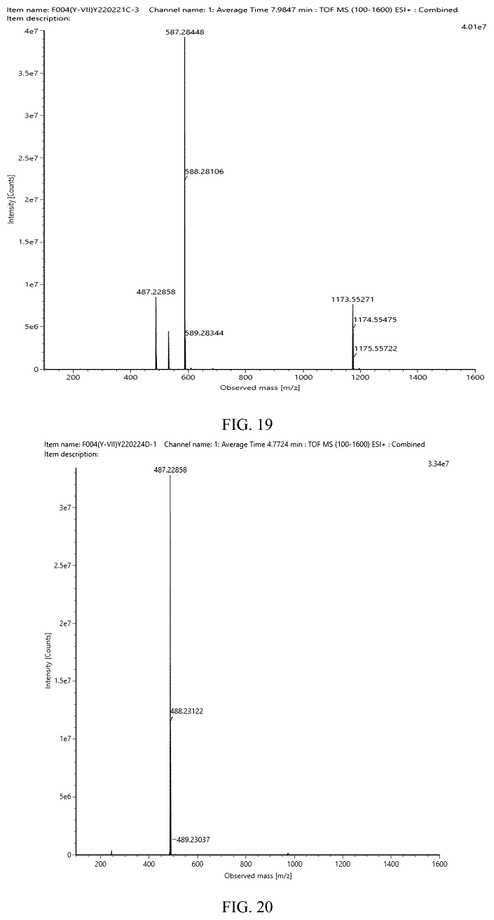

The intermediate B, (S)-4,4-difluoro-1-glycylpyrrolidine-2-carbonitrile hydrochloride (1.11 g, 2.7 mmol), HATU (1.06 g, 2.8 mmol) and DIPEA (1.1 g, 8.1 mmol) were sequentially dissolved in 10 mL of DMF to carry out a reaction in an external bath at 25° C. After the reaction was completed based on monitoring by TLC, 30 mL of purified water was added to the system, extraction was performed with 3 mL of DCM for two times, and organic phases were combined, dried with anhydrous sodium sulfate, concentrated and subjected to column purification to obtain an intermediate C with a yield of 73.4%. A theoretical molecular weight is 586.2715, a measured molecular weight is 586.28448, and mass spectrometry results are consistent with that of the target product. FIG. 19 is a mass spectrogram of the intermediate C.

### (6) Preparation of an Intermediate D

The intermediate C was dissolved in 10 mL of acetonitrile, p-toluenesulfonic acid monohydrate (1.54 g, 8.1 mmol) was added, and a resulting mixture was placed in an external bath at 65° C. to carry out a reaction. After the reaction was completed based on monitoring by TLC, a crude product of an intermediate D was obtained. A theoretical molecular weight is 486.2191, a measured molecular weight is 486.22858, and mass spectrometry results are consistent with that of the target product. FIG. 20 is a mass spectrogram of the intermediate D.

### (7) Preparation of an Intermediate E

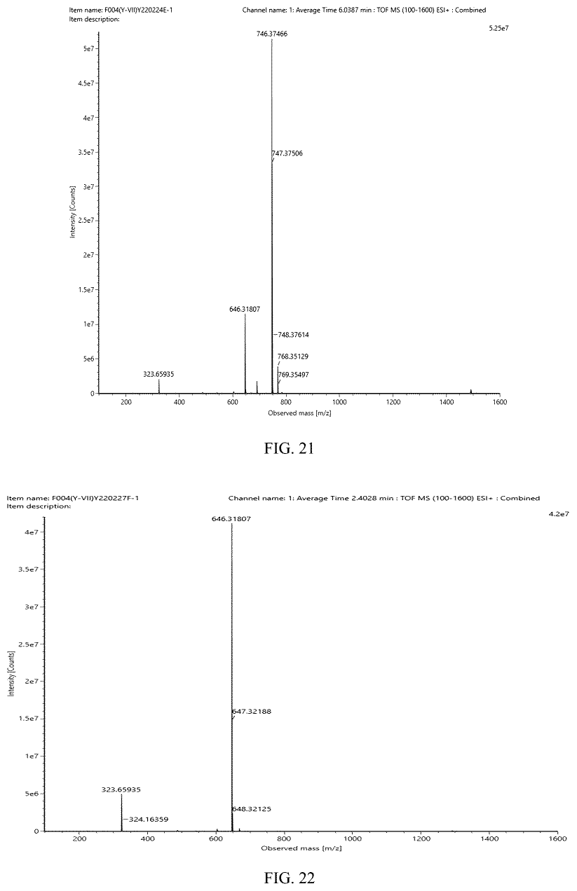

The intermediate D was dissolved in 10 mL of DMF, and DIPEA (2.71 g, 21.1 mmol) and t-Boc-N-amido-PEG2-NHS-ester (1.22 g, 6.3 mmol) were sequentially added to carry out a reaction in an external bath at 25° C. After the reaction was completed based on monitoring by HPLC, 30 mL of purified water was added to the system, extraction was performed with 30 mL of DCM for two times, and organic phases were combined, dried, concentrated and subjected to column purification to obtain an intermediate E with a yield of 64.7% in two steps. A theoretical molecular weight is 745.3611, a measured molecular weight is 745.37466, and mass spectrometry results are consistent with that of the target product. FIG. 21 is a mass spectrogram of the intermediate E.

### (8) Preparation of an Intermediate F

The intermediate E was dissolved in 10 mL of acetonitrile, p-toluenesulfonic acid monohydrate (2.42 g, 12.7 mmol) was added, and a reaction was carried out in an external bath at 65° C. After the reaction was completed based on monitoring by HPLC, the system was concentrated to obtain a crude product of an intermediate F. A theoretical molecular weight is 645.3086, a measured molecular weight is 645.31807, and mass spectrometry results are consistent with that of the target product. FIG. 22 is a mass spectrogram of the intermediate F.

### (9) Preparation of an Intermediate G

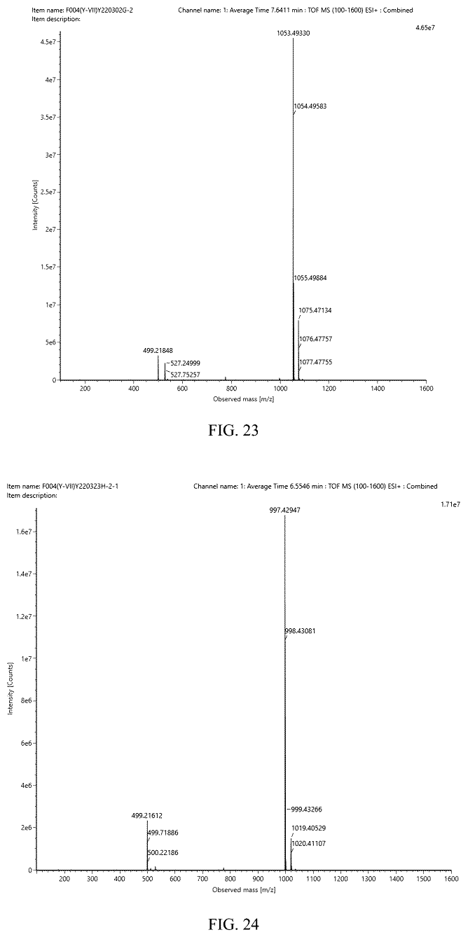

The intermediate F and DIPEA (1.28 g, 10.1 mmol) were sequentially dissolved in 5 mL of DMF to obtain a system (1). HATU (0.84 g, 2.2 mmol) and Fmoc-Glu(OtBu)OH (0.61 g, 2.2 mmol) were sequentially dissolved in 5 mL of DMF to obtain a system (2). The system (2) was stirred at 25° C. for 1 h and then added to the system (1) to carry out a reaction at 25° C. After the reaction was completed based on monitoring by TLC, 20 mL of purified water was added to the system, extraction was performed with 20 mL of DCM for two times, and organic phases were combined, washed with saturated sodium chloride for one time, concentrated and subjected to column purification to obtain an intermediate G with a yield of 63.6% in two steps. A theoretical molecular weight is 1052.4819, a measured molecular weight is 1052.49330, and mass spectrometry results are consistent with that of the target product. FIG. 23 is a mass spectrogram of the intermediate G.

### (10) Preparation of an Intermediate H

The intermediate G was dissolved in 10 mL of acetonitrile, p-toluenesulfonic acid monohydrate (2.87 g, 15.1 mmol) was added, and a reaction was carried out at 65° C. After the reaction was completed based on monitoring by HPLC, the system was concentrated and subjected to column purification to obtain an intermediate H. A theoretical molecular weight is 996.4193, a measured molecular weight is 996.42947, and mass spectrometry results are consistent with that of the target product. FIG. 24 is a mass spectrogram of the intermediate H.

### (11) Preparation of an Intermediate I

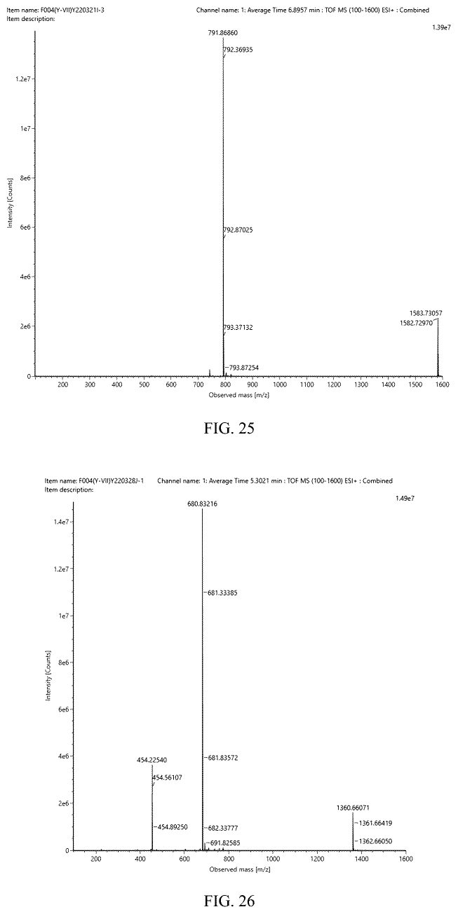

The intermediate H and DIPEA (1.62 g, 10.1 mmol) were dissolved in 10 mL of DMF, and HATU (1.37 g, 3.6 mmol) and NHS (1.28 g, 5.85 mmol) were sequentially added and stirred to carry out a reaction at 30° C. so as to obtain a system (1). RGDfK (2.17 g, 3.6 mmol) was dissolved in 10 mL of DMSO to obtain a system (2). After the reaction of the system (1) was completed based on monitoring by TLC, the system (2) was added to the system (1) in three batches at an interval of 15 min for each batch. After the addition was completed, a reaction was carried out in an external bath at 30° C. After the reaction was completed based on monitoring by HPLC, the system was concentrated and subjected to preparative purification to obtain an intermediate I with a yield of 34.2%. A theoretical molecular weight is 1581.7216, a measured molecular weight is 1581.7372, and mass spectrometry results are consistent with that of the target product. FIG. 25 is a mass spectrogram of the intermediate I.

### (12) Preparation of an Intermediate J (Namely, a Compound of Formula (I-14))

The intermediate I was dissolved in 30 mL of DMF, and 2 mL of piperidine was added to carry out a reaction at 25° C. After the reaction was completed based on monitoring by HPLC, 200 mL of MTBE was added to the system for crystallization. Then, standing was performed, a supernatant was sucked out, and the remaining system was concentrated to obtain a crude product of an intermediate J (namely, a compound of Formula (I-14)), which was directly used in the next step. A theoretical molecular weight is 1359.6536, a measured molecular weight is 1359.66432, and mass spectrometry results are consistent with that of the target product. FIG. 26 is a mass spectrogram of the intermediate J.

### (13) Preparation of an Intermediate Q

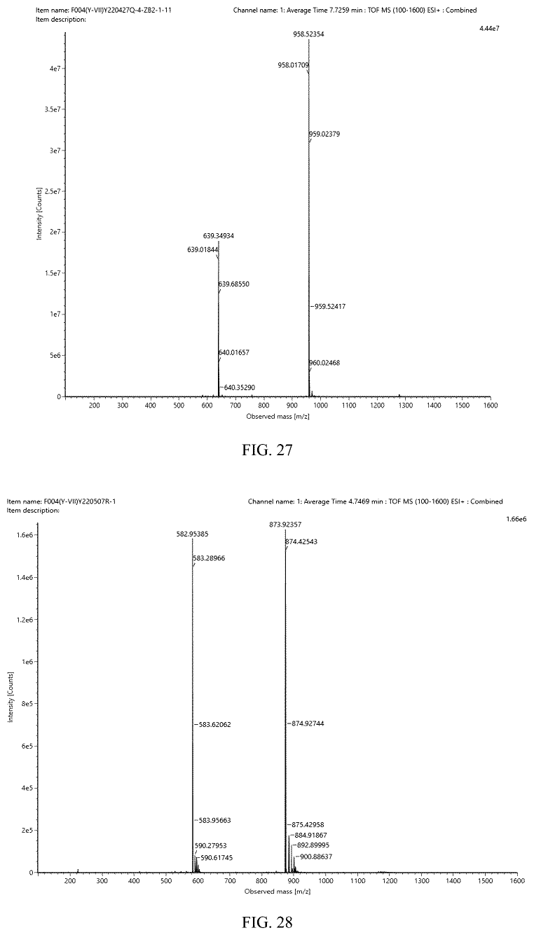

The intermediate J was dissolved in 20 mL of DMF, and DIPEA (0.81 g, 5.0 mmol) and DOTA-TRIS-TBU-NHS Ester (0.50 g, 1.0 mmol) were sequentially added. After a reaction was completed based on monitoring by HPLC, the system was concentrated and subjected to preparative purification to obtain an intermediate Q with a yield of 15.7% in two steps. A theoretical molecular weight is 1914.0215, a measured molecular weight is 1914.03418, and mass spectrometry results are consistent with that of the target product. FIG. 27 is a mass spectrogram of the intermediate Q.

### (14) Preparation of a Compound of Formula (V-14)

The intermediate Q was dissolved in 10 mL of TFA, and a reaction was carried out in an external bath at 25° C. After the reaction was completed based on monitoring by HPLC, 100 mL of MTBE was added to the system for crystallization, standing was performed, and a supernatant was sucked out. Then the remaining system was concentrated, concentrated with MTBE until no obvious TFA residues were remained, and subjected to preparative purification to obtain a compound of Formula (V-14). A theoretical molecular weight is 1745.8337, a measured molecular weight is 1745.84714, and mass spectrometry results are consistent with that of the target product. FIG. 28 is a mass spectrogram of the compound of Formula (V-14).

### Example 3: Preparation of a Compound of Formula (V-23)

A synthesis route is as follows.

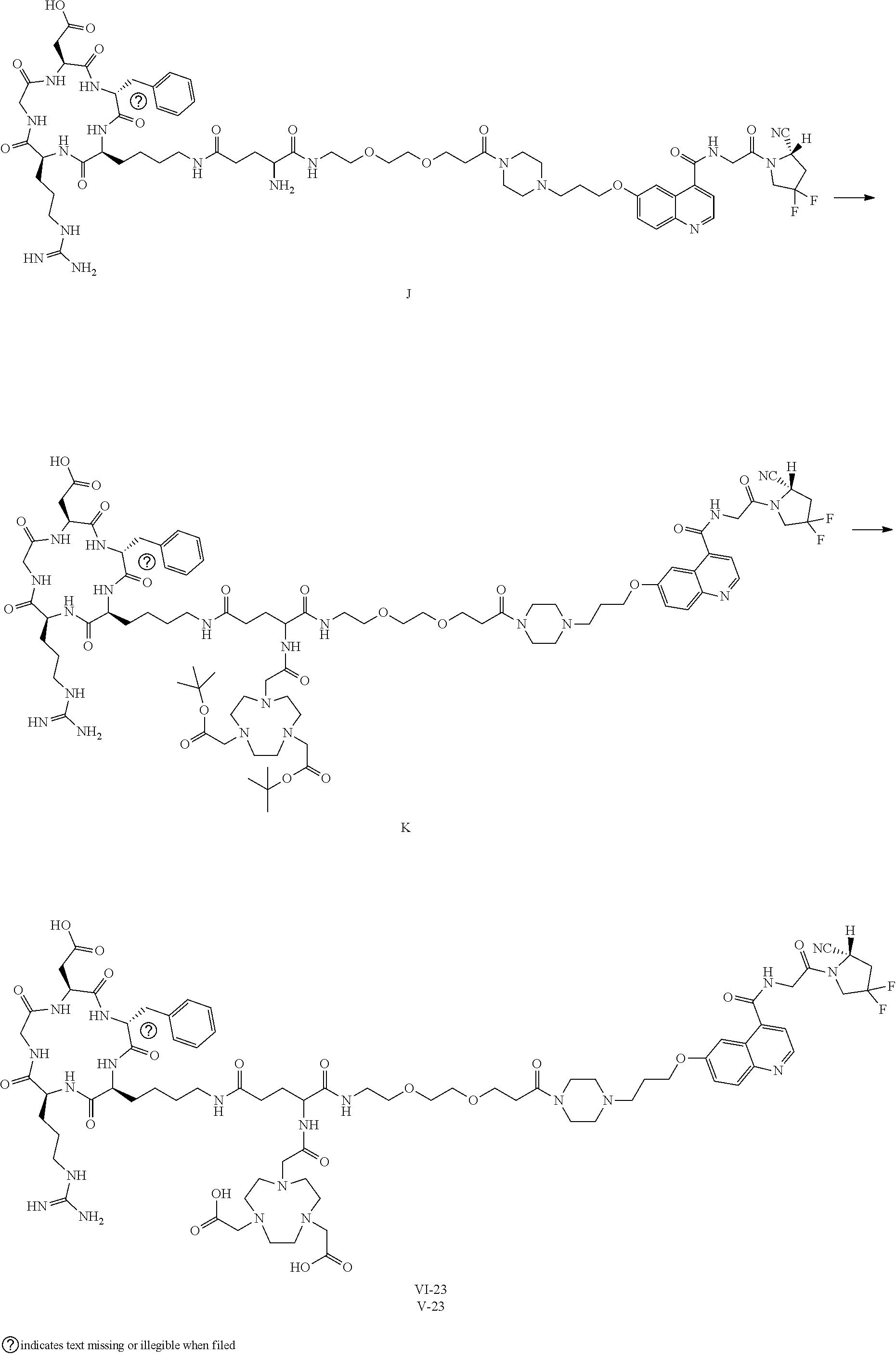

### (1) Preparation of an Intermediate K

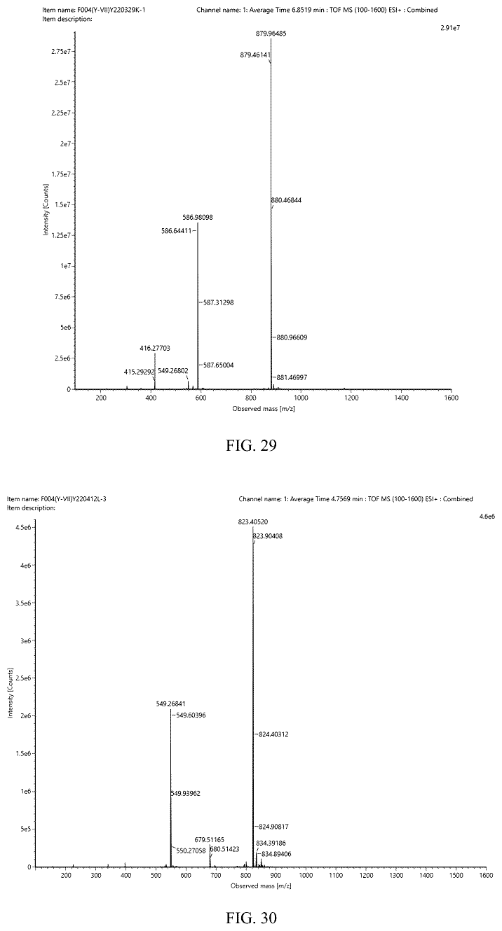

The intermediate J prepared by the method in Example 2 was dissolved in 30 mL of DMF, DIPEA (0.97 g, 7.5 mmol) and 2 eq of NOTA-Bis-TBU-NHS Ester (calculated based on the intermediate J) were added, and a reaction was carried out in an external bath at 25° C. After the reaction was completed based on monitoring by HPLC, the system was concentrated and subjected to preparative purification to obtain an intermediate K with a yield of 25.1% in two steps. A theoretical molecular weight is 1756.9112, a measured molecular weight is 1756.92282, and mass spectrometry results are consistent with that of the target product. FIG. 29 is a mass spectrogram of the intermediate K.

### (2) Preparation of a Compound of Formula (V-23)

The intermediate K was dissolved in 30 mL of TFA, and a reaction was carried out in an external bath at 25° C. After the reaction was completed based on monitoring by HPLC, 200 mL of MTBE was added to the system for crystallization, standing was performed, and a supernatant was sucked out. Then the remaining system was concentrated, concentrated with MTBE until no obvious TFA residues were remained, and subjected to preparative purification to obtain a compound of Formula (V-23) with a yield of 14.2%. A theoretical molecular weight is 1644.7860, a measured molecular weight is 1644.8104, and mass spectrometry results are consistent with that of the target product. FIG. 30 is a mass spectrogram of the compound of Formula (V-23).

### Example 4: Preparation of a Compound of Formula (I-25) and a Compound of Formula (V-25)

A synthesis route is as follows.

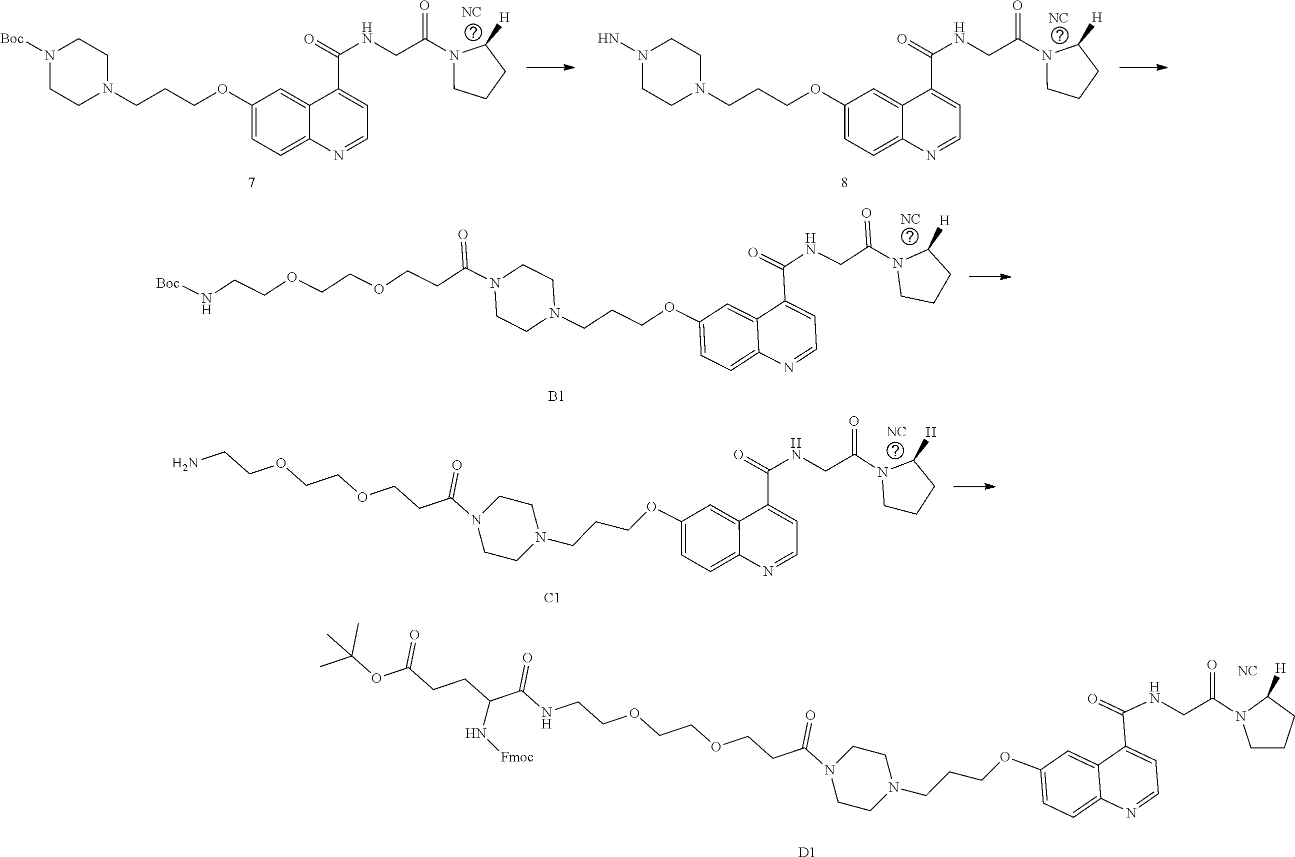

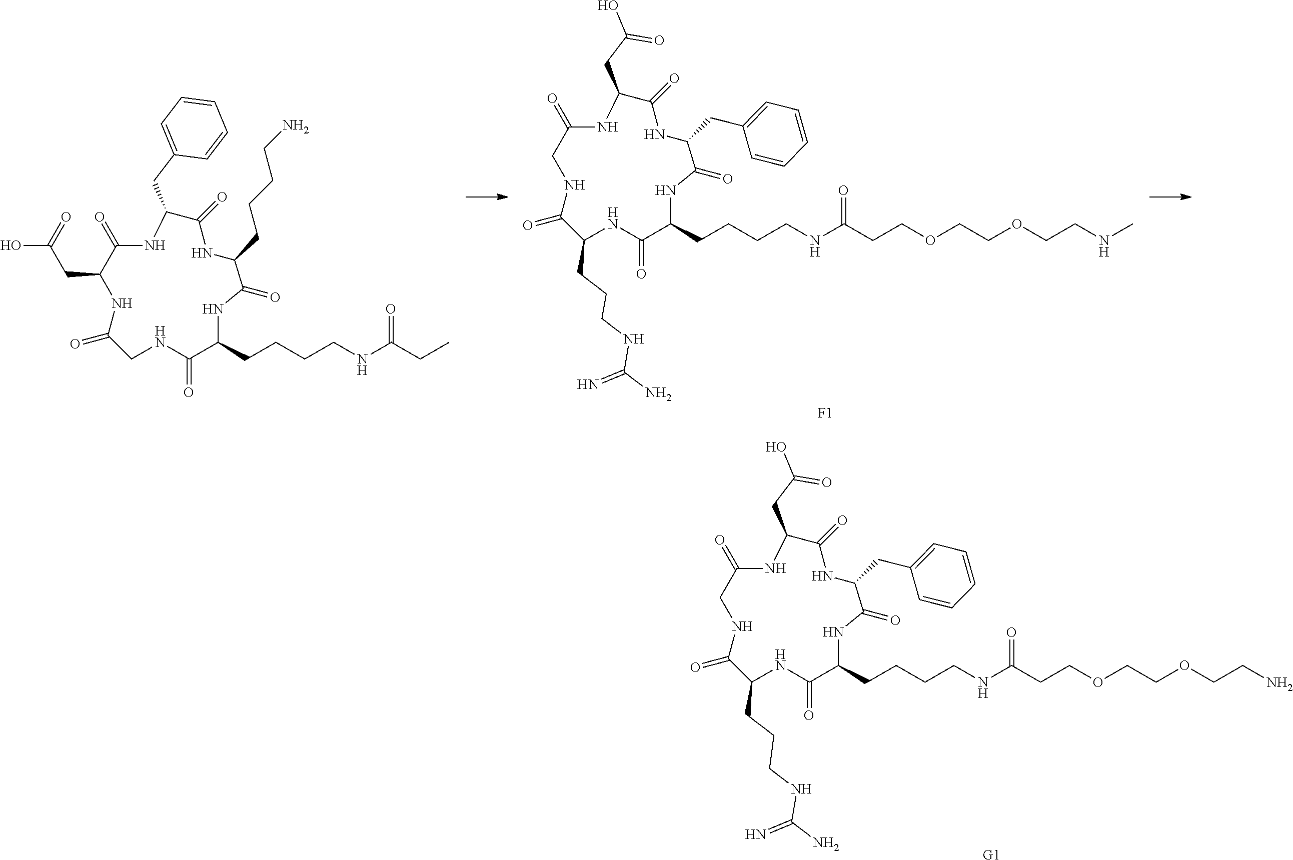 [SMILES: CCC(=O)NCCCC[C@@H]1NC(=O)[C@H](CCCCN)NC(=O)[C@@H](Cc2ccccc2)NC(=O)[C@H](CC(=O)O)NC(=O)CNC1=O ; CNCCOCCOCCC(=O)NCCCC[C@@H]1NC(=O)[C@@H](Cc2ccccc2)NC(=O)[C@H](CC(=O)O)NC(=O)CNC(=O)[C@H](CCCNC(=N)N)NC1=O ; "F1" ; N=C(N)NCCC[C@@H]1NC(=O)[C@H](CCCCNC(=O)CCOCCOCCN)NC(=O)[C@@H](Cc2ccccc2)NC(=O)[C@H](CC(=O)O)NC(=O)CNC1=O ; "G1"]

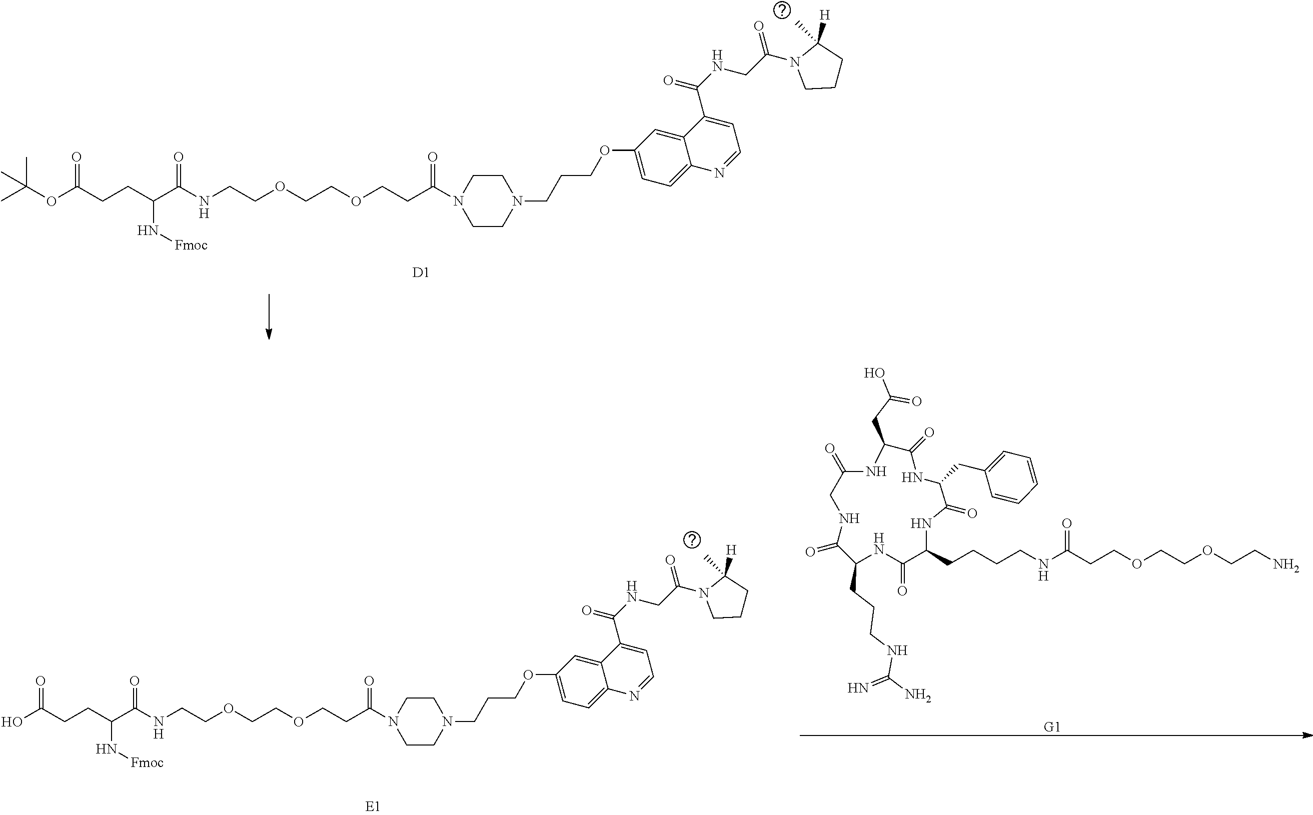

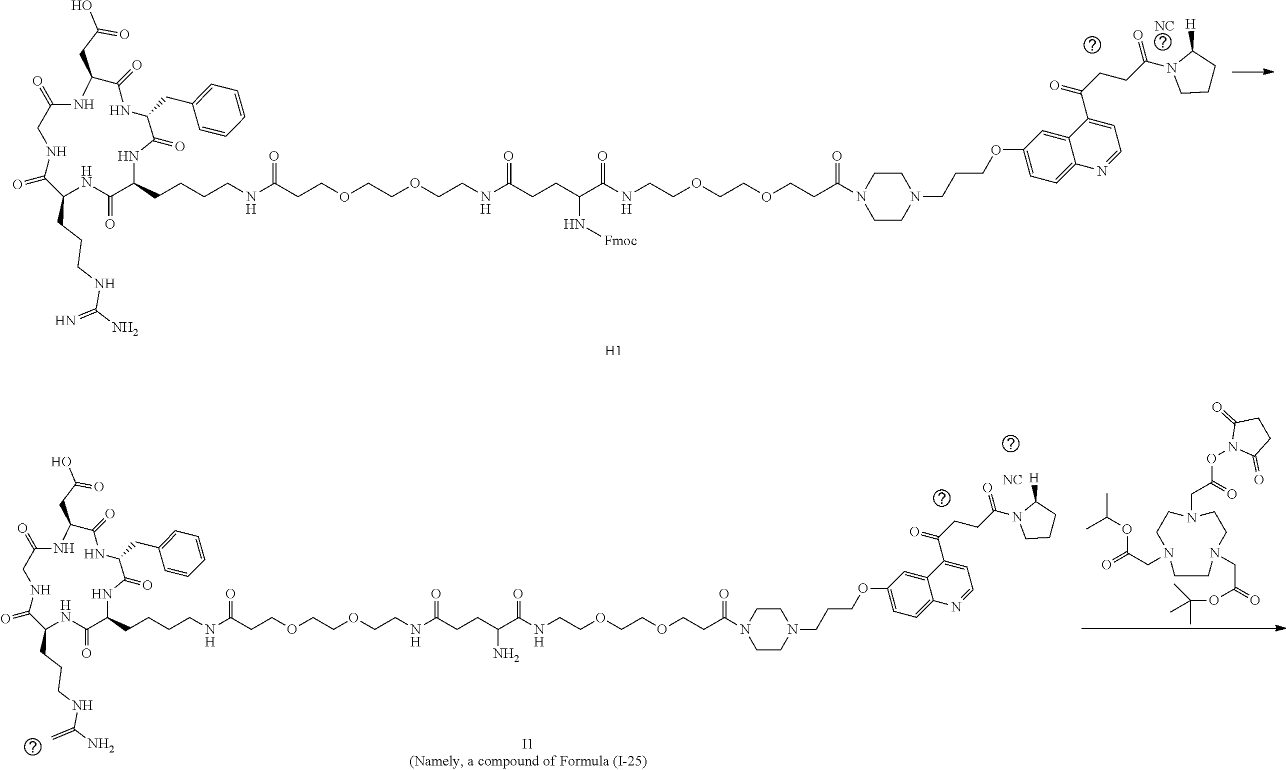

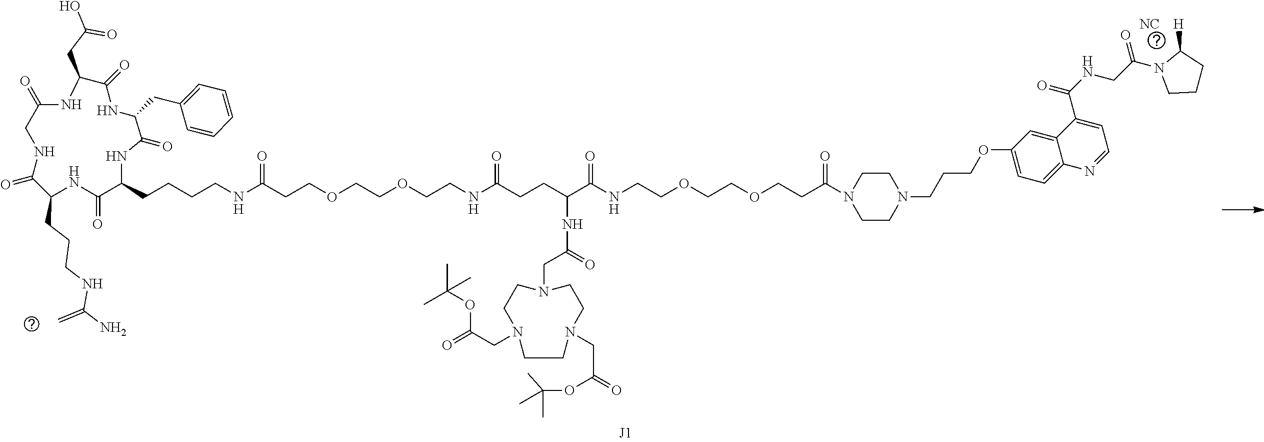

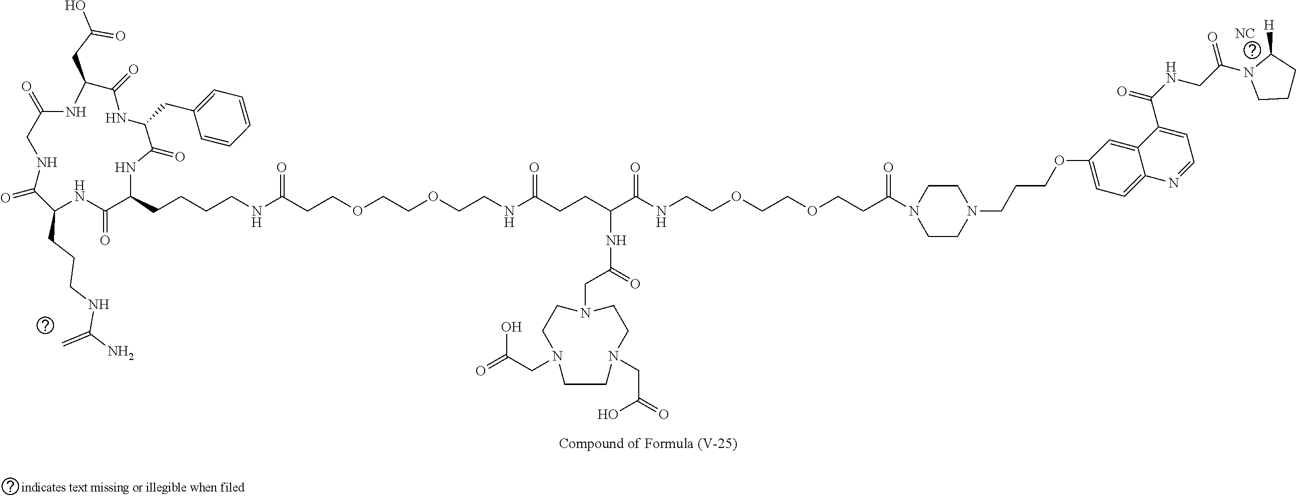

### (1) Preparation of an Intermediate B1

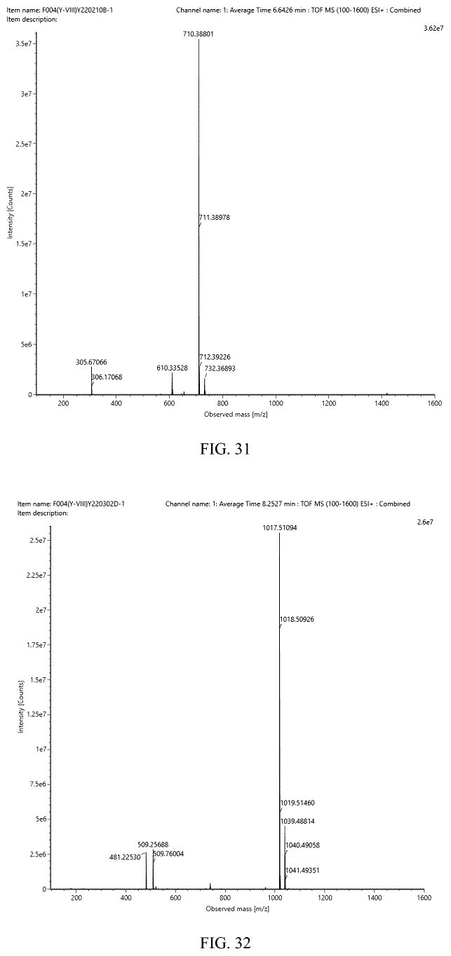

The compound 7 (2.50 g, 4.5 mmol), p-toluenesulfonic acid monohydrate (2.58 g, 13.6 mmol) and 25 mL of acetonitrile were added into a reaction flask to carry out a reaction at 65° C. for 1 h. After the reaction was completed based on monitoring by TLC (with methanol and dichloromethane at a ratio of 5.1), evaporation was performed under reduced pressure at 40° C. 14 ml of DMF and DIPEA (3.05 g, 23.6 mmol) were added and stirred at 25° C. to carry out a reaction, numbered (1), and that is to say, a piperazinyl protective group of the compound 7 was removed to obtain an intermediate 8. N-tert-butyl oxy carbonyl-dipolyethylene glycol-carboxylic acid (1.62 g, 4.8 mmol), HATU (2.60 g, 6.8 mmol) and 10 mL of DMF were added into another reaction flask to carry out a reaction, numbered (2), at 25° C. for 30 min, and the reaction system (2) was added to the reaction system (1) to carry out a reaction for 1 h. Evaporation was performed under reduced pressure at 40° C. 50 mL of purified water was added, extraction was performed with DCM for two times at 50 mL each time, and the DCM was combined. Drying was performed with anhydrous sodium sulfate, and filtration and evaporation were performed to obtain a crude product. The crude product was purified by column chromatography to obtain 1.68 g of a target product. A theoretical molecular weight is 709.3799, a measured molecular weight is 709.38801, and mass spectrometry results are consistent with that of the target product. FIG. 31 is a mass spectrogram of the intermediate B1.

### (2) Preparation of an Intermediate D1

The intermediate B1, p-toluenesulfonic acid monohydrate (1.61 g, 8.5 mmol) and 20 mL of acetonitrile were added into a reaction flask to carry out a reaction at 65° C. for 1 h, and evaporation was performed under reduced pressure at 40° C. 20 ml of DMF and DIPEA (1.83 g, 14.2 mmol) were added and stirred at 25° C. to carry out a reaction, numbered (1), and that is to say, a piperazinyl protective group of the intermediate B1 was removed to obtain an intermediate C1. Fmoc-O-tert butyl-L-glutamic acid (1.43 g, 3.4 mmol), HATU (1.29 g, 3.4 mmol) and 20 mL of DMF were added into another reaction flask to carry out a reaction, numbered (2), at 25° C. for 30 min, and the reaction system (2) was added to the reaction system (1) to carry out a reaction for 1 h. Evaporation was performed under reduced pressure at 40° C. to obtain a crude product, and the crude product was purified by column chromatography to obtain 1.19 g of a target product. A theoretical molecular weight is 1016.5008, a measured molecular weight is 1016.51094, and mass spectrometry results are consistent with that of the target product. FIG. 32 is a mass spectrogram of the intermediate D1.

### (3) Preparation of an Intermediate G1

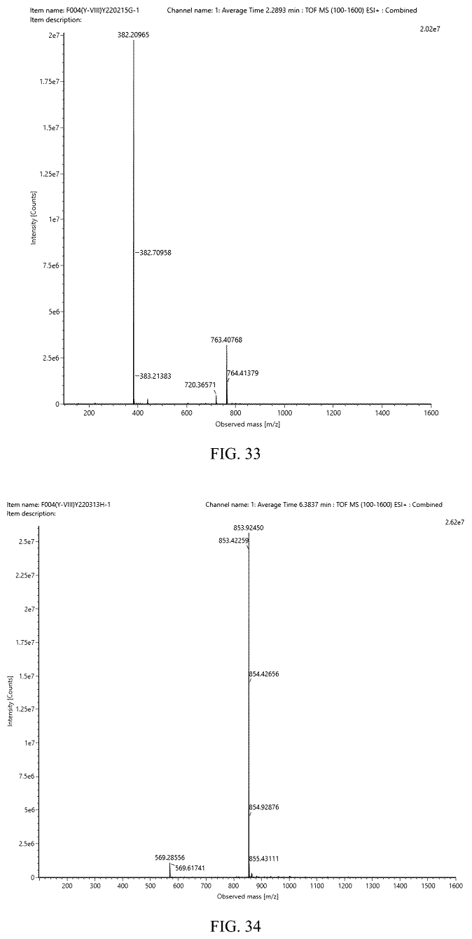

c(RGDfK) (1.00 g, 1.7 mmol), t-Boc-N-amido-PEG2-NHS ester (0.74 g, 1.9 mmol), DIPEA (0.44 g, 3.4 mmol) and 20 mL of DMF were added into a reaction flask to carry out a reaction at 30° C. for 20 h. Evaporation was performed under reduced pressure at 40° C., 10 mL of methanol was added, and 60 mL of MTBE was added dropwise to precipitate a solid so as to obtain an intermediate F1. Suction filtration was performed, and vacuum drying was performed at 40° C. for 2 h. The solid intermediate F1 was added into a reaction flask, 30 mL of TFA and 1.5 mL of purified water were added to carry out a reaction at 30° C. for 1 h, and cooling was performed to 0-5° C. 200 mL of MTBE was added dropwise and stirred at 0-5° C. for 30 min, suction filtration was performed, rinsing was performed with MTBE, and vacuum drying was performed at 40° C. to obtain a product. A theoretical molecular weight is 762.4024, a measured molecular weight is 762.40768, and mass spectrometry results are consistent with that of the target product. FIG. 33 is a mass spectrogram of the intermediate G1.

### (4) Preparation of an Intermediate H1

The intermediate D1, p-toluenesulfonic acid monohydrate (0.34 g, 1.8 mmol) and 20 mL of acetonitrile were added into a reaction flask to carry out a reaction at 65° C. for 4 h, and evaporation was performed under reduced pressure at 40° C. 20 mL of DMF, DIPEA (0.36 g, 2.8 mmol), DCC (0.14 g, 0.7 mmol) and NHS (0.08 g, 0.7 mmol) were added to carry out a reaction at 35° C. for 15-20 h to obtain an intermediate E1. Cooling was performed to 25° C., the intermediate G1 was added to carry out a reaction for 1 h, and evaporation was performed under reduced pressure at 40° C. to obtain a crude product. The crude product was subjected to preparative liquid chromatography to obtain 66.5 mg of a target product. A theoretical molecular weight is 1704.8300, a measured molecular weight is 1704.84518, and mass spectrometry results are consistent with that of the target product. FIG. 34 is a mass spectrogram of the intermediate H1.

### (5) Preparation of an Intermediate I1 (Namely, a Compound of Formula (I-25))

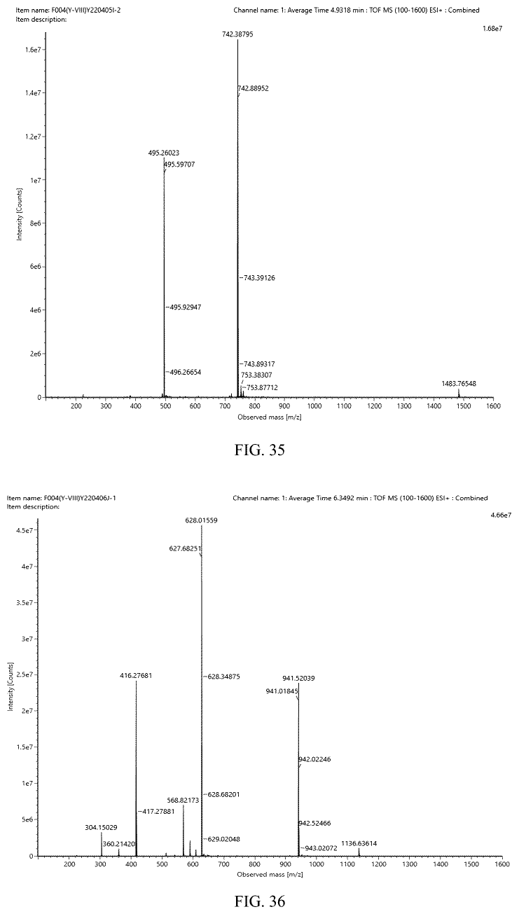

The intermediate H1, 0.5 mL of piperidine and 2 mL of DMF were added into a reaction flask to carry out a reaction at 25° C. for 1 h, 10 mL of ethyl acetate was added dropwise for crystallization, stirring was performed for 30 min, suction filtration was performed, and a solid was subjected to vacuum drying at 40° C. for 2 h to obtain 50.8 mg of an intermediate I1 (namely, a compound of Formula (I-25)). A theoretical molecular weight is 1482.7619, a measured molecular weight is 1482.7759, and mass spectrometry results are consistent with that of the target product. FIG. 35 is a mass spectrogram of the intermediate I1.

### (6) Preparation of an Intermediate J1

The intermediate I1, NOTA-Bis-TBU-NHS Ester, DIPEA (0.010 g, 0.08 mmol) and 2 mL of DMF were added into a reaction flask to carry out a reaction at 25° C. for 1 h, and evaporation was performed under reduced pressure at 40° C. 2 mL of ethyl acetate was added, 2 mL of MTBE was added for crystallization, stirring was performed for 20 min, suction filtration was performed, and a solid was subjected to vacuum drying at 40° C. to obtain 43.2 mg of a product. A theoretical molecular weight is 1880.0196, a measured molecular weight is 1880.0369, and mass spectrometry results are consistent with that of the target product. FIG. 36 is a mass spectrogram of the intermediate J1.

### (7) Preparation of a Compound of Formula (V-25)

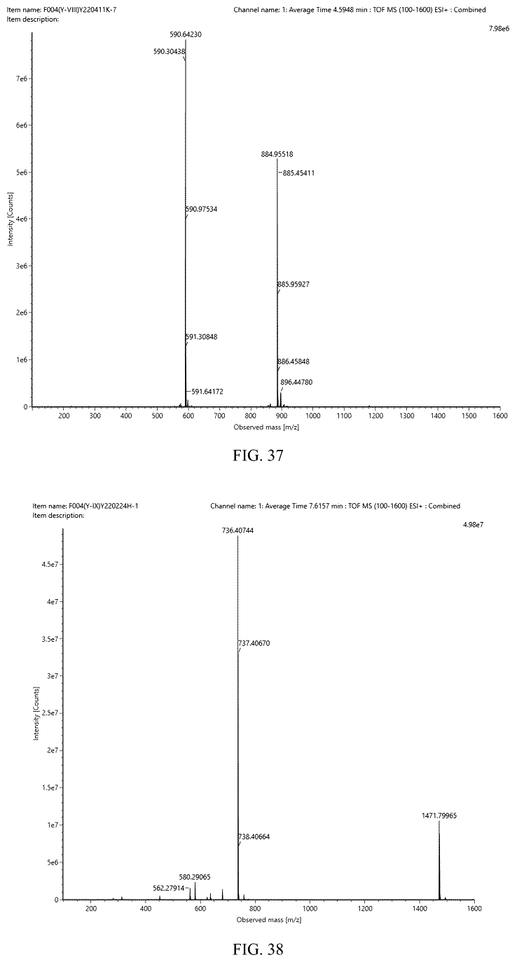

The intermediate J1 and 2 mL of trifluoroacetic acid were added into a reaction flask to carry out a reaction at 25° C. for 1 h, and evaporation was performed under reduced pressure at 40° C. to obtain a crude product. The crude product was purified by preparative liquid chromatography and then freeze-dried to obtain a product (namely, a compound of Formula (V-25)). A theoretical molecular weight is 1767.8944, a measured molecular weight is 1767.91036, and mass spectrometry results are consistent with that of the target product. FIG. 37 is a mass spectrogram of the compound of Formula (V-25).

### Example 5: Preparation of a Compound of Formula (I-3) and a Compound of Formula (V-26)

A synthesis route is as follows.

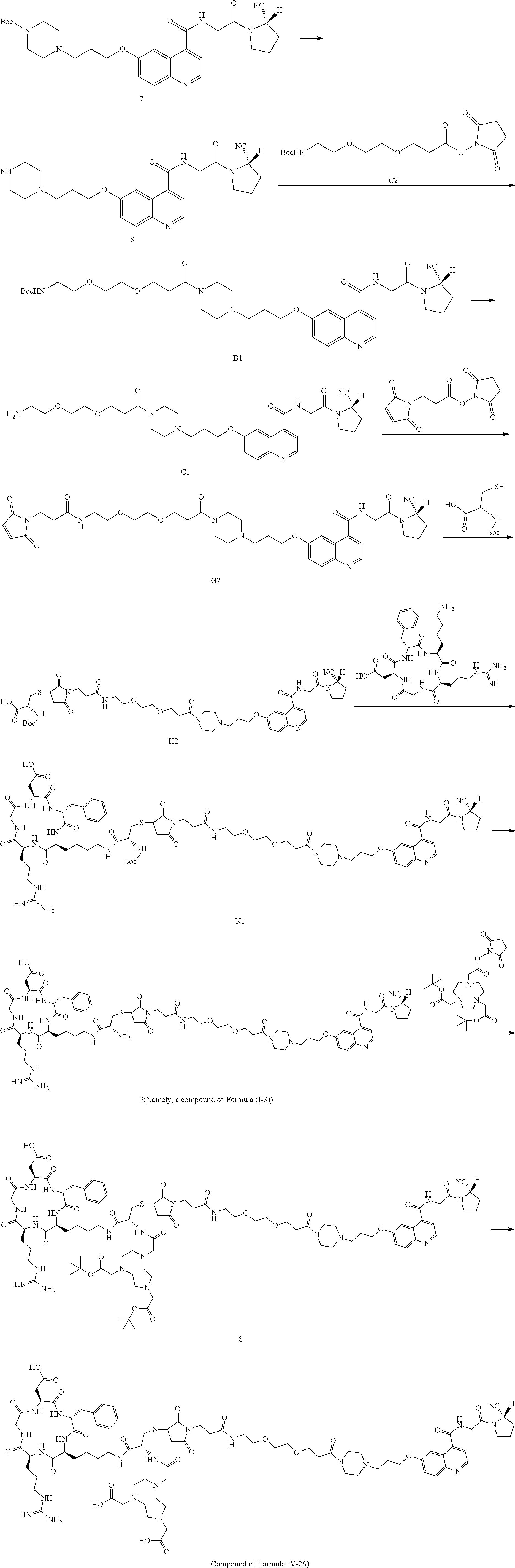

### (1) Preparation of an Intermediate B1

The starting material 7 (5.50 g, 10 mmol) and p-toluenesulfonic acid (5.71 g, 30 mmol) were weighed and added into a reaction flask, 10 mL of acetonitrile was added, and a resulting mixture was stirred and heated to 65° C. to carry out a reaction for 1 h to remove a protective group on a piperazine ring so as to obtain an intermediate 8. After the reaction was completed based on detection by TLC (with a developing agent including dichloromethane and methanol at a ratio of 5:1), evaporation was performed under reduced pressure at 40° C. 4 mL of DMF and DIPEA (9.05 g, 70 mmol) were added and stirred for dissolution, and t-Boc-N-amido-PEG2-NHS ester (intermediate C2, 5.62 g, 15 mmol) was added to carry out a reaction at 25° C. for 3 h. After the reaction was completed based on detection by TLC (with a developing agent including dichloromethane and methanol at a ratio of 5:1), evaporation was performed under reduced pressure at 40° C. 5 mL of purified water was added, extraction was performed with DCM for two times at 5 mL each time, and organic phases were combined and dried with anhydrous sodium sulfate (1 m/m) for 30 min. Suction filtration was performed, and a mother liquid was evaporated under reduced pressure at 40° C. to obtain a crude product.

Purification was performed by column chromatography to obtain an intermediate B1. The product was directly used for feeding in the next step.

### (2) Preparation of an Intermediate G2

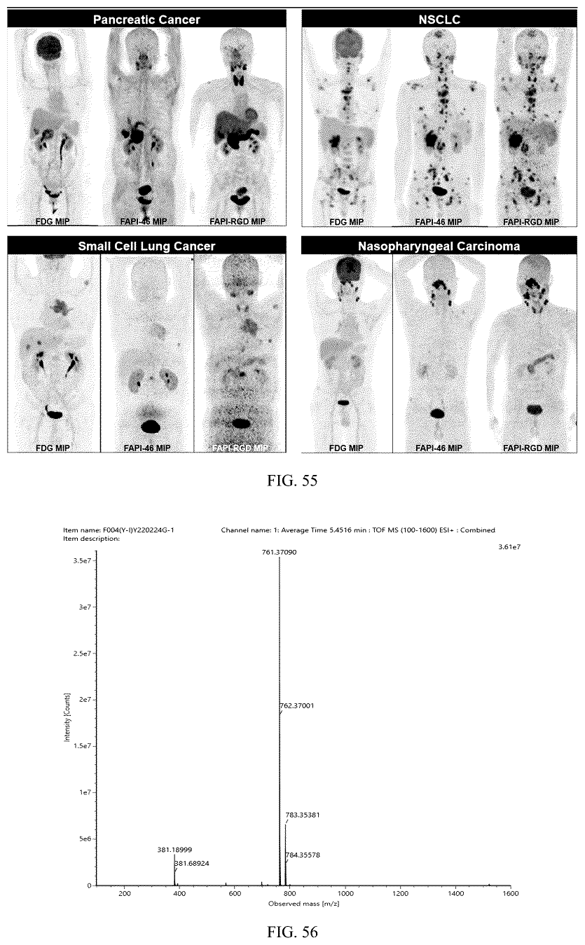

The intermediate B1 and p-toluenesulfonic acid (5.14 g, 27 mmol) were weighed and added into a reaction flask, 10 mL of acetonitrile was added, and a resulting mixture was stirred and heated to 65° C. to carry out a reaction for 1 h to remove a protective group so as to obtain an intermediate C1. After the reaction was completed based on detection by TLC (with a developing agent including dichloromethane and methanol at a ratio of 10:1), evaporation was performed under reduced pressure at 40° C. DIPEA (5.82 g, 45 mmol), 10 mL of DMF, 3-maleimide propionate and N-hydroxysuccinimide ester (2.88 g, 10.8 mmol) were added to carry out a reaction at room temperature for 1 h. After the reaction was completed based on detection by a TLC plate (with a developing agent including dichloromethane and methanol at a ratio of 10:1), evaporation was performed under reduced pressure at 40° C. Purification was performed by column chromatography to obtain an intermediate G2. A theoretical molecular weight of the target product is 760.35443, a molecular weight measured by liquid chromatography-mass spectrometry is 760.37090, and mass spectrometry results are consistent with that of the target product. FIG. 56 is a mass spectrogram of the intermediate G2.

### (3) Preparation of an Intermediate N1

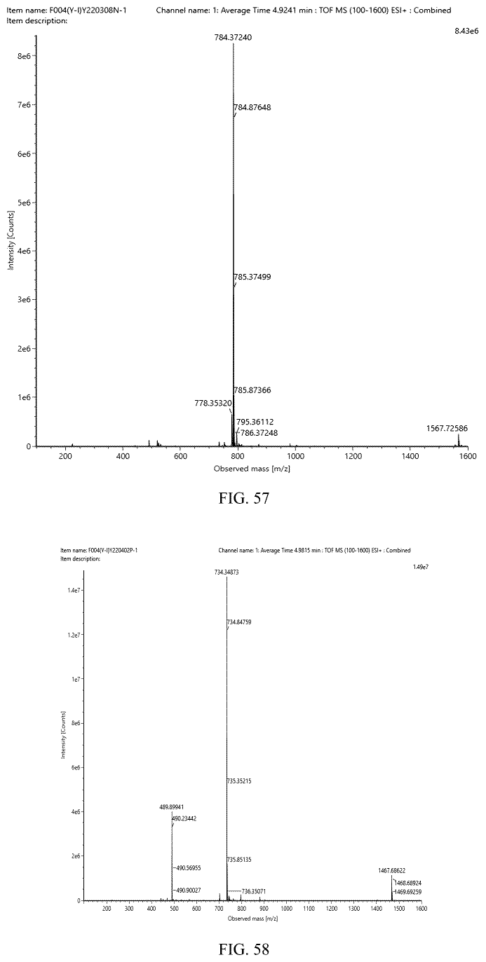

The intermediate G2 and Boc-cysteine (1.77 g, 8 mmol) were weighed and added into a reaction flask, and 10 mL of DMF was added to carry out a reaction at 25° C. for 2 h to obtain an intermediate H2. After the reaction was completed based on detection by TLC (with a developing agent including dichloromethane and methanol at a ratio of 5:1), DCC (1.98 g, 9.6 mmol) and NHS (1.86 g, 9.6 mmol) were added to carry out a reaction at 35° C. for 2 h. After the reaction was completed based on detection by TLC (with a developing agent including dichloromethane and methanol at a ratio of 5:1), a cyclic peptide Cyclo(Arg-Gly-Asp-DPhe-Lys) (4.83 g, 8 mmol) and DIPEA (3.10 g, 24 mmoleq) were added to carry out a reaction at 25° C. for 1 h. After the reaction was completed based on detection by TLC (with a developing agent including dichloromethane and methanol at a ratio of 5:1), evaporation was performed under reduced pressure at 40° C. to obtain a crude product. The crude product was purified by preparative liquid chromatography to obtain an intermediate N1. A theoretical molecular weight of the target product is 1566.72893, a molecular weight measured by liquid chromatography-mass spectrometry is 1566.74480, and mass spectrometry results are consistent with that of the target product. FIG. 57 is a mass spectrogram of the intermediate N1.

### (4) Preparation of an Intermediate P (Namely, a Compound of Formula (I-3))

The intermediate N1 was weighed and added into a reaction flask, and 2 mL of thioanisole, 2 mL of 1,2-ethyldithiol and 20 mL of trifluoroacetic acid were added to carry out a reaction at room temperature for 1 h under the protection of nitrogen. 20 mL of methyl tert-butyl ether was added until a solid was precipitated, suction filtration was performed, and the solid was subjected to vacuum drying at 40° C. for 1 h to obtain an intermediate P. A theoretical molecular weight of the target product is 1466.67650, a molecular weight measured by liquid chromatography-mass spectrometry is 1466.69746, and mass spectrometry results are consistent with that of the target product. FIG. 58 is a mass spectrogram of the intermediate P.

### (5) Preparation of a Compound of Formula (V-26)

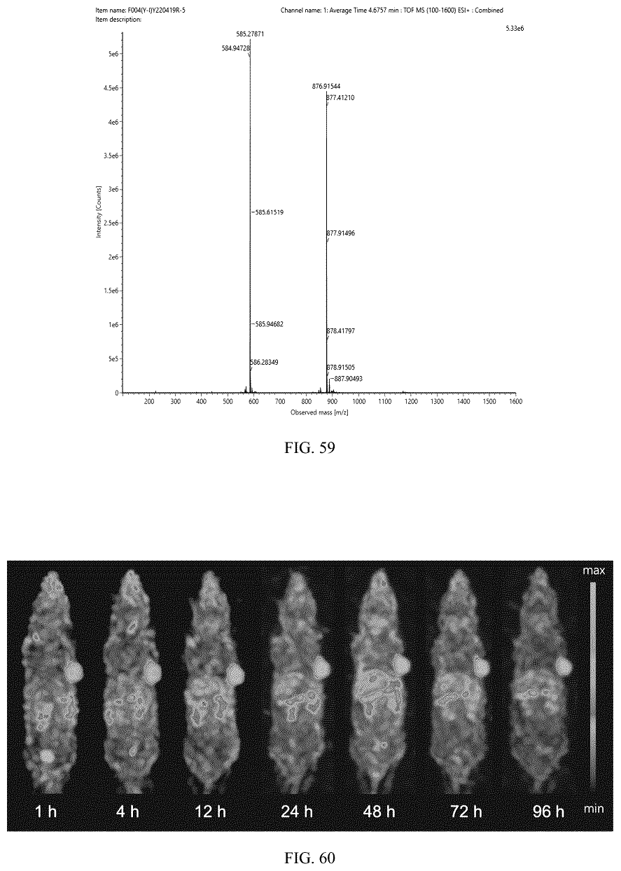

The intermediate P and NOTA-Bis-TBU-NHS Ester were weighed and added into a reaction flask, and 40 mL of DMF was added to carry out a reaction at room temperature for 1 h. 4 mL of DIPEA was added to carry out a reaction at room temperature for 3 h. Evaporation was performed under reduced pressure at 40° C. to obtain an intermediate S. 30 mL of trifluoroacetic acid was added and stirred at room temperature for 1 h. Evaporation was performed under reduced pressure at 40° C. to obtain a crude product, and the crude product was purified by preparative liquid chromatography to obtain a product (namely, a compound of Formula (V-26)). A theoretical molecular weight of the target product is 1751.80898, a molecular weight measured by liquid chromatography-mass spectrometry is 1751.83088, and mass spectrometry results are consistent with that of the target product. FIG. 59 is a mass spectrogram of the compound of Formula (V-26).

### Example 6: Preparation of a Compound of Formula (V-30)

A synthesis route is as follows.

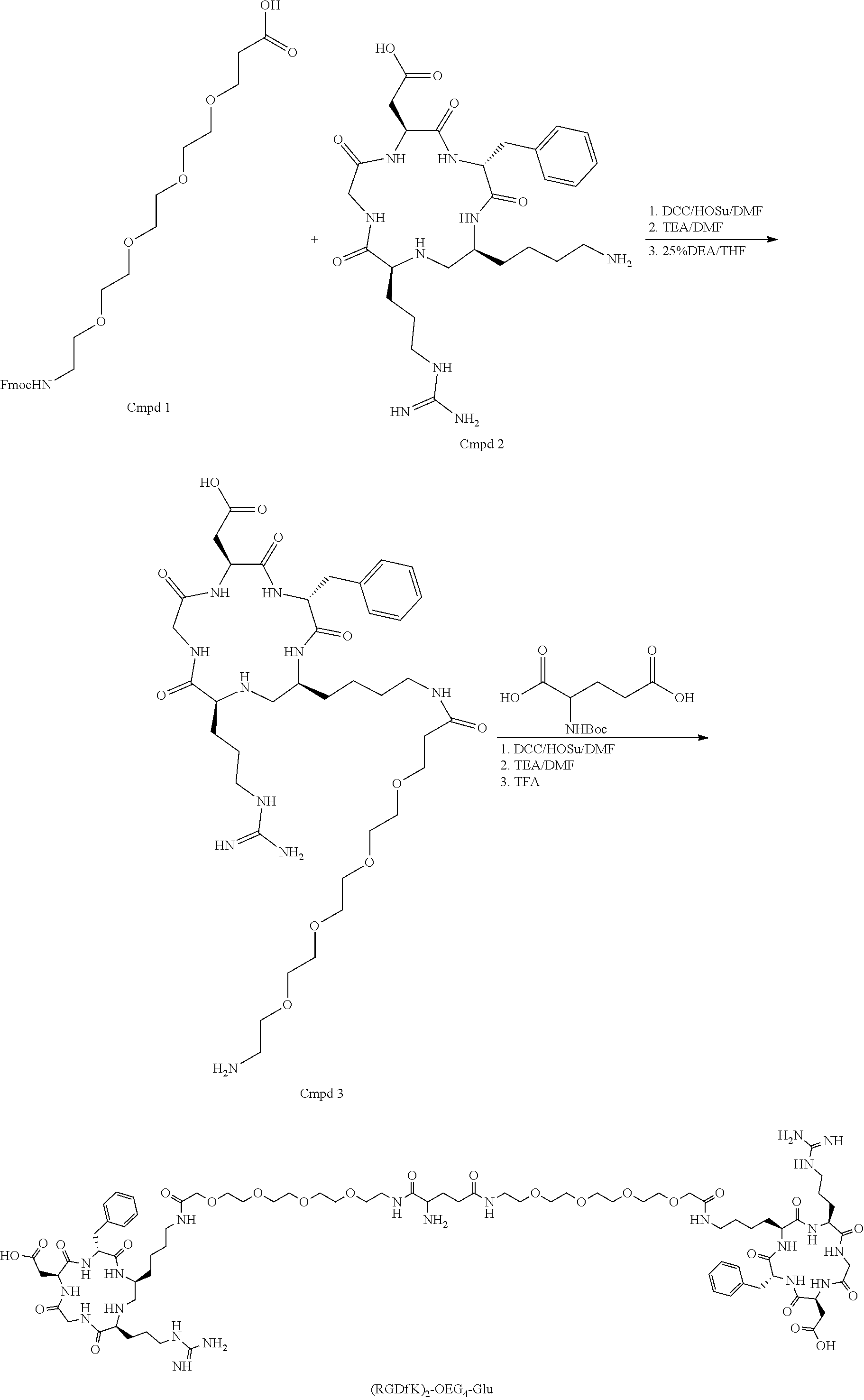

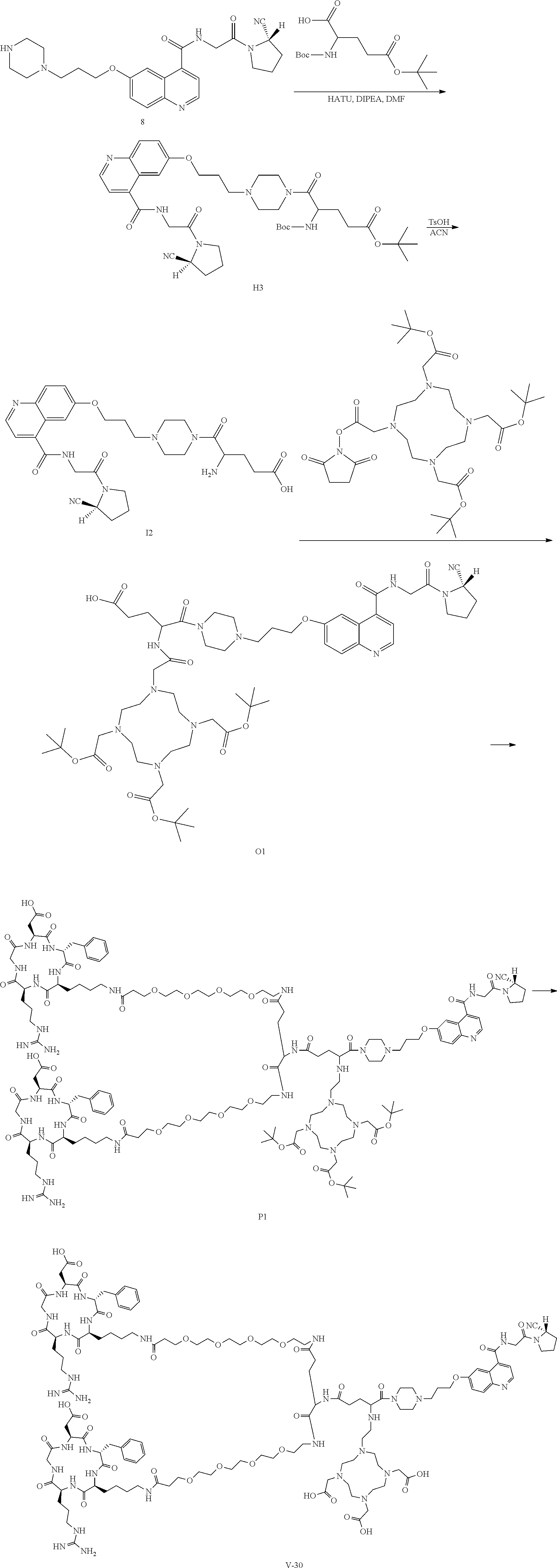

### (1) Preparation of an Intermediate Cmpd3

Fmoc-PEG4-CH₂CH₂COOH (namely, a compound Cmpd1, 1.46 g, 3.0 mmol) was dissolved in DMF, and then DCC (0.68 g, 3.3 mmol) and HOSu (0.38 g, 3.3 mmol) were added to carry out a reaction at room temperature for 6 h. Filtration was added, TEA (0.90 g, 9.0 mmol) was added into a filtrate, and then Cyclo(RGDfK) (namely, a compound Cmpd2, 2.23 g, 3.6 mmol) was added to carry out a reaction at room temperature for 3 h. A reaction solution was spin-dried, dissolved in 25% DEA/THF to carry out a reaction at room temperature for 4 h, concentrated until a small amount of the solution was remained, and then added into 10 times the volume of ethyl ether until a large amount of a solid was precipitated. Then filtration was performed to obtain a crude product Cyclo(RGDfK)-PEG4, and the crude product was purified by reverse-phase preparative liquid chromatography to obtain a fine product Cyclo(RGDfK)-PEG4 (namely, an intermediate Cmpd3), where an eluent (including a solution A: 0.1% TFA in H₂O, and a solution B: acetonitrile) was used.

### (2) Synthesis of an Intermediate (RGDfK)₂-PEG₄-Glu

Boc-Glu-OH (0.4 g, 2.0 mmol) was dissolved in DMF, and then DCC (0.45 g, 2.2 mmol) and HOSu (0.25 g, 2.2 mmol) were added to carry out a reaction at room temperature for 6 h. Filtration was added, TEA (0.60 g, 6.0 mmol) was added into a filtrate, and then the Cyclo(RGDfK)-PEG4 (namely, the intermediate Cmpd3, 2.61 g, 2.4 mmol) was added to carry out a reaction at room temperature for 3 h. A reaction solution was spin-dried, dissolved in TFA to carry out a reaction at room temperature for 10 min, and then added into 10 times the volume of ethyl ether until a large amount of a solid was precipitated. Then filtration was performed to obtain a crude product 2(RGDfK)-PEG4-Glu, and the crude product was purified by reverse-phase preparative liquid chromatography to obtain purified (RGDfK)₂-PEG4-Glu, where an eluent (including a solution A: 0.1% TFA in H₂O, and a solution B: acetonitrile) was used. Then, the pH of the purified (RGDfK)₂-PEG4-Glu was adjusted to neutral with TEA, the reverse-phase preparative liquid chromatography was performed again, and freeze-drying was performed to obtain a final product (RGDfK)₂-PEG4-Glu.

### (3) Preparation of an Intermediate H3

The intermediate 8 (0.45 g, 1 mmol) prepared by the method in Example 1, Fmoc-O-tert butyl-L-glutamic acid (0.42 g, 1 mmol), HATU (0.38 g, 1 mmol) and DIPEA (0.58 g, 4.5 mmol) were sequentially dissolved in 10 mL of DMF to carry out a reaction in an external bath at 25° C. After the reaction was completed based on monitoring by HPLC, 20 mL of purified water was added to the reaction system, extraction was performed with 20 mL of DCM for two times, and organic phases were combined, dried with anhydrous sodium sulfate, concentrated and subjected to column purification to obtain an intermediate H3 with a crude yield of 97% in two steps. A theoretical molecular weight is 735.3956, a measured molecular weight is 735.40744, and mass spectrometry results are consistent with that of the target product. FIG. 38 is a mass spectrogram of the intermediate H3.

### (4) Preparation of an Intermediate 12

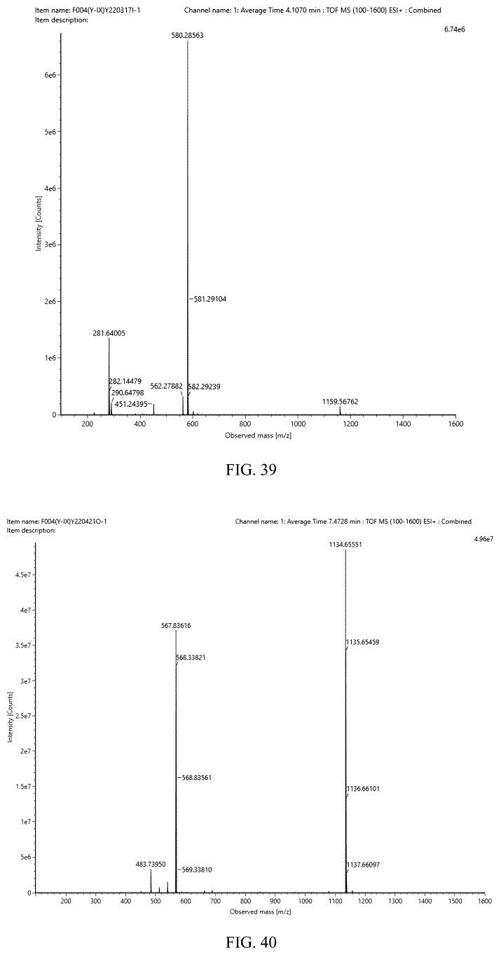

The intermediate H3 was dissolved in 20 mL of acetonitrile, p-toluenesulfonic acid monohydrate (0.65 g, 3.4 mmol) was added, and a reaction was carried out in an external bath at 70° C. After the reaction was completed based on monitoring by HPLC, the acetonitrile in the system was concentrated, and a product was directly used in the next step. A theoretical molecular weight is 579.2805, a measured molecular weight is 579.28563, and mass spectrometry results are consistent with that of the target product. FIG. 39 is a mass spectrogram of the intermediate 12.

### (5) Preparation of an Intermediate O1

The intermediate 12 and DIPEA (0.64 g, 5.0 mmol) were dissolved in 10 mL of DMF, and DOTA-TRIS-TBU-NHS Ester (1.7 g, 2.5 mmol) was added. After a reaction was completed based on monitoring by HPLC, the solvent in the system was concentrated, and the remaining system was subjected to preparative purification to obtain an intermediate O1 with a yield of 27.15% in two steps. A theoretical molecular weight is 1133.6485, a measured molecular weight is 1133.65551, and mass spectrometry results are consistent with that of the target product. FIG. 40 is a mass spectrogram of the intermediate O1.

### (6) Preparation of an Intermediate P1

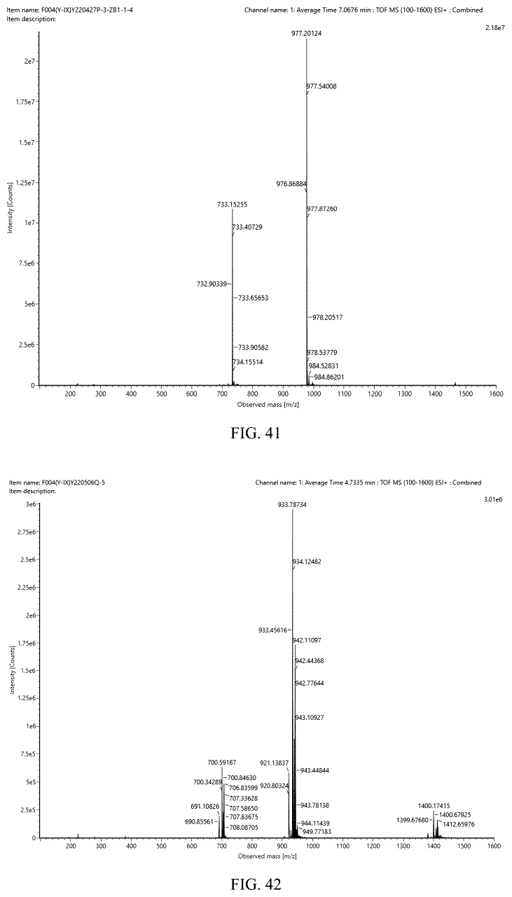

The intermediate O1 was dissolved in 5 mL of DMF, and HATU (0.076 g, 0.2 mmol) was added and stirred at room temperature for 1 h to obtain a system (1). DIPEA (0.090 g, 0.7 mmol) and the (RGDfK)₂-PEG4-Glu (0.24 g, 0.13 mmol) were dissolved in 5 mL of DMSO to obtain a system (2). The system (1) was added to the system (2) and stirred at 28° C. After a reaction was completed based on monitoring by HPLC, the DMF was concentrated, 100 mL of MTBE was added for crystallization, and standing was performed. Then, a supernatant was poured out, and a remaining oily compound was subjected to preparative purification to obtain an intermediate P1 with a yield of 17.09%. A theoretical molecular weight is 2927.5797, a measured molecular weight is 2927.60652, and mass spectrometry results are consistent with that of the target product. FIG. 41 is a mass spectrogram of the intermediate P1.

### (7) Preparation of a Compound V-30

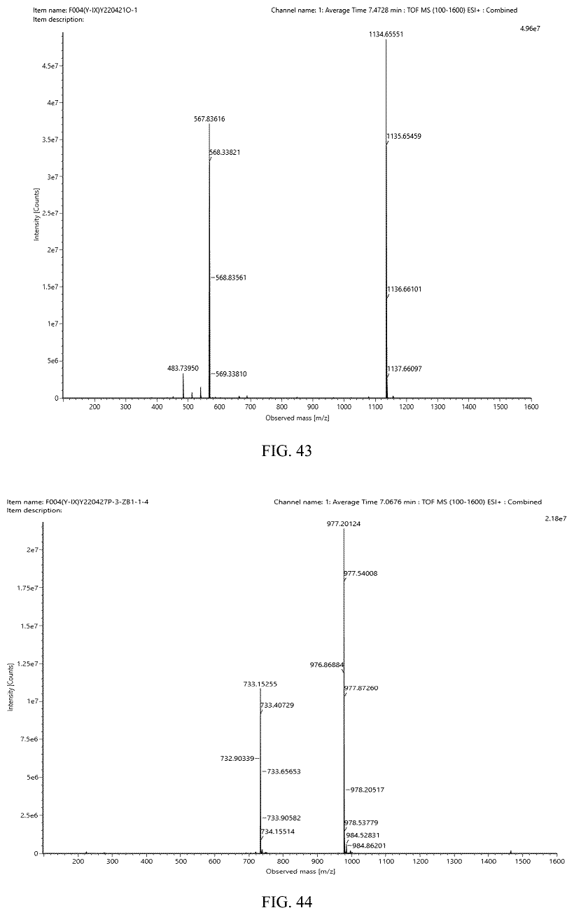

The intermediate P1 was dissolved in 5 mL of TFA, and a reaction was carried out in an external bath at 25° C. After the reaction was completed based on monitoring by HPLC, 25 mL of MTBE was added to the system for crystallization, standing was performed, and a supernatant was sucked out. Then the remaining system was concentrated with MTBE until no obvious TFA residues were remained, and subjected to preparative purification to obtain V-30 with a yield of 32.13%. A theoretical molecular weight is 2759.3919, a measured molecular weight is 2759.40972, and mass spectrometry results are consistent with that of the target product. FIG. 42 is a mass spectrogram of the V-30.

### Example 7: Preparation of a Compound of Formula (V-35)

A synthesis route is as follows.

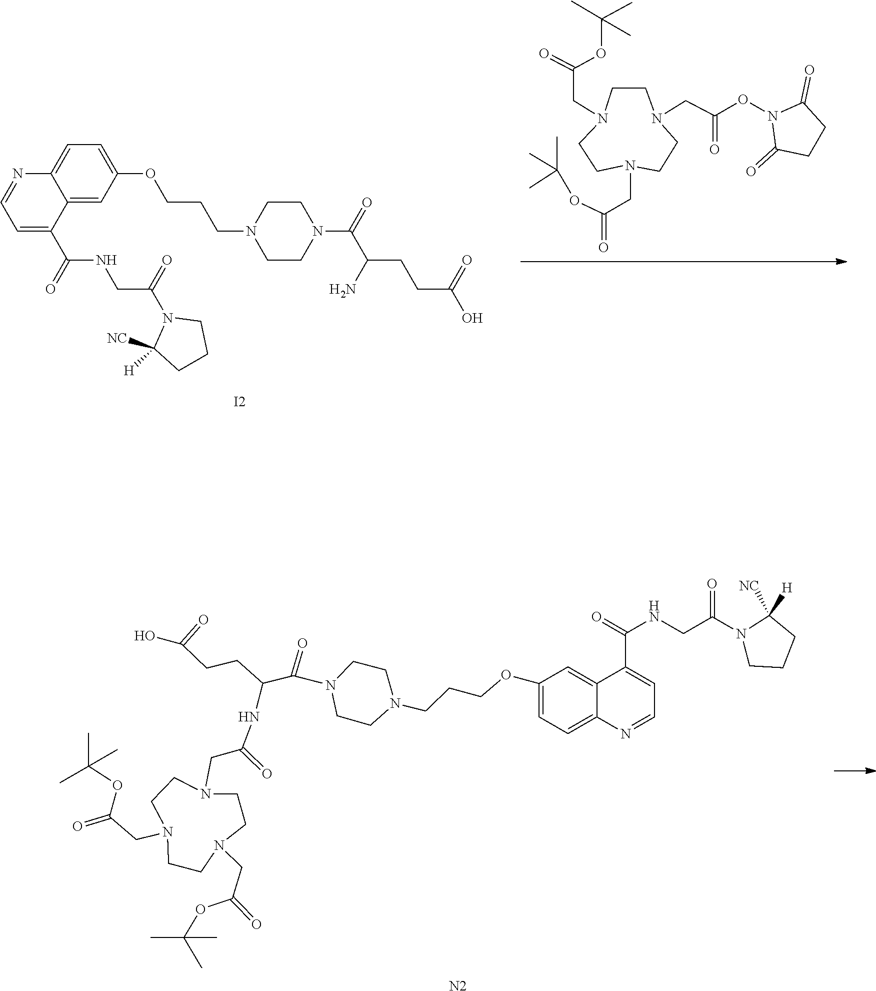 [SMILES: N#C[C@@H]1CCCN1C(=O)CNC(=O)c1ccnc2ccc(OCCCN3CCN(C(=O)C(N)CCC(=O)O)CC3)cc12 ; CC(C)(C)OC(=O)CN1CCN(CC(=O)ON2C(=O)CCC2=O)CCN(CC(=O)OC(C)(C)C)CC1 ; "I2" ; CC(C)(C)OC(=O)CN1CCN(CC(=O)NC(CCC(=O)O)C(=O)N2CCN(CCCOc3ccc4nccc(C(=O)NCC(=O)N5CCC[C@H]5C#N)c4c3)CC2)CCN(CC(=O)OC(C)(C)C)CC1 ; "N2"]

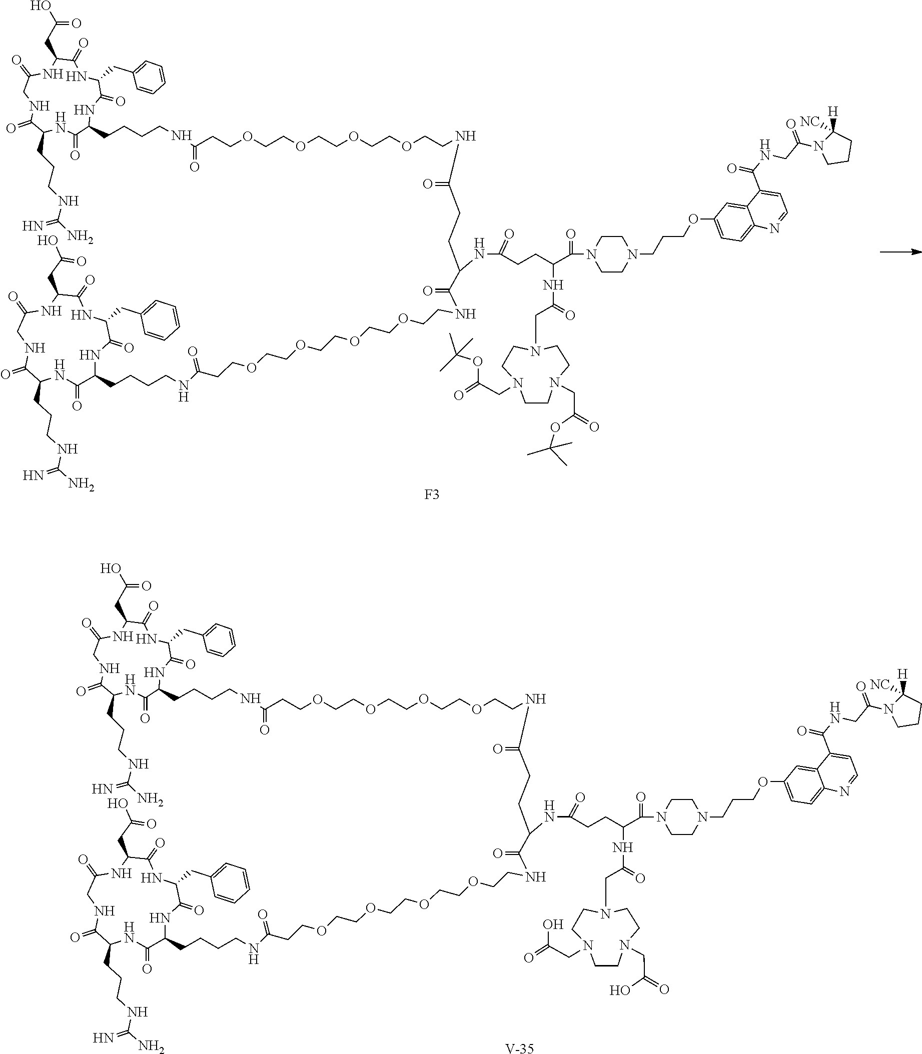

### (1) Preparation of an Intermediate N2

The intermediate 12 prepared by the method in Example 6 and DIPEA (3.90 g, 30 mmol) were dissolved in 10 mL of DMF, and NOTA-Bis-TBU-NHS Ester (7.65 g, 15 mmol) was added to the system. After a reaction was completed based on monitoring by HPLC, the DMF in the system was concentrated, and the remaining system was subjected to preparative purification to obtain an intermediate N2 with a yield of 22.88% in two steps. A theoretical molecular weight is 976.5382, a measured molecular weight is 976.56026, and mass spectrometry results are consistent with that of the target product. FIG. 43 is a mass spectrogram of the intermediate N2.

### (2) Preparation of an Intermediate F3

The intermediate N2 was dissolved in 10 mL of DMF, and HATU (0.46 g, 1.2 mmol) was added to carry out a reaction in an external bath at 30° C. for 1 h to obtain a system (1). c(RGDfK)₂-PEG4-Glu (1.54 g, 0.8 mmol) and DIPEA (0.62 g, 4.8 mmol) were dissolved in 5 mL of DMF and 5 mL of DMSO to obtain a system (2). The system (1) was added to the system (2) and stirred in an external bath at 30° C. After a reaction was completed based on monitoring by HPLC, the solvent was concentrated, and the remaining system was subjected to preparative purification to obtain an intermediate F3 with a yield of 15.34%. A theoretical molecular weight is 2770.4894, a measured molecular weight is 2770.49229, and mass spectrometry results are consistent with that of the target product. FIG. 44 is a mass spectrogram of the intermediate F3.

### (3) Preparation of a Compound of Formula (V-35)

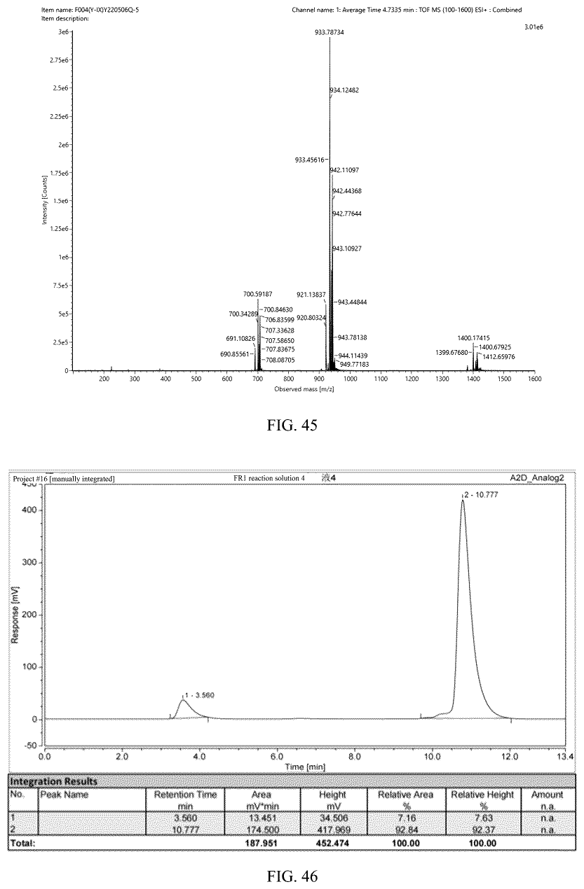

The intermediate F3 was dissolved in 20 mL of TFA, and a reaction was carried out in an external bath at 25° C. After the reaction was completed based on monitoring by HPLC, 50 mL of MTBE was added to the system for crystallization, standing was performed, and a supernatant was poured out. Then the remaining system was concentrated with MTBE until no obvious TFA residues were remained in the system, and subjected to preparative purification to obtain a compound of Formula (V-35) with a yield of 2.89%. A theoretical molecular weight is 2658.3442, a measured molecular weight is 2658.36508, and mass spectrometry results are consistent with that of the target product. FIG. 45 is a mass spectrogram of the compound of Formula (V-35).

### Example 8: Preparation of a Compound of Formula (I-16), a Compound of Formula (I-40) and a Compound of Formula (V-40)

A synthesis route is as follows.

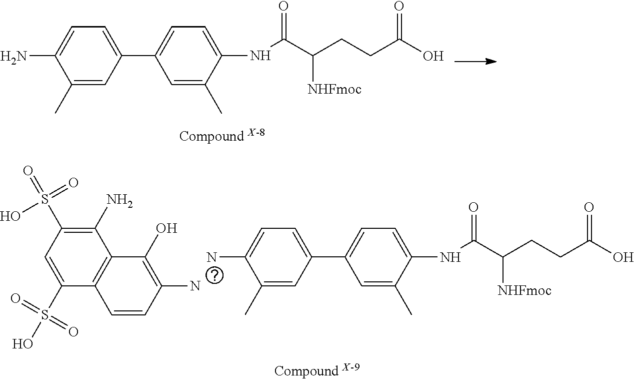

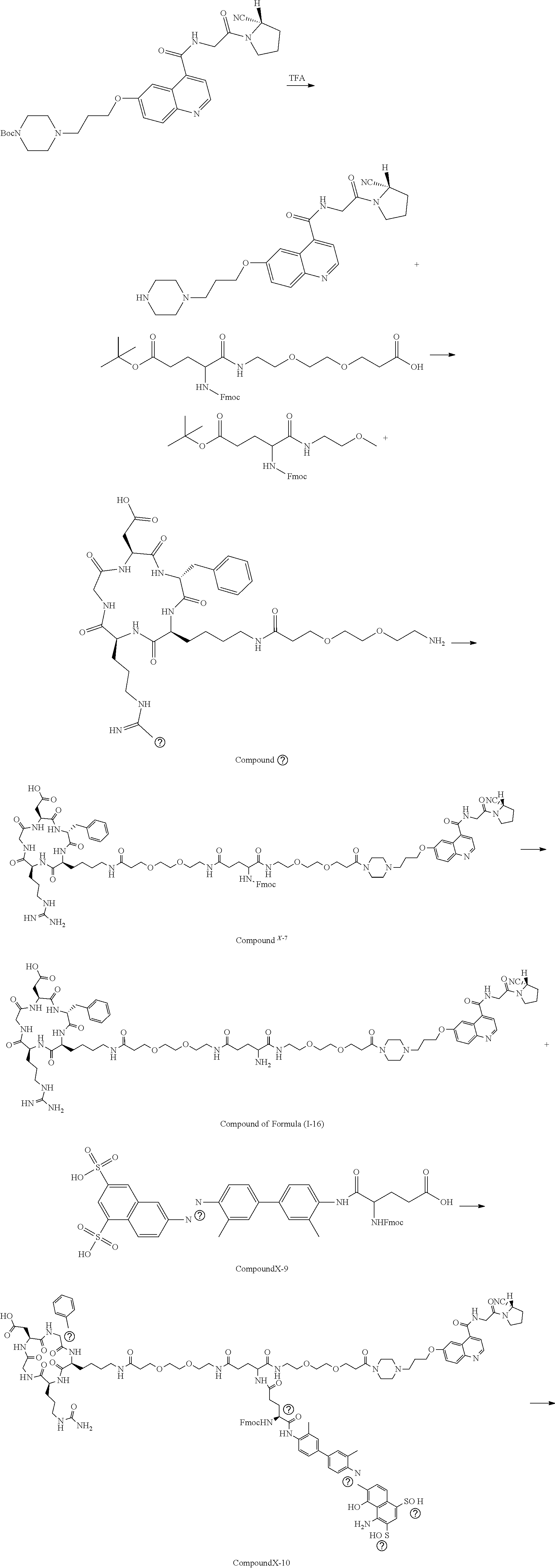

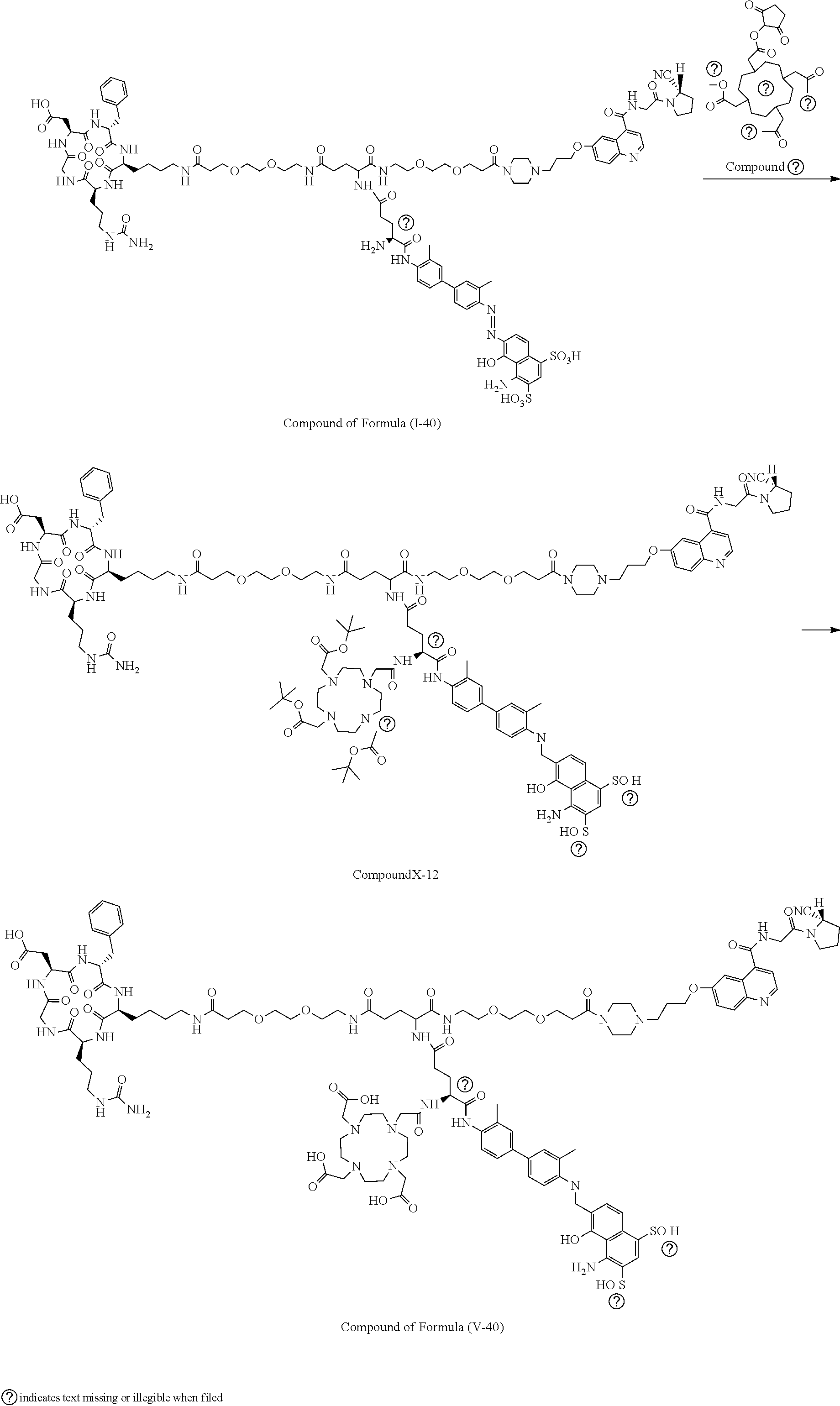

With reference to preparative examples/methods provided in Examples 1-7, a compound of Formula (I-16), a compound of Formula (I-40) and a compound of Formula (V-40) were prepared in Example 8. On the basis/inspiration of Examples 1-7, corresponding substitutions of raw materials can be made by persons skilled in the art in combination with the above preparation route, which are not repeatedly described herein.

### Examples 9-45: Preparation of Other Dual-Targeting Compounds

With reference to preparative examples/methods provided in Examples 1-8, a compound of Formula (I-2), a compound of Formula (I-4), a compound of Formula (I-5), a compound of Formula (I-6), a compound of Formula (I-7), a compound of Formula (I-8), a compound of Formula (I-9), a compound of Formula (I-10), a compound of Formula (I-11), a compound of Formula (I-12), a compound of Formula (I-13), a compound of Formula (I-17), a compound of Formula (I-18), a compound of Formula (I-19), a compound of Formula (I-20), a compound of Formula (I-21), a compound of Formula (I-27), a compound of Formula (I-28), a compound of Formula (I-29), a compound of Formula (I-30), a compound of Formula (I-31), a compound of Formula (I-32), a compound of Formula (I-33), a compound of Formula (I-34), a compound of Formula (I-35), a compound of formula (I-36), a compound of formula (I-37), a compound of Formula (I-38), a compound of Formula (I-39), a compound of Formula (II-1), a compound of Formula (II-2), a compound of Formula (II-3), a compound of Formula (II-4), a compound of Formula (II-5), a compound of Formula (II-6), a compound of Formula (II-7) and a compound of Formula (II-8) were prepared in Examples 9-45. On the basis/inspiration of Examples 1-8, corresponding substitutions of raw materials can be made by persons skilled in the art, such as substituting c(RGDfK) with c(RGDyK), substituting c(RGDyK) with c(RGDfK) and substituting (S)-difluoropyrrolidine-2-carbonitrile hydrochloride with (S)-4,4-difluoropyrrolidine-2-carbonitrile hydrochloride. Structures of compounds in relevant preparation examples are described in the foregoing contents of the present application, which are not repeatedly described herein.

### Examples 46-83: Preparation of Other Compounds Capable of being Labeled with a Radionuclide

With reference to the preparative examples/methods provided in Examples 1-8, a compound of Formula (V-2), a compound of Formula (V-3), a compound of Formula (V-4), a compound of Formula (V-5), a compound of Formula (V-6), a compound of Formula (V-7), a compound of Formula (V-8), a compound of Formula (V-9), a compound of Formula (V-10), a compound of Formula (V-11), a compound of Formula (V-12), a compound of Formula (V-13), a compound of Formula (V-16), a compound of Formula (V-17), a compound of Formula (V-18), a compound of Formula (V-19), a compound of Formula (V-20), a compound of Formula (V-21), a compound of Formula (V-22), a compound of Formula (V-27), a compound of Formula (V-28), a compound of Formula (V-29), a compound of Formula (V-31), a compound of Formula (V-32), a compound of Formula (V-33), a compound of Formula (V-34), a compound of formula (V-36), a compound of formula (V-37), a compound of Formula (V-38), a compound of Formula (V-39), a compound of Formula (VI-1), a compound of Formula (VI-2), a compound of Formula (VI-3), a compound of Formula (VI-4), a compound of Formula (VI-5), a compound of Formula (VI-6), a compound of Formula (VI-7) and a compound of Formula (VI-8) were prepared in Examples 46-83. On the basis/inspiration of Examples 1-8, corresponding substitutions of raw materials can be made by persons skilled in the art, such as substituting c(RGDfK) with c(RGDyK), substituting c(RGDyK) with c(RGDfK) and substituting (S)-difluoropyrrolidine-2-carbonitrile hydrochloride with (S)-4,4-difluoropyrrolidine-2-carbonitrile hydrochloride. Structures of compounds in relevant preparation examples are described in the foregoing contents of the present application, which are not repeatedly described herein.

### Examples 84-133: Other Preparative Examples

With reference to the preparation methods in Examples 1-83, FAPI-RGD compounds of Formula (V) in Table 1 or FAPI-RGD compounds of Formula (VI) in Table 2 below were prepared.

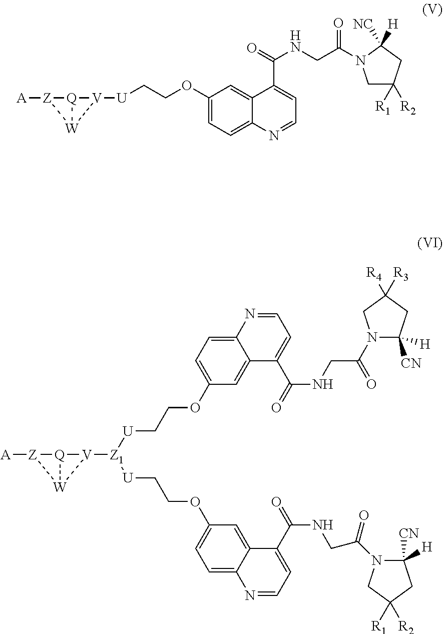

TABLE 1

| Example | | | |
|---|---|---|---|
| of | | | |
| Formula | | | |
| (V) | A | Z | Q |
| 84 | 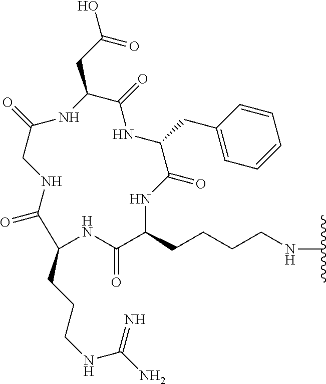 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 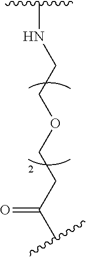 | 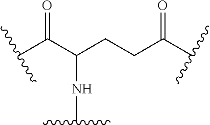 [SMILES: *NC(CCC(*)=O)C(*)=O] |
| 85 | 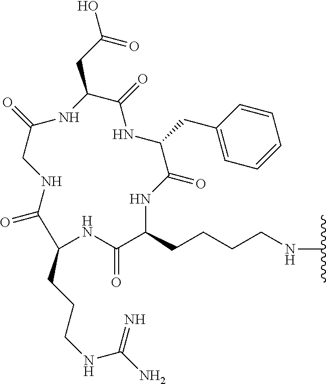 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 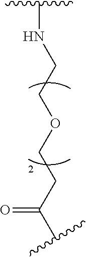 | 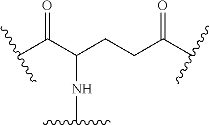 [SMILES: *NC(CCC(*)=O)C(*)=O] |
| 86 | 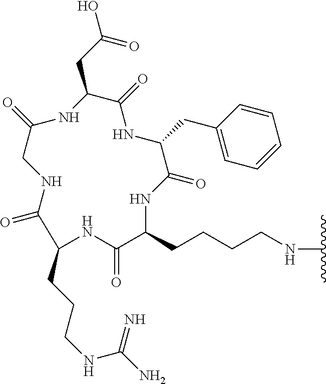 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 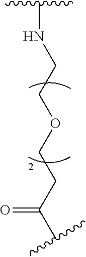 | 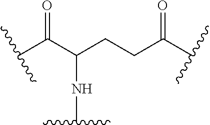 [SMILES: *NC(CCC(*)=O)C(*)=O] |
| 87 | 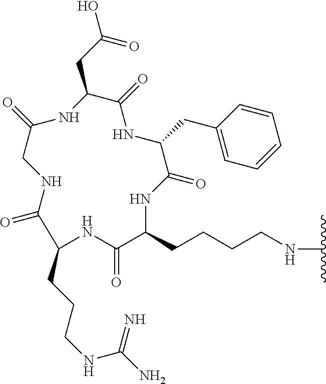 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 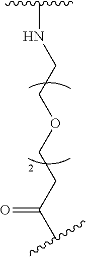 | 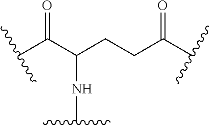 [SMILES: *NC(CCC(*)=O)C(*)=O] |
| 88 | 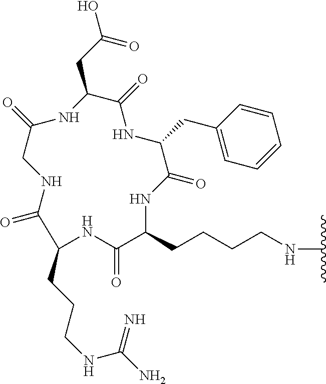 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 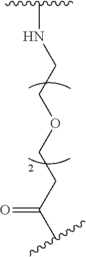 | 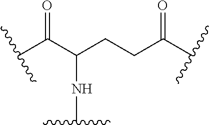 [SMILES: *NC(CCC(*)=O)C(*)=O] |
| 89 | 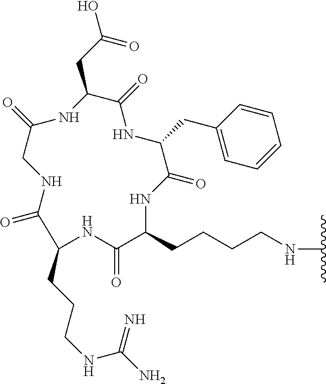 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 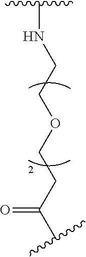 | 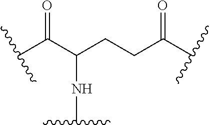 [SMILES: *NC(CCC(*)=O)C(*)=O] |
| 90 | 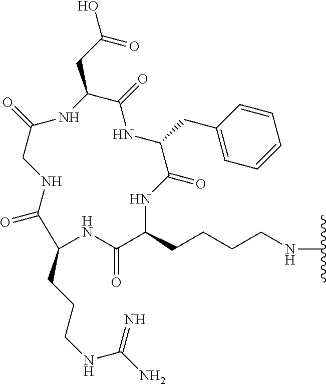 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 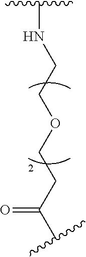 | 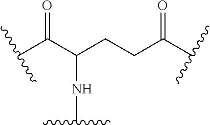 [SMILES: *NC(CCC(*)=O)C(*)=O] |
| 91 | 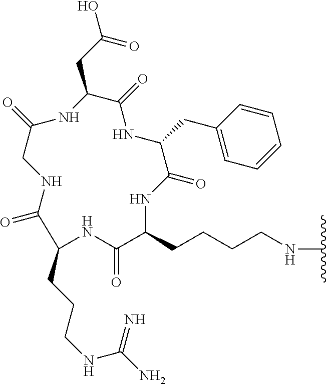 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 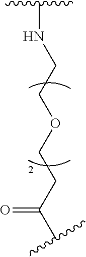 | 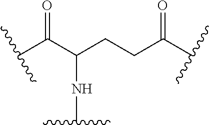 [SMILES: *NC(CCC(*)=O)C(*)=O] |
| 92 | 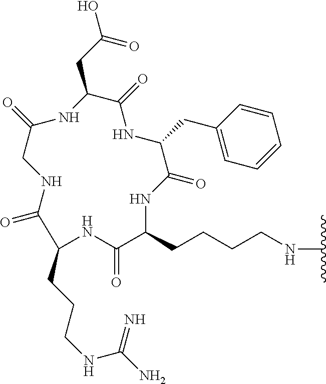 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 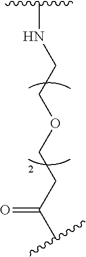 | 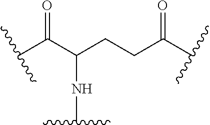 [SMILES: *NC(CCC(*)=O)C(*)=O] |
| 93 | 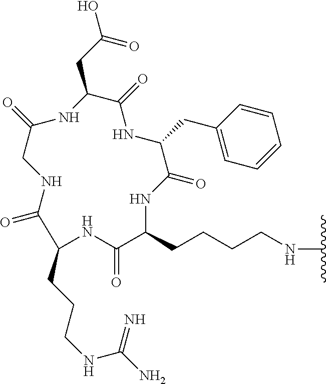 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 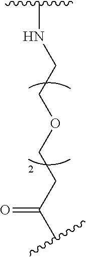 | 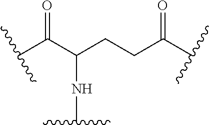 [SMILES: *NC(CCC(*)=O)C(*)=O] |
| 94 | 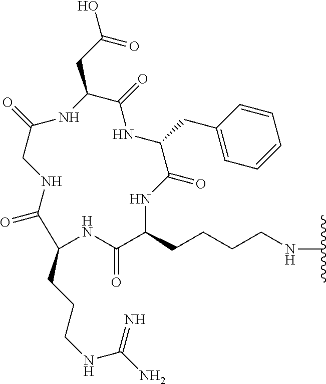 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 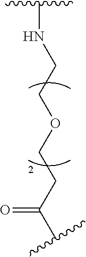 | 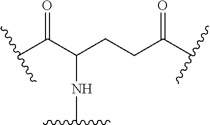 [SMILES: *NC(CCC(*)=O)C(*)=O] |
| 95 | 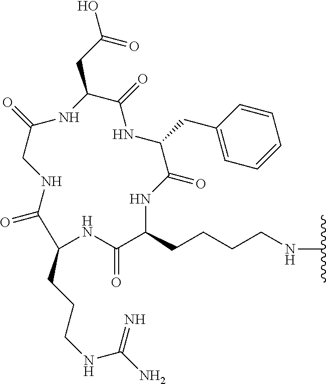 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 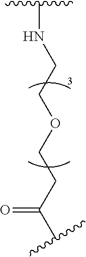 | 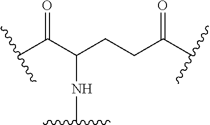 [SMILES: *NC(CCC(*)=O)C(*)=O] |
| 96 | 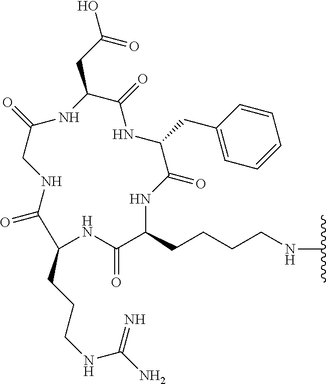 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 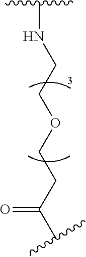 | 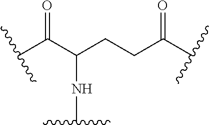 [SMILES: *NC(CCC(*)=O)C(*)=O] |
| 97 | 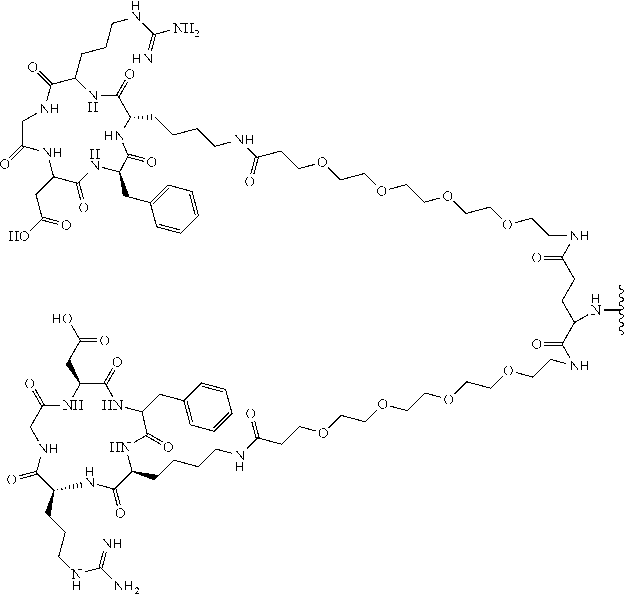 | — | 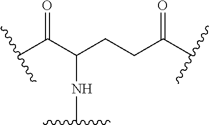 [SMILES: *NC(CCC(*)=O)C(*)=O] |
| 98 | 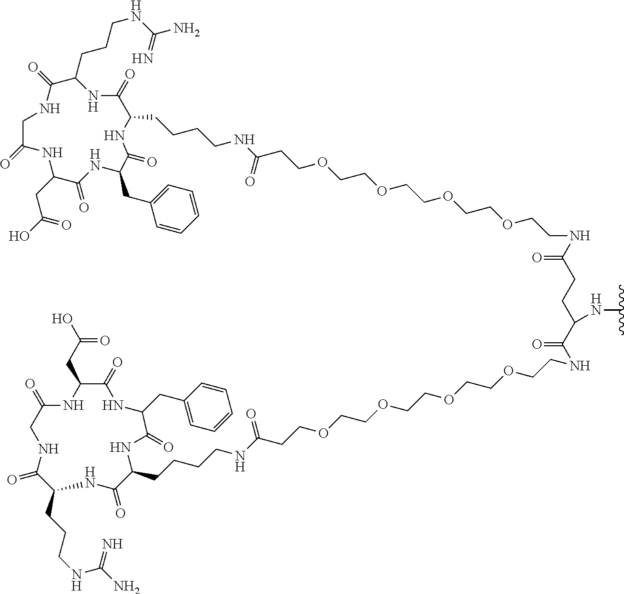 | — | 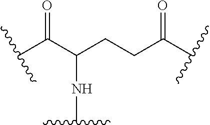 [SMILES: *NC(CCC(*)=O)C(*)=O] |
| 99 | 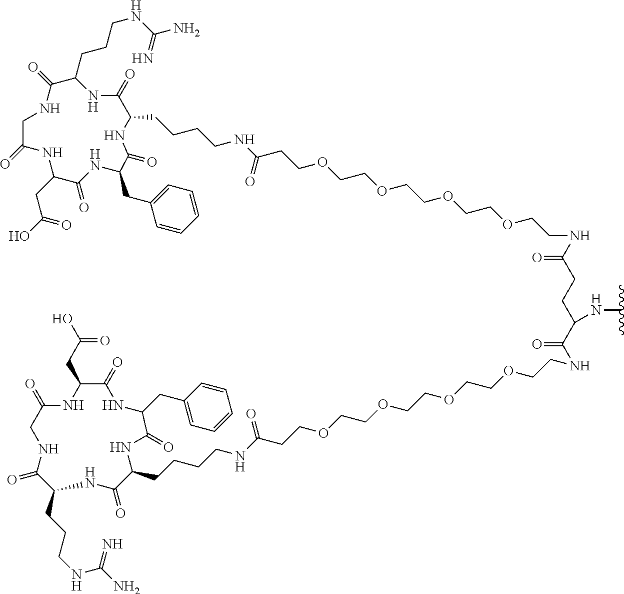 | 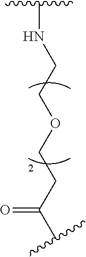 | 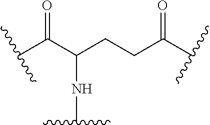 [SMILES: *NC(CCC(*)=O)C(*)=O] |
| 100 | 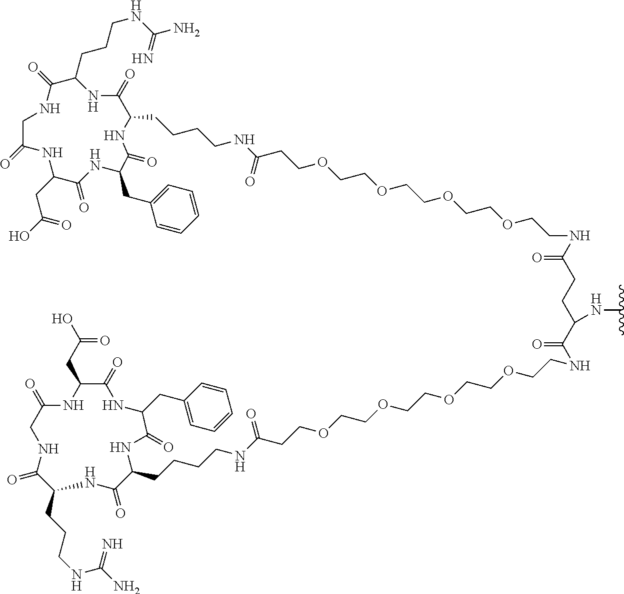 | 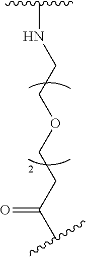 | 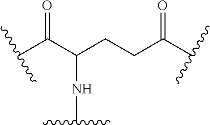 [SMILES: *NC(CCC(*)=O)C(*)=O] |
| 101 | 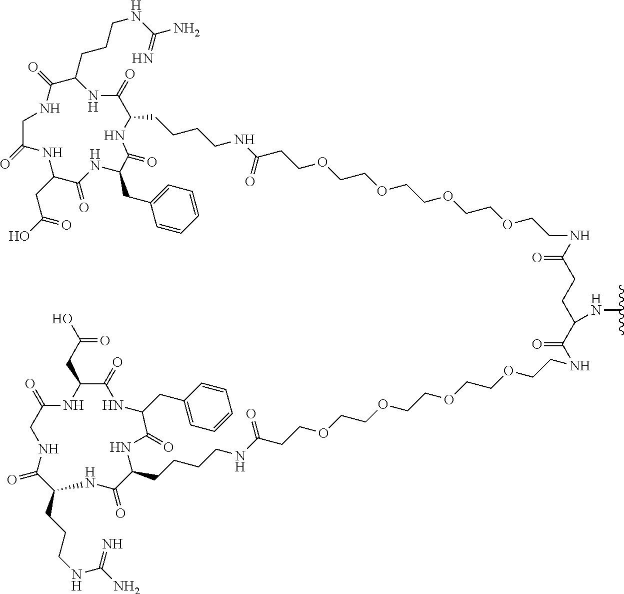 | 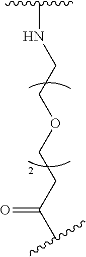 | 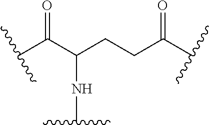 [SMILES: *NC(CCC(*)=O)C(*)=O] |
| 102 | 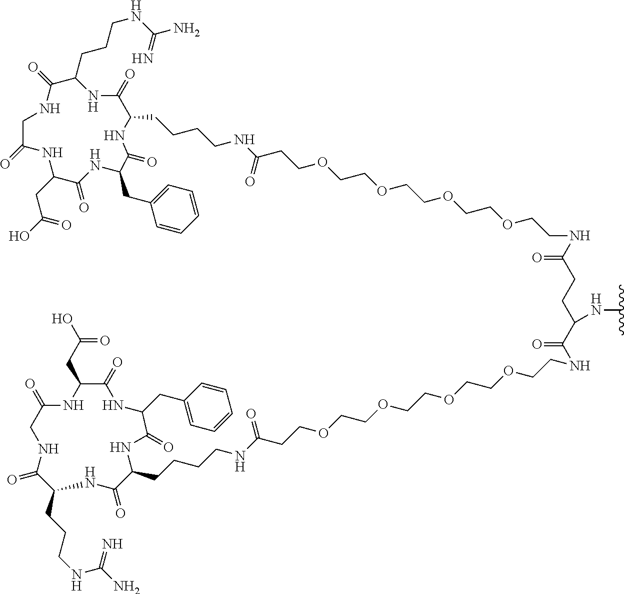 | 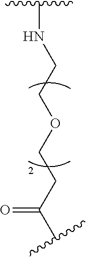 | 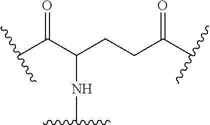 [SMILES: *NC(CCC(*)=O)C(*)=O] |
| 103 | 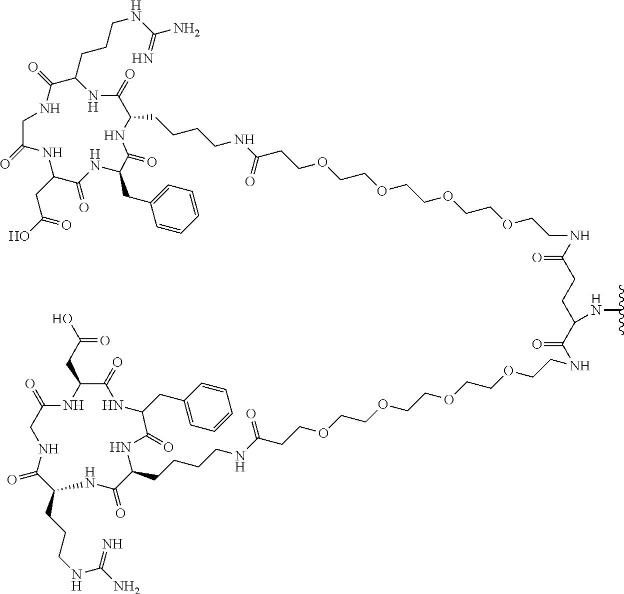 | 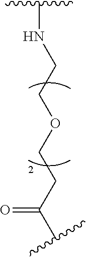 | 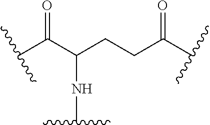 [SMILES: *NC(CCC(*)=O)C(*)=O] |
| 104 | 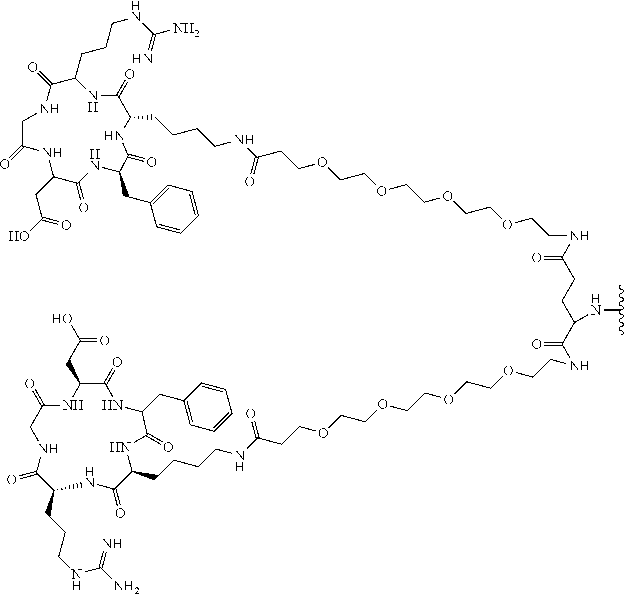 | 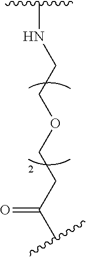 | 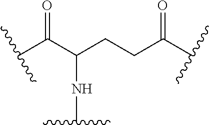 [SMILES: *NC(CCC(*)=O)C(*)=O] |
| 105 | 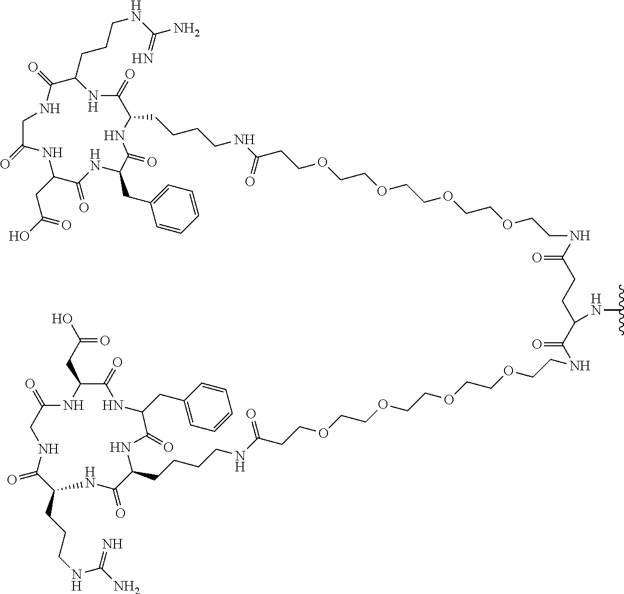 | — | 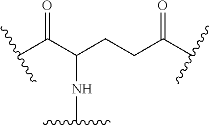 [SMILES: *NC(CCC(*)=O)C(*)=O] |
| 106 | 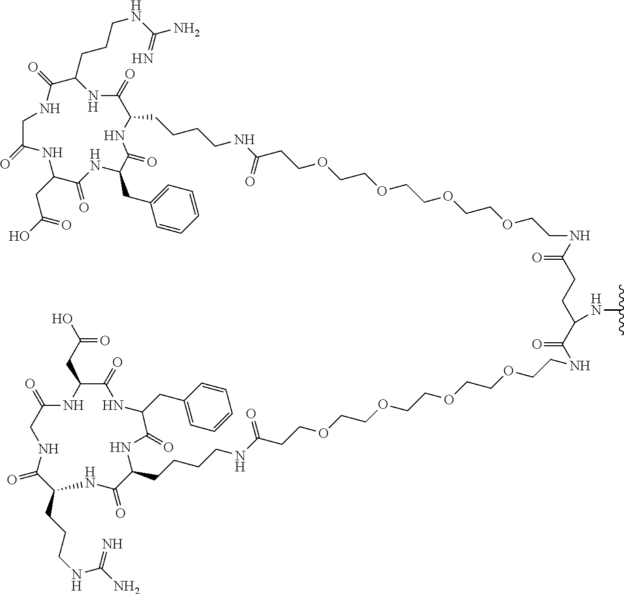 | — | 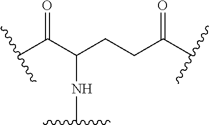 [SMILES: *NC(CCC(*)=O)C(*)=O] |
| Example | | | |
| of | | | |
| Formula | | | R₁/ |
| (V) | V | U | R₂ |
| 84 | 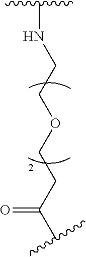 | 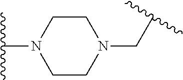 [SMILES: *CN1CCN(*)CC1] | H/H |
| 85 | 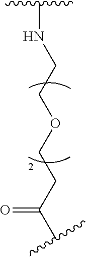 | 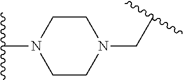 [SMILES: *CN1CCN(*)CC1] | F/F |
| 86 | 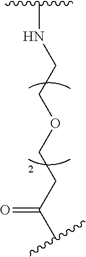 | 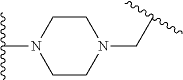 [SMILES: *CN1CCN(*)CC1] | H/H |
| 87 | 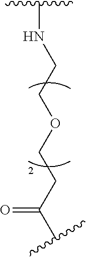 | 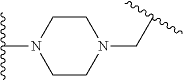 [SMILES: *CN1CCN(*)CC1] | F/F |
| 88 | 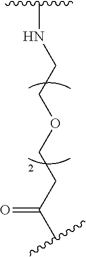 | 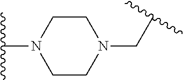 [SMILES: *CN1CCN(*)CC1] | F/F |
| 89 | 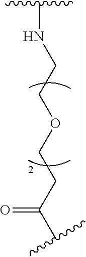 | 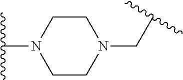 [SMILES: *CN1CCN(*)CC1] | H/H |
| 90 | 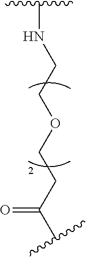 | 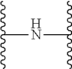 [SMILES: *N*] | H/H |
| 91 | 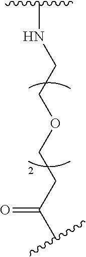 | 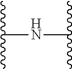 [SMILES: *N*] | F/F |
| 92 | 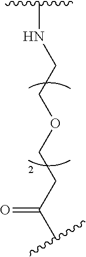 | 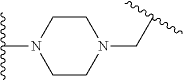 [SMILES: *CN1CCN(*)CC1] | H/H |
| 93 | 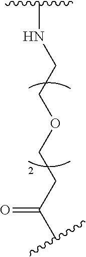 | 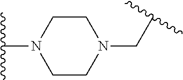 [SMILES: *CN1CCN(*)CC1] | F/F |
| 94 | 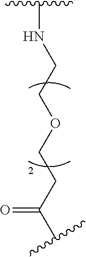 | 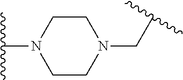 [SMILES: *CN1CCN(*)CC1] | F/F |
| 95 | 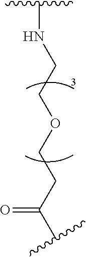 | 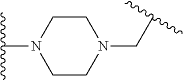 [SMILES: *CN1CCN(*)CC1] | H/H |
| 96 | 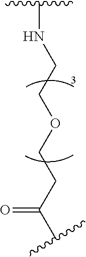 | 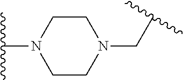 [SMILES: *CN1CCN(*)CC1] | F/F |
| 97 | 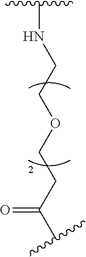 | 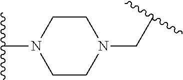 [SMILES: *CN1CCN(*)CC1] | H/H |
| 98 | 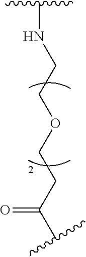 | 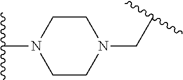 [SMILES: *CN1CCN(*)CC1] | F/F |
| 99 | 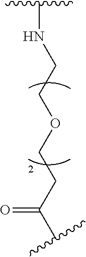 | 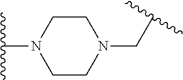 [SMILES: *CN1CCN(*)CC1] | H/H |
| 100 | 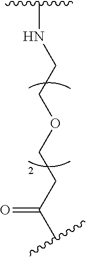 | 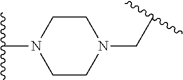 [SMILES: *CN1CCN(*)CC1] | F/F |
| 101 | 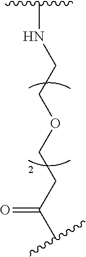 | 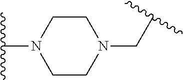 [SMILES: *CN1CCN(*)CC1] | H/H |
| 102 | 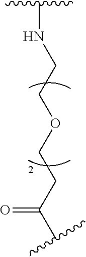 | 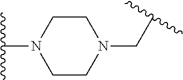 [SMILES: *CN1CCN(*)CC1] | F/F |
| 103 | 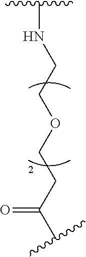 | 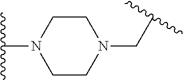 [SMILES: *CN1CCN(*)CC1] | H/H |
| 104 | 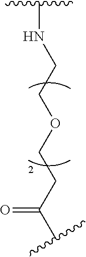 | 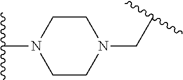 [SMILES: *CN1CCN(*)CC1] | F/F |
| 105 | 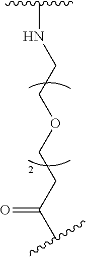 | 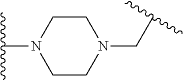 [SMILES: *CN1CCN(*)CC1] | H/H |
| 106 | 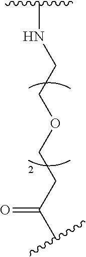 | 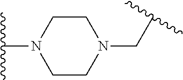 [SMILES: *CN1CCN(*)CC1] | F/F |

| Example | |
|---|---|
| of | |
| Formula | |
| (V) | W |
| 84 | 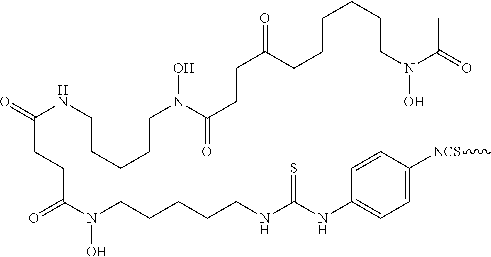 |
| 85 | 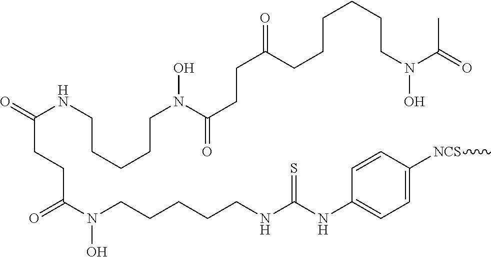 |
| 86 | 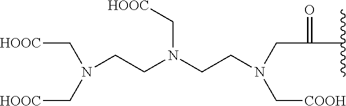 [SMILES: *C(=O)CN(CCN(CCN(CC(=O)O)CC(=O)O)CC(=O)O)CC(=O)O] |
| 87 | 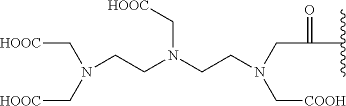 [SMILES: *C(=O)CN(CCN(CCN(CC(=O)O)CC(=O)O)CC(=O)O)CC(=O)O] |
| 88 | 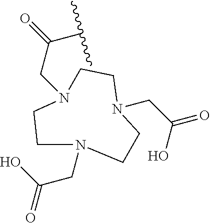 [SMILES: *C(=O)CN1CCN(CC(=O)O)CCN(CC(=O)O)CC1] |
| 89 | 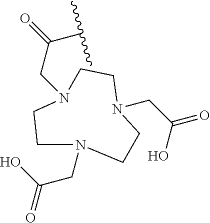 [SMILES: *C(=O)CN1CCN(CC(=O)O)CCN(CC(=O)O)CC1] |
| 90 | 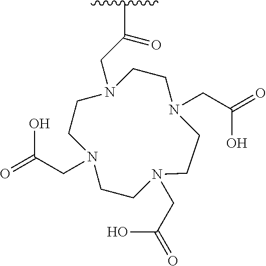 [SMILES: *C(=O)CN1CCN(CC(=O)O)CCN(CC(=O)O)CCN(CC(=O)O)CC1] |
| 91 | 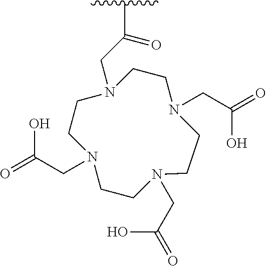 [SMILES: *C(=O)CN1CCN(CC(=O)O)CCN(CC(=O)O)CCN(CC(=O)O)CC1] |
| 92 | 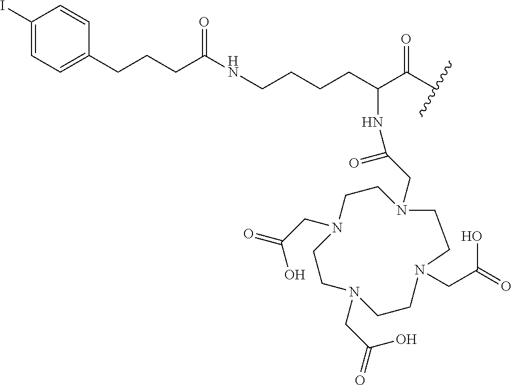 [SMILES: *C(=O)C(CCCCNC(=O)CCCc1ccc(I)cc1)NC(=O)CN1CCN(CC(=O)O)CCN(CC(=O)O)CCN(CC(=O)O)CC1] |
| 93 | 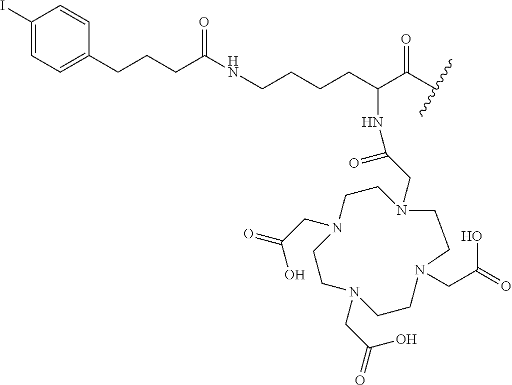 [SMILES: *C(=O)C(CCCCNC(=O)CCCc1ccc(I)cc1)NC(=O)CN1CCN(CC(=O)O)CCN(CC(=O)O)CCN(CC(=O)O)CC1] |
| 94 | 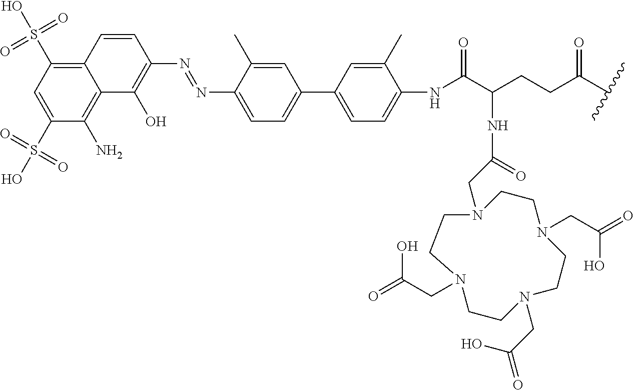 [SMILES: *C(=O)CCC(NC(=O)CN1CCN(CC(=O)O)CCN(CC(=O)O)CCN(CC(=O)O)CC1)C(=O)Nc1ccc(-c2ccc(/N=N/c3ccc4c(S(=O)(=O)O)cc(S(=O)(=O)O)c(N)c4c3O)c(C)c2)cc1C] |
| 95 | 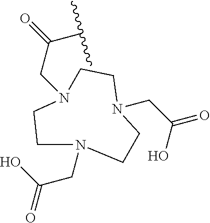 [SMILES: *C(=O)CN1CCN(CC(=O)O)CCN(CC(=O)O)CC1] |
| 96 | 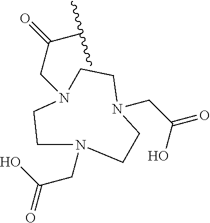 [SMILES: *C(=O)CN1CCN(CC(=O)O)CCN(CC(=O)O)CC1] |
| 97 | 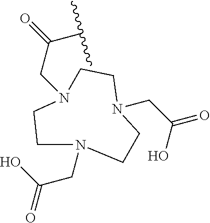 [SMILES: *C(=O)CN1CCN(CC(=O)O)CCN(CC(=O)O)CC1] |
| 98 | 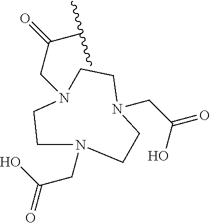 [SMILES: *C(=O)CN1CCN(CC(=O)O)CCN(CC(=O)O)CC1] |
| 99 | 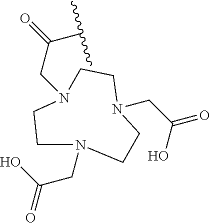 [SMILES: *C(=O)CN1CCN(CC(=O)O)CCN(CC(=O)O)CC1] |
| 100 | 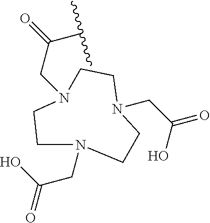 [SMILES: *C(=O)CN1CCN(CC(=O)O)CCN(CC(=O)O)CC1] |
| 101 | 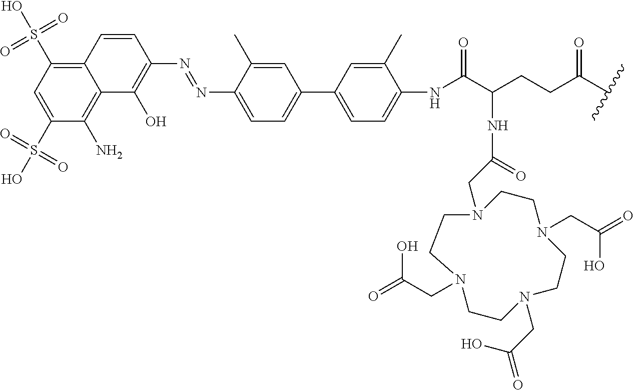 [SMILES: *C(=O)CCC(NC(=O)CN1CCN(CC(=O)O)CCN(CC(=O)O)CCN(CC(=O)O)CC1)C(=O)Nc1ccc(-c2ccc(/N=N/c3ccc4c(S(=O)(=O)O)cc(S(=O)(=O)O)c(N)c4c3O)c(C)c2)cc1C] |
| 102 | 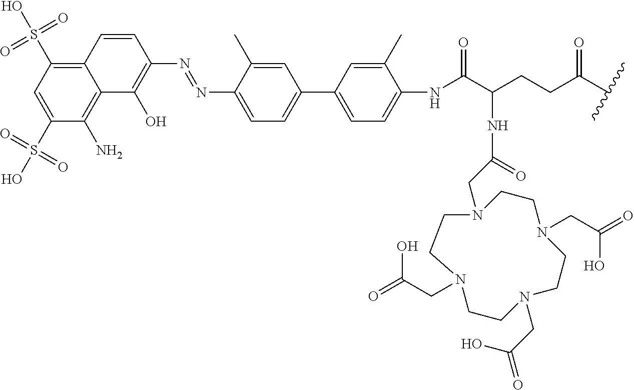 [SMILES: *C(=O)CCC(NC(=O)CN1CCN(CC(=O)O)CCN(CC(=O)O)CCN(CC(=O)O)CC1)C(=O)Nc1ccc(-c2ccc(/N=N/c3ccc4c(S(=O)(=O)O)cc(S(=O)(=O)O)c(N)c4c3O)c(C)c2)cc1C] |
| 103 | 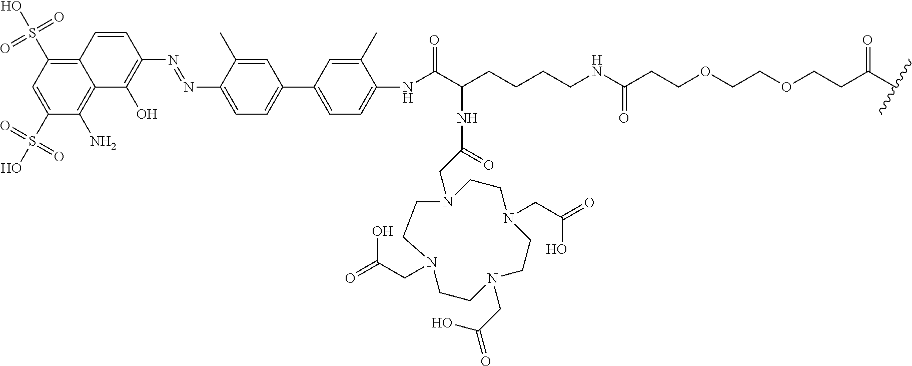 [SMILES: *C(=O)CCOCCOCCC(=O)NCCCCC(NC(=O)CN1CCN(CC(=O)O)CCN(CC(=O)O)CCN(CC(=O)O)CC1)C(=O)Nc1ccc(-c2ccc(/N=N/c3ccc4c(S(=O)(=O)O)cc(S(=O)(=O)O)c(N)c4c3O)c(C)c2)cc1C] |
| 104 | 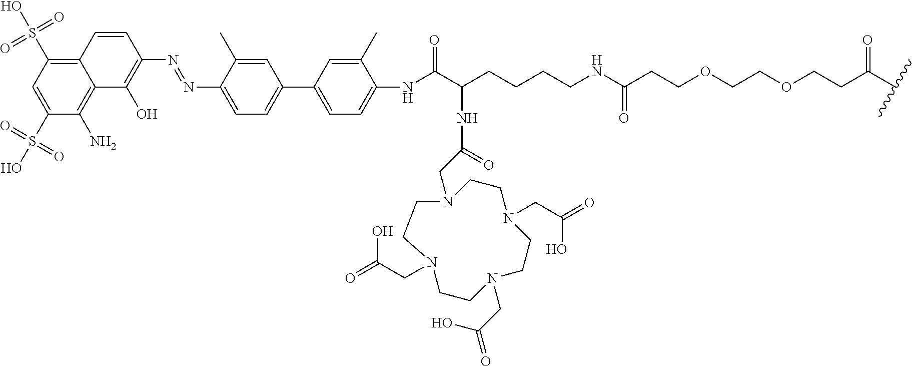 [SMILES: *C(=O)CCOCCOCCC(=O)NCCCCC(NC(=O)CN1CCN(CC(=O)O)CCN(CC(=O)O)CCN(CC(=O)O)CC1)C(=O)Nc1ccc(-c2ccc(/N=N/c3ccc4c(S(=O)(=O)O)cc(S(=O)(=O)O)c(N)c4c3O)c(C)c2)cc1C] |
| 105 | 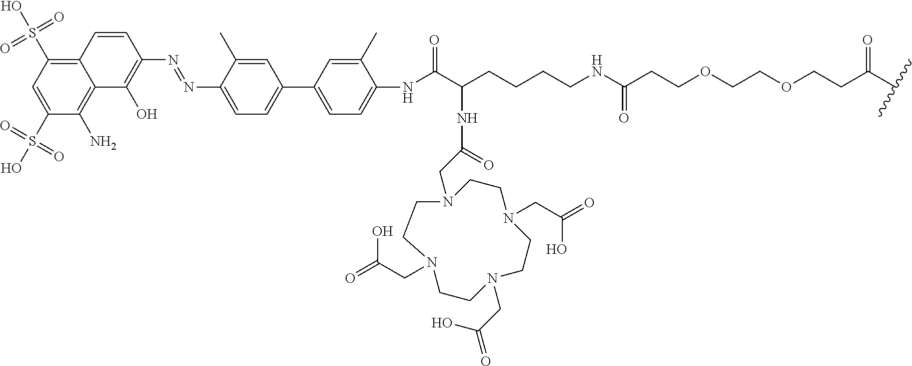 [SMILES: *C(=O)CCOCCOCCC(=O)NCCCCC(NC(=O)CN1CCN(CC(=O)O)CCN(CC(=O)O)CCN(CC(=O)O)CC1)C(=O)Nc1ccc(-c2ccc(/N=N/c3ccc4c(S(=O)(=O)O)cc(S(=O)(=O)O)c(N)c4c3O)c(C)c2)cc1C] |
| 106 | 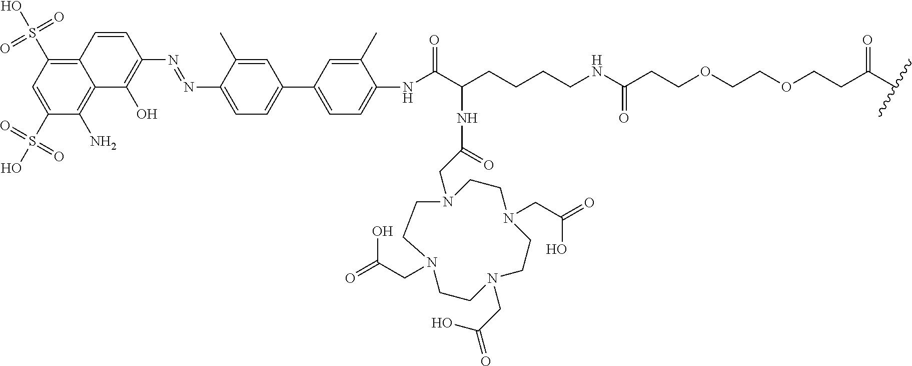 [SMILES: *C(=O)CCOCCOCCC(=O)NCCCCC(NC(=O)CN1CCN(CC(=O)O)CCN(CC(=O)O)CCN(CC(=O)O)CC1)C(=O)Nc1ccc(-c2ccc(/N=N/c3ccc4c(S(=O)(=O)O)cc(S(=O)(=O)O)c(N)c4c3O)c(C)c2)cc1C] |

TABLE 2

| Example | | | | |
|---|---|---|---|---|
| of | | | | |
| Formula | | | | |
| (VI) | A | Z2 | Q | V |
| 107 | 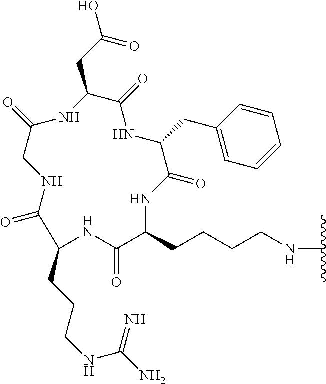 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 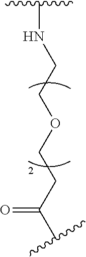 | 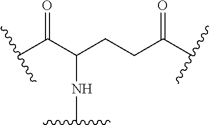 [SMILES: *NC(CCC(*)=O)C(*)=O] | 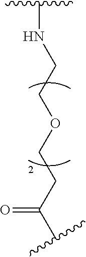 |
| 108 | 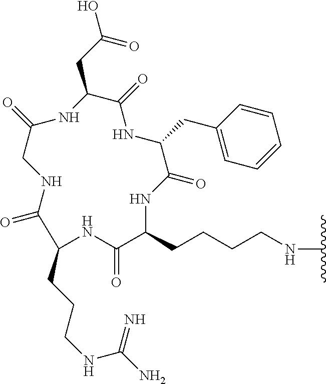 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 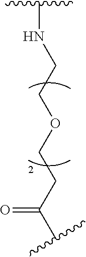 | 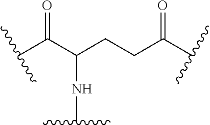 [SMILES: *NC(CCC(*)=O)C(*)=O] | 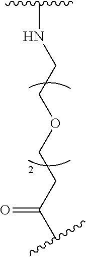 |
| 109 | 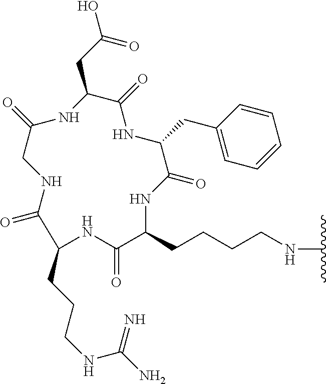 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 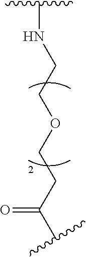 | 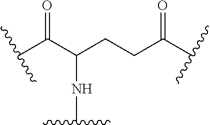 [SMILES: *NC(CCC(*)=O)C(*)=O] | 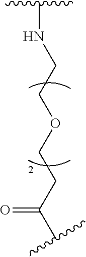 |
| 110 | 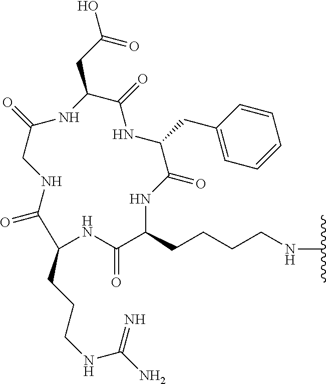 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 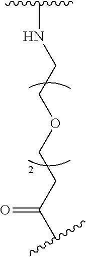 | 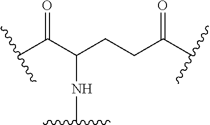 [SMILES: *NC(CCC(*)=O)C(*)=O] | 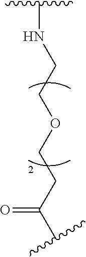 |
| 111 | 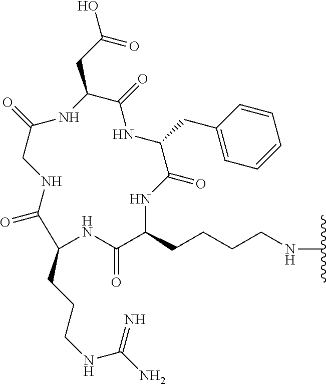 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 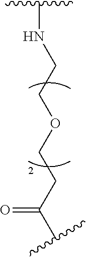 | 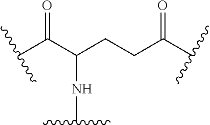 [SMILES: *NC(CCC(*)=O)C(*)=O] | 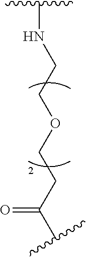 |
| 112 | 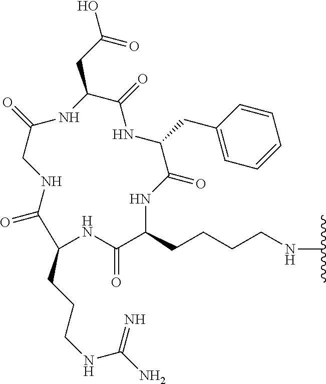 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 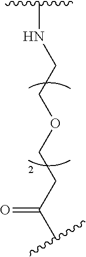 | 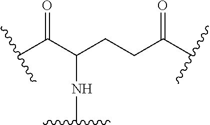 [SMILES: *NC(CCC(*)=O)C(*)=O] | 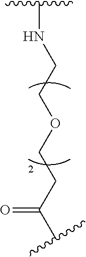 |
| 113 | 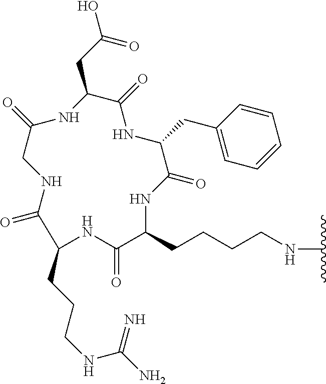 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 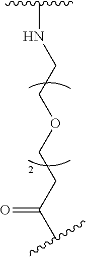 | 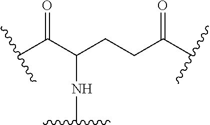 [SMILES: *NC(CCC(*)=O)C(*)=O] | 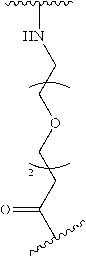 |
| 114 | 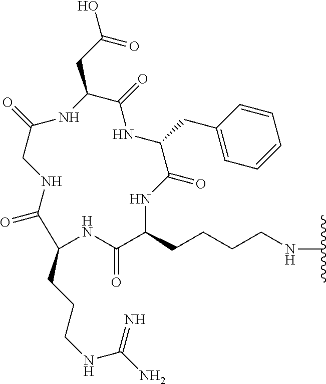 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 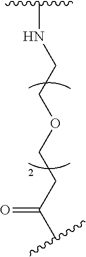 | 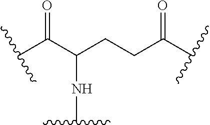 [SMILES: *NC(CCC(*)=O)C(*)=O] | 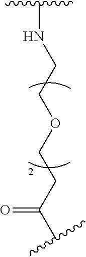 |
| 115 | 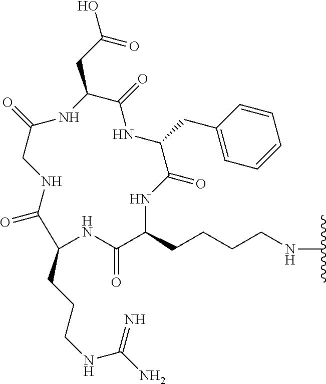 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 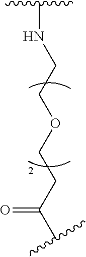 | 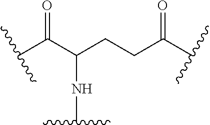 [SMILES: *NC(CCC(*)=O)C(*)=O] | 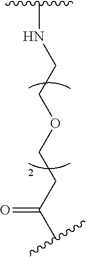 |
| 116 | 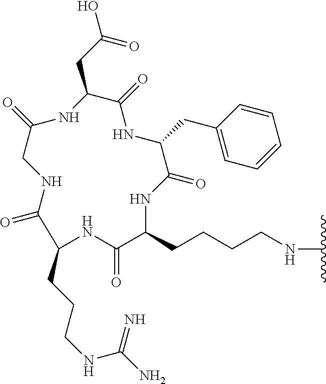 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 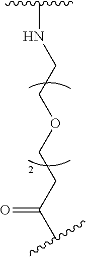 | 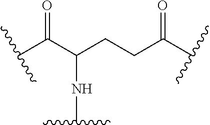 [SMILES: *NC(CCC(*)=O)C(*)=O] | 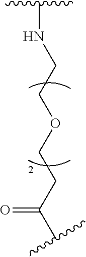 |
| 117 | 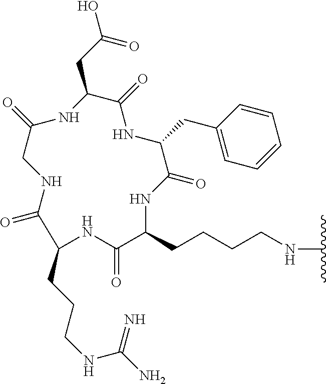 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 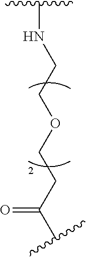 | 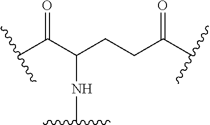 [SMILES: *NC(CCC(*)=O)C(*)=O] | 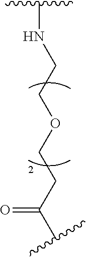 |
| 118 | 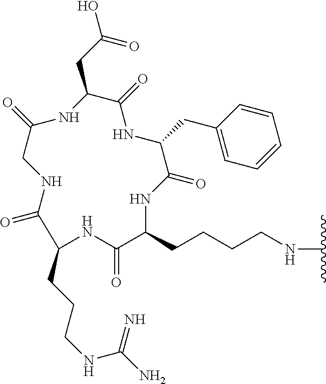 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O] | 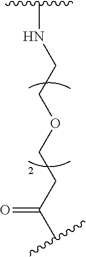 | 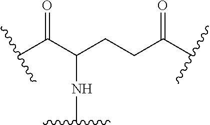 [SMILES: *NC(CCC(*)=O)C(*)=O] | 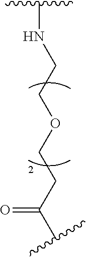 |
| 119 | 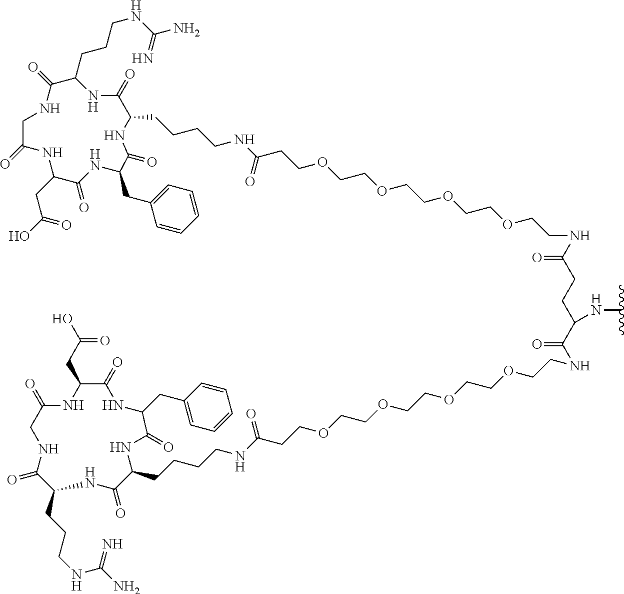 | — | 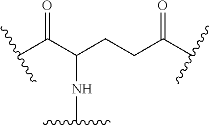 [SMILES: *NC(CCC(*)=O)C(*)=O] | 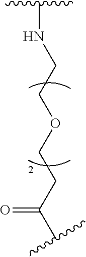 |
| 120 | 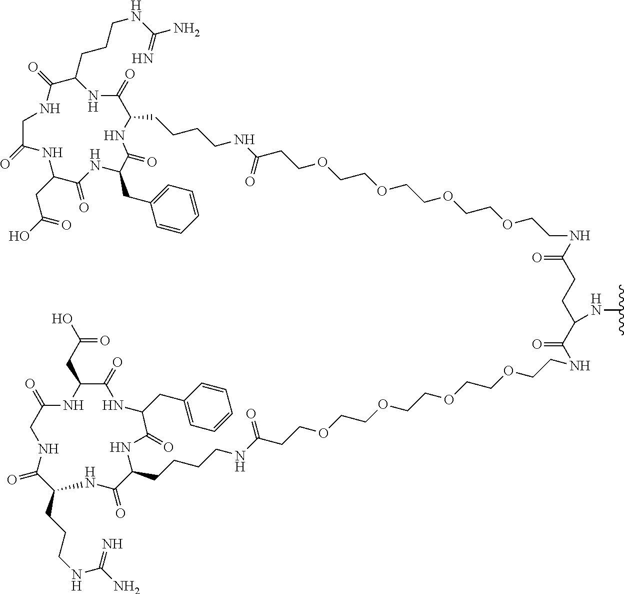 | — | 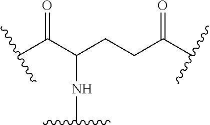 [SMILES: *NC(CCC(*)=O)C(*)=O] | 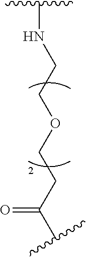 |
| 121 | 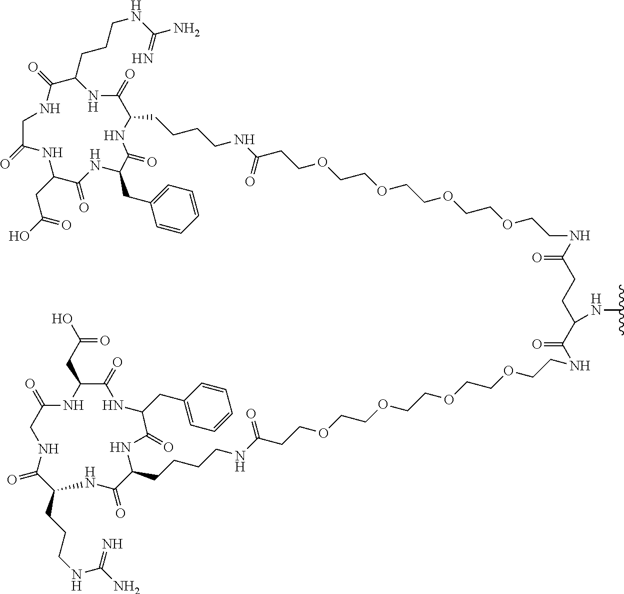 | — | 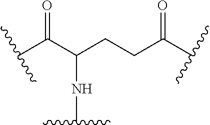 [SMILES: *NC(CCC(*)=O)C(*)=O] | 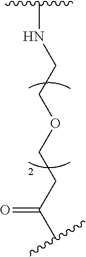 |
| 122 | 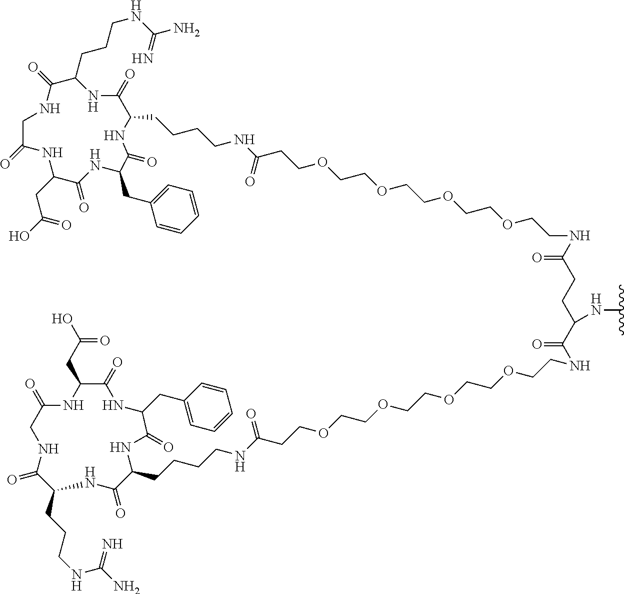 | — | 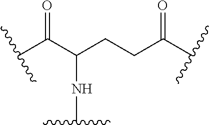 [SMILES: *NC(CCC(*)=O)C(*)=O] | 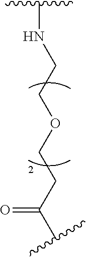 |
| 123 | 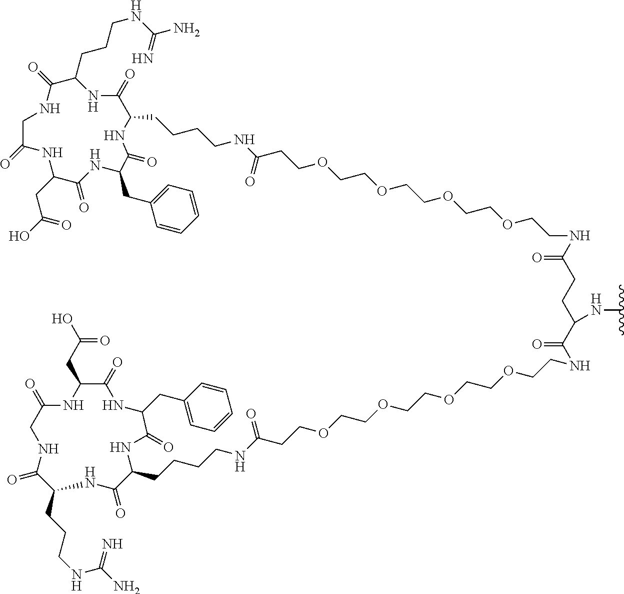 | — | 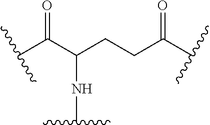 [SMILES: *NC(CCC(*)=O)C(*)=O] | 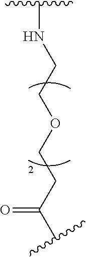 |
| 124 | 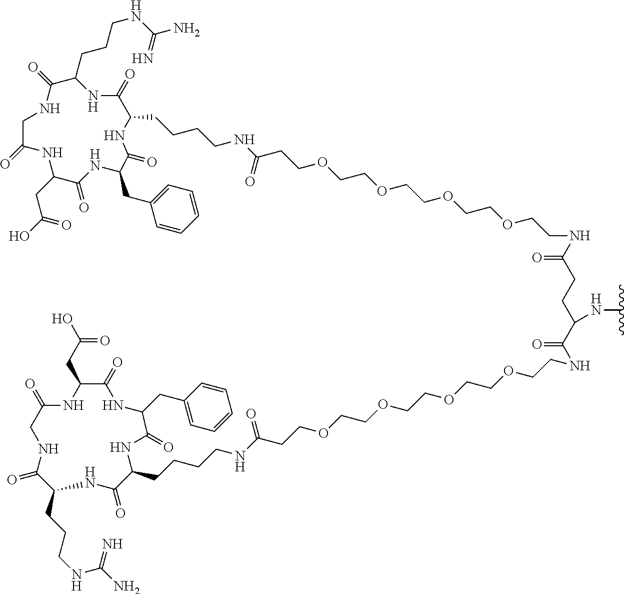 | 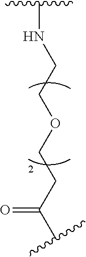 | 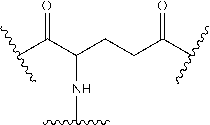 [SMILES: *NC(CCC(*)=O)C(*)=O] | 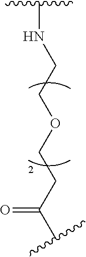 |
| 125 | 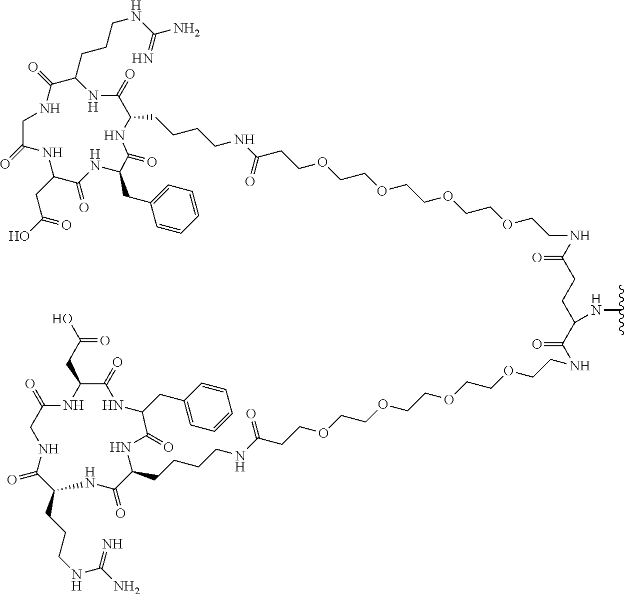 | 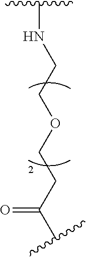 | 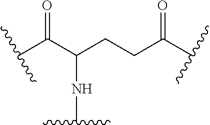 [SMILES: *NC(CCC(*)=O)C(*)=O] | 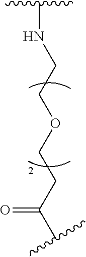 |
| 126 | 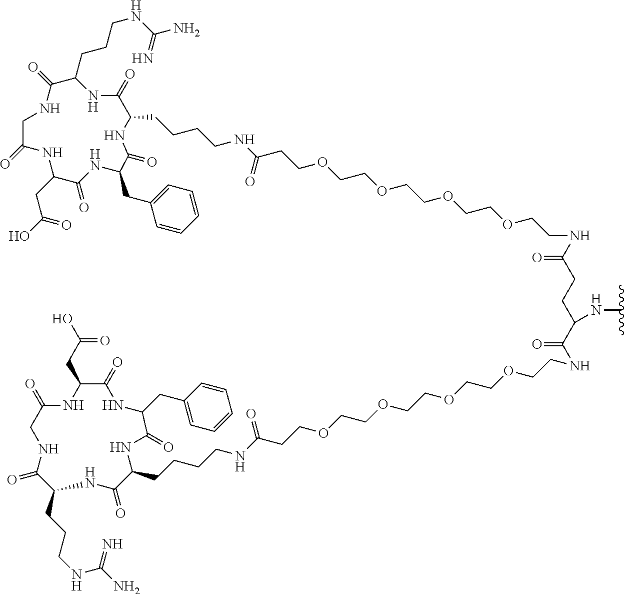 | 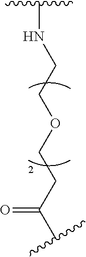 | 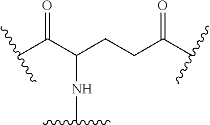 [SMILES: *NC(CCC(*)=O)C(*)=O] | 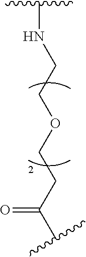 |
| 127 | 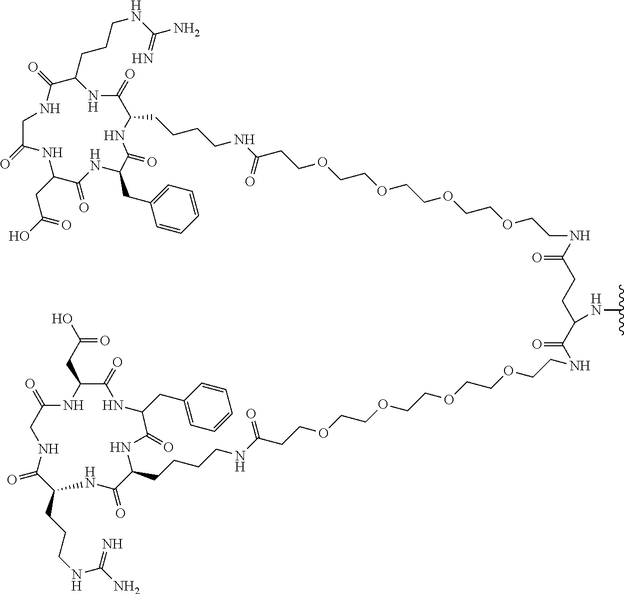 | 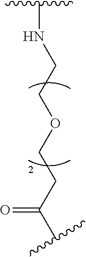 | 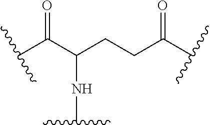 [SMILES: *NC(CCC(*)=O)C(*)=O] | 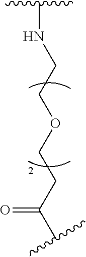 |
| 128 | 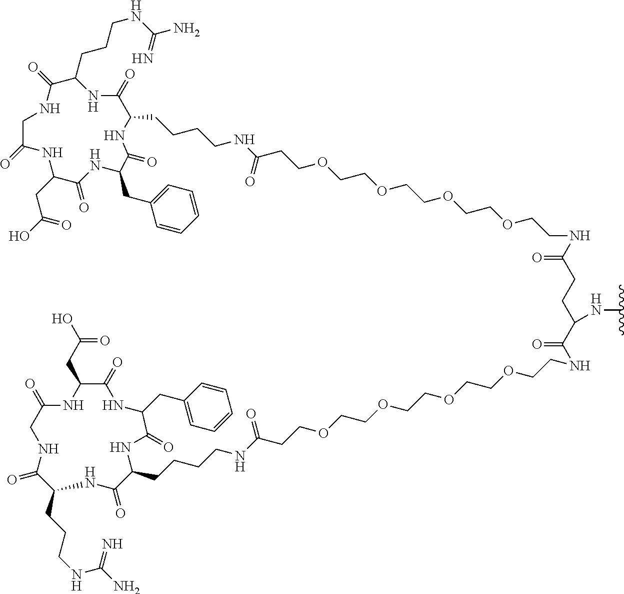 | 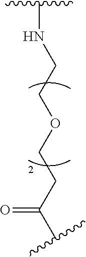 | 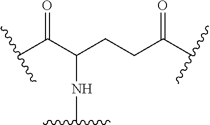 [SMILES: *NC(CCC(*)=O)C(*)=O] | 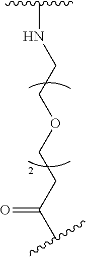 |
| 129 | 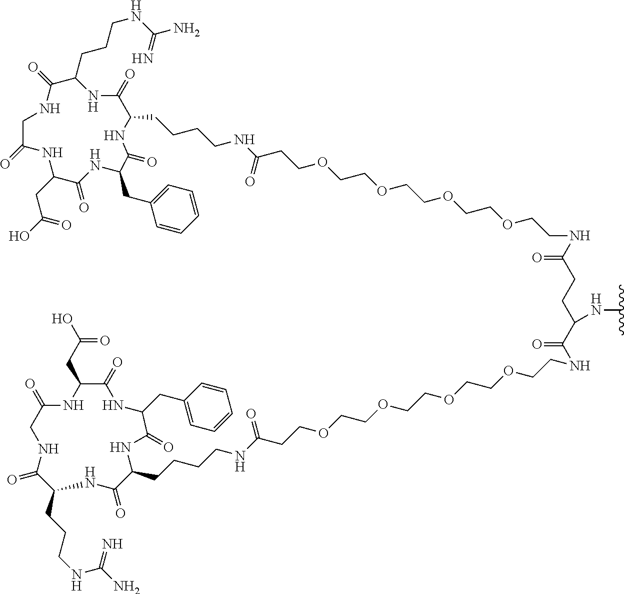 | — | 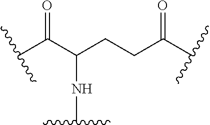 [SMILES: *NC(CCC(*)=O)C(*)=O] | 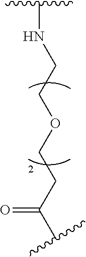 |
| 130 | 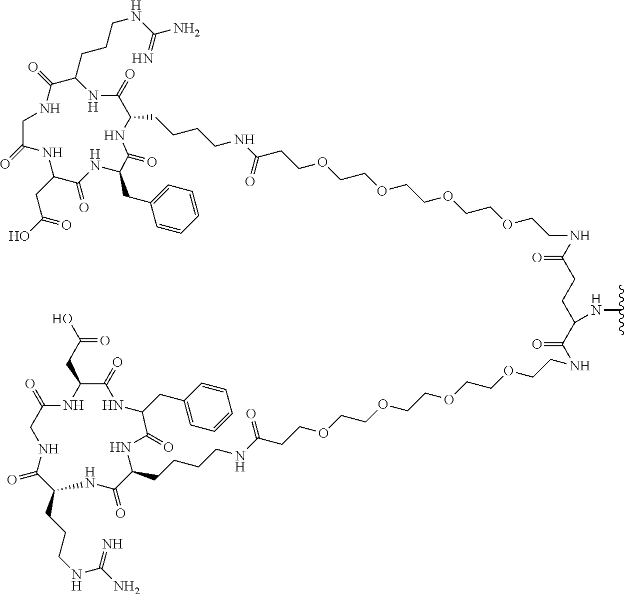 | — | 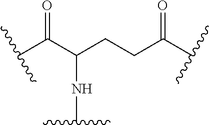 [SMILES: *NC(CCC(*)=O)C(*)=O] | 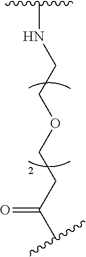 |
| 131 | 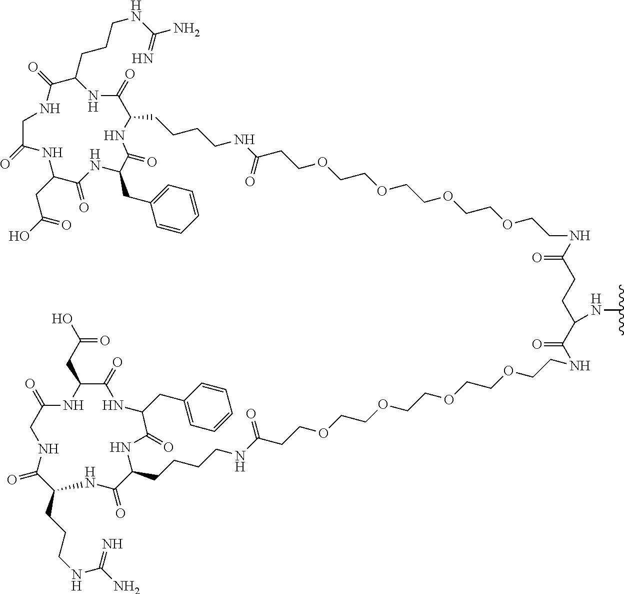 | — | 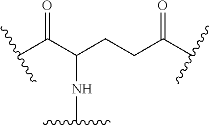 [SMILES: *NC(CCC(*)=O)C(*)=O] | 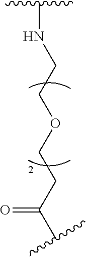 |
| 132 | 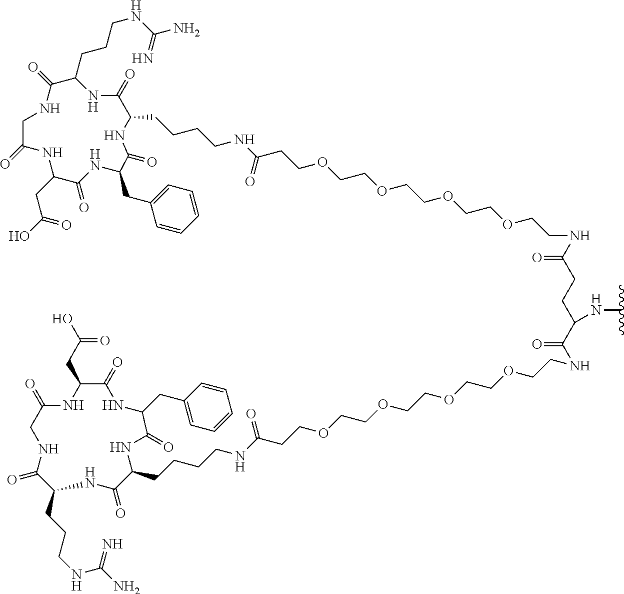 | — | 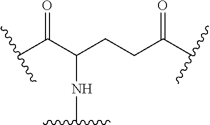 [SMILES: *NC(CCC(*)=O)C(*)=O] | 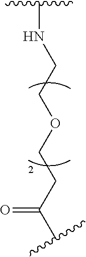 |
| 133 | 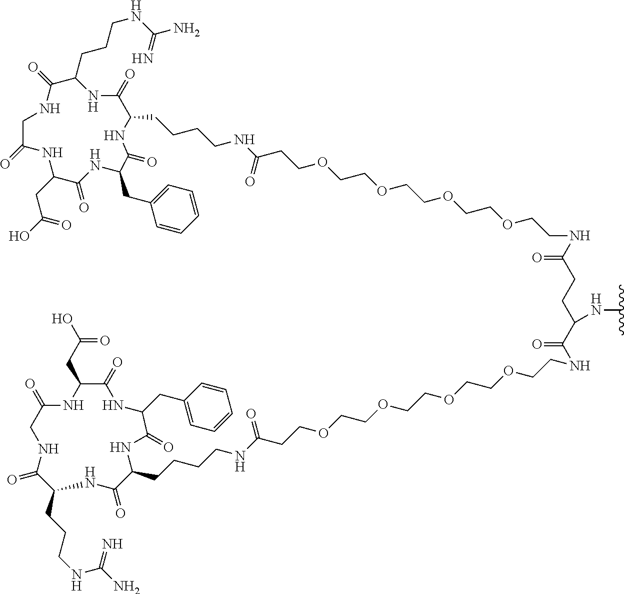 | — | 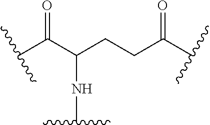 [SMILES: *NC(CCC(*)=O)C(*)=O] | 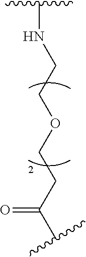 |

| Example | | | |
|---|---|---|---|
| of | | | |
| Formula | | | R₁/R₂; |
| (VI) | Z1 | U | R₃/R₄ |
| 107 | 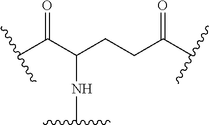 [SMILES: *NC(CCC(*)=O)C(*)=O] | 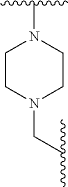 [SMILES: *CN1CCN(*)CC1] | F/F; F/F |
| 108 | 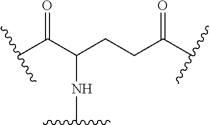 [SMILES: *NC(CCC(*)=O)C(*)=O] | 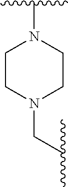 [SMILES: *CN1CCN(*)CC1] | H/H; H/H |
| 109 | 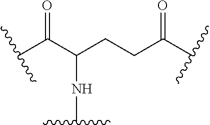 [SMILES: *NC(CCC(*)=O)C(*)=O] | 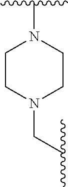 [SMILES: *CN1CCN(*)CC1] | F/F; F/F |
| 110 | 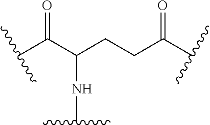 [SMILES: *NC(CCC(*)=O)C(*)=O] | 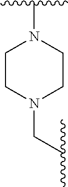 [SMILES: *CN1CCN(*)CC1] | H/H; H/H |
| 111 | 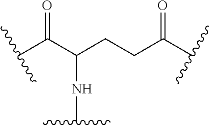 [SMILES: *NC(CCC(*)=O)C(*)=O] | 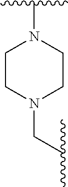 [SMILES: *CN1CCN(*)CC1] | F/F; F/F |
| 112 | 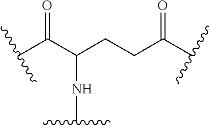 [SMILES: *NC(CCC(*)=O)C(*)=O] | 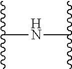 [SMILES: *N*] | F/F; F/F |
| 113 | 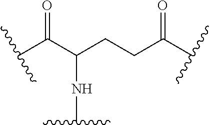 [SMILES: *NC(CCC(*)=O)C(*)=O] | 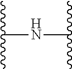 [SMILES: *N*] | H/H; H/H |
| 114 | 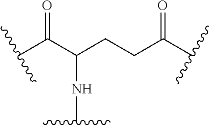 [SMILES: *NC(CCC(*)=O)C(*)=O] | 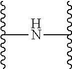 [SMILES: *N*] | F/F; F/F |
| 115 | 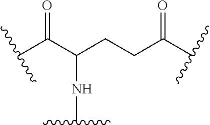 [SMILES: *NC(CCC(*)=O)C(*)=O] | 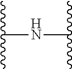 [SMILES: *N*] | H/H; H/H |
| 116 | 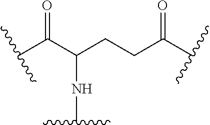 [SMILES: *NC(CCC(*)=O)C(*)=O] | 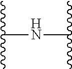 [SMILES: *N*] | F/F; F/F |
| 117 | 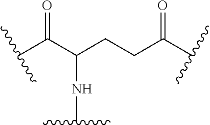 [SMILES: *NC(CCC(*)=O)C(*)=O] | 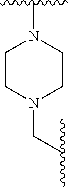 [SMILES: *CN1CCN(*)CC1] | H/H; H/H |
| 118 | 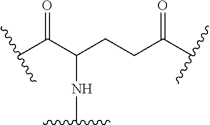 [SMILES: *NC(CCC(*)=O)C(*)=O] | 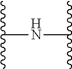 [SMILES: *N*] | H/H; H/H |
| 119 | 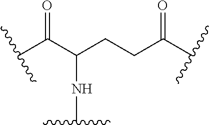 [SMILES: *NC(CCC(*)=O)C(*)=O] | 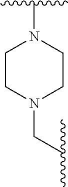 [SMILES: *CN1CCN(*)CC1] | F/F; F/F |
| 120 | 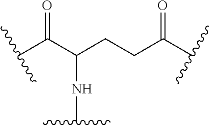 [SMILES: *NC(CCC(*)=O)C(*)=O] | 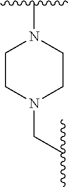 [SMILES: *CN1CCN(*)CC1] | H/H; H/H |
| 121 | 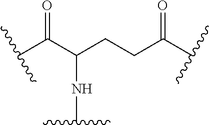 [SMILES: *NC(CCC(*)=O)C(*)=O] | 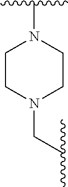 [SMILES: *CN1CCN(*)CC1] | H/H; H/H |
| 122 | 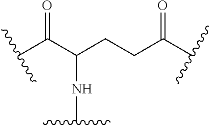 [SMILES: *NC(CCC(*)=O)C(*)=O] | 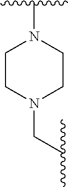 [SMILES: *CN1CCN(*)CC1] | F/F; F/F |
| 123 | 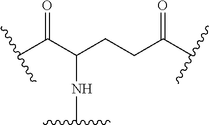 [SMILES: *NC(CCC(*)=O)C(*)=O] | 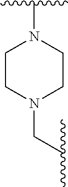 [SMILES: *CN1CCN(*)CC1] | F/F; F/F |
| 124 | 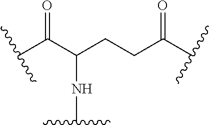 [SMILES: *NC(CCC(*)=O)C(*)=O] | 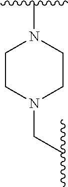 [SMILES: *CN1CCN(*)CC1] | F/F; F/F |
| 125 | 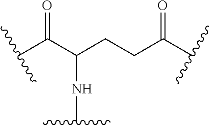 [SMILES: *NC(CCC(*)=O)C(*)=O] | 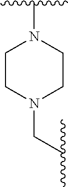 [SMILES: *CN1CCN(*)CC1] | H/H; H/H |
| 126 | 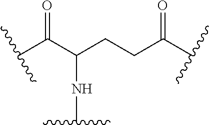 [SMILES: *NC(CCC(*)=O)C(*)=O] | 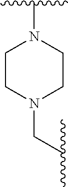 [SMILES: *CN1CCN(*)CC1] | H/H; H/H |
| 127 | 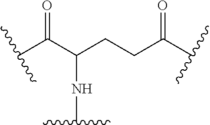 [SMILES: *NC(CCC(*)=O)C(*)=O] | 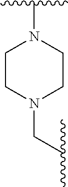 [SMILES: *CN1CCN(*)CC1] | H/H; H/H |
| 128 | 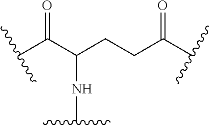 [SMILES: *NC(CCC(*)=O)C(*)=O] | 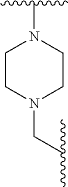 [SMILES: *CN1CCN(*)CC1] | H/H; H/H |
| 129 | 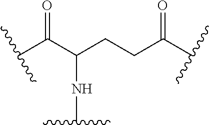 [SMILES: *NC(CCC(*)=O)C(*)=O] | 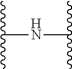 [SMILES: *N*] | H/H; H/H |
| 130 | 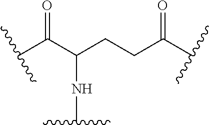 [SMILES: *NC(CCC(*)=O)C(*)=O] | 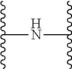 [SMILES: *N*] | H/H; H/H |
| 131 | 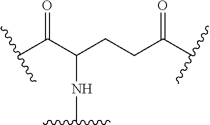 [SMILES: *NC(CCC(*)=O)C(*)=O] | 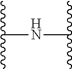 [SMILES: *N*] | H/H; H/H |
| 132 | 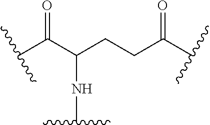 [SMILES: *NC(CCC(*)=O)C(*)=O] | 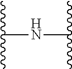 [SMILES: *N*] | H/H; H/H |
| 133 | 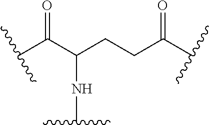 [SMILES: *NC(CCC(*)=O)C(*)=O] | 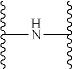 [SMILES: *N*] | H/H; H/H |

| Example | |
|---|---|
| of | |
| Formula | |
| (VI) | W |
| 107 | 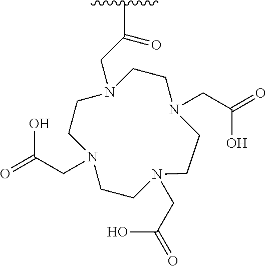 [SMILES: *C(=O)CN1CCN(CC(=O)O)CCN(CC(=O)O)CCN(CC(=O)O)CC1] |
| 108 | 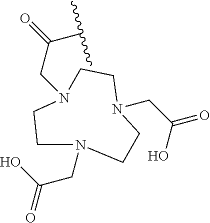 [SMILES: *C(=O)CN1CCN(CC(=O)O)CCN(CC(=O)O)CC1] |
| 109 | 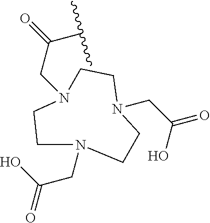 [SMILES: *C(=O)CN1CCN(CC(=O)O)CCN(CC(=O)O)CC1] |
| 110 | 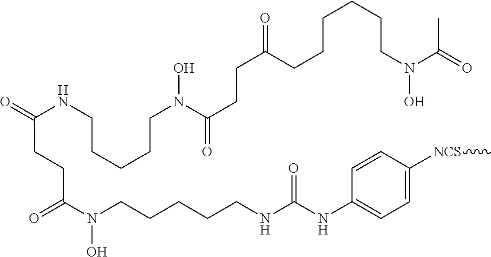 |
| 111 | 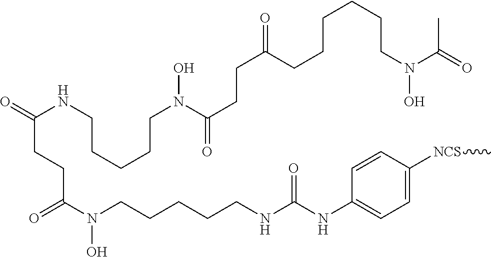 |
| 112 | 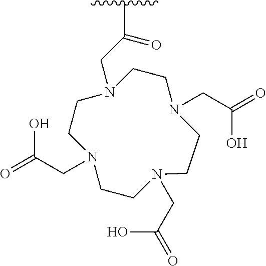 [SMILES: *C(=O)CN1CCN(CC(=O)O)CCN(CC(=O)O)CCN(CC(=O)O)CC1] |
| 113 | 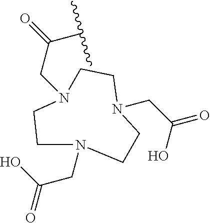 [SMILES: *C(=O)CN1CCN(CC(=O)O)CCN(CC(=O)O)CC1] |
| 114 | 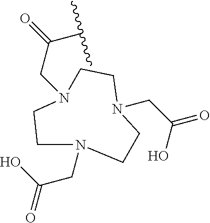 [SMILES: *C(=O)CN1CCN(CC(=O)O)CCN(CC(=O)O)CC1] |
| 115 | 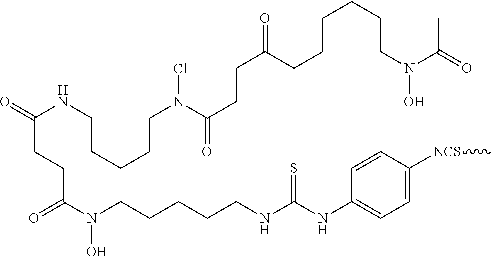 |
| 116 | 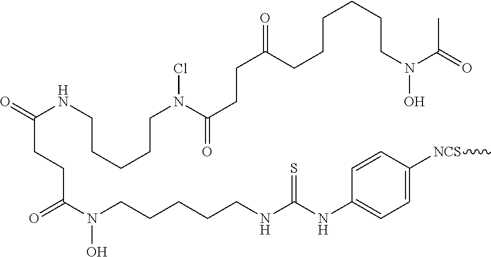 |
| 117 | 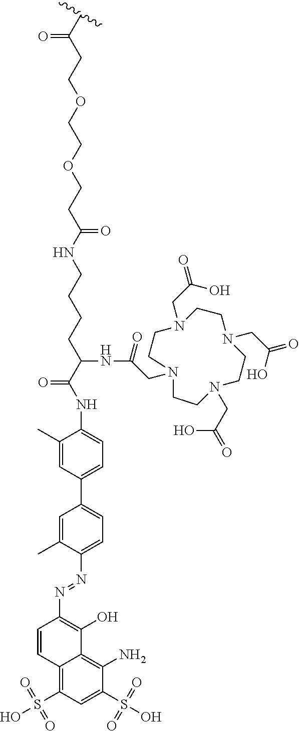 [SMILES: *C(=O)CCOCCOCCC(=O)NCCCCC(NC(=O)CN1CCN(CC(=O)O)CCN(CC(=O)O)CCN(CC(=O)O)CC1)C(=O)Nc1ccc(-c2ccc(/N=N/c3ccc4c(S(=O)(=O)O)cc(S(=O)(=O)O)c(N)c4c3O)c(C)c2)cc1C] |
| 118 | 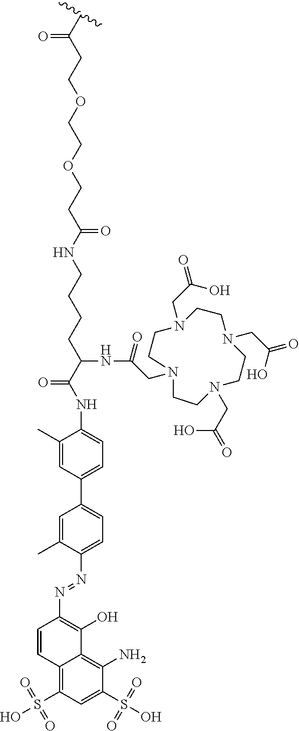 [SMILES: *C(=O)CCOCCOCCC(=O)NCCCCC(NC(=O)CN1CCN(CC(=O)O)CCN(CC(=O)O)CCN(CC(=O)O)CC1)C(=O)Nc1ccc(-c2ccc(/N=N/c3ccc4c(S(=O)(=O)O)cc(S(=O)(=O)O)c(N)c4c3O)c(C)c2)cc1C] |
| 119 | 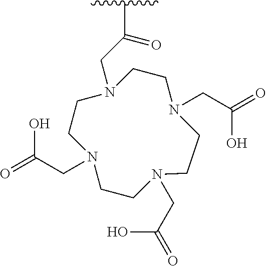 [SMILES: *C(=O)CN1CCN(CC(=O)O)CCN(CC(=O)O)CCN(CC(=O)O)CC1] |
| 120 | 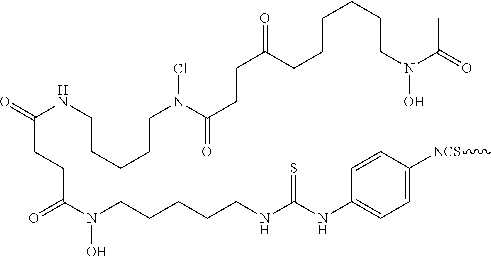 |
| 121 | 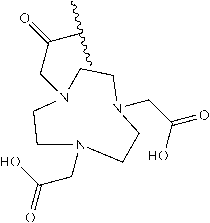 [SMILES: *C(=O)CN1CCN(CC(=O)O)CCN(CC(=O)O)CC1] |
| 122 | 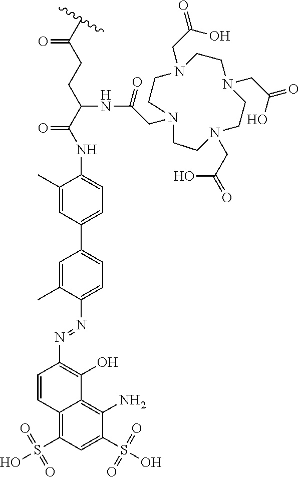 [SMILES: *C(=O)CCC(NC(=O)CN1CCN(CC(=O)O)CCN(CC(=O)O)CCN(CC(=O)O)CC1)C(=O)Nc1ccc(-c2ccc(/N=N/c3ccc4c(S(=O)(=O)O)cc(S(=O)(=O)O)c(N)c4c3O)c(C)c2)cc1C] |
| 123 | 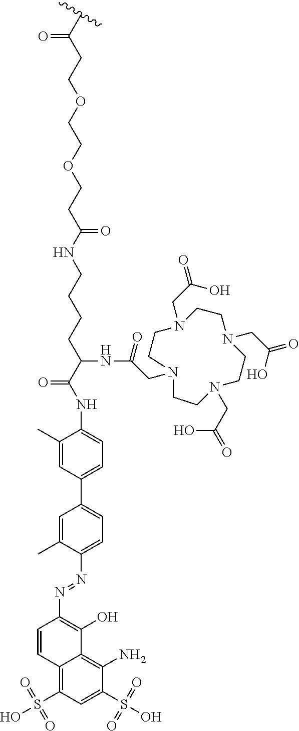 [SMILES: *C(=O)CCOCCOCCC(=O)NCCCCC(NC(=O)CN1CCN(CC(=O)O)CCN(CC(=O)O)CCN(CC(=O)O)CC1)C(=O)Nc1ccc(-c2ccc(/N=N/c3ccc4c(S(=O)(=O)O)cc(S(=O)(=O)O)c(N)c4c3O)c(C)c2)cc1C] |
| 124 | 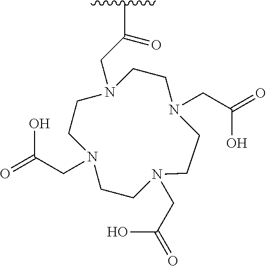 [SMILES: *C(=O)CN1CCN(CC(=O)O)CCN(CC(=O)O)CCN(CC(=O)O)CC1] |
| 125 | 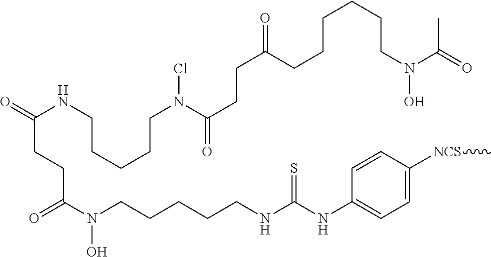 |
| 126 | 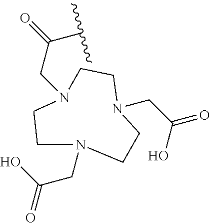 [SMILES: *C(=O)CN1CCN(CC(=O)O)CCN(CC(=O)O)CC1] |
| 127 | 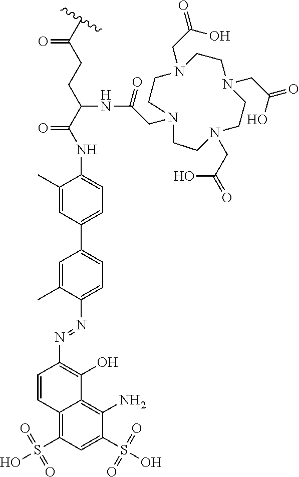 [SMILES: *C(=O)CCC(NC(=O)CN1CCN(CC(=O)O)CCN(CC(=O)O)CCN(CC(=O)O)CC1)C(=O)Nc1ccc(-c2ccc(/N=N/c3ccc4c(S(=O)(=O)O)cc(S(=O)(=O)O)c(N)c4c3O)c(C)c2)cc1C] |
| 128 | 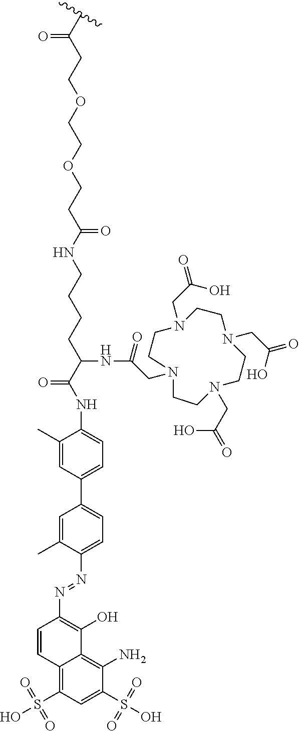 [SMILES: *C(=O)CCOCCOCCC(=O)NCCCCC(NC(=O)CN1CCN(CC(=O)O)CCN(CC(=O)O)CCN(CC(=O)O)CC1)C(=O)Nc1ccc(-c2ccc(/N=N/c3ccc4c(S(=O)(=O)O)cc(S(=O)(=O)O)c(N)c4c3O)c(C)c2)cc1C] |
| 129 | 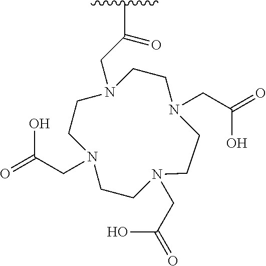 [SMILES: *C(=O)CN1CCN(CC(=O)O)CCN(CC(=O)O)CCN(CC(=O)O)CC1] |
| 130 | 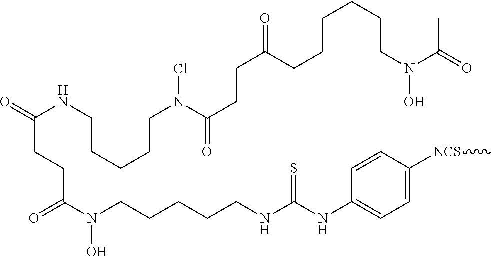 |
| 131 | 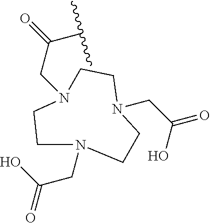 [SMILES: *C(=O)CN1CCN(CC(=O)O)CCN(CC(=O)O)CC1] |
| 132 | 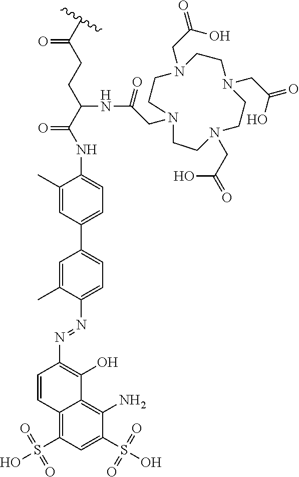 [SMILES: *C(=O)CCC(NC(=O)CN1CCN(CC(=O)O)CCN(CC(=O)O)CCN(CC(=O)O)CC1)C(=O)Nc1ccc(-c2ccc(/N=N/c3ccc4c(S(=O)(=O)O)cc(S(=O)(=O)O)c(N)c4c3O)c(C)c2)cc1C] |
| 133 | 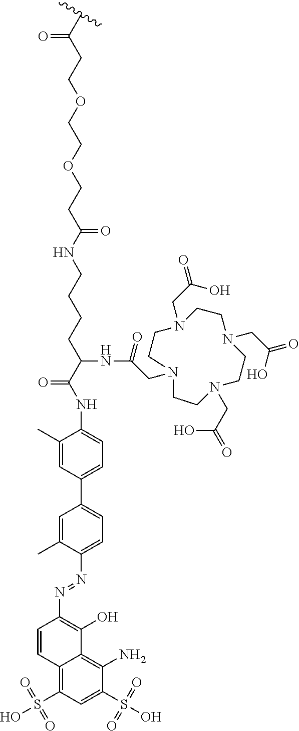 [SMILES: *C(=O)CCOCCOCCC(=O)NCCCCC(NC(=O)CN1CCN(CC(=O)O)CCN(CC(=O)O)CCN(CC(=O)O)CC1)C(=O)Nc1ccc(-c2ccc(/N=N/c3ccc4c(S(=O)(=O)O)cc(S(=O)(=O)O)c(N)c4c3O)c(C)c2)cc1C] |

### Example 134: General Preparation Method for a Radionuclide Marker

### (1) Wet Method

In this example, a general preparation method (wet method) for a radionuclide marker (with Ga-68 as an example) was elaborated with the compound of Formula (V-1) as an example. A hydrochloric acid solution of about 18.5-1,850 MBq of ⁶⁸GaCl₃ (rinsed with a germanium-gallium generator) was added to an acetic acid-acetate solution (1.0 g/L) containing 0.5 mL of the compound of Formula (V-1) prepared in Example 1 in a centrifuge tube, and a reaction was carried out at 37° C. for 20 min. A small C₁₈ separation column was taken, slowly rinsed with 10 mL of anhydrous ethanol first, and then rinsed with 10 mL of water. After being diluted with 10 ml of water, a labeled solution was loaded to the separation column, treated with 10 ml of water to remove unlabeled 68Ga ions first, and then rinsed with 0.3 mL of an ethanol solution of 10 mM HCl. A rinsed solution was collected, diluted with normal saline and subjected to sterile filtration to obtain an injection of a ⁶⁸Ga-labeled compound of Formula (V-1) (namely, ⁶⁸Ga-FAPI-RGD (V-1)).

### (2) Freeze-Drying Method

In this example, a general preparation method (freeze-drying method) for a radionuclide marker (with Ga-68 as an example) was elaborated with the compound of Formula (V-1) as an example. A hydrochloric acid solution of about 18.5-1,850 MBq ⁶⁸GaCl₃ (rinsed with a germanium-gallium generator) was added into a freeze-dried medical kit containing the compound of Formula (V-1), and mixed uniformly to carry out a reaction at 37° C. for 20 min. A small C₁₈ separation column was taken, slowly rinsed with 10 mL of anhydrous ethanol first, and then rinsed with 10 mL of water. After being diluted with 10 mL of water, a labeled solution was loaded to the separation column, treated with 10 mL of water to remove unlabeled ⁶⁸Ga ions first, and then rinsed with 0.3 mL of an ethanol solution of 10 mM HCl to obtain a rinsed solution of a complex. The rinsed solution was diluted with normal saline and subjected to sterile filtration to obtain an injection of a ⁶⁸Ga-labeled compound of Formula (V-1) (namely, ⁶⁸Ga-FAPI-RGD (V-1)).

Other compounds capable of being labeled with a radionuclide provided by the present invention are labeled by the above general labeling methods. For example, the compound of Formula (V-2), the compound of Formula (V-3), the compound of Formula (V-4), the compound of Formula (V-5), the compound of Formula (V-6), the compound of Formula (V-7), the compound of Formula (V-8), the compound of Formula (V-9), the compound of Formula (V-10), the compound of Formula (V-11), the compound of Formula (V-12), the compound of Formula (V-13), the compound of Formula (V-14), the compound of Formula (V-16), the compound of Formula (V-17), the compound of Formula (V-18), the compound of Formula (V-19), the compound of Formula (V-20), the compound of Formula (V-21), the compound of Formula (V-22), the compound of Formula (V-23), the compound of Formula (V-25), the compound of Formula (V-26), the compound of Formula (V-27), the compound of Formula (V-28), the compound of Formula (V-29), the compound of Formula (V-30), the compound of Formula (V-31), the compound of Formula (V-32), the compound of Formula (V-33), the compound of Formula (V-34), the compound of Formula (V-35), the compound of formula (V-36), the compound of formula (V-37), the compound of Formula (V-38), the compound of Formula (V-39), the compound of Formula (V-40), the compound of Formula (VI-1), the compound of Formula (VI-2), the compound of Formula (VI-3), the compound of Formula (VI-4), the compound of Formula (VI-5), the compound of Formula (VI-6), the compound of Formula (VI-7) and the compound of Formula (VI-8) are labeled with ⁶⁸Ga.

In addition, the compounds capable of being labeled with a radionuclide (such as the compound of Formula (V-1), the compound of Formula (V-2), the compound of Formula (V-3), the compound of Formula (V-4), the compound of Formula (V-5), the compound of Formula (V-6), the compound of Formula (V-7), the compound of Formula (V-8), the compound of Formula (V-9), the compound of Formula (V-10), the compound of Formula (V-11), the compound of Formula (V-12), the compound of Formula (V-13), the compound of Formula (V-14), the compound of Formula (V-16), the compound of Formula (V-17), the compound of Formula (V-18), the compound of Formula (V-19), the compound of Formula (V-20), the compound of Formula (V-21), the compound of Formula (V-22), the compound of Formula (V-23), the compound of Formula (V-25), the compound of Formula (V-26), the compound of Formula (V-27), the compound of Formula (V-28), the compound of Formula (V-29), the compound of Formula (V-30), the compound of Formula (V-31), the compound of Formula (V-32), the compound of Formula (V-33), the compound of Formula (V-34), the compound of Formula (V-35), the compound of formula (V-36), the compound of formula (V-37), the compound of Formula (V-38), the compound of Formula (V-39), the compound of Formula (V-40), the compound of Formula (VI-1), the compound of Formula (VI-2), the compound of Formula (VI-3), the compound of Formula (VI-4), the compound of Formula (VI-5), the compound of Formula (VI-6), the compound of Formula (VI-7) and the compound of Formula (VI-8), same below) provided by the present invention can also be labeled with reference to an 18-F labeling method provided by patents CN102123739B and/or CN102066974B. In addition, the compounds capable of being labeled with a radionuclide provided by the present invention can also be labeled with reference to other labeling methods provided in the prior art (including, but not limited to, the methods provided in the present invention). The radionuclide includes, but is not limited to: ¹⁸F, ⁵¹Cr, ⁶⁴Cu, ⁶⁷Cu, ⁶⁷Ga, ⁶⁸Ga, ⁸⁹Zr, ¹¹¹In, ^{99m}Tc, ¹⁸⁶Re, ¹⁸⁸Re, ¹³⁹La, ¹⁴⁰La, ¹⁷⁵Yb, ¹⁵³Sm, ¹⁶⁶Ho, ⁸⁶Y, ⁹⁰Y, ¹⁴⁹Pm, ¹⁶⁵Dy, ¹⁶⁹Er, ¹⁷⁷Lu, ⁴⁷Sc, ¹⁴²Pr, ¹⁵⁹Gd, ²¹²Bi, ²¹³Bi, ⁷²As, ⁷²Se, ⁹⁷Ru, ¹⁰⁹Pd, ¹⁰⁵Rh, ¹⁰¹mRh, ¹¹⁹Sb, ¹²⁸Ba, ¹²³I, ¹²⁴I, ¹³¹I, ¹⁹⁷Hg, ²¹¹At, ¹⁵¹Eu, ¹⁵³Eu, ¹⁶⁹Eu, ²⁰¹Tl, ²⁰³Pb, ²¹²Pb, ¹⁹⁸Au, ²²⁵Ac, ²²⁷Th or ¹⁹⁹Ag and the like.

### Experimental Example 135: Analysis and Application Effect of a Compound ⁶⁸Ga-FAPI-RGD (V-1)

(1) Analysis and Identification by HPLC An HPLC system was as follows: SHIMADZULC-20A; and a C18 chromatographic column (YMC, 3 μm, 4.6*150 mm) used for analysis. A detection wavelength was 254 nm, a flow rate was 1 mL/min, and a rinsing gradient was as follows: at 0-3 min, 10% of acetonitrile and 90% of water (50 mM ammonium acetate) were retained unchanged; at 3-16 min, the system was increased to include 90% of acetonitrile and 10% of water (50 mM ammonium acetate); at 16-18 min, 90% of acetonitrile and 10% of water (50 mM ammonium acetate) were retained; at 18-20 min, the system was reduced to include 10% of acetonitrile and 90% of water (50 mM ammonium acetate); and at 20-22 min, 10% of acetonitrile and 90% of water (50 mM ammonium acetate) were retained. HPLC quality control results of ⁶⁸Ga-FAPI-RGD (V-1) are shown in FIG. 46.

### (2) MicroPET Imaging in Tumor-Bearing Mice

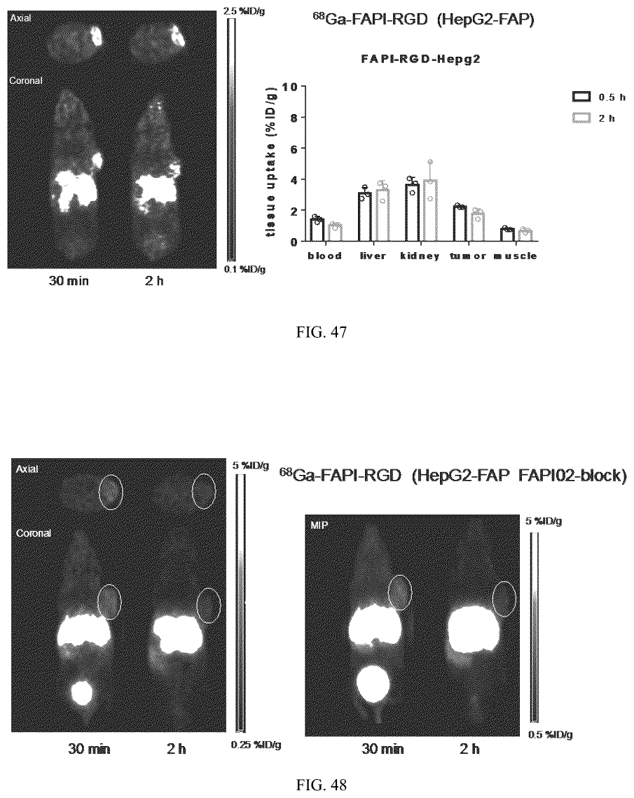

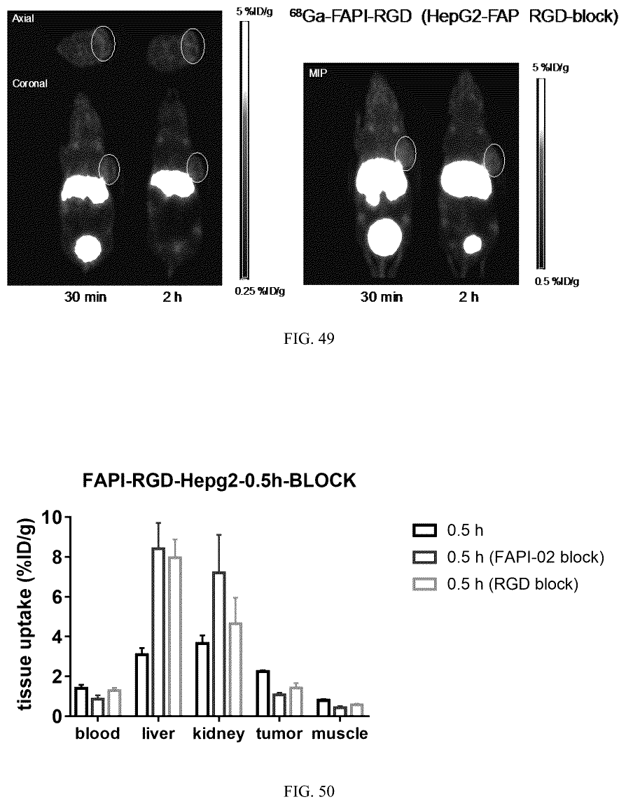

HepG2-FAP tumor-bearing mice were administered with 7.4 MBq of a compound ⁶⁸Ga-FAPI-RGD (V-1) by tail intravenous injection, and then subjected to MicroPET imaging under anesthetization with isoflurane after the administration for 0-120 min, respectively. Results are shown in FIG. 47. FIG. 47 shows a representative coronal MicroPET image of the HepG2-FAP tumor-bearing mice (n=3) at different time points after the intravenous injection of the ⁶⁸Ga-FAPI-RGD (V-1). At image acquisition time points (30 min and 2 h), tumors are clearly visible. A specific binding property of the ⁶⁸Ga-FAPI-RGD (V-1) to integrin and FAP in vivo is proven by a blocking experiment. In addition, the ⁶⁸Ga-FAPI-RGD (V-1) was co-injected into HepG2-FAP tumor-bearing mice with RGD or FAPI-02. MicroPET imaging results and organ uptake results are shown in FIG. 48, FIG. 49 and FIG. 50. As can be seen from FIG. 48 to FIG. 49, uptake of the ⁶⁸Ga-FAPI-RGD (V-1) in tumors can be reduced after co-injection with the RGD or the FAPI-02. As can be seen from FIG. 50, uptake values (% ID/g) of vital organs and tumors are obtained after the injection for 0.5 h, and uptake of the ⁶⁸Ga-FAPI-RGD (V-1) in tumors is partially inhibited by the RGD or the FAPI-02. The blocking experiment proves that the ⁶⁸Ga-FAPI-RGD (V-1) can achieve specific targeting of tumors in vivo by binding to integrin and FAP.

### Experimental Example 136: Analysis and Application Effect of a Compound ⁶⁸Ga-FAPI-RGD (V-25)

### (1) Stability Analysis

With reference the method in Example 134, a ⁶⁸Ga-labeled compound of Formula (V-25) (namely, a compound ⁶⁸Ga-FAPI-RGD (V-25)) was prepared. 20 μL of a solution of ⁶⁸Ga-FAPI-RGD (V-25) (with an activity of 3.7 MBq/20 μL) was sucked and added into a centrifuge tube containing 100 μL of normal saline or PBS (with a pH value of 7.4) for co-incubation at 37° C. for 0.5 h, 1 h and 4 h to obtain a co-incubation solution. 20 μL of the co-incubation solution was taken, filtered with a 0.22 μm needle type filter membrane, and analyzed by HPLC to obtain a radiochemical purity.

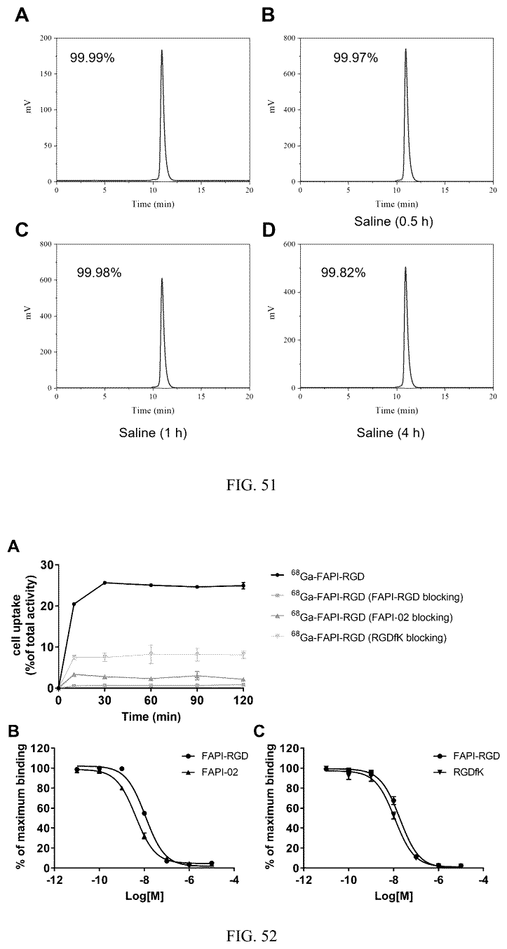

Test results are shown in FIG. 51. The results show that the compound ⁶⁸Ga-FAPI-RGD (V-25) is not obviously decomposed after incubation in the normal saline, and has a radiochemical purity of greater than 99%, indicating that the ⁶⁸Ga-FAPI-RGD (V-25) prepared by the present invention has excellent stability.

### (2) Uptake and Blocking

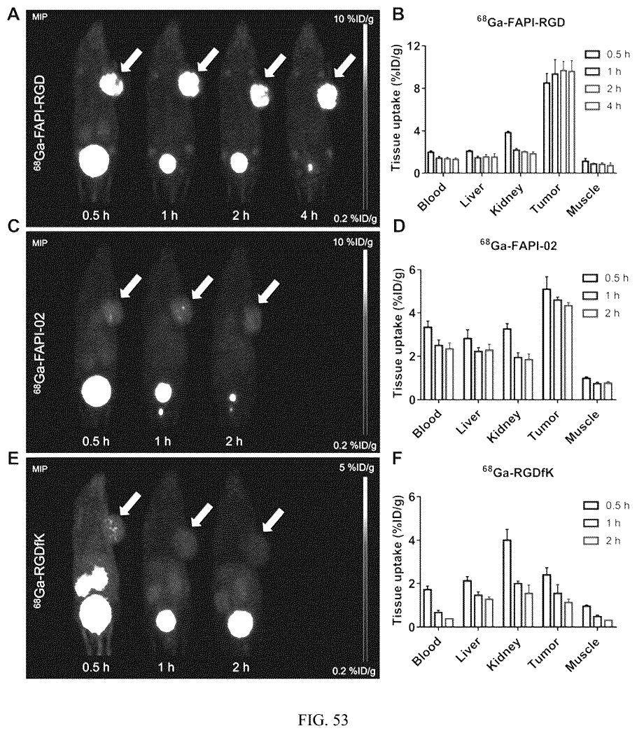

A cellular uptake experiment of the compound ⁶⁸Ga-FAPI-RGD (V-25) was carried out in HT1080-FAP tumor cells. Test results are shown in Part A of FIG. 52. The results show that the ⁶⁸Ga-FAPI-RGD (V-25) has rapid cellular uptake, has maximum uptake after incubation for 30 min, and is maintained at a similar uptake level for 2 h.

In addition, “FAPI-02” and “C(RGDfK)” as well as “FAPI-RGD” were also used in the experiment to carry out a blocking experiment. Test results are shown in Part A of FIG. 52. The results show that cellular uptake of the ⁶⁸Ga-FAPI-RGD (V-25) can be partially inhibited by the C(RGDfK) or the FAPI-02, and can be completely blocked by the FAPI-RGD (with reference to Part A of FIG. 52).

### (3) Affinity

A cell binding experiment was carried out in HT1080-FAP and U87MG tumor cells, and test results are shown in Part B and Part C of FIG. 52, respectively. In the HT1080-FAP cell experiment, the compound ⁶⁸Ga-FAPI-RGD (V-25) and ⁶⁸Ga-FAPI-02 were measured to have an IC₅₀ value of 11.17 nM and 4.14 nM, respectively. In the HT1080-FAP cell experiment, the compound ⁶⁸Ga-FAPI-RGD (V-25) and ⁶⁸Ga-C(RGDfK) were measured to have an IC₅₀ value of 18.93 nM and 11.49 nM, respectively. The experimental results show that compared with corresponding monomers, the compound ⁶⁸Ga-FAPI-RGD (V-25) has similar affinity to corresponding receptors, such as FAP and integrin αᵥβ₃.

### (4) MicroPET Imaging in Tumor-Bearing Mice

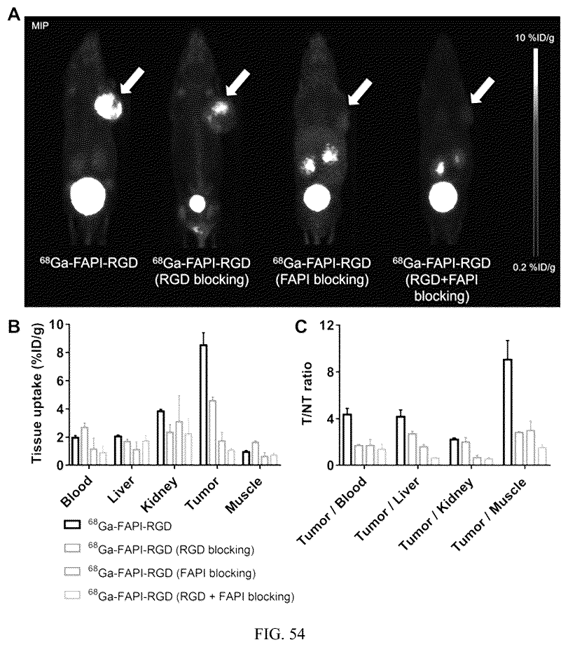

HT1080-FAP tumor-bearing mice, which were randomly grouped, were administered with 7.4 MBq of the compound ⁶⁸Ga-FAPI-RGD (V-25), ⁶⁸Ga-FAPI-02 and ⁶⁸Ga-C(RGDfK) by tail intravenous injection, respectively. Then, under anaesthesia with isoflurane, the ⁶⁸Ga-FAPI-RGD (V-25) group was subjected to MicroPET imaging after administration for 0-240 min, respectively, and the other groups were subjected to MicroPET imaging after administration for 0-120 min, respectively. Results are shown in FIG. 53. In FIG. 53, Part A, Part C and Part E show maximum-density MicroPET projection images of the HT1080-FAP tumor-bearing mice (n=3) at different time points after the intravenous injection of the three groups of mice, respectively, and Part B, Part D and Part F show uptake in various organs or tissues (such as blood, liver, kidney, tumors and muscles) of the three groups of mice at different time points after the injection, respectively, where three uptake amounts in each group correspond to, from left to right, 0.5 h, 1 h and 2 h after the injection, respectively. FIG. 53 shows that tumors are clearly visible at image acquisition time points, and the tumor uptake of ⁶⁸Ga-FAPI-RGD (V-25) is higher than that of ⁶⁸Ga-FAPI-02 and ⁶⁸Ga-C(RGDfK). A specific binding property of the ⁶⁸Ga-FAPI-RGD (V-25) to integrin αᵥβ₃ and FAP in vivo is proven by a blocking experiment. The ⁶⁸Ga-FAPI-RGD (V-25) and C(RGDfK) or FAPI-02 were co-injected into HT1080-FAP tumor-bearing mice, and MicroPET imaging results and organ uptake results are shown in FIG. 54. In FIG. 54, four images in Part A correspond to, from left to right, images obtained by separate injection of the ⁶⁸Ga-FAPI-RGD (V-25), co-injection of the ⁶⁸Ga-FAPI-RGD (V-25) with the C(RGDfK), co-injection of the ⁶⁸Ga-FAPI-RGD (V-25) with the FAPI-02, and co-injection of the ⁶⁸Ga-FAPI-RGD (V-25) with the C(RGDfK) and the FAPI-02, respectively; and Part B and Part C show uptake of the ⁶⁸Ga-FAPI-RGD (V-25) in various organs or tissues (such as blood, liver, kidney, tumors and muscles) of the mice after injection by the four different injection methods and a target/non-target ratio, respectively, where four histograms for each organ or tissue in Part B and Part C correspond to, from left to right, the four injection methods in Part A, respectively. As can be seen from FIG. 54, the uptake of ⁶⁸Ga-FAPI-RGD (V-25) in tumors can be reduced by co-injection of the ⁶⁸Ga-FAPI-RGD (V-25) with the RGD or the FAPI-02, and the uptake of ⁶⁸Ga-FAPI-RGD (V-25) in tumors can be further reduced by co-injection of the ⁶⁸Ga-FAPI-RGD (V-25) with the RGD and the FAPI-02. A blocking experiment proves that the ⁶⁸Ga-FAPI-RGD (V-25) can achieve specific targeting of tumors by binding to integrin and FAP in vivo.

### (5) PET/CT Imaging in Tumor Patients

In a clinical trial in the real world, a patient with pancreatic cancer (one), a patient with non small cell lung cancer (one), a patient with small cell lung cancer (one) and a patient with nasopharyngeal carcinoma (one) were used as subjects. An intravenous injection dose (1.8-2.2 MBq [0.05-0.06 mCi]/kg) of the ⁶⁸Ga-FAPI-RGD (V-25) was calculated based on a body weight of each subject. After intravenous injection was performed for 3 h, data was obtained by a hybrid PET/CT scanner (Discovery MI, GE Health care, Milwaukee, WI, USA). Imaging results are shown in FIG. 55. A maximum standard uptake value (SUVₘₐₓ) was automatically calculated by using a region of interest (ROI) drawn on a transaxial image. The SUVₘₐₓ value of the ⁶⁸Ga-FAPI-RGD (V-25) targeting two targets is higher than that of ⁶⁸Ga-FAPI-46 targeting the single target, FAP, in different types of tumors, and the SUVₘₐₓ value is increased by about 30-50%, proving that the design of targeting two targets can improve the number and utilization efficiency of effective receptors in tumors, thereby improving the uptake in tumors.

### Example 137: Analysis and Application Effect of a ¹⁷⁷Lu-Radiolabeled Compound V-40 (Namely, a Compound ¹⁷⁷Lu-FAPI-RGD (V-40))

The compound V-40 prepared in Example 8 was radiolabeled with ¹⁷⁷Lu by a conventional technical means in the art to obtain a compound ¹⁷⁷Lu-FAPI-RGD (V-40). A SPECT imaging experiment was carried out to observe the distribution of a tracer in tumor mice. 37 MBq of the compound ¹⁷⁷Lu-FAPI-RGD (V-40) was injected into U87MG tumor mice. After the injection for 1 h, 4 h, 12 h, 24 h, 48 h, 72 h and 96 h, the U87MG tumor mice were anesthetized and placed on a SPECT scanner for static SPECT scanning. Results are as shown in FIG. 60. The compound ¹⁷⁷Lu-FAPI-RGD (V-40) has obvious uptake in tumors after the injection in the U87MG tumor mice for 1 h, which is significantly higher than the uptake in all organs except for the bladder. With prolongation of the injection time, the uptake in tumors is increased and is remained high after lasting for 96 h, while the uptake in the bladder and other organs is gradually decreased, proving that the probe has excellent uptake and retention in tumors and has great potential for treatment of glioma tumors.

### Example 138: Other Examples

In the present invention, the stability analysis of a compound ⁶⁸Ga-FAPI-RGD (V-2) (namely, a ⁶⁸Ga-labeled complex of the compound of Formula (2), the same below), a compound ⁶⁸Ga-FAPI-RGD (V-3), a compound ⁶⁸Ga-FAPI-RGD (V-4), a compound ⁶⁸Ga-FAPI-RGD (V-5), a compound ⁶⁸Ga-FAPI-RGD (V-6), a compound ⁶⁸Ga-FAPI-RGD (V-7), a compound ⁶⁸Ga-FAPI-RGD (V-8), a compound ⁶⁸Ga-FAPI-RGD (V-9), a compound ⁶⁸Ga-FAPI-RGD (V-10), a compound ⁶⁸Ga-FAPI-RGD (V-11), a compound ⁶⁸Ga-FAPI-RGD (V-12), a compound ⁶⁸Ga-FAPI-RGD (V-13), a compound ⁶⁸Ga-FAPI-RGD (V-14), a compound ⁶⁸Ga-FAPI-RGD (V-16), a compound ⁶⁸Ga-FAPI-RGD (V-17), a compound ⁶⁸Ga-FAPI-RGD (V-18), a compound ⁶⁸Ga-FAPI-RGD (V-19), a compound ⁶⁸Ga-FAPI-RGD (V-20), a compound ⁶⁸Ga-FAPI-RGD (V-21), a compound ⁶⁸Ga-FAPI-RGD (V-22), a compound ⁶⁸Ga-FAPI-RGD (V-23), a compound ⁶⁸Ga-FAPI-RGD (V-26), a compound ⁶⁸Ga-FAPI-RGD (V-27), a compound ⁶⁸Ga-FAPI-RGD (V-28), a compound ⁶⁸Ga-FAPI-RGD (V-29), a compound ⁶⁸Ga-FAPI-RGD (V-30), a compound ⁶⁸Ga-FAPI-RGD (V-31), a compound ⁶⁸Ga-FAPI-RGD (V-32), a compound ⁶⁸Ga-FAPI-RGD (V-33), a compound ⁶⁸Ga-FAPI-RGD (V-34), a compound ⁶⁸Ga-FAPI-RGD (V-35), a compound ⁶⁸Ga-FAPI-RGD (V-36), a compound ⁶⁸Ga-FAPI-RGD (V-37), a compound ⁶⁸Ga-FAPI-RGD (V-38), a compound ⁶⁸Ga-FAPI-RGD (V-39), a compound ⁶⁸Ga-FAPI-RGD (VI-1), a compound ⁶⁸Ga-FAPI-RGD (VI-2), a compound ⁶⁸Ga-FAPI-RGD (VI-3), a compound ⁶⁸Ga-FAPI-RGD (VI-4), a compound ⁶⁸Ga-FAPI-RGD (VI-5), a compound ⁶⁸Ga-FAPI-RGD (VI-6), a compound ⁶⁸Ga-FAPI-RGD (VI-7) and a compound ⁶⁸Ga-FAPI-RGD (VI-8) (collectively referred to as FAPI-RGD radiolabeled compounds in the present invention) was also verified by using a relevant analysis method with reference to the experiment (1) in Example 136, respectively. Results show that all the radiolabeled compounds of the dual-targeting compound for fibroblast activation protein (FAP) and integrin αᵥβ₃ provided by the present invention can achieve good stability.

In the present invention, uptake and blocking experiments and an affinity experiment of the FAPI-RGD radiolabeled compounds were also further verified by using relevant methods with reference to the experiments (2) and (3) in Example 136. Experimental results show that the FAPI-RGD radiolabeled compounds provided by the present invention can exhibit rapid cellular uptake in corresponding cell models, and the corresponding cellular uptake can be blocked by corresponding monomers/dimeric compounds. In addition, the FAPI-RGD radiolabeled compounds provided by the present invention can also exhibit similar affinity to corresponding receptors, such as FAP and integrins αᵥβ₃.

In the present invention, MicroPET imaging of the FAPI-RGD radiolabeled compounds in tumor-bearing mice was furthermore verified by using a relevant method with reference to the experiment (4) in Example 136, where an experimental group was subjected to MicroPET imaging at 0-240 min after administration, respectively. Results show maximum-density MicroPET projection images of animal models at different time points after intravenous injection, tumors are clearly visible at image acquisition time points, and the tumor uptake of the experimental group (namely, the FAPI-RGD radiolabeled compounds provided by the present invention) is higher than that of corresponding monomers.

In summary, the FAPI-RGD dual-targeting structure provided by the present invention has high affinity to both an FAP target and an integrin αᵥβ₃ target, can synergistically target the FAP target and the integrin αᵥβ₃ target in tumors, has excellent pharmacokinetics, high uptake in tumors and a long retention time in tumors, and is expected to be used for diagnosis or treatment of diseases characterized by overexpression of fibroblast activation protein (FAP) and/or integrin αᵥβ₃.

Although the present invention is described in detail by general description, specific embodiments and tests above, it is obvious to persons skilled in the art that some modifications or improvements can be made on the basis of the present invention. Therefore, the modifications or improvements made without deviating from the spirit of the present invention fall within the scope of protection required by the present invention.

1. A dual-targeting compound, wherein the dual-targeting compound comprises a specific binding ligand structure of fibroblast activation protein (FAP) and integrin αᵥβ₃, and the dual-targeting compound has a structural formula shown in Formula (I) or Formula (II): 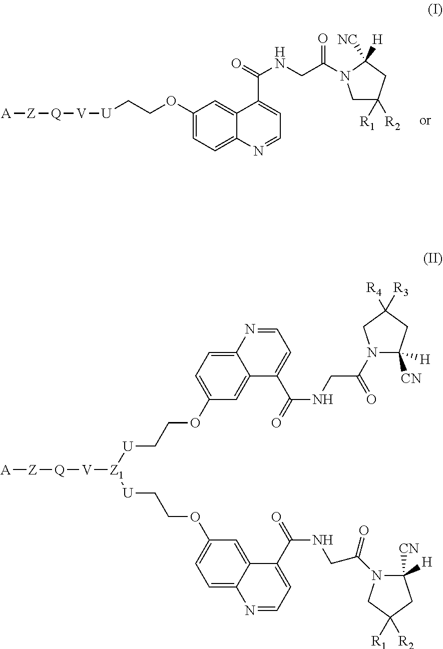
wherein
R₁, R₂, R₃ and R₄ may be independently selected from H or F, and the R₁, the R₂, the R₃ and the R₄ may be the same or different;
Z, Q, V and U are a same connection structure or different connection structures and are independently selected from —NH—, 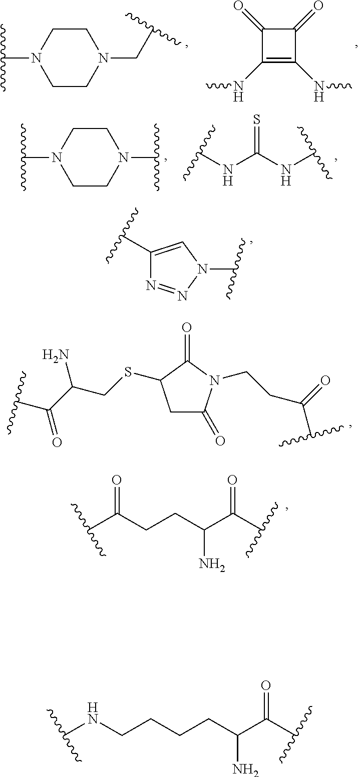
or substitution structures based on —(CH₂)ₙ—, respectively;
Z₁ is 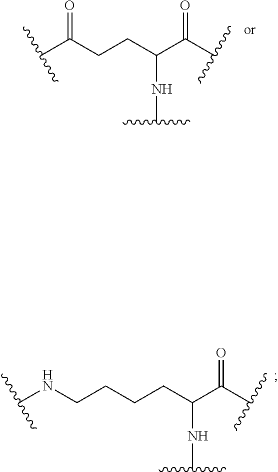 [SMILES: *NC(CCC(*)=O)C(*)=O ; *NCCCCC(N*)C(*)=O]
when the Z, the Q, the V and the U are substitution structures based on —(CH₂)ₙ—, n is an integer selected from 0 to 30, each —CH₂— is separately substituted with or without —O—, —NH—, —(CO)—, —NH—(CO)—, —CH(NH₂)— or —(CO)—NH—, and substitution occurs when no two adjacent —CH₂— groups are substituted;
A is a ligand structure specifically bound to integrin αᵥβ₃, and the structure is shown in Formula (III) or Formula (IV): 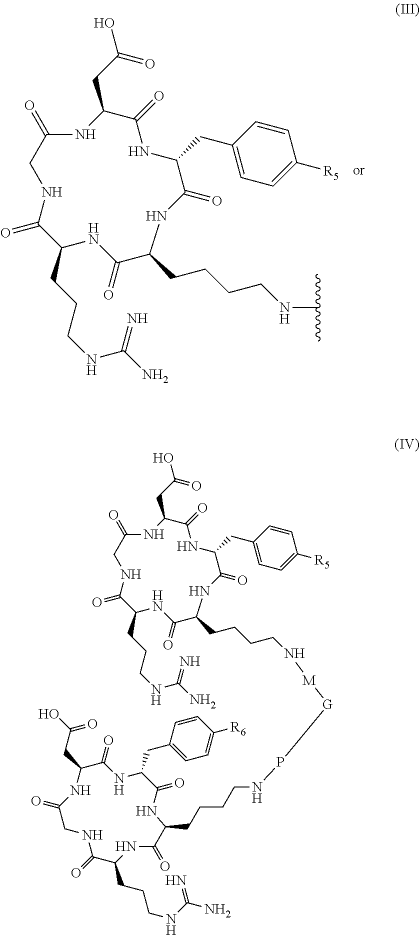
in Formula (III), R₅ is selected from H or OH;
in Formula (IV), R₅ and R₆ are the same or different, and are independently selected from H or OH; when M and P are substitution structures based on —(CH₂)ₙ—, n is an integer selected from 0 to 30, each —CH₂— is separately substituted with or without —O—, —NH—, —(CO)—, —NH—(CO)—, —CH(NH₂)— or —(CO)—NH—, and substitution occurs when no two adjacent —CH₂— groups are substituted; and G is selected form

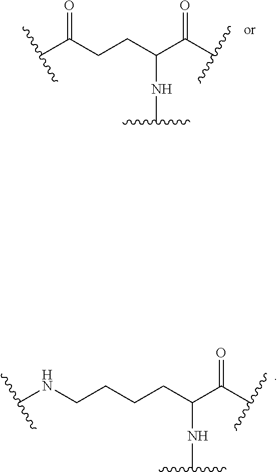 [SMILES: *NC(CCC(*)=O)C(*)=O ; *NCCCCC(N*)C(*)=O]

2. The dual-targeting compound according to claim 1, wherein the Z in Formula (I) or Formula (II) is —NH—CH₂—(CH₂—O—CH₂)₂—CH₂—(CO)—, —NH—CH₂—(CH₂—O—CH₂)₃—CH₂—(CO)—, —NH—CH₂—(CH₂—O—CH₂)₄—CH₂—(CO)—, 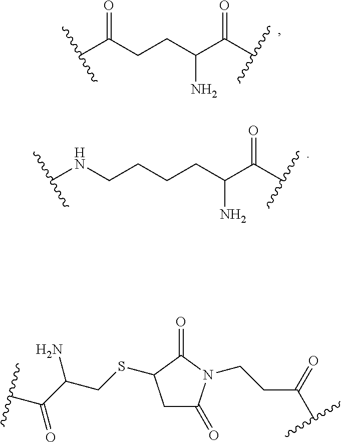 [SMILES: *C(=O)CCC(N)C(*)=O ; *NCCCCC(N)C(*)=O ; *C(=O)CCN1C(=O)CC(SCC(N)C(*)=O)C1=O]
or —(CH₂)₀—.

3. The dual-targeting compound according to claim 1, wherein the Q in Formula (I) or Formula (II) is

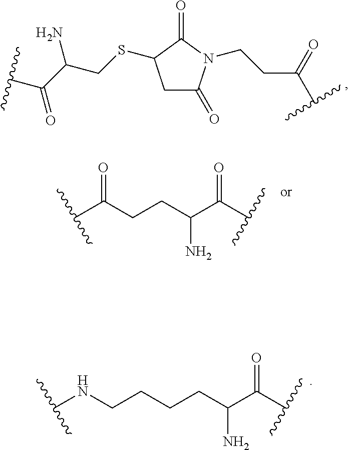 [SMILES: *C(=O)CCN1C(=O)CC(SCC(N)C(*)=O)C1=O ; *C(=O)CCC(N)C(*)=O ; *NCCCCC(N)C(*)=O]

4. The dual-targeting compound according to claim 1, wherein the V in Formula (I) or Formula (II) is —NH—CH₂—(CH₂—O—CH₂)₂—CH₂—(CO)—, —NH—CH₂—(CH₂—O—CH₂)₃—CH₂—(CO)—, —NH—CH₂—(CH₂—O—CH₂)₄—CH₂—(CO)— or —(CH₂)₀—.

5. The dual-targeting compound according to claim 1, wherein the U in Formula (I) or Formula (II) is —NH— or

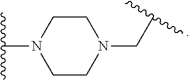 [SMILES: *CN1CCN(*)CC1]

6. The dual-targeting compound according to claim 1, wherein the Z₁ in Formula (II) is

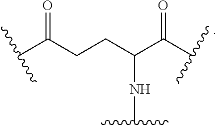 [SMILES: *NC(CCC(*)=O)C(*)=O]

7. A dual-targeting compound capable of being labeled with a radionuclide, wherein the dual-targeting compound capable of being labeled with a radionuclide is formed by connecting amino in any one of the Z, Q or V structures of Formula (I) or Formula (II) according to claim 1 with a nuclide chelating group, and has a general formula shown in Formula V or Formula VI below: 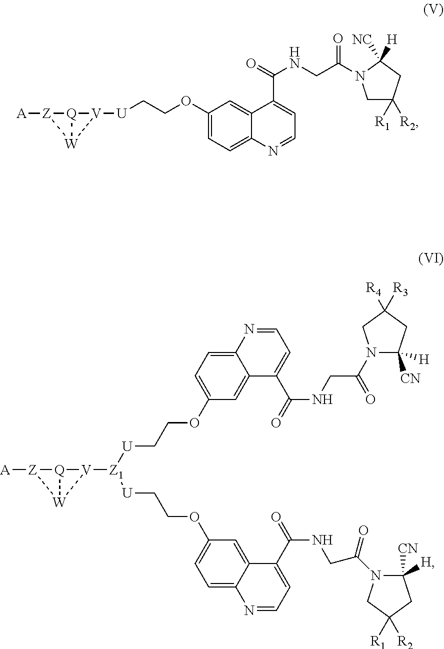
wherein W is a fragment with a nuclide chelating group, and is derived from any one of 1,4,7,10-tetraazacyclododecane-N,N′,N,N′-tetraacetic acid (DOTA), ethylenediamine tetraacetic acid (EDTA), 1,4,7-triazacyclononane-1,4,7-triacetic acid (NOTA), triethylenetetraamine (TETA), iminodiacetic acid, diethylenetriamine-N,N,N′,N′,N″-pentaacetic acid (DTPA), bis(carboxymethyl)imidazole glycinate or 6-hydrazinopyridine-3-carboxylic acid (HYNIC); or the W has any one of the following structures: 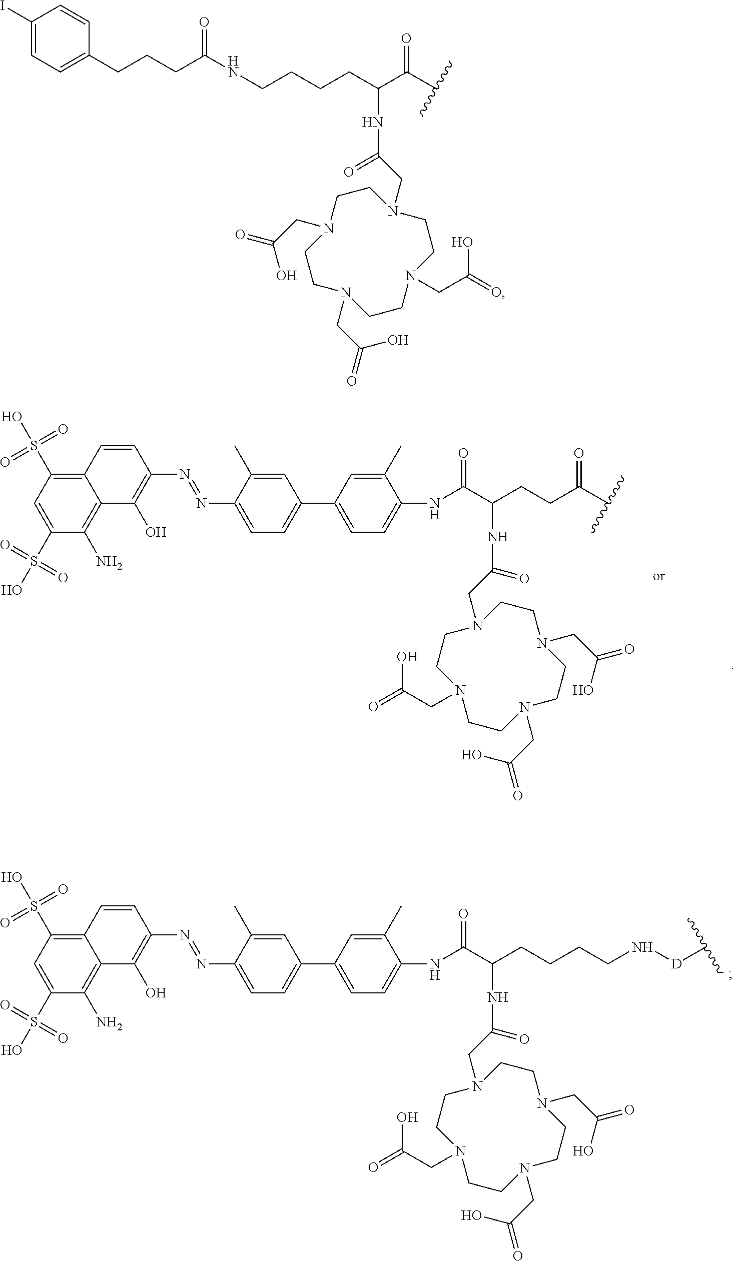
and D in the structures is a substitution structure based on —(CH₂)ₚ—, p is an integer selected from 0 to 30, each —CH₂— is separately substituted with or without —O—, —NH—, —(CO)—, —NH—(CO)—, —CH(NH₂)— or —(CO)—NH—, and substitution occurs when no two adjacent —CH₂— groups are substituted.

8. The dual-targeting compound according to claim 7, wherein the compound of Formula (V) has any one of the following structures as shown in Formula (V-1) to Formula (V-40):

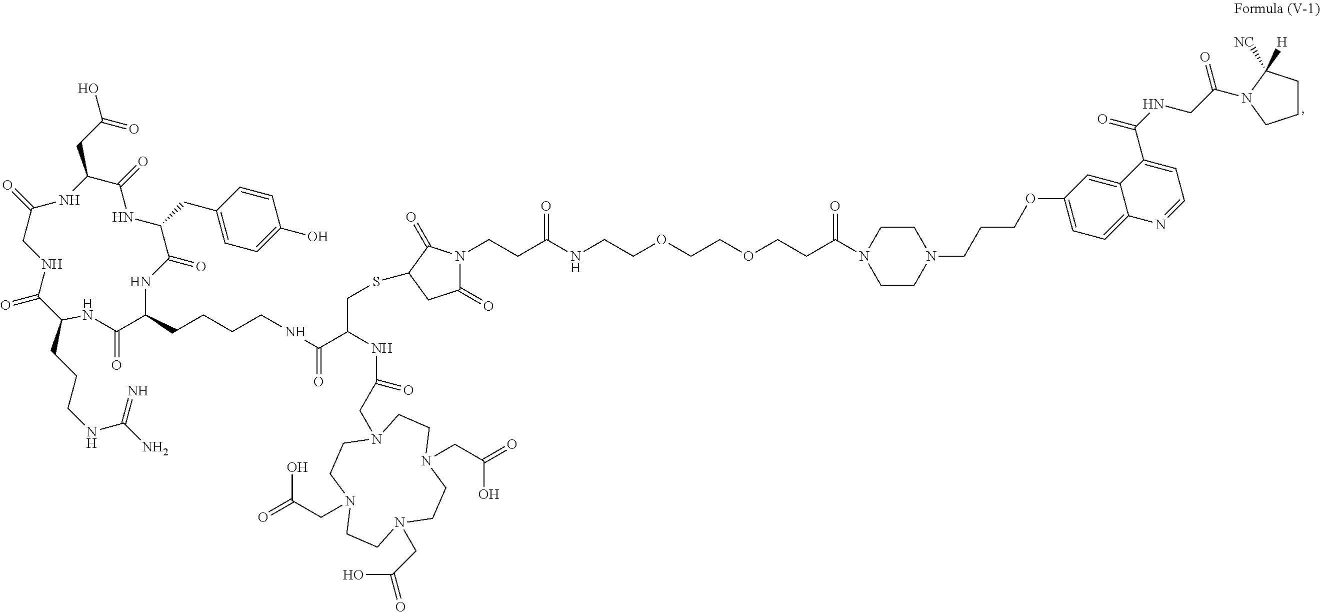 [SMILES: N#C[C@@H]1CCCN1C(=O)CNC(=O)c1ccnc2ccc(OCCCN3CCN(C(=O)CCOCCOCCNC(=O)CCN4C(=O)CC(SCC(NC(=O)CN5CCN(CC(=O)O)CCN(CC(=O)O)CCN(CC(=O)O)CC5)C(=O)NCCCC[C@@H]5NC(=O)[C@@H](Cc6ccc(O)cc6)NC(=O)[C@H](CC(=O)O)NC(=O)CNC(=O)[C@H](CCCNC(=N)N)NC5=O)C4=O)CC3)cc12]

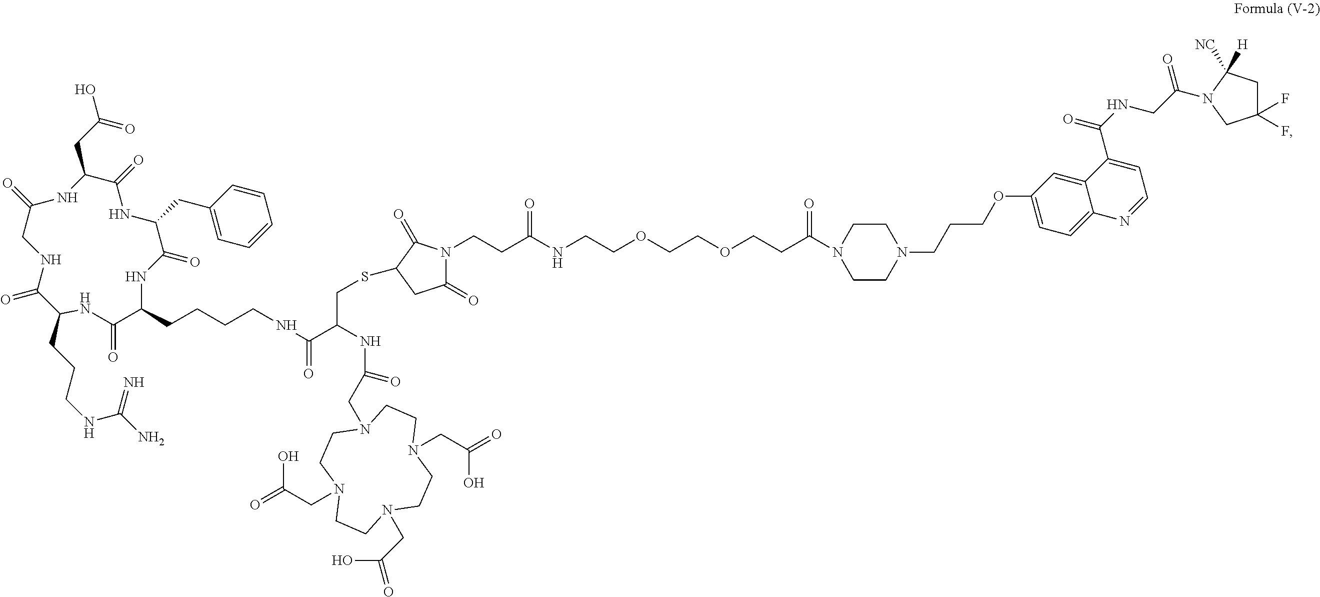 [SMILES: N#C[C@@H]1CC(F)(F)CN1C(=O)CNC(=O)c1ccnc2ccc(OCCCN3CCN(C(=O)CCOCCOCCNC(=O)CCN4C(=O)CC(SCC(NC(=O)CN5CCN(CC(=O)O)CCN(CC(=O)O)CCN(CC(=O)O)CC5)C(=O)NCCCC[C@@H]5NC(=O)[C@@H](Cc6ccccc6)NC(=O)[C@H](CC(=O)O)NC(=O)CNC(=O)[C@H](CCCNC(=N)N)NC5=O)C4=O)CC3)cc12]

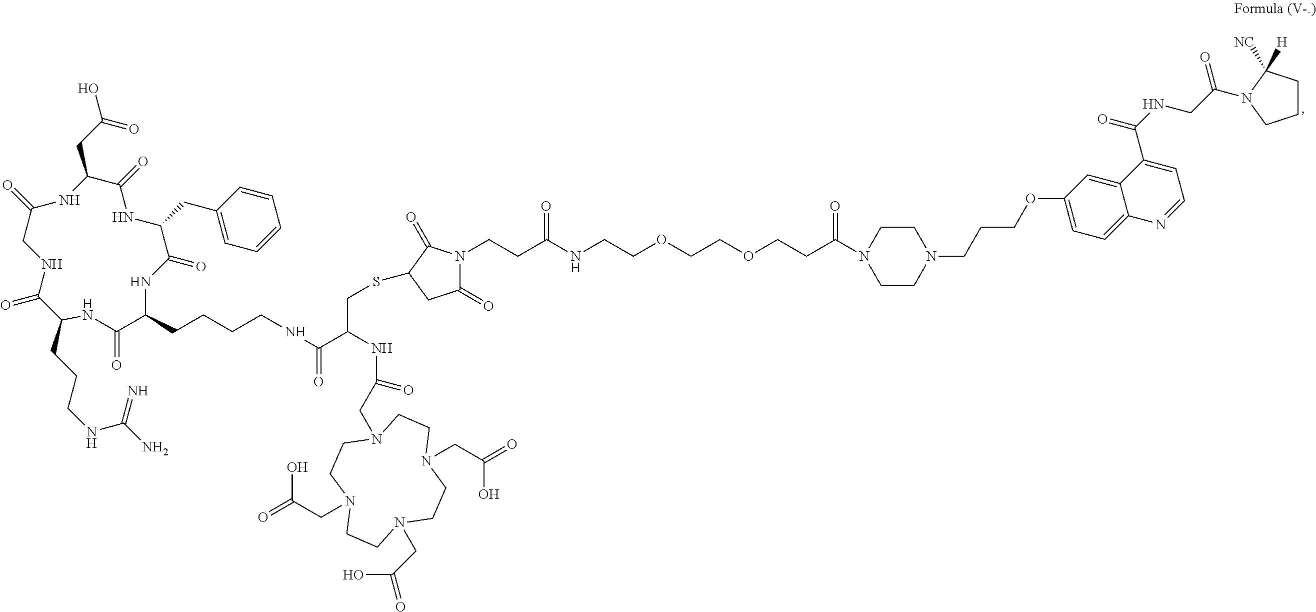 [SMILES: N#C[C@@H]1CCCN1C(=O)CNC(=O)c1ccnc2ccc(OCCCN3CCN(C(=O)CCOCCOCCNC(=O)CCN4C(=O)CC(SCC(NC(=O)CN5CCN(CC(=O)O)CCN(CC(=O)O)CCN(CC(=O)O)CC5)C(=O)NCCCC[C@@H]5NC(=O)[C@@H](Cc6ccccc6)NC(=O)[C@H](CC(=O)O)NC(=O)CNC(=O)[C@H](CCCNC(=N)N)NC5=O)C4=O)CC3)cc12]

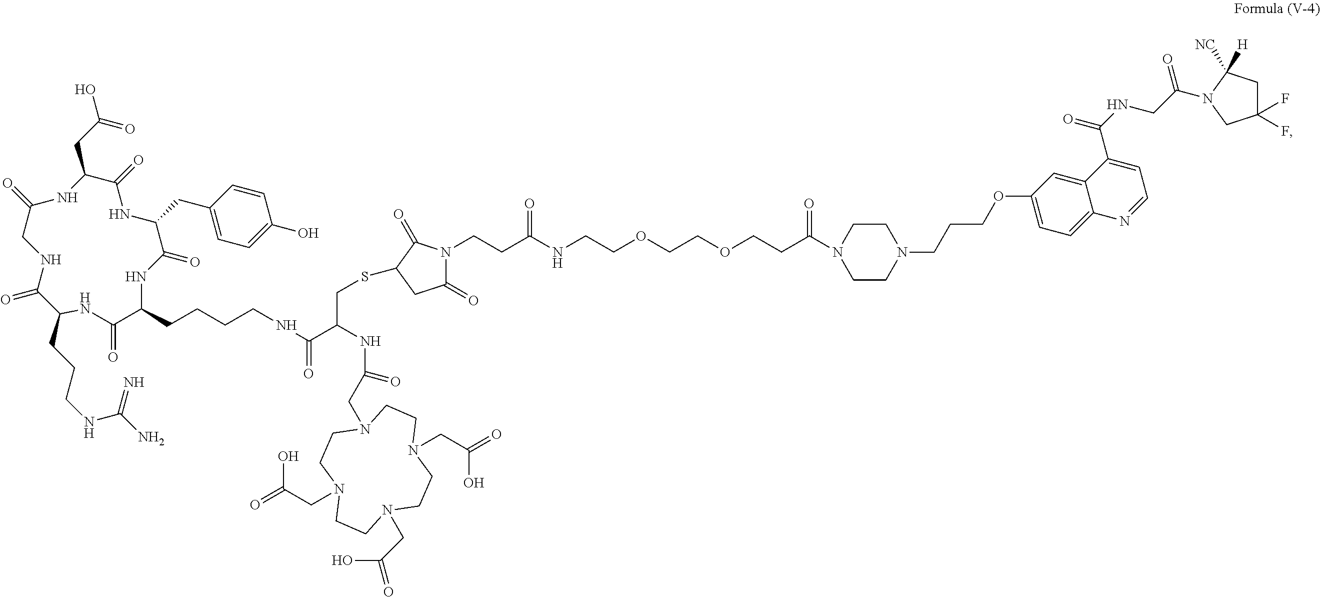 [SMILES: N#C[C@@H]1CC(F)(F)CN1C(=O)CNC(=O)c1ccnc2ccc(OCCCN3CCN(C(=O)CCOCCOCCNC(=O)CCN4C(=O)CC(SCC(NC(=O)CN5CCN(CC(=O)O)CCN(CC(=O)O)CCN(CC(=O)O)CC5)C(=O)NCCCC[C@@H]5NC(=O)[C@@H](Cc6ccc(O)cc6)NC(=O)[C@H](CC(=O)O)NC(=O)CNC(=O)[C@H](CCCNC(=N)N)NC5=O)C4=O)CC3)cc12]

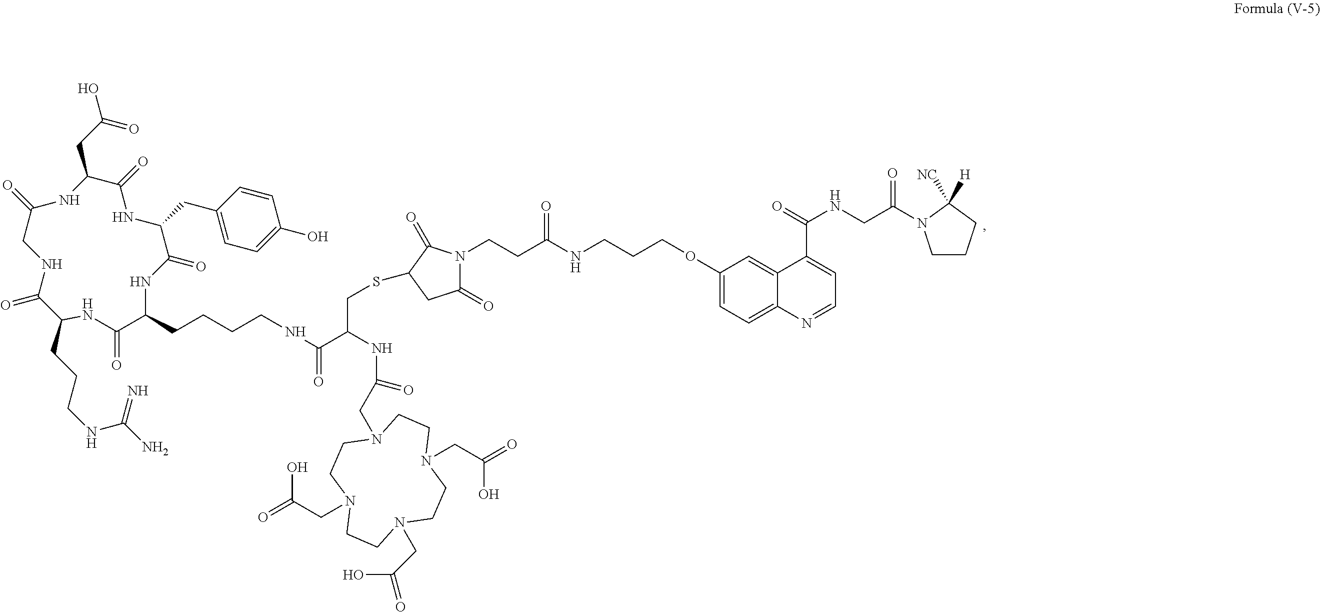 [SMILES: N#C[C@@H]1CCCN1C(=O)CNC(=O)c1ccnc2ccc(OCCCNC(=O)CCN3C(=O)CC(SCC(NC(=O)CN4CCN(CC(=O)O)CCN(CC(=O)O)CCN(CC(=O)O)CC4)C(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)C3=O)cc12]

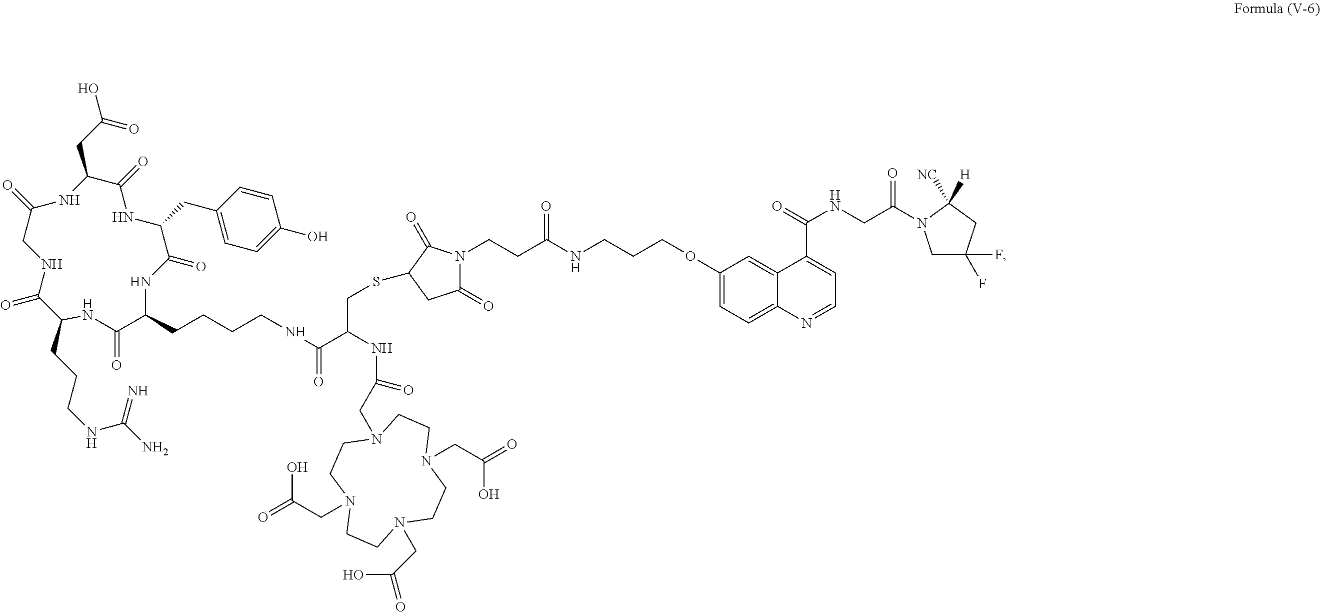 [SMILES: N#C[C@@H]1CC(F)(F)CN1C(=O)CNC(=O)c1ccnc2ccc(OCCCNC(=O)CCN3C(=O)CC(SCC(NC(=O)CN4CCN(CC(=O)O)CCN(CC(=O)O)CCN(CC(=O)O)CC4)C(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)C3=O)cc12]

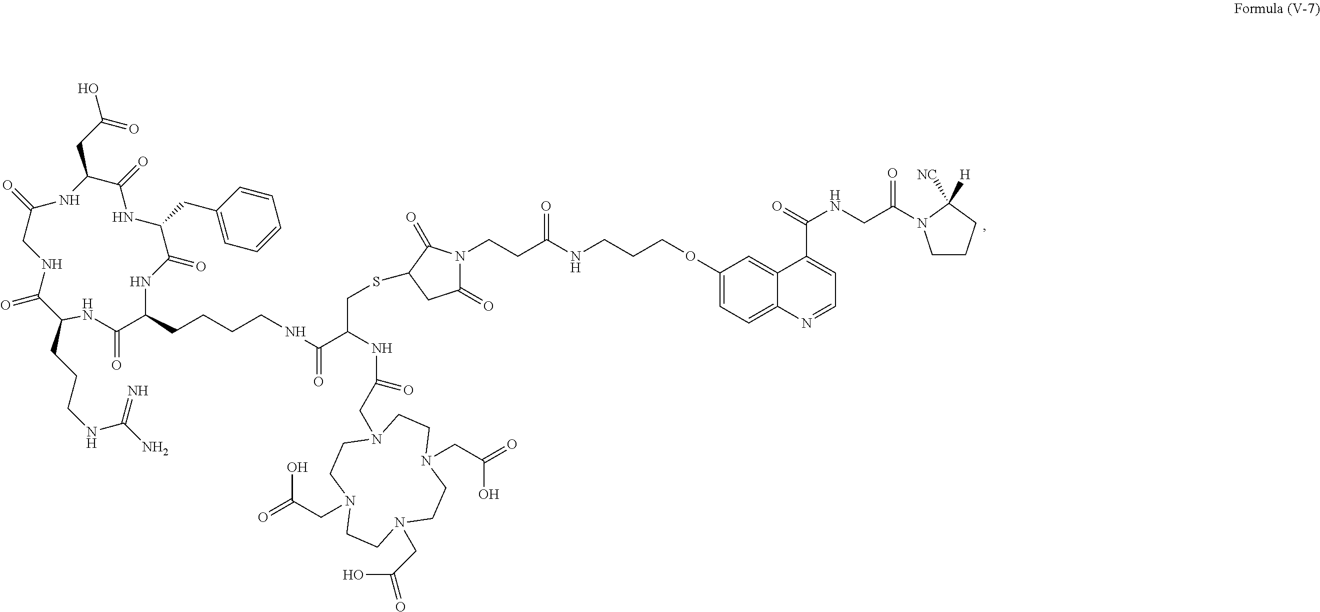 [SMILES: N#C[C@@H]1CCCN1C(=O)CNC(=O)c1ccnc2ccc(OCCCNC(=O)CCN3C(=O)CC(SCC(NC(=O)CN4CCN(CC(=O)O)CCN(CC(=O)O)CCN(CC(=O)O)CC4)C(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)C3=O)cc12]

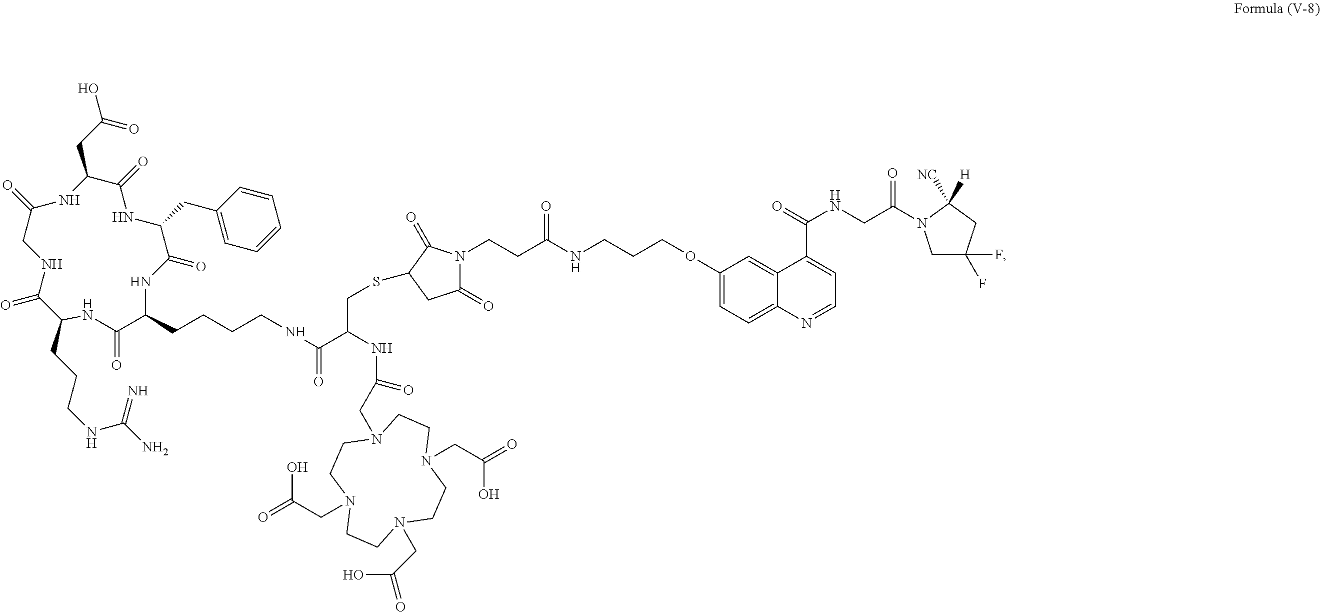 [SMILES: N#C[C@@H]1CC(F)(F)CN1C(=O)CNC(=O)c1ccnc2ccc(OCCCNC(=O)CCN3C(=O)CC(SCC(NC(=O)CN4CCN(CC(=O)O)CCN(CC(=O)O)CCN(CC(=O)O)CC4)C(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)C3=O)cc12]

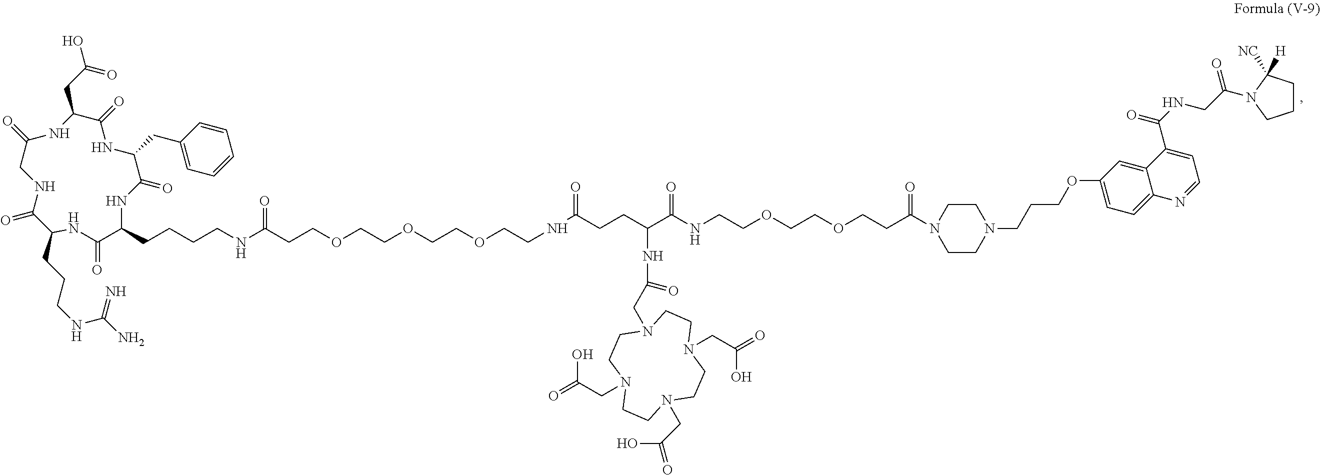 [SMILES: N#C[C@@H]1CCCN1C(=O)CNC(=O)c1ccnc2ccc(OCCCN3CCN(C(=O)CCOCCOCCNC(=O)C(CCC(=O)NCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

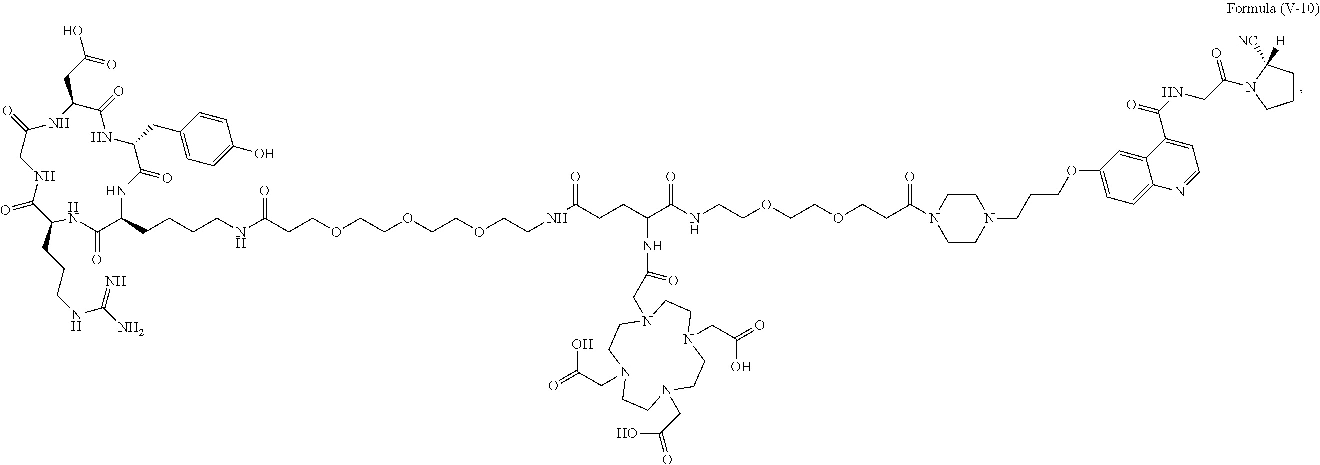 [SMILES: N#C[C@@H]1CCCN1C(=O)CNC(=O)c1ccnc2ccc(OCCCN3CCN(C(=O)CCOCCOCCNC(=O)C(CCC(=O)NCCOCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

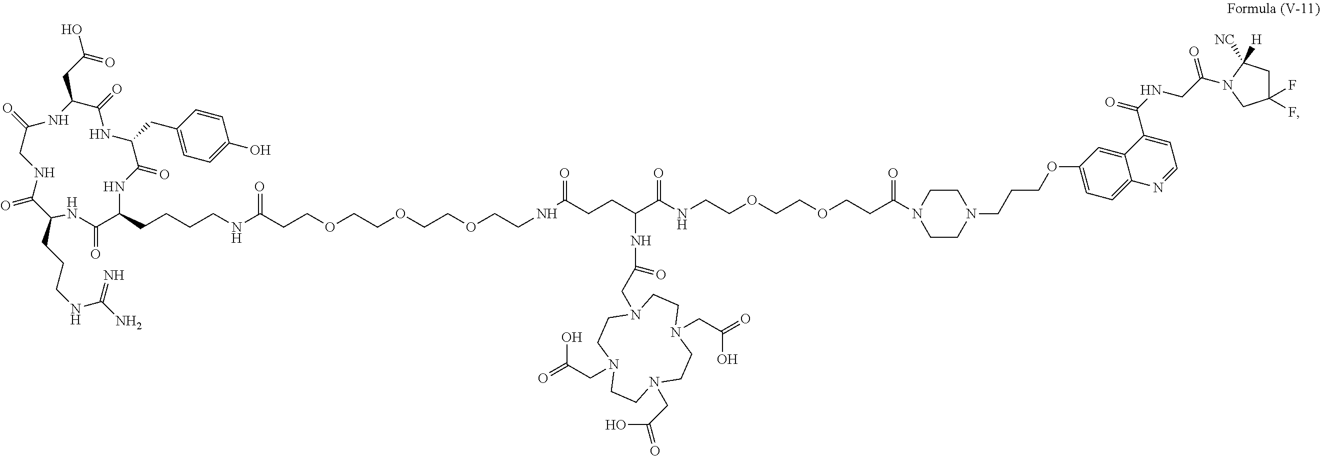 [SMILES: N#C[C@@H]1CC(F)(F)CN1C(=O)CNC(=O)c1ccnc2ccc(OCCCN3CCN(C(=O)CCOCCOCCNC(=O)C(CCC(=O)NCCOCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

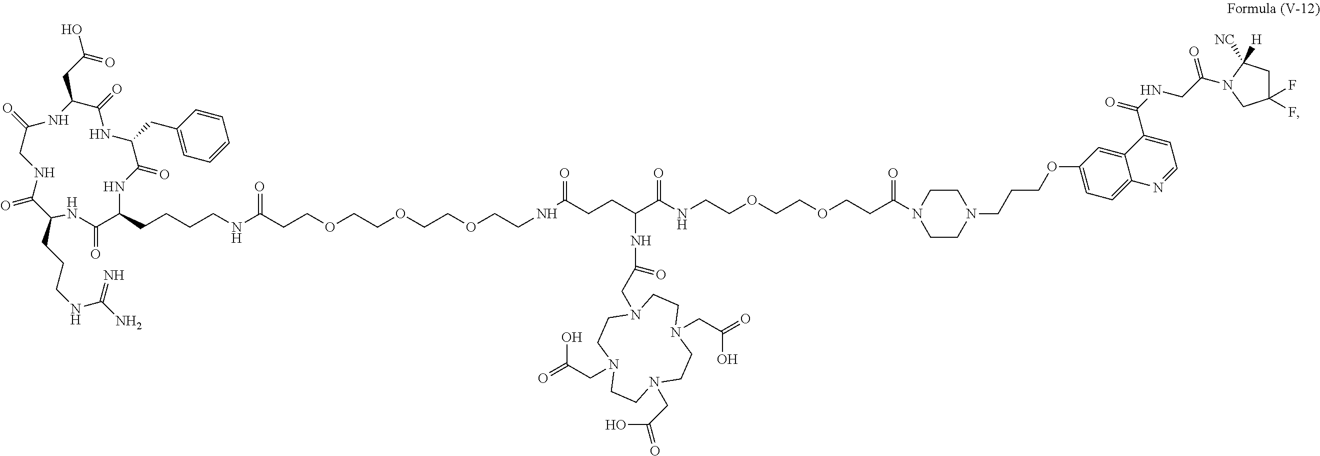 [SMILES: N#C[C@@H]1CC(F)(F)CN1C(=O)CNC(=O)c1ccnc2ccc(OCCCN3CCN(C(=O)CCOCCOCCNC(=O)C(CCC(=O)NCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

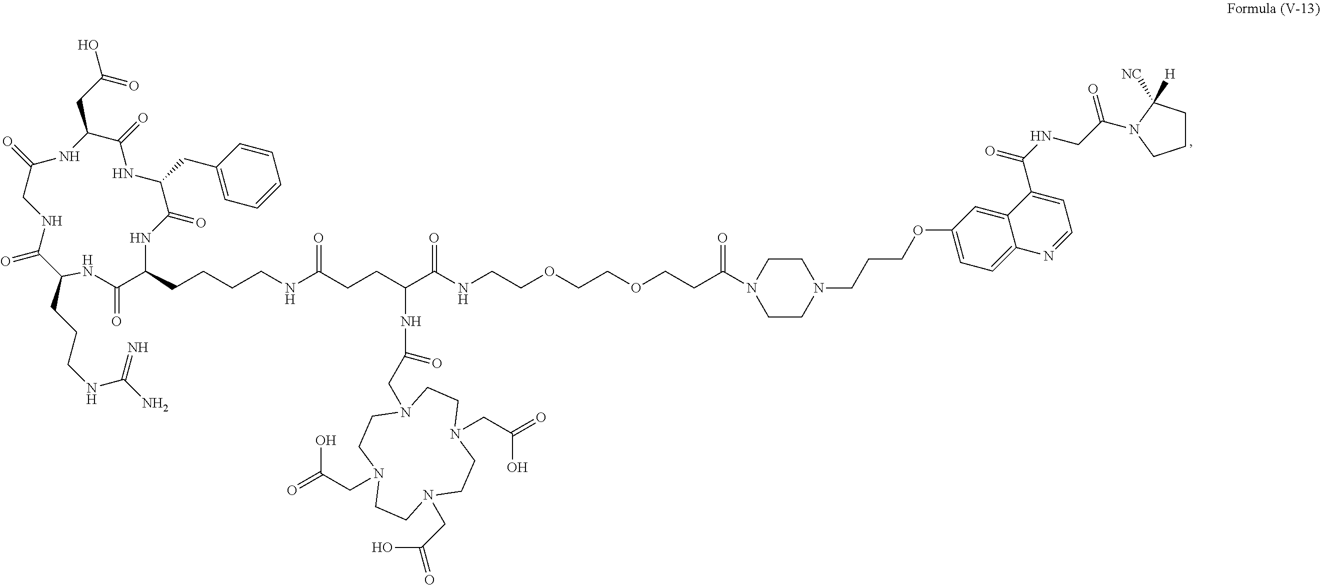 [SMILES: N#C[C@@H]1CCCN1C(=O)CNC(=O)c1ccnc2ccc(OCCCN3CCN(C(=O)CCOCCOCCNC(=O)C(CCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

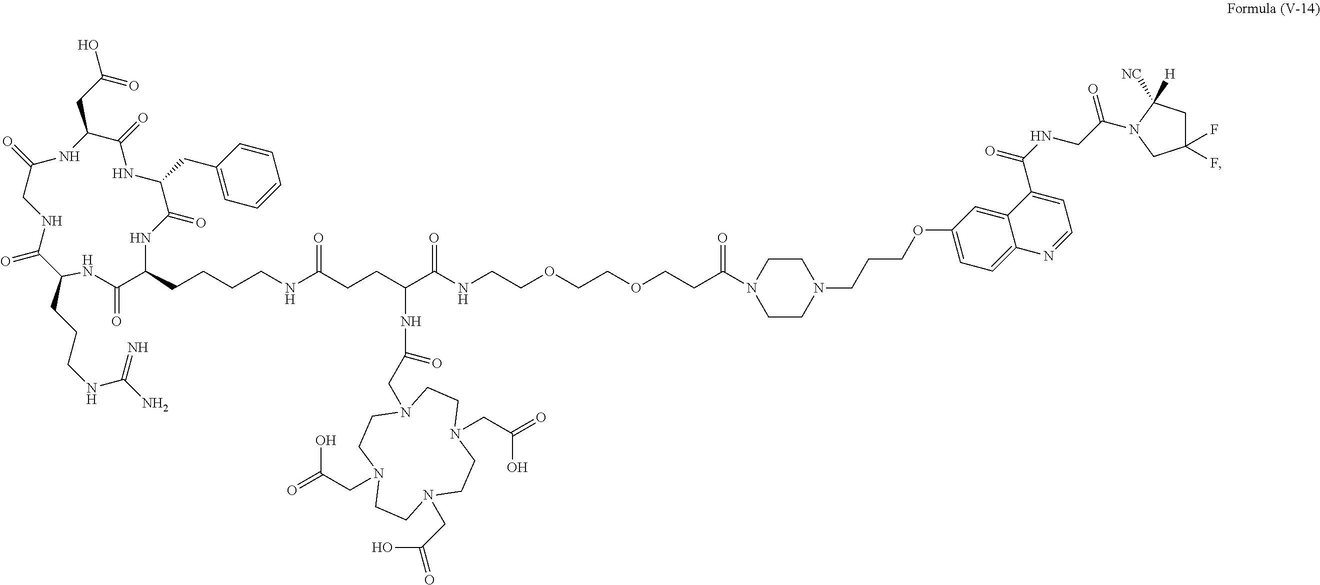 [SMILES: N#C[C@@H]1CC(F)(F)CN1C(=O)CNC(=O)c1ccnc2ccc(OCCCN3CCN(C(=O)CCOCCOCCNC(=O)C(CCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

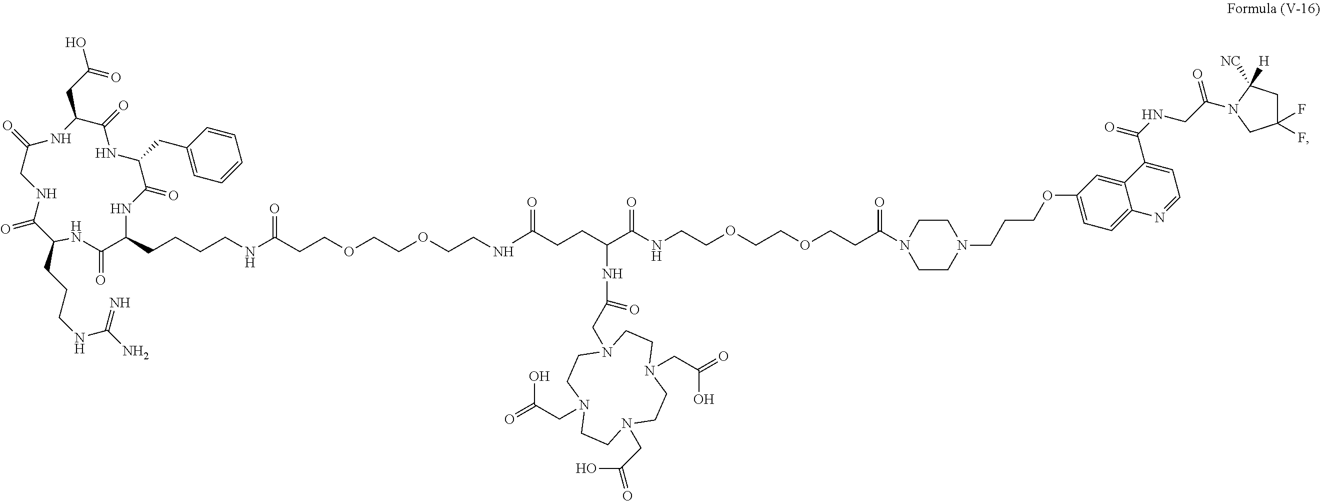 [SMILES: N#C[C@@H]1CC(F)(F)CN1C(=O)CNC(=O)c1ccnc2ccc(OCCCN3CCN(C(=O)CCOCCOCCNC(=O)C(CCC(=O)NCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

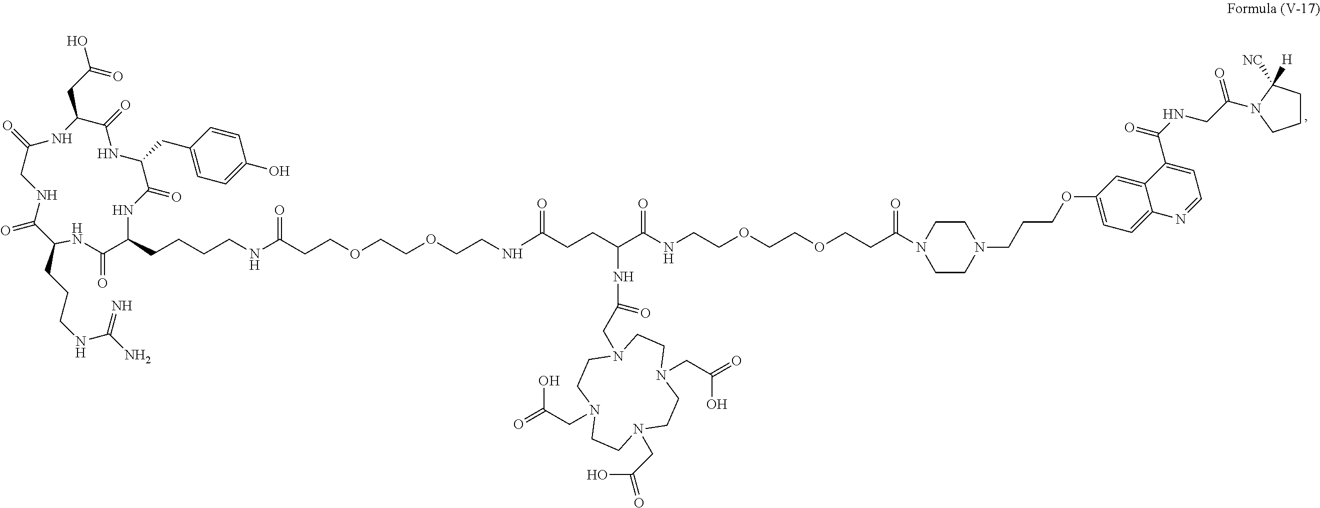 [SMILES: N#C[C@@H]1CCCN1C(=O)CNC(=O)c1ccnc2ccc(OCCCN3CCN(C(=O)CCOCCOCCNC(=O)C(CCC(=O)NCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

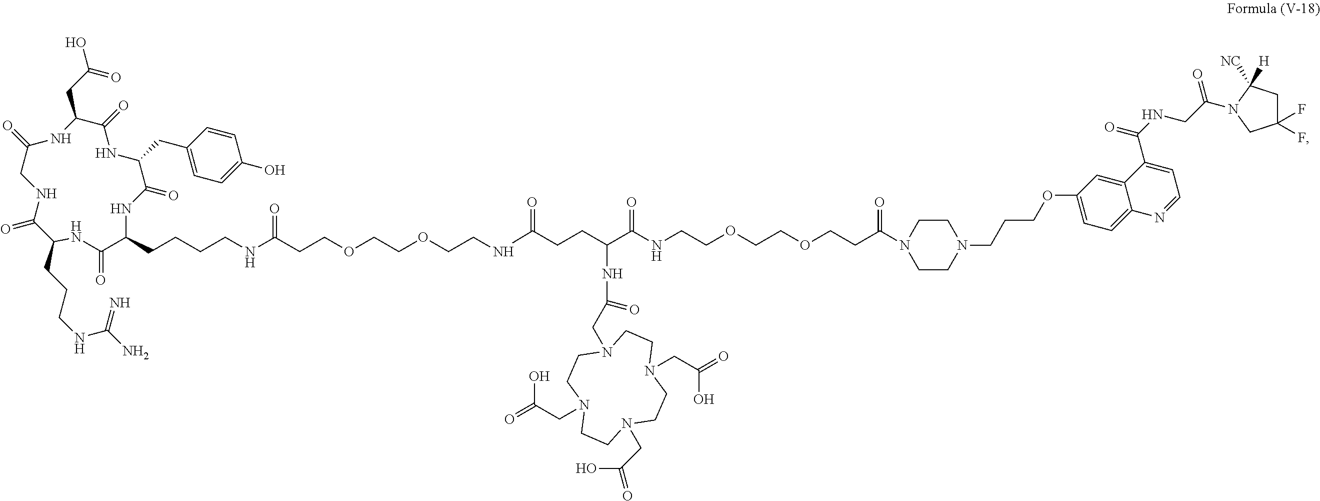 [SMILES: N#C[C@@H]1CC(F)(F)CN1C(=O)CNC(=O)c1ccnc2ccc(OCCCN3CCN(C(=O)CCOCCOCCNC(=O)C(CCC(=O)NCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

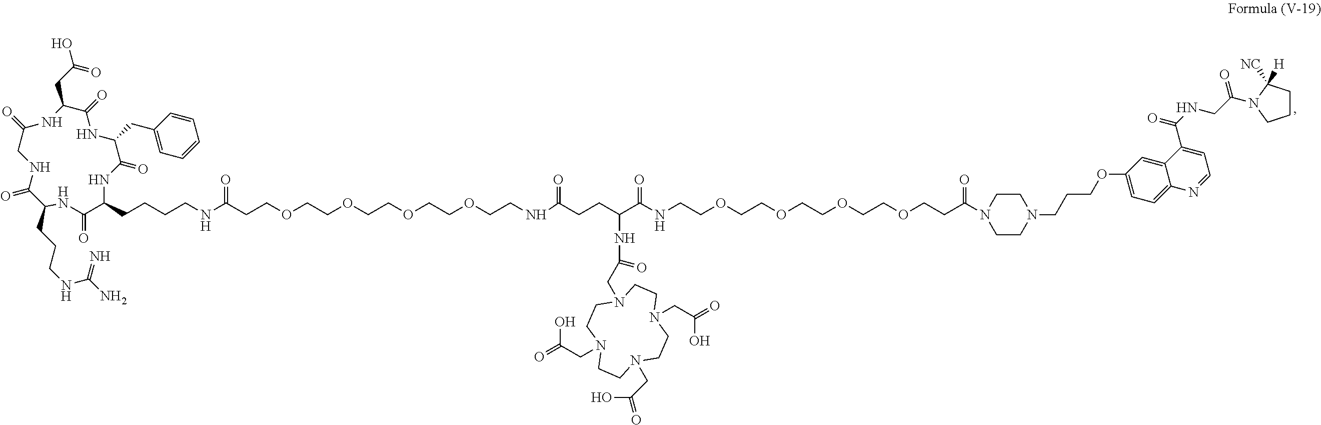 [SMILES: N#C[C@@H]1CCCN1C(=O)CNC(=O)c1ccnc2ccc(OCCCN3CCN(C(=O)CCOCCOCCOCCOCCNC(=O)C(CCC(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

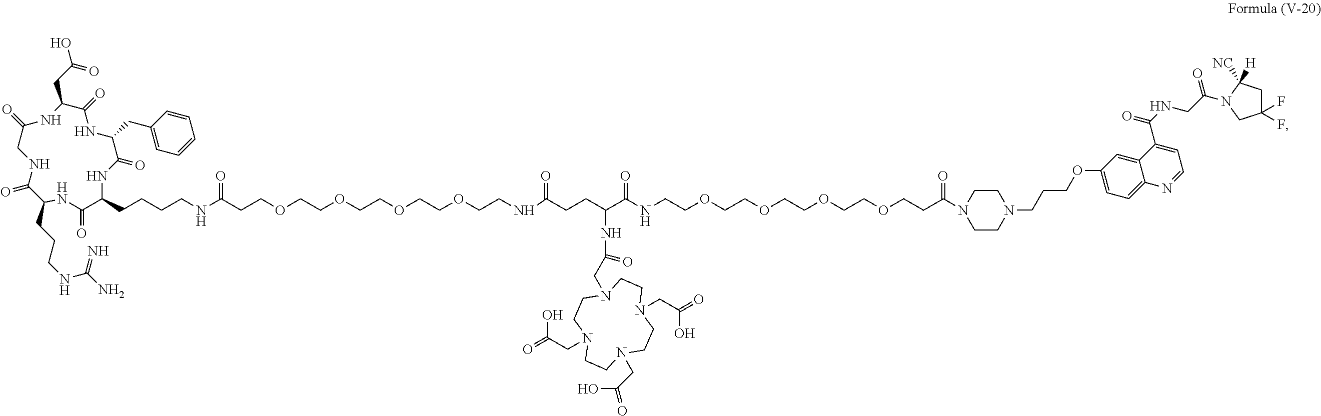 [SMILES: N#C[C@@H]1CC(F)(F)CN1C(=O)CNC(=O)c1ccnc2ccc(OCCCN3CCN(C(=O)CCOCCOCCOCCOCCNC(=O)C(CCC(=O)NCCOCCOCCOCCOCCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

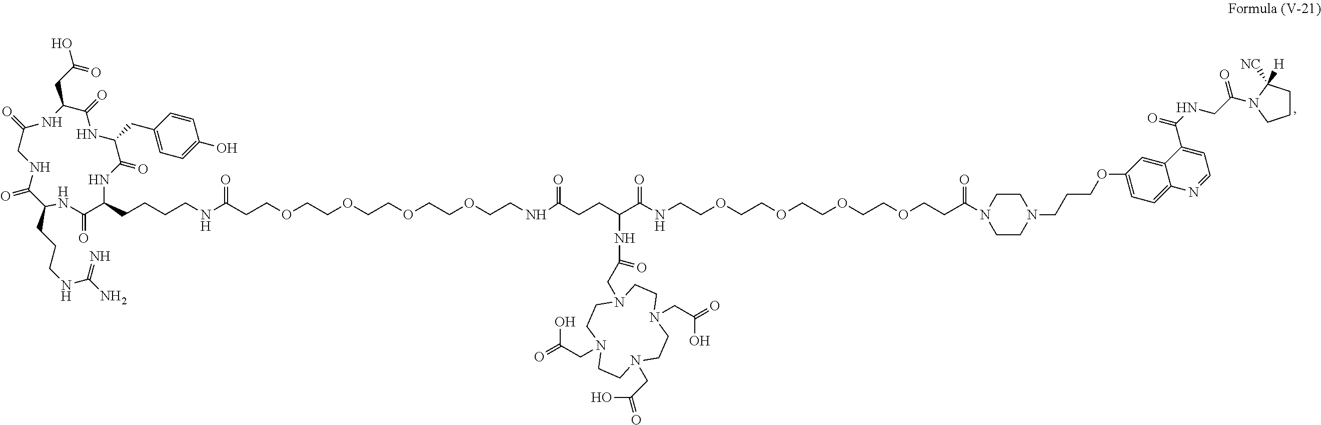 [SMILES: N#C[C@@H]1CCCN1C(=O)CNC(=O)c1ccnc2ccc(OCCCN3CCN(C(=O)CCOCCOCCOCCOCCNC(=O)C(CCC(=O)NCCOCCOCCOCCOCCC(=O)NCCCC[C@@H]4NC(=O)[C@@H](Cc5ccc(O)cc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)CC3)cc12]

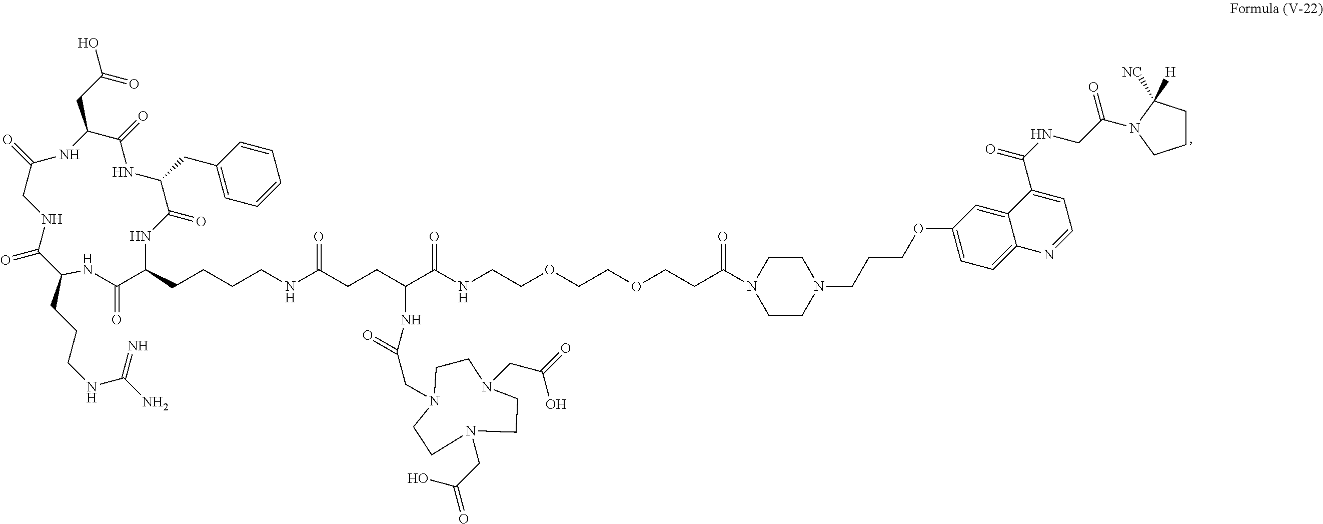 [SMILES: N#C[C@@H]1CCCN1C(=O)CNC(=O)c1ccnc2ccc(OCCCN3CCN(C(=O)CCOCCOCCNC(=O)C(CCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CC4)CC3)cc12]

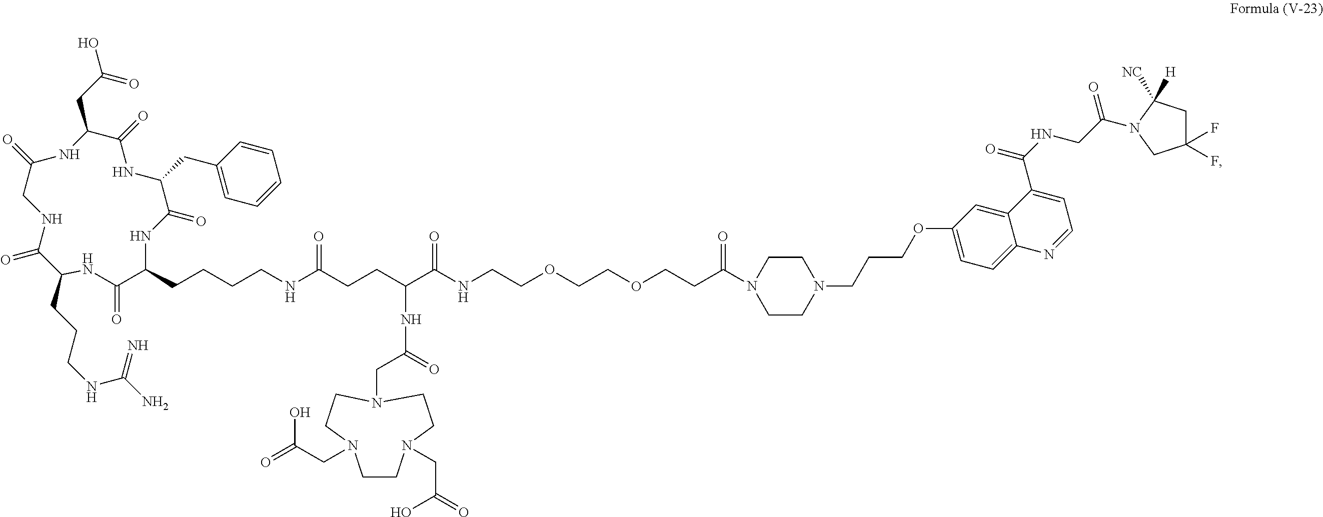 [SMILES: N#C[C@@H]1CC(F)(F)CN1C(=O)CNC(=O)c1ccnc2ccc(OCCCN3CCN(C(=O)CCOCCOCCNC(=O)C(CCC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CC4)CC3)cc12]

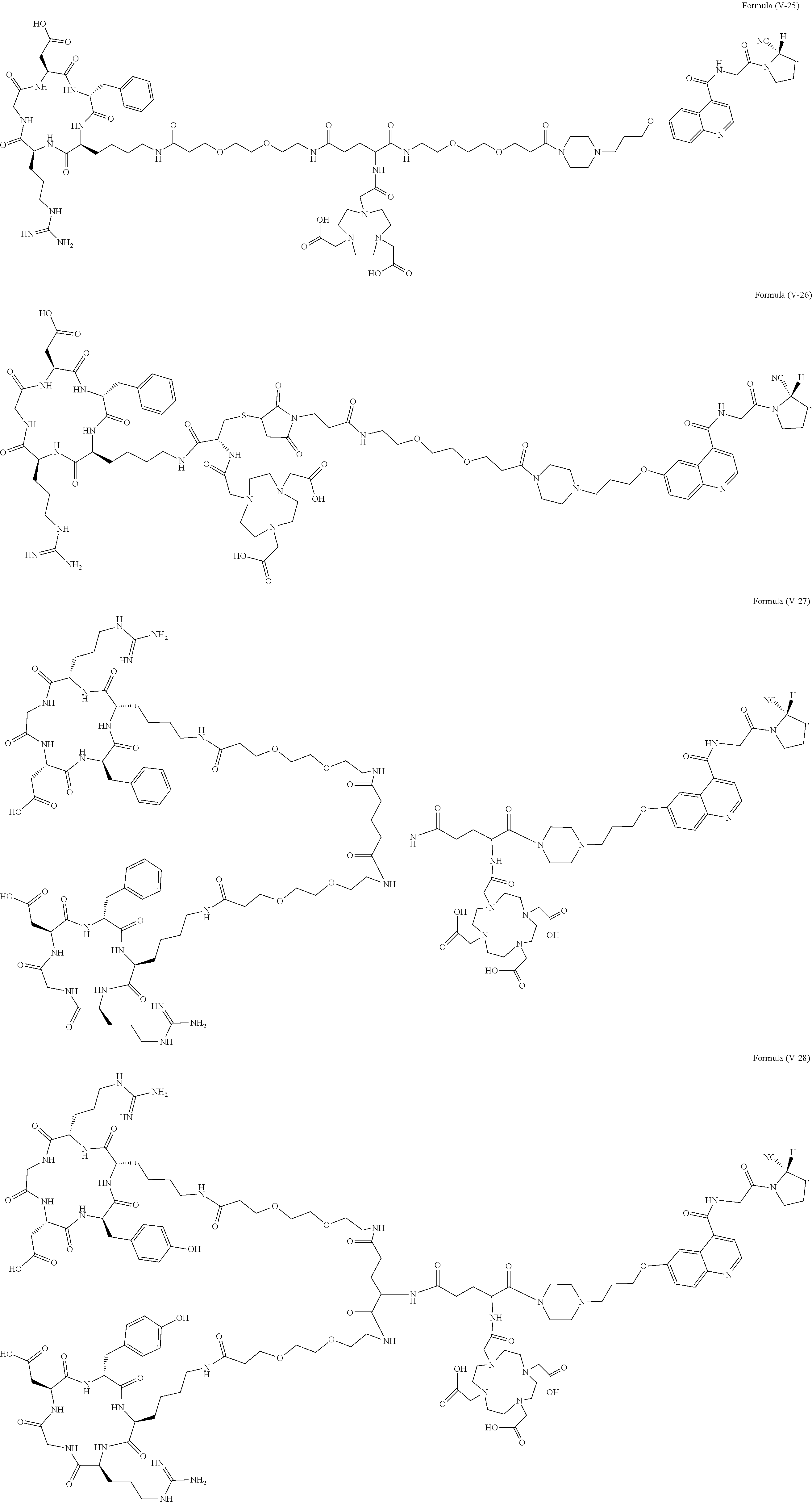

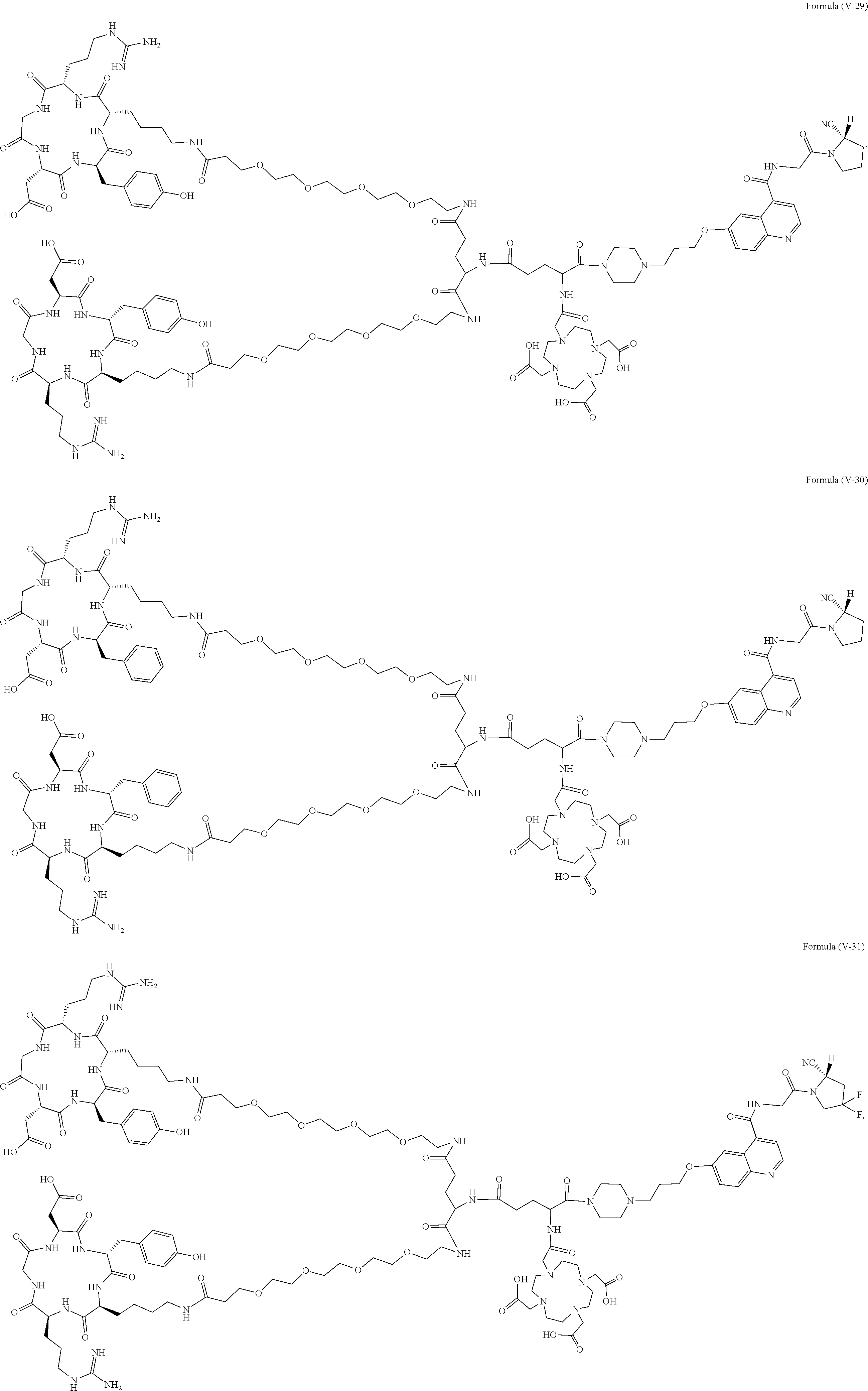

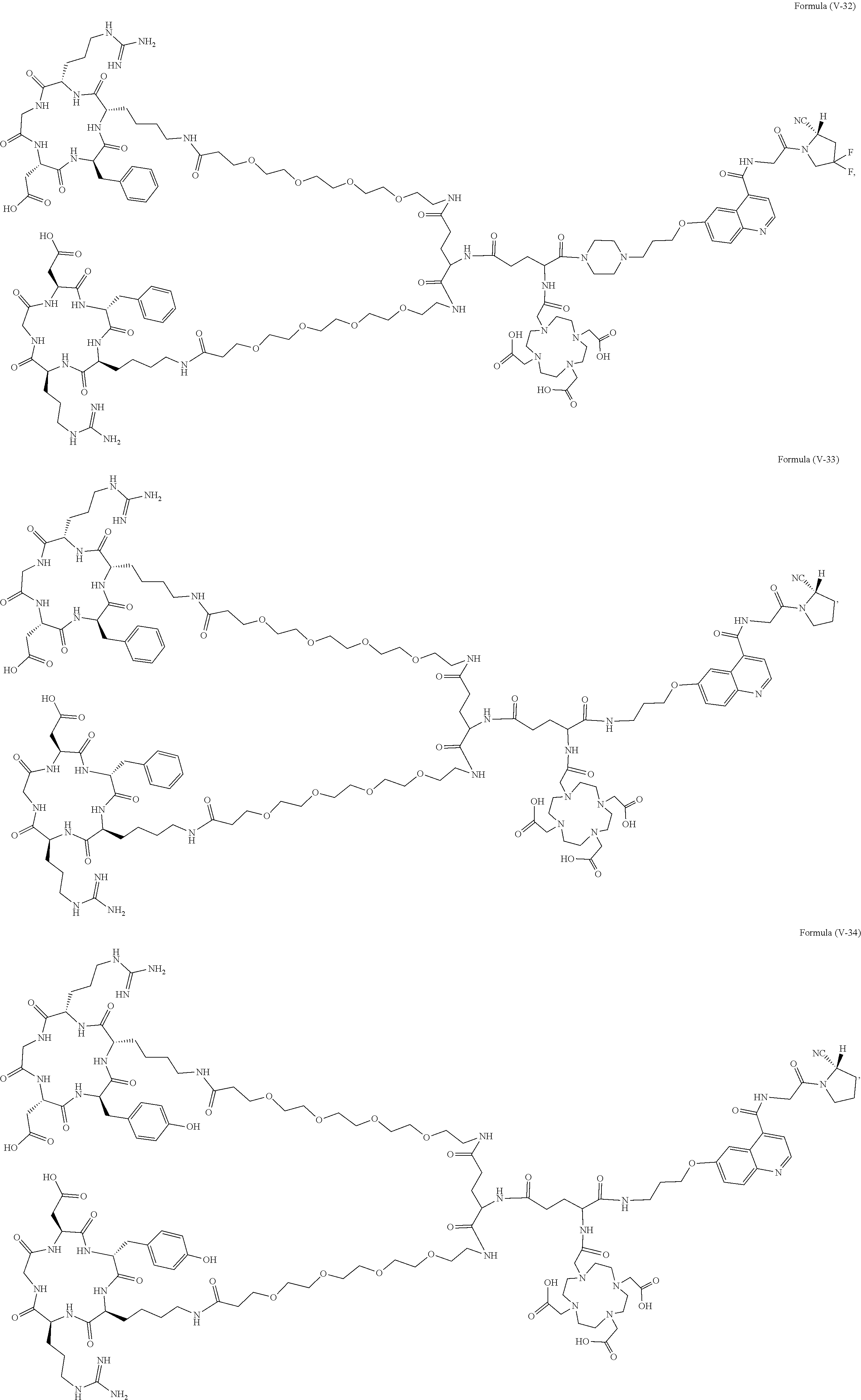

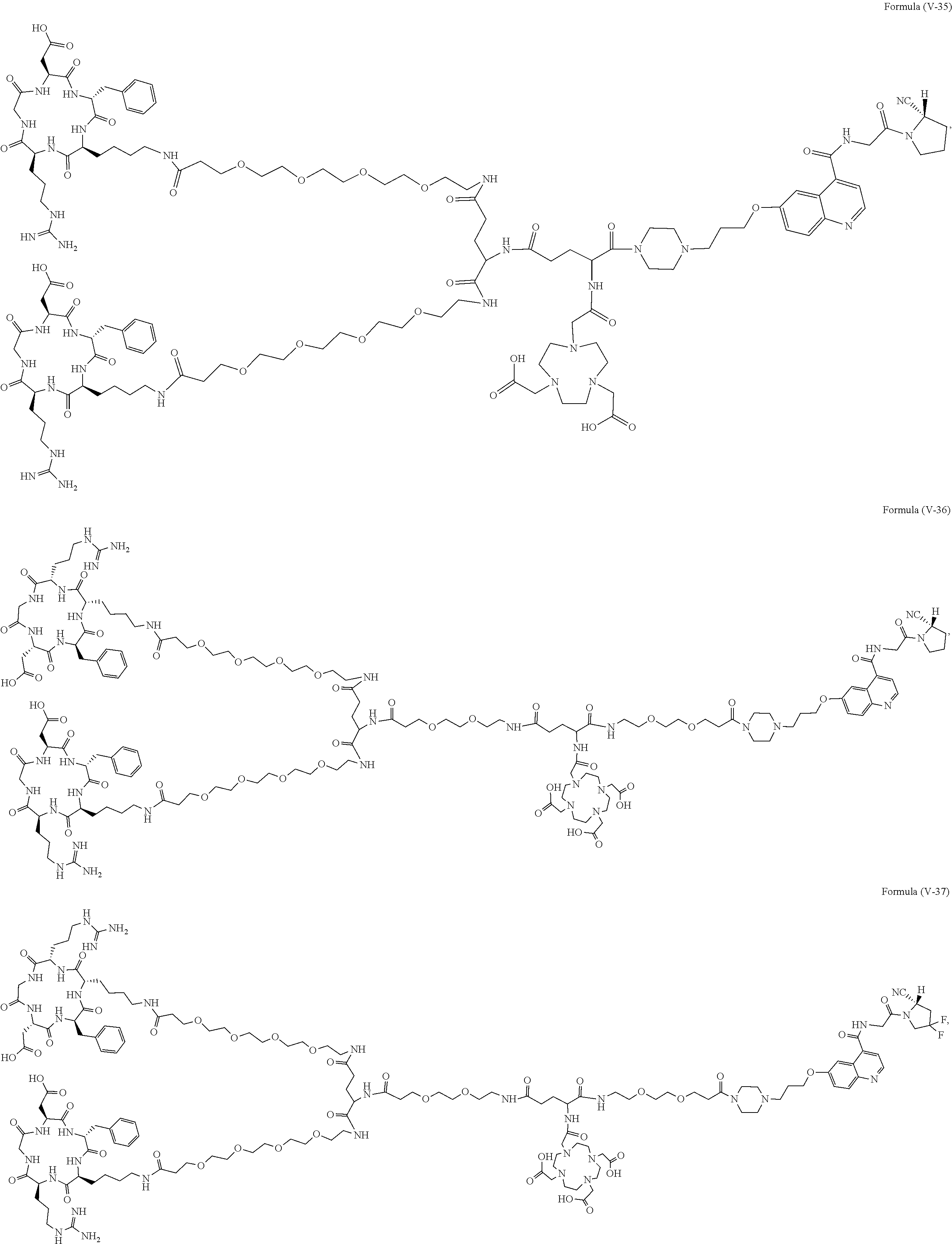

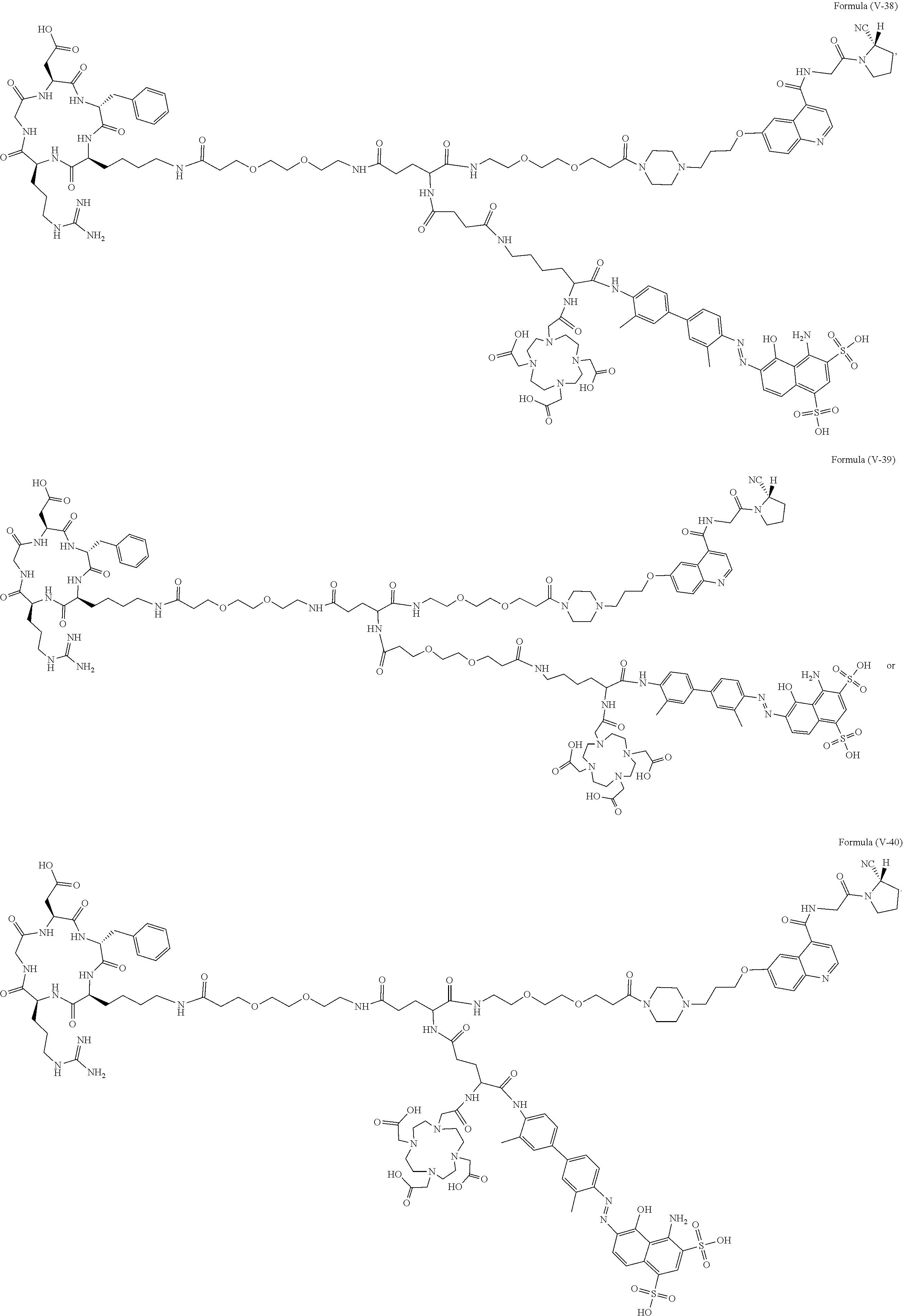

9. The dual-targeting compound according to claim 7, wherein the compound of Formula (VI) has any one of the following structures as shown in Formula (VI-1) to Formula (VI-8):

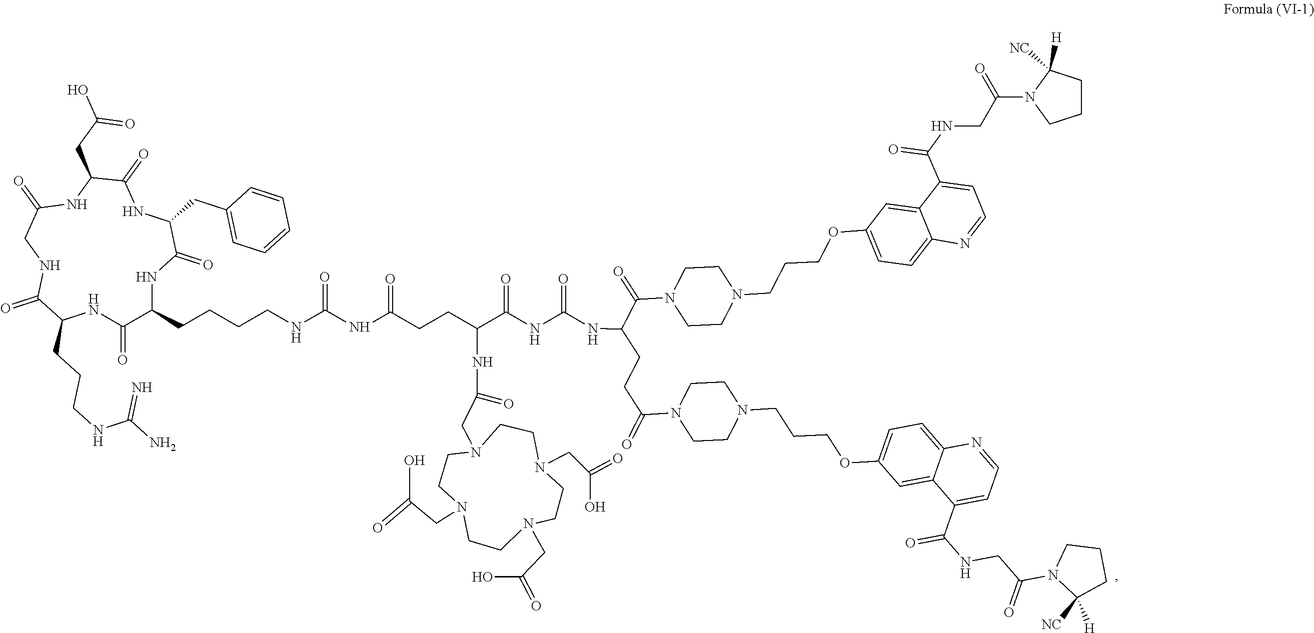 [SMILES: N#C[C@@H]1CCCN1C(=O)CNC(=O)c1ccnc2ccc(OCCCN3CCN(C(=O)CCC(NC(=O)NC(=O)C(CCC(=O)NC(=O)NCCCC[C@@H]4NC(=O)[C@@H](Cc5ccccc5)NC(=O)[C@H](CC(=O)O)NC(=O)CNC(=O)[C@H](CCCNC(=N)N)NC4=O)NC(=O)CN4CCN(CC(=O)O)CCN(CC(=O)O)CCN(CC(=O)O)CC4)C(=O)N4CCN(CCCOc5ccc6nccc(C(=O)NCC(=O)N7CCC[C@H]7C#N)c6c5)CC4)CC3)cc12]

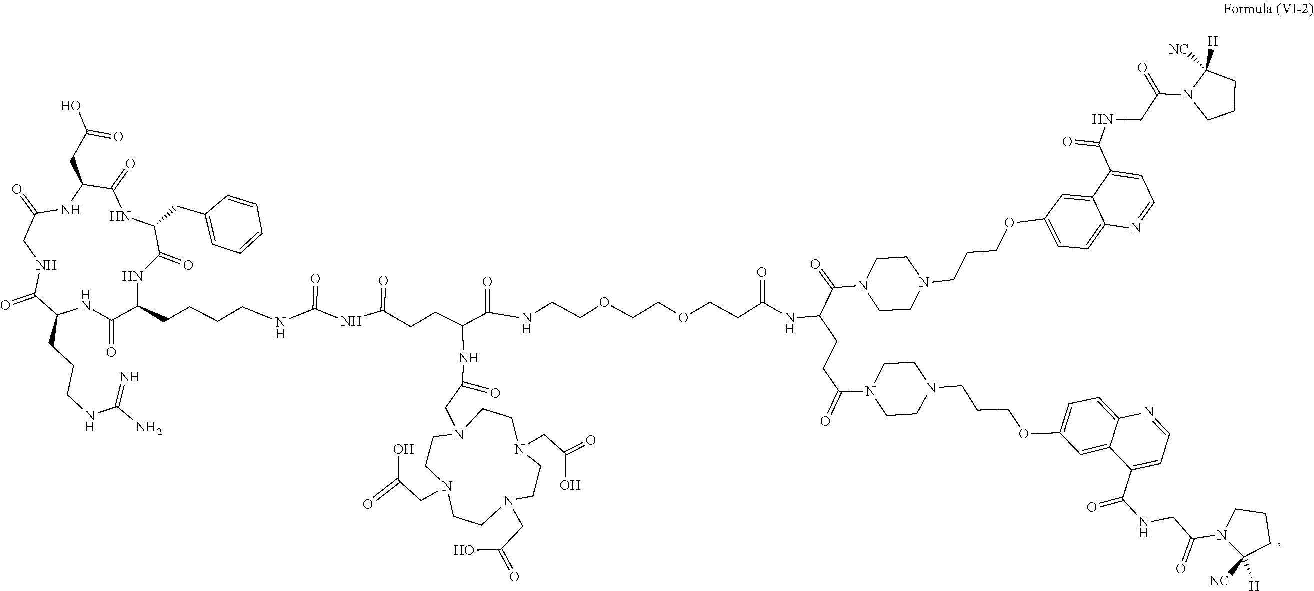

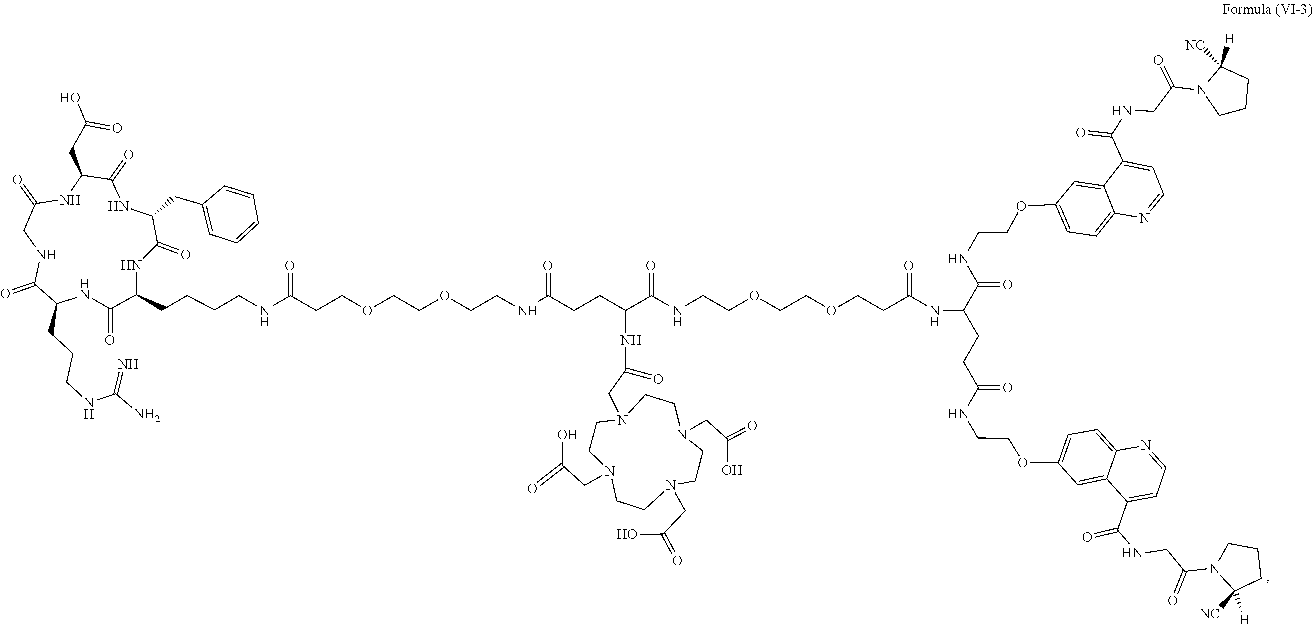

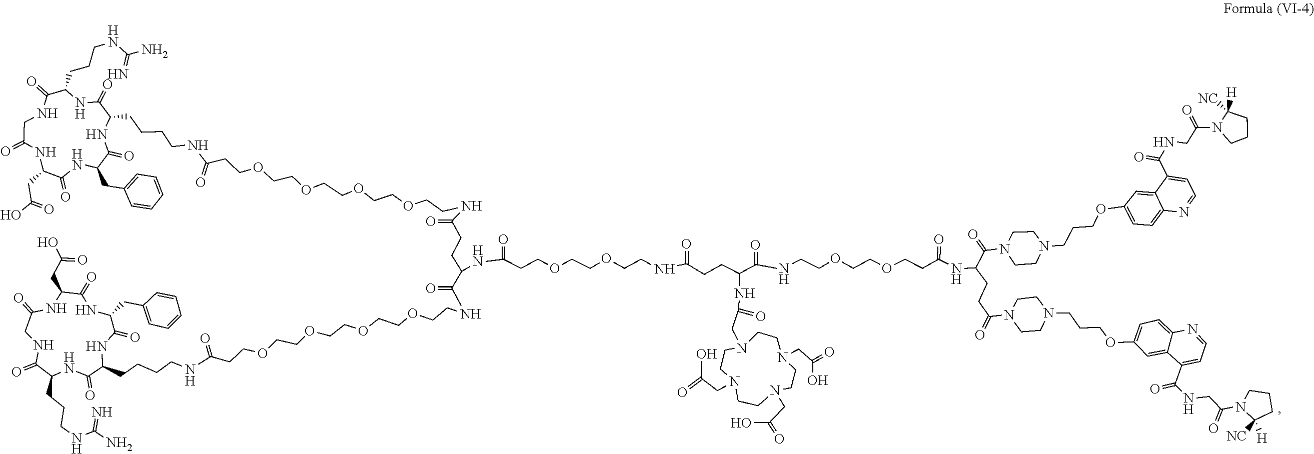

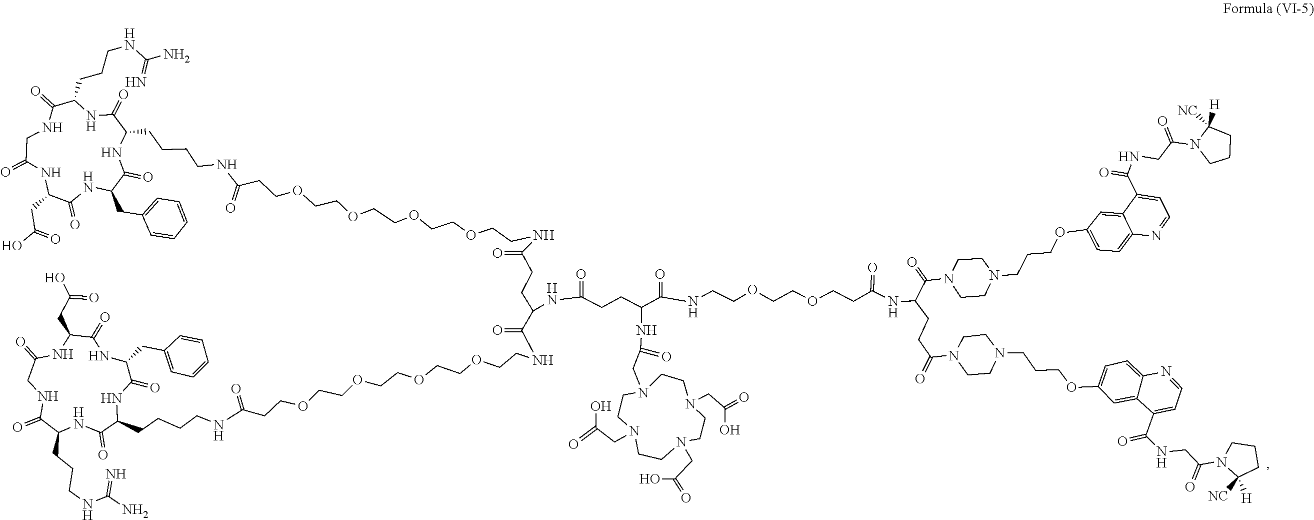

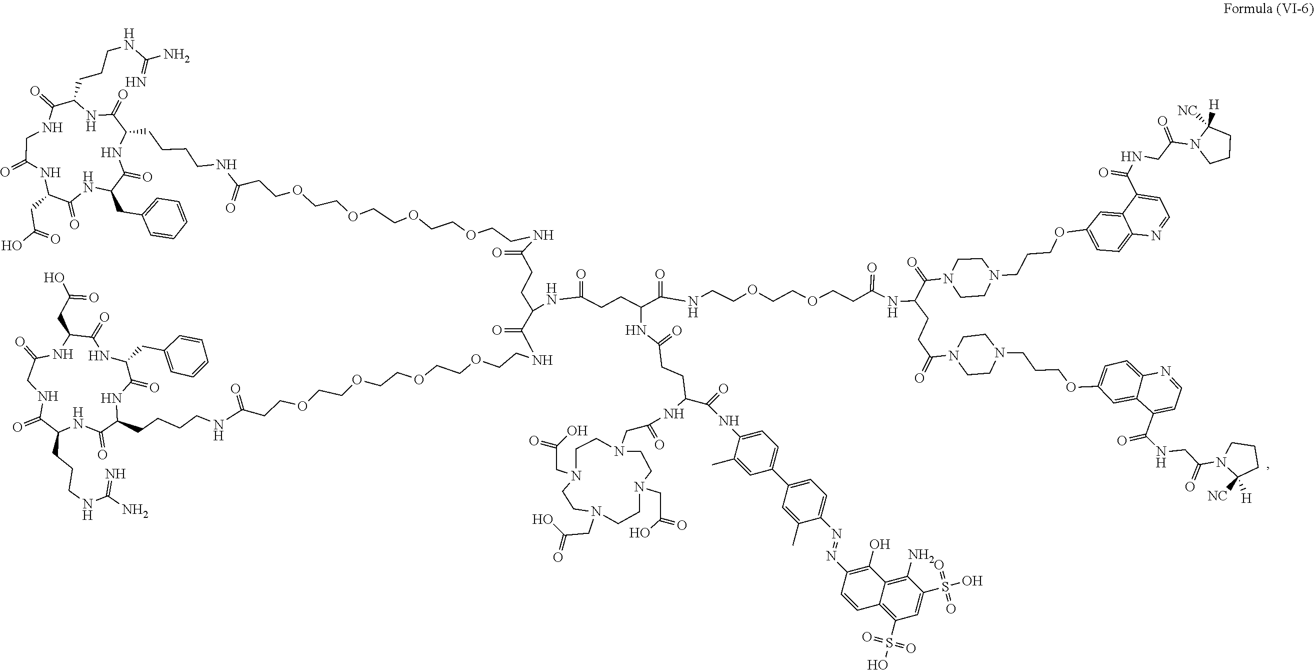

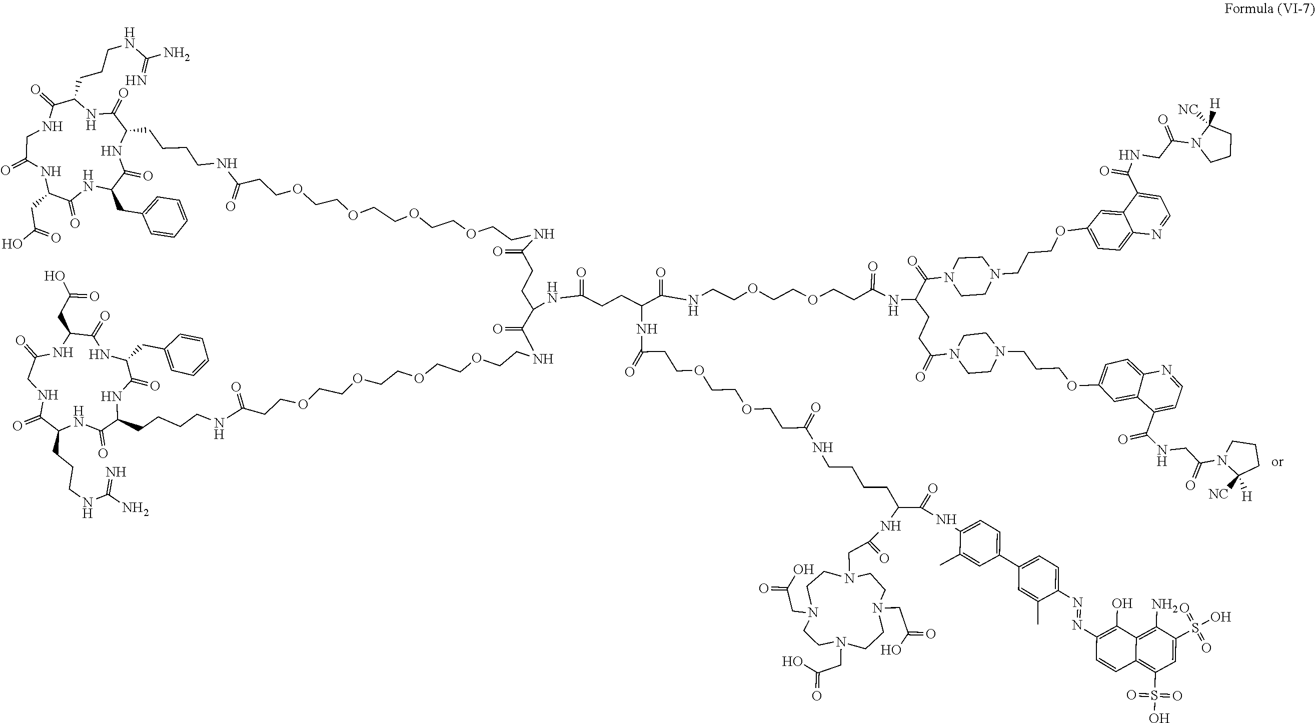

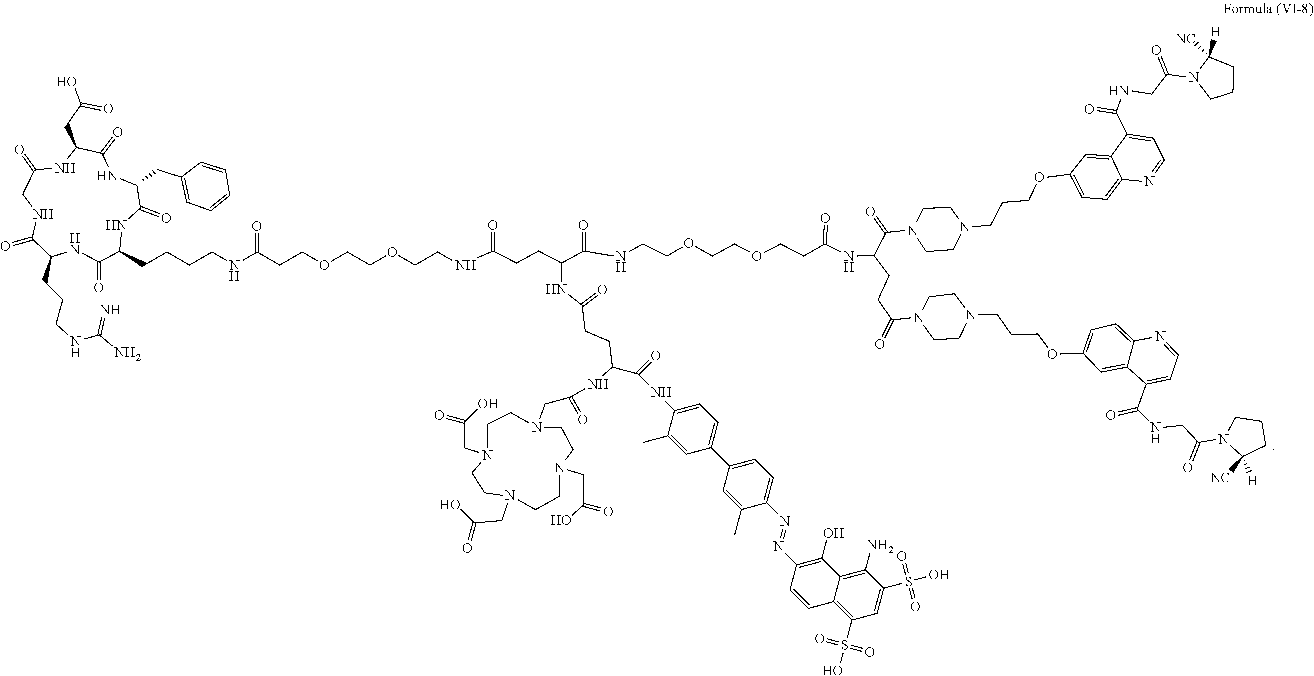

10. A radionuclide-labeled dual-targeting compound, wherein the radionuclide-labeled dual-targeting compound is obtained by labeling the dual-targeting compound according to claim 7 with a radionuclide; preferably, the radionuclide is selected from isotopes emitting α rays, isotopes emitting β rays, isotopes emitting γ rays, isotopes emitting Auger electrons or isotopes emitting X rays; more preferably, the radionuclide is selected from any one of ¹⁸F, ⁵¹Cr, ⁶⁴Cu, ⁶⁷Cu, ⁶⁷Ga, ⁶⁸Ga, ⁸⁹Zr, ¹¹¹In, ^{99m}Tc, ¹⁸⁶Re, ¹⁸⁸Re, ¹³⁹La, ¹⁴⁰La, ¹⁷⁵Yb, ¹⁵³Sm, ¹⁶⁶Ho, ⁸⁶Y, ⁹⁰Y, ¹⁴⁹Pm, ¹⁶⁵Dy, ¹⁶⁹Er, ¹⁷⁷Lu, ⁴⁷Sc, ¹⁴²Pr, ¹⁵⁹Gd, ²¹²Bi, ²¹³Bi, ⁷²As, ⁷²Se, ⁹⁷Ru, ¹⁰⁹Pd, ¹⁰⁵Rh, ^{101m}Rh, ¹¹⁹Sb, ¹²⁸Ba, ¹²³I, ¹²⁴I, ¹³¹I, ¹⁹⁷Hg, ²¹¹At, ¹⁵¹Eu, ¹⁵³Eu, ¹⁶⁹Eu, ²⁰¹Tl, ²⁰³Pb, ²¹²Pb, ¹⁹⁸Au, ²²⁵Ac, ²²⁷Th or ¹⁹⁹Ag; and more preferably, the radionuclide is ¹⁸F, ⁶⁴Cu, ⁶⁸Ga, ⁸⁹Zr, ⁹⁰Y, ¹¹¹In, ^{99m}Tc, ¹⁷⁷Lu, ¹⁸⁸Re or ²²⁵Ac.

11. A method for preparing the dual-targeting compound capable of being labeled with a radionuclide shown in Formula (V) according to claim 7, comprising: first carrying out an amide condensation reaction between carboxyl of 6-hydroxyquinoline-4-carboxylic acid and amino of glycine tert-butyl ester; then connecting BOC-protected piperazinyl by an alkyl chain at a hydroxyl position of an amide condensation product; removing Boc and tert-butyl protective groups under acidic conditions, and then introducing a Boc protective group to a piperazine ring; then carrying out an amide condensation reaction with (S)-pyrrolidine-2-carbonitrile hydrochloride or (S)-4,4-difluoropyrrolidine-2-carbonitrile hydrochloride; after removing the Boc protective group, carrying out a condensation reaction with N-Boc-3-[2-(2-aminoethoxy)ethoxy]propionic acid; then removing the Boc protective group, and sequentially carrying out a reaction with maleimide propionate and cysteine with a protective group, or then carrying gout a reaction with glutamic acid or lysine with a protective group; then introducing RGD (c(RGDyK), c(RGDfK) or c(RGDyK)/c(RGDfK) with a PEG short chain) by an activated ester reaction to obtain a dual-targeting compound; and finally, carrying out a reaction between the dual-targeting compound and a nuclide chelating agent to obtain the dual-targeting compound capable of being labeled with a radionuclide.

12. A method for preparing the radionuclide-labeled dual-targeting compound according to claim 10, comprising: first carrying out an amide condensation reaction between carboxyl of 6-hydroxyquinoline-4-carboxylic acid and amino of glycine tert-butyl ester; then connecting BOC-protected piperazinyl by an alkyl chain at a hydroxyl position of an amide condensation product; removing Boc and tert-butyl protective groups under acidic conditions, and then introducing a Boc protective group to a piperazine ring; then carrying out an amide condensation reaction with (S)-pyrrolidine-2-carbonitrile hydrochloride or (S)-4,4-difluoropyrrolidine-2-carbonitrile hydrochloride; after removing the Boc protective group, carrying out a condensation reaction with N-Boc-3-[2-(2-aminoethoxy)ethoxy]propionic acid; then removing the Boc protective group, and sequentially carrying out a reaction with maleimide propionate and cysteine with a protective group, or then carrying gout a reaction with glutamic acid or lysine with a protective group; then introducing RGD (c(RGDyK), c(RGDfK) or c(RGDyK)/c(RGDfK) with a PEG short chain) by an activated ester reaction to obtain a dual-targeting compound; carrying out a reaction between the dual-targeting compound and a nuclide chelating agent to obtain a dual-targeting compound capable of being labeled with a radionuclide; and carrying out a reaction between the compound capable of being labeled with a radionuclide and a compound containing a radionuclide by an existing wet labeling method or a freeze-drying labeling method to prepare and obtain the radionuclide-labeled targeting compound.

13. A pharmaceutical composition, comprising: the dual-targeting compound according to claim 1, the dual-targeting compound capable of being labeled with a radionuclide, wherein
the dual-targeting compound capable of being labeled with a radionuclide is formed by connecting amino in any one of the Z, Q or V structures of Formula (I) or Formula (II) according to claim 1 with a nuclide chelating group, and has a general formula shown in Formula (V) or Formula (VI) below: 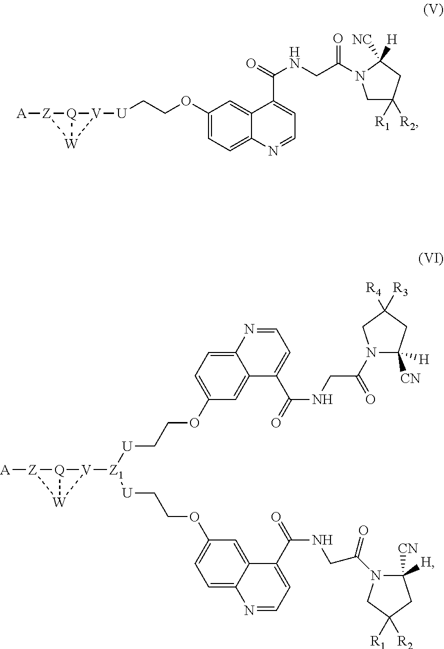
wherein W is a fragment with a nuclide chelating group, and is derived from any one of 1,4,7,10-tetraazacyclododecane-N,N′,N,N′-tetraacetic acid (DOTA), ethylenediamine tetraacetic acid (EDTA), 1,4,7-triazacyclononane-1,4,7-triacetic acid (NOTA), triethylenetetraamine (TETA), iminodiacetic acid, diethylenetriamine-N,N,N′,N′,N″-pentaacetic acid (DTPA), bis(carboxymethyl)imidazole glycinate or 6-hydrazinopyridine-3-carboxylic acid (HYNIC); or the W has any one of the following structures: 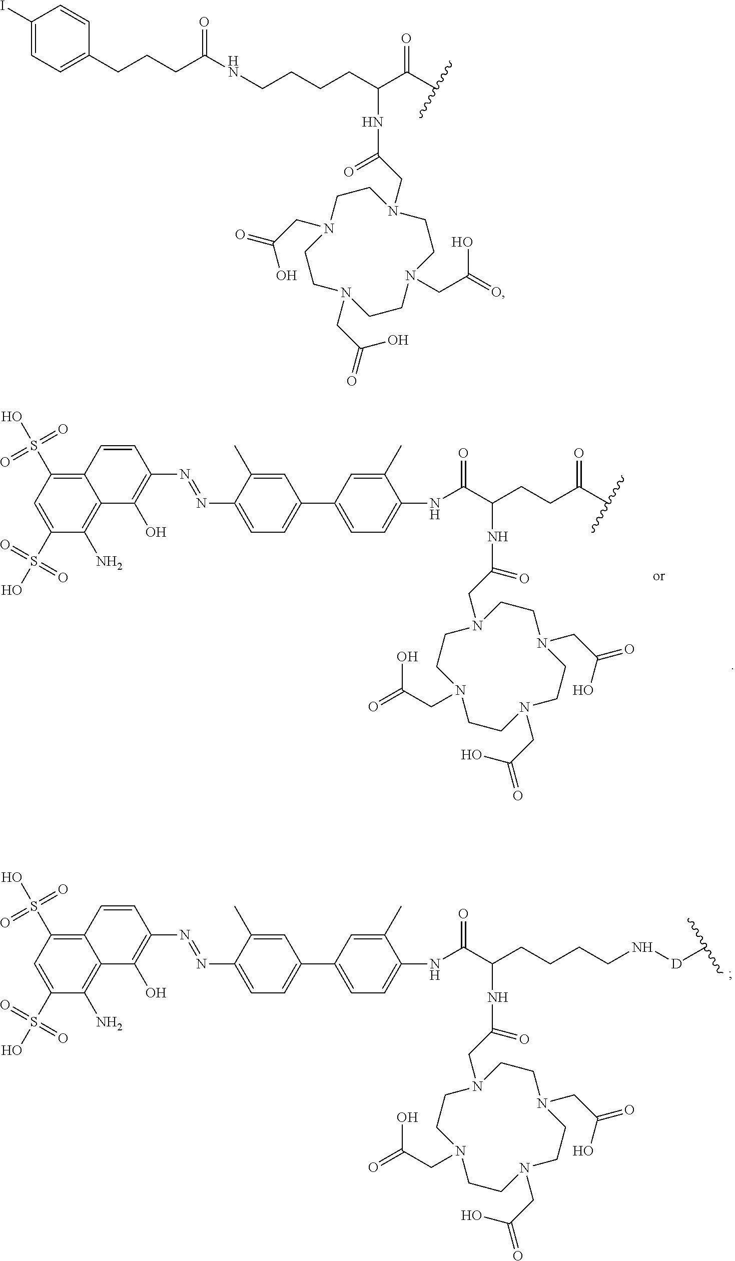
and D in the structures is a substitution structure based on —(CH₂)ₚ—, p is an integer selected from 0 to 30, each —CH₂— is separately substituted with or without —O—, —NH—, —(CO)—, —NH—(CO)—, —CH(NH₂)— or —(CO)—NH—, and substitution occurs when no two adjacent —CH₂— groups are substituted, and wherein
the dual-targeting compound capable of being labeled with a radionuclide; preferably, the radionuclide is selected from isotopes emitting α rays, isotopes emitting β rays, isotopes emitting γ rays, isotopes emitting Auger electrons or isotopes emitting X rays; more preferably, the radionuclide is selected from any one of ¹⁸F, ⁵¹Cr, ⁶⁴Cu, ⁶⁷Cu, ⁶⁷Ga, ⁶⁸Ga, ⁸⁹Zr, ¹¹¹In, ^{99m}Tc, ¹⁸⁶Re, ¹⁸⁸Re, ¹³⁹La ¹⁴⁰La, ¹⁷⁵Yb, ¹⁵³Sm, ¹⁶⁶Ho, ⁸⁶Y, ⁹⁰Y, ¹⁴⁹Pm, ¹⁶⁵Dy, ¹⁶⁹Er, ¹⁷⁷Lu, ⁴⁷Sc, ¹⁴²Pr, ¹⁵⁹Gd, ²¹²Bi, ²¹³Bi, ⁷²As, ⁷²Se, ⁹⁷Ru, ¹⁰⁹Pd, ¹⁰⁵Rh, ^{101m}Rh, ¹¹⁹Sb, ¹²⁸Ba, ¹²³I, ¹²⁴I, ¹³¹I, ¹⁹⁷Hg, ²¹¹At, ¹⁵¹Eu, ¹⁵³Eu, ¹⁶⁹Eu, ²⁰¹Tl, ²⁰³Pb, ²¹²Pb, ¹⁹⁸Au, ²²⁵Ac, ²²⁷Th or ¹⁹⁹Ag; and more preferably, the radionuclide is ¹⁸F, ⁶⁴Cu, ⁶⁸Ga, ⁸⁹Zr, ⁹⁰Y, ¹¹¹In, ^{99m}Tc, ¹⁷⁷Lu, ¹⁸⁸Re or ²²⁵Ac,
or any pharmaceutically acceptable tautomers, racemates, hydrates, solvates or salts thereof.

14. A pharmaceutical composition, composed of: any pharmaceutically acceptable carrier and/or excipient, and the dual-targeting compound according to claim 1,
wherein the dual-targeting compound capable of being labeled with a radionuclide is formed by connecting amino in any one of the Z, Q or V structures of Formula (I) or Formula (II) according to claim 1 with a nuclide chelating group, and has a general formula shown in Formula (V) or Formula (VI) below, 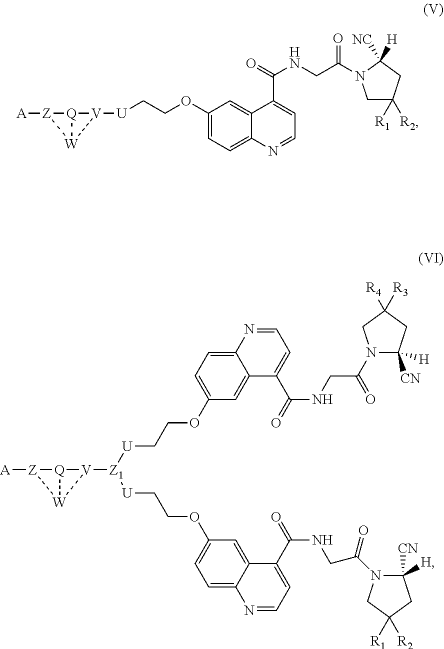
wherein W is a fragment with a nuclide chelating group, and is derived from any one of 1,4,7,10-tetraazacyclododecane-N,N′,N,N′-tetraacetic acid (DOTA), ethylenediamine tetraacetic acid (EDTA), 1,4,7-triazacyclononane-1,4,7-triacetic acid (NOTA), triethylenetetraamine (TETA), iminodiacetic acid, diethylenetriamine-N,N,N′,N′,N″-pentaacetic acid (DTPA), bis(carboxymethyl)imidazole glycinate or 6-hydrazinopyridine-3-carboxylic acid (HYNIC); or the W has any one of the following structures: 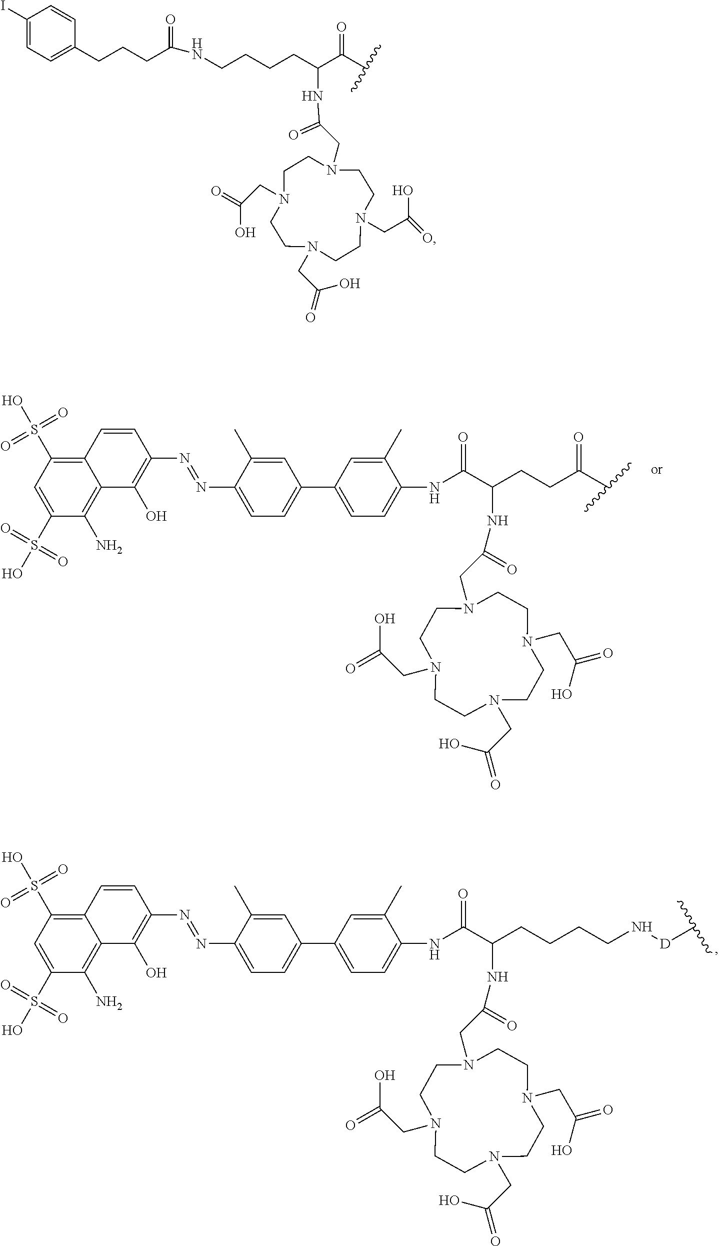
and D in the structures is a substitution structure based on —(CH₂)ₚ—, p is an integer selected from 0 to 30, each —CH₂— is separately substituted with or without —O—, —NH—, —(CO)—, —NH—(CO)—, —CH(NH₂)— or —(CO)—NH—, and substitution occurs when no two adjacent —CH₂— groups are substituted, and wherein
the dual-targeting compound capable of being labeled with a radionuclide: preferably, the radionuclide is selected from isotopes emitting α rays, isotopes emitting β rays, isotopes emitting γ rays, isotopes emitting Auger electrons or isotopes emitting X rays; more preferably, the radionuclide is selected from any one of ¹⁸F, ⁵¹Cr, ⁶⁴Cu, ⁶⁷Cu, ⁶⁷Ga, ⁶⁸Ga, ⁸⁹Zr, ¹¹¹In, ^{99m}Tc, ¹⁸⁶Re, ¹⁸⁸Re, ¹³⁹La ¹⁴⁰La, ¹⁷⁵Yb, ¹⁵³Sm, ¹⁶⁶Ho, ⁸⁶Y, ⁹⁰Y, ¹⁴⁹Pm, ¹⁶⁵Dy, ¹⁶⁹Er, ¹⁷⁷Lu, ⁴⁷Sc, ¹⁴²Pr, ¹⁵⁹Gd, ²¹²Bi, ²¹³Bi, ⁷²As, ⁷²Se, ⁹⁷Ru, ¹⁰⁹Pd, ¹⁰⁵Rh, ^{101m}Rh, ¹¹⁹Sb, ¹²⁸Ba, ¹²³I, ¹²⁴I, ¹³¹I, ¹⁹⁷Hg, ²¹¹At, ¹⁵¹Eu, ¹⁵³Eu, ¹⁶⁹Eu, ²⁰¹Tl, ²⁰³Pb, ²¹²Pb, ¹⁹⁸Au, ²²⁵Ac, ²²⁷Th or ¹⁹⁹Ag; and more preferably, the radionuclide is ¹⁸F, ⁶⁴Cu, ⁶⁸Ga, ⁸⁹Zr, ⁹⁰Y, ¹¹¹In, ^{99m}Tc, ¹⁷⁷Lu, ¹⁸⁸Re or ²²⁵Ac,
or any pharmaceutically acceptable tautomers, racemates, hydrates, solvates or salts thereof.

15. Use of the dual-targeting compound according to claim 1,
wherein the dual-targeting compound capable of being labeled with a radionuclide is formed by connecting amino in any one of the Z, Q or V structures of Formula (I) or Formula (II) according to claim 1 with a nuclide chelating group, and has a general formula shown in Formula (V) or Formula (VI) below: 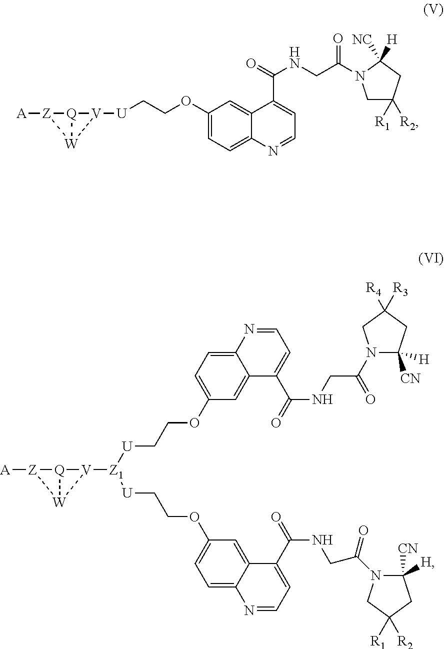
wherein W is a fragment with a nuclide chelating group, and is derived from any one of 1,4,7,10-tetraazacyclododecane-N,N′,N,N′-tetraacetic acid (DOTA), ethylenediamine tetraacetic acid (EDTA), 1,4,7-triazacyclononane-1,4,7-triacetic acid (NOTA), triethylenetetraamine (TETA), iminodiacetic acid, diethylenetriamine-N,N,N′,N′,N″-pentaacetic acid (DTPA), bis(carboxymethyl)imidazole glycinate or 6-hydrazinopyridine-3-carboxylic acid (HYNIC); or the W has any one of the following structures: 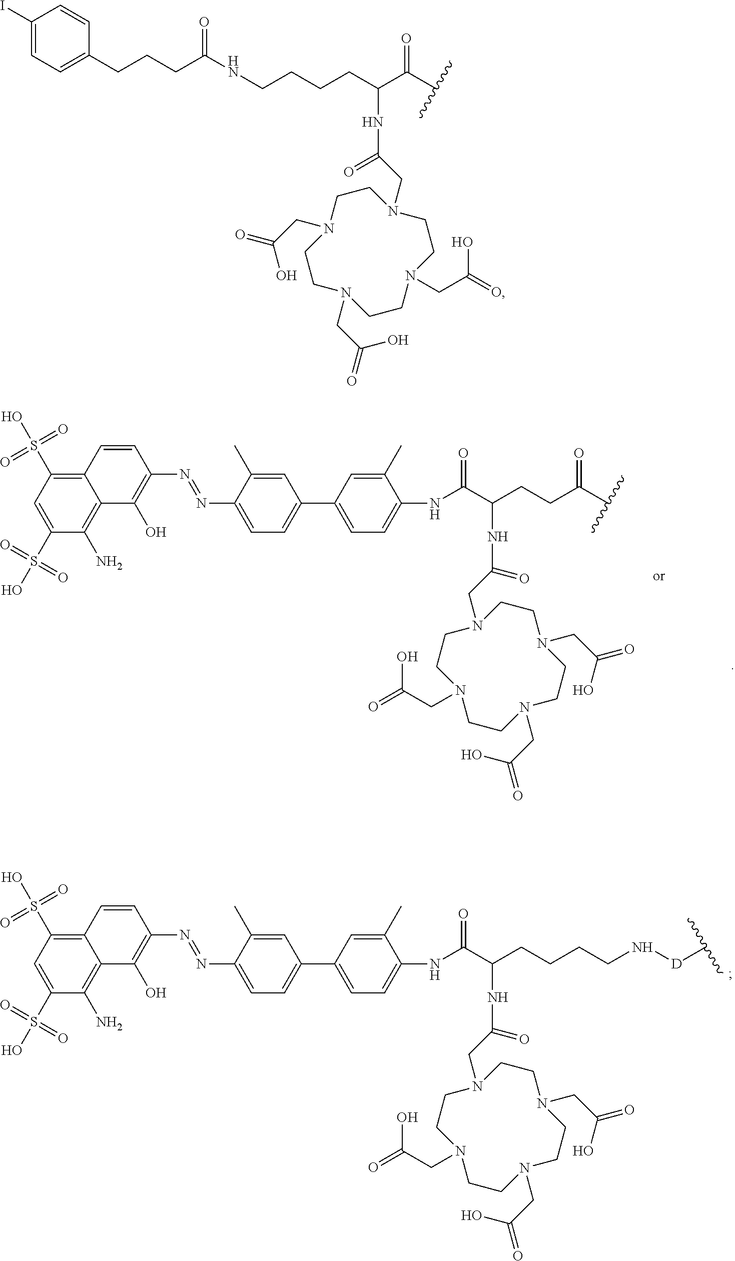
and D in the structures is a substitution structure based on —(CH₂)ₚ—, p is an integer selected from 0 to 30, each —CH₂— is separately substituted with or without —O—, —NH—, —(CO)—, —NH—(CO)—, —CH(NH₂)— or —(CO—NH—, and substitution occurs when no two adjacent —CH₂— groups are substituted, and wherein,
the dual-targeting compound capable of being labeled with a radionuclide; preferably, the radionuclide is selected from isotopes emitting α rays, isotopes emitting β rays, isotopes emitting γ rays, isotopes emitting Auger electrons or isotopes emitting X rays; more preferably, the radionuclide is selected from any one of ¹⁸F, ⁵¹Cr, ⁶⁴Cu, ⁶⁷Cu, ⁶⁷Ga, ⁶⁸Ga, ⁸⁹Zr, ¹¹¹In, ^{99m}Tc, ¹⁸⁶Re, ¹⁸⁸Re, ¹³⁹La, ¹⁴⁰La, ¹⁷⁵Yb, ¹⁵³Sm, ¹⁶⁶Ho, ⁸⁶Y, ⁹⁰Y, ¹⁴⁹Pm, ¹⁶⁵Dy, ¹⁶⁹Er, ¹⁷⁷Lu, ⁴⁷Sc, ¹⁴²Pr, ¹⁵⁹Gd, ²¹²Bi, ²¹³Bi, ⁷²As, ⁷²Se, ⁹⁷Ru, ¹⁰⁹Pd, ¹⁰⁵Rh, ^{101m}Rh, ¹¹⁹Sb, ¹²⁸Ba, ¹²³I, ¹²⁴I, ¹³¹I, ¹⁹⁷Hg, ²¹¹At, ¹⁵¹Eu, ¹⁵³Eu, ¹⁶⁹Eu, ²⁰¹Tl, ²⁰³Pb, ²¹²Pb, ¹⁹⁸Au, ²²⁵Ac, ²²⁷Th or ¹⁹⁹Ag; and more preferably, the radionuclide is ¹⁸F, ⁶⁴Cu, ⁶⁸Ga, ⁸⁹Zr, ⁹⁰Y, ¹¹¹In, ^{99m}Tc, ¹⁷⁷Lu, ¹⁸⁸Re or ²²⁵Ac,
or any pharmaceutically acceptable tautomers, racemates, hydrates, solvates or salts thereof, or a pharmaceutical composition thereof in preparation of drugs for diagnosis or treatment of diseases characterized by overexpression of fibroblast activation protein (FAP) and/or integrin αᵥβ₃ in animals or human individuals.

16. The use according to claim 15, wherein the diseases characterized by overexpression of fibroblast activation protein (FAP) and/or integrin αᵥβ₃ comprise, but are not limited to: cancer, chronic inflammation, atherosclerosis, fibrosis, tissue remodeling and scar diseases; and preferably, the cancer is further selected from breast cancer, pancreatic cancer, small intestine cancer, colon cancer, rectal cancer, lung cancer, head and neck cancer, ovarian cancer, hepatocellular carcinoma, esophageal cancer, hypopharyngeal carcinoma, nasopharyngeal carcinoma, laryngeal carcinoma, myeloma cells, bladder cancer, cholangiocellular carcinoma, clear cell renal cell carcinoma, neuroendocrine neoplasm, carcinogenic osteomalacia, sarcoma, cancer of unknown primary (CUP), thymic carcinoma, glioma, neuroglioma, astrocytoma, cervical cancer or prostate cancer.

17. A kit, comprising or composed of: (1) the dual-targeting compound according to claim 1, the dual-targeting compound capable of being labeled with a radionuclide,
wherein the dual-targeting compound capable of being labeled with a radionuclide is formed by connecting amino in any one of the Z, Q or V structures of Formula (I) or Formula (II) according to claim 1 with a nuclide chelating group, and has a general formula shown in Formula (V) or Formula (VI) below: 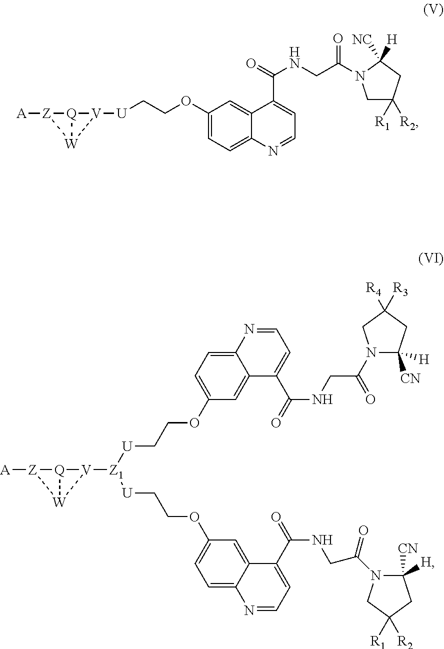
wherein W is a fragment with a nuclide chelating group, and is derived from any one of 1,4,7,10-tetraazacyclododecane-N,N′,N,N′-tetraacetic acid (DOTA), ethylenediamine tetraacetic acid (EDTA), 1,4,7-triazacyclononane-1,4,7-triacetic acid (NOTA), triethylenetetraamine (TETA), iminodiacetic acid, diethylenetriamine-N,N,N′,N′,N″-pentaacetic acid (DTPA), bis(carboxymethyl)imidazole glycinate or 6-hydrazinopyridine-3-carboxylic acid (HYNIC); or the W has any one of the following structures: 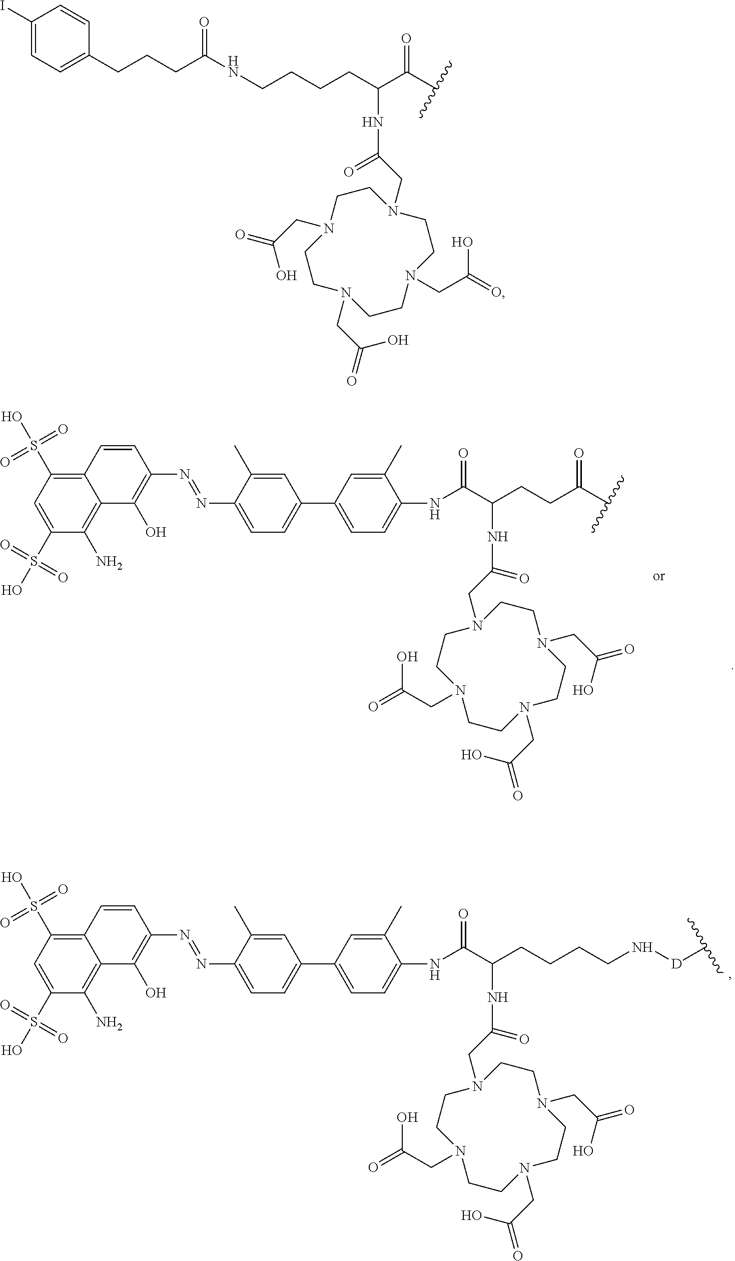
and D in the structures is a substitution structure based on —(CH₂)ₚ—, p is an integer selected from 0 to 30, each —CH₂— is separately substituted with or without —O—, —NH—, —(CO)—, —NH—(CO)—, —CH(NH₂)— or —(CO)—NH—, and substitution occurs when no two adjacent —CH₂— groups are substituted, and wherein
the dual targeting compound capable of being labeled with a radionuclide, preferably, the radionuclide is selected from isotopes emitting α rays, isotopes emitting β rays, isotopes emitting γ rays, isotopes emitting Auger electrons or isotones emitting X rays; more preferably, the radionuclide is selected from any one of ¹⁸F, ⁵¹Cr, ⁶⁴Cu, ⁶⁷Cu, ⁶⁷Ga, ⁶⁸Ga, ⁸⁹Zr, ¹¹¹In, ^{99m}Tc, ¹⁸⁶Re, ¹⁸⁸Re, ¹³⁹La, ¹⁴⁰La, ¹⁷⁵Yb, ¹⁵³Sm, ¹⁶⁶Ho, ⁸⁶Y, ⁹⁰Y, ¹⁴⁹Pm, ¹⁶⁵Dy, ¹⁶⁹Er, ¹⁷⁷Lu, ⁴⁷Sc, ¹⁴²Pr, ¹⁵⁹Gd, ²¹²Bi, ²¹³Bi, ⁷²As, ⁷²Se, ⁹⁷Ru, ¹⁰⁹Pd, ¹⁰⁵Rh, ^{101m}Rh, ¹¹⁹Sb, ¹²⁸Ba, ¹²³I, ¹²⁴I, ¹³¹I, ¹⁹⁷Hg, ²¹¹At, ¹⁵¹Eu, ¹⁵³Eu, ¹⁶⁹Eu, ²⁰¹Tl, ²⁰³Pb, ²¹²Pb, ¹⁹⁸Au, ²²⁵Ac, ²²⁷Th or ¹⁹⁹Ag, and more preferably, the radionuclide is ¹⁸F, ⁶⁴Cu, ⁶⁸Ga, ⁸⁹Zr, ⁹⁰Y, ¹¹¹In, ^{99m}Tc, ¹⁷⁷Lu, ¹⁸⁸Re or ²²⁵Ac,
or any pharmaceutically acceptable tautomers, racemates, hydrates, solvates or salts thereof, or a pharmaceutical composition thereof;
and (2) an instruction for diagnosing diseases.
